(19) United States
(12) Patent Application Publication
(10) Pub. No.: US 2010/0022531 A1
(43) Pub. Date: Jan. 28, 2010

(54) NOVEL COMPOUNDS AS P2X7 MODULATORS AND USES THEREOF

(75) Inventors: John Kincaid, San Francisco, CA (US); Yeyu Cao, Foster City, CA (US); Carl Kaub, San Mateo, CA (US); David Lonergan, San Marcos, CA (US); Michael G. Kelly, Thousand Oaks, CA (US)

Correspondence Address: KLAUBER & JACKSON, 411 HACKENSACK AVENUE, HACKENSACK, NJ, 07601, US

(73) Assignee: RENOVIS, INC., South San Francisco, CA (US)

(21) Appl. No.: 11/991,485

(22) PCT Filed: Sep. 1, 2006
(86) PCT No.: PCT/US2006/034236

§ 371 Date: Sep. 2, 2009

Related U.S. Application Data
(60) Provisional application No. 60/713,231, filed on Sep. 1, 2005.
(60) Provisional application No. 60/714,676, filed on Sep. 7, 2005.
(60) Provisional application No. 60/717,463, filed on Sep. 15, 2005.
(60) Provisional application No. 60/753,957, filed on Dec. 23, 2005.
(60) Provisional application No. 60/754,223, filed on Dec. 28, 2005.

(51) Int. Cl. A61K 31/5377; C07D 471/04; C07D 471/12; C07D 265/36; A61K 31/495; A61K 31/519; A61P 25/00; A61P 25/02

(52) U.S. Cl. 514/234.2; 544/279; 544/105; 514/249; 514/264.1

(57) ABSTRACT

Compounds are disclosed that have a formula represented by the following (1), the compounds may be prepared as pharmaceutical compositions, and may be used for the prevention and treatment of a variety of conditions in mammals including humans, including by way of non-limiting example, pain, inflammation, traumatic injury, and others.

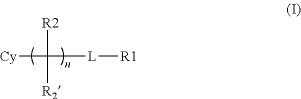

### FIELD OF THE INVENTION

This invention relates to novel compounds that are capable of modulating P2X₇ receptor activity, and to pharmaceutical compositions containing such compounds. This invention also relates to methods for preventing and/or treating conditions that are causally related to aberrant P2X₇ activity, such as inflammation-related conditions in mammals, comprising (but not limited to) rheumatoid arthritis, osteoarthritis, Parkinson's disease, uveitis, asthma, cardiovascular conditions including myocardial infarction, the treatment and prophylaxis of pain syndromes (acute and chronic or neuropathic), traumatic brain injury, acute spinal cord injury, neurodegenerative disorders, inflammatory bowel disease and autoimmune disorders, using the compounds and pharmaceutical compositions of the invention.

### BACKGROUND OF THE INVENTION

Cell surface receptors for ATP can be divided into metabotropic (P2Y/P2U) and ionotropic (P2X) classes. The metabotropic class belongs to the superfamily of G protein-coupled receptors with seven transmembrane segments. The ionotropic class members (P2X₁-P2X₆) are ligand-gated ion channels, currently thought to be multisubunit proteins with two transmembrane domains per subunit (Buell et al, Europ. J. Neurosci. 8:2221 (1996)). P2Z receptors have been distinguished from other P2 receptors in three primary ways (Buisman et al, Proc. Natl. Acad. Sci. USA 85:7988 (1988); Cockcroft et al, Nature 279:541 (1979); Steinberg et al, J. Biol. Chem. 262:3118 (1987)). First, activation of P2Z receptors leads not only to an inward ionic current, but also to cell permeabilization. Second, 3′-O-(4-benzoyl)benzoyl ATP (BZATP) is the most effective agonist, and ATP itself is of rather low potency. Third, responses are strongly inhibited by extracellular magnesium ions, which has been interpreted to indicate that ATP⁴- is the active agonist (DiVirgilio, Immunol. Today 16:524 (1995)).

A seventh member of the P2X receptor family has been isolated from a rat cDNA library and, when expressed in human embryonic kidney (HEK293) cells, exhibits the above three properties (Surprenant et al, Science 272:735 (1996)). This receptor, (rP2X₇) thus corresponds to the P2Z receptor. rP2X₇ is structurally related to other members of the P2X family but it has a longer cytoplasmic C-terminus domain (there is 35-40% amino acid identity in the corresponding region of homology, but the C-terminus is 239 amino acids long in the rP2X₇ receptor compared with 27-20 amino acids in the others). The rP2X₇ receptor functions both as a channel permeable to small cations and as a cytolytic pore. Brief applications of ATP (1-2 s) transiently open the channel, as is the case of other P2X receptors. Repeated or prolonged applications of agonist cause cell permeabilization reducing the extracellular magnesium concentration potentiates this effect. The unique C-terminal domain of rP2X₇ is required for cell permeabilization and the lytic actions of ATP (Suprenant et al, Science 272:735 (1996)).

The P2Z/rP2X₇ receptor has been implicated in lysis of antigen-presenting cells by cytotoxic T lymphocytes, in the mitogenic stimulation of human T lymphocytes, as well as in the formation of multinucleated giant cells (Blanchard et al, Blood 85:3173 (1995); Falzoni et al, J. Clin. Invest. 95:1207 (1995); Baricolrdi et al, Blood 87:682 (1996)). Certain functional differences exist between rodent and man (Hickman et al, Blood 84:2452 (1994)). The human macrophage P2X₇ receptor (P2X₇) has now been cloned and its functional properties determined (Rassendren et al, J. Biol. Chem. 272:5482 (1997). When compared with the rat P2X₇ receptor, elicited cation-selective currents in the human P2X₇ receptor required higher concentrations of agonists, were more potentiated by removal of extracellular magnesium ions, and revised more rapidly on agonist removal. Expression of chimeric molecules indicated that some of the differences between rat and human P2X₇ receptors could be revised by exchanging the respective C-terminal domains of the receptor proteins.

It has been reported that certain compounds act as P2X₇ antagonists. For example, WO99/29660 and WO99/29661 disclose that certain adamantane derivatives exhibit P2X₇ antagonistic activity having therapeutic efficacy in the treatment of rheumatoid arthritis and psoriasis. Similarly, WO99/29686 discloses that certain heterocyclic derivatives are P2X₇ receptor antagonists and are useful as immunosuppressive agents and treating rheumatoid arthritis, asthma, septic shock and atheroscelerosis. Finally, WO00/71529 discloses certain substituted phenyl compounds exhibiting immunosuppressing activity. All of the references described herein are incorporated herein by reference in their entirety.

It is an object of this invention to provide a novel series of compounds, which can modify the activity of P2X₇ receptor and thus the release of the mediators of inflammation. It is further an object of this invention to provide a series of compounds that can treat or alleviate symptoms of inflammation caused due to the activation of P2X₇ receptor. A still further object of this invention is to provide pharmaceutical compositions that are effective in the treatment or prevention of a variety of disease states, including the diseases associated with the central nervous system, cardiovascular conditions, chronic pulmonary obstructive disease COPD), inflammatory bowel disease, rheumatoid arthritis, osteoarthritis, and other diseases where an inflammatory component is present.

### SUMMARY OF THE INVENTION

Compounds of formulae I-XIX, and their pharmaceutical compositions are disclosed as therapeutic agents useful for the treatment of conditions in mammals associated with abnormal or aberrant activity of the P2X₇ receptor, including inflammatory-mediated conditions such as (but not limited to) arthritis, myocardial infarction, the treatment and prophylaxis of pain syndromes (acute and chronic [neuropathic]), traumatic brain injury, acute spinal cord injury, neurodegenerative disorders, inflammatory bowel disease and immune dysfunctions such as autoimmune disorders.

It has now been found that the present compounds are capable of mediating the activity of the P2X₇ receptor. This finding leads to novel compounds having therapeutic value. It also leads to pharmaceutical compositions having the compounds of the present invention as active ingredients and to their use to treat, prevent or ameliorate a range of conditions in mammals such as but not limited to inflammation of various genesis or etiology, for example rheumatoid arthritis, cardiovascular disease, inflammatory bowel disease, acute, chronic, inflammatory and neuropathic pain, dental pain and headache (such as migraine, cluster headache and tension headache) and other conditions causally related to inflammation or immune dysfunction.

The compounds of the present invention are also useful for the treatment of inflammatory pain and associated hyperalgesia and allodynia. They are also useful for the treatment of neuropathic pain and associated hyperalgesis and allodynia (e.g. trigeminal or herpetic neuralgia, diabetic neuropathy, causalgia, sympathetically maintained pain and deafferentation syndromes such as brachial plexus avulsion). The compounds of the present invention are also useful as anti-inflammatory agents for the treatment of arthritis, and as agents to treat Parkinson's Disease, uveitis, asthma, myocardial infarction, traumatic brain injury, spinal cord injury, neurodegenerative disorders, inflammatory bowel disease and autoimmune disorders, renal disorders, obesity, eating disorders, cancer, schizophrenia, epilepsy, sleeping disorders, cognition, depression, anxiety, blood pressure, lipid disorders, and atherosclerosis.

In one aspect, this invention provides compounds which are capable of modulating the activity of the P2X₇ receptor, in vivo. In a further aspect, the compounds of the invention are capable of antagonizing (suppressing or inhibiting) the activity of the P2X₇ receptor, and thereby treating those conditions, representative ones of which are causally related to aberrant P2X₇ activity.

Accordingly, in a first aspect of the invention, compounds are disclosed that are capable of modulating the activity of the P2X₇ receptor in vivo, having a formula (I):

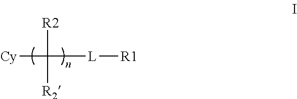

wherein
Cy is a group having a formula:

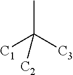

wherein C¹, C² and C³ taken together with the C atom to which they are attached to form a bi or tri cyloalkyl or cycloheteroalkyl ring system of 7-13 atoms; and wherein the ring system is substituted or unsubstituted;
L is

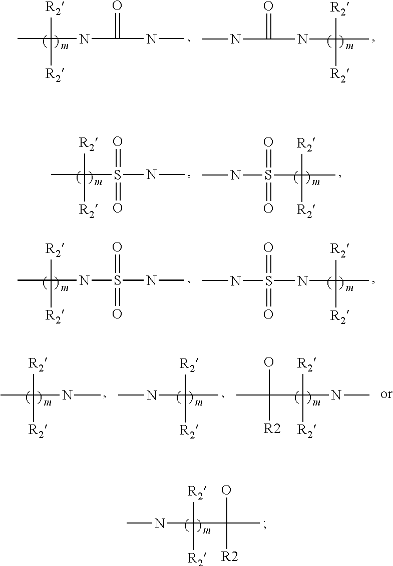

and wherein, when feasible, the —N— or —O atom of the “L” linker is independently substituted with hydrogen or substituted or unsubstituted C₁-C₆ alkyl;
m is 0, 1, 2 or 3;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
n is 0, 1, 2 or 3;
each of R² and R^{2′} is independently selected from hydrogen, substituted or unsubstituted C₁-C₆ alkyl; and
wherein R² and R^{2′} taken together with the C atom to which they are attached may join together to form a cyloalkyl or cycloheteroalkyl ring system of 3-8 atoms; and wherein the ring system is substituted or unsubstituted;
each of R⁴ is independently selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
or a pharmaceutically acceptable salt, solvate or prodrug thereof;
and stereoisomers, isotopic variants and tautomers thereof.

In a second aspect of the invention, compounds are disclosed that are capable of modulating the activity of the P2X₇ receptor in vivo, having a formula (I), as described above, and wherein L is

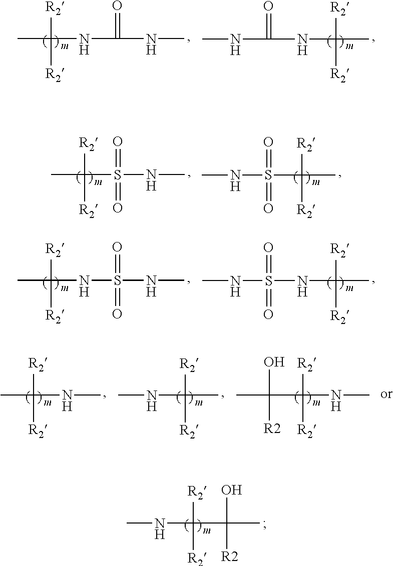

and wherein m, n, R¹, R², R^{2′} and R⁴ are as described above.

In a further embodiment, with respect to compounds of formula I, L may be

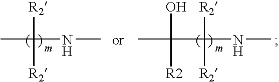

and wherein m, n, R², and R^{2′} are as described above.

In a further embodiment, with respect to compounds of formula I, Cy may be substituted or unsubstituted

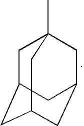

In a further embodiment, with respect to compounds of formula I, R¹ may be substituted or unsubstituted

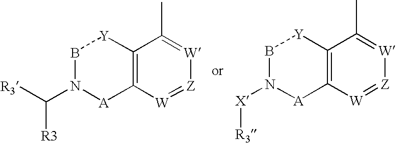

and wherein
A is selected from CR^{2′}R^{2″}, CO, and CS;
B is selected from CR^{2′}, CR^{2′}R^{2″}, CO, and CS;
Y is independently selected from CR^{2′} and CR^{2′}R^{2″};
W, W′ and Z are independently selected from CR⁴ and N, provided that all three of W, W′ and Z cannot be N at the same time;
X′ is selected from —CO—, —CO—NH—, —SO—, —SO₂—, and —SO₂NH—;
R³ is hydrogen or a functional group selected from acyl, substituted acyl, substituted or unsubstituted acylamino, substituted or unsubstituted alkyl, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio; or R³ is a 4-9 membered carbocyclic or heterocyclic ring which can be optionally substituted with at least one substituent selected from a R⁴ group;
each of R^{2′}, R^{2″} and R^{3′} is independently selected from hydrogen, substituted or unsubstituted C₁-C₆ alkyl;
R^{3″} is a group selected from alkyl, substituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl, substituted or unsubstituted bicycloaryl and substituted or unsubstituted bicycloheteroalkyl, or R^{3″} is a 4-9 membered carbocyclic or heterocyclic ring which can be optionally substituted with at least one substituent selected from a R⁴ group;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
and the dotted bond is a single or a double bond.

In a further aspect, the present invention provides pharmaceutical compositions comprising a compound of the invention, and a pharmaceutical carrier, excipient or diluent. In this aspect of the invention, the pharmaceutical composition can comprise one or more of the compounds described herein. Moreover, the compounds of the present invention useful in the pharmaceutical compositions and treatment methods disclosed herein, are all pharmaceutically acceptable as prepared and used.

In a further aspect of the invention, this invention provides a method of treating a mammal susceptible to or afflicted with a condition from among those listed herein, and particularly, such condition as may be associated with e.g. inflammation, such as rheumatoid arthritis, osteoarthritis, uveitis, asthma, myocardial infarction, traumatic brain injury; septic shock, atherosclerosis, chronic pulmonary obstructive disease (COPD), acute spinal cord injury, inflammatory bowel disease and immune dysfunction, including autoimmune disorders, which method comprises administering an effective amount of one or more of the pharmaceutical compositions just described.

In yet another method of treatment aspect, this invention provides a method of treating a mammal susceptible to or afflicted with a condition that is causally related to aberrant P2X₇ receptor activity, and that for example, gives rise to pain responses or that relates to imbalances in the maintenance of basal activity of sensory nerves. The amine compounds of the invention have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with conditions that are causally related to abnormal activity of the P2X₇ receptor, such as neurodegenerative diseases and disorders including, for example, Parkinson's disease, multiple sclerosis; diseases and disorders which are mediated by or result in neuroinflammation such as, for example traumatic brain injury and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; obesity; lipid disorders; cancer; blood pressure; spinal cord injury; and cardiovascular and renal disorders method comprises administering an effective condition-treating or condition-preventing amount of one or more of the pharmaceutical compositions just described.

In additional aspects, this invention provides methods for synthesizing the compounds of the invention, with representative synthetic protocols and pathways disclosed later on herein.

Other objects and advantages will become apparent to those skilled in the art from a consideration of the ensuing detailed description.

### DETAILED DESCRIPTION OF THE INVENTION

### Definitions

When describing the compounds, pharmaceutical compositions containing such compounds and methods of using such compounds and compositions, the following terms have the following meanings unless otherwise indicated. It should also be understood that, consistent with the scope of the present invention, any of the moieties defined herein and/or set forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, C₂-C₆ alkenyl, C₃-C₆ alkynyl, C₁-C₆ alkoxy, aryl and di-C₁-C₆ alkylamino.

“Acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a radical —NR′C(O)R, where R′ is hydrogen, alkyl, cycloalkyl, cycloheteroalkyl, aryl, arylalkyl, heteroalkyl, heteroaryl, heteroarylalkyl and R is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)R where R is hydrogen, alkyl, aryl or cycloalkyl.

“Substituted alkenyl” includes those groups recited in the definition of “substituted”, herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonylamino” refers to the group —NRC(O)OR′ where R is hydrogen, alkyl, aryl or cycloalkyl, and R′ is alkyl or cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkenylene, alkynyl and alkynylene. Aliphatic groups typically have from 1 or 2 to about 12 carbon atoms.

“Alkyl” refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyl” as defined below.

“Substituted alkyl” includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

“Alkylene” refers to divalent saturated aliphatic hydrocarbyl groups particularly having up to about 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” includes those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenyl” refers to monovalent olefinically unsaturated hydrocarbyl groups preferably having up to about 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH—C(CH₃)—) and the like.

“Alkynyl” refers to acetylenically unsaturated hydrocarbyl groups particularly having up to about 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” includes those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkanoyl” as used herein, which can include “acyl”, refers to the group R—C(O)—, where R is hydrogen or alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Substituted Aryl” includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Fused Aryl” refers to an aryl having two of its ring carbon in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group alkyl-NR′—, wherein R′ is selected from hydrogen and alkyl.

“Arylamino” refers to the group aryl-NR′—, wherein R′ is selected from hydrogen, aryl and heteroaryl.

“Alkoxyamino” refers to a radical —N(H)OR where R represents an alkyl or cycloalkyl group as defined herein.

“Alkoxycarbonyl” refers to a radical —C(O)-alkoxy where alkoxy is as defined herein.

“Alkylarylamino” refers to a radical —NRR′ where R represents an alkyl or cycloalkyl group and R′ is an aryl as defined herein.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein. Representative examples include, but are not limited to, methylthio, ethylthio, propylthio, butylthio, and the like.

“Amino” refers to the radical —NH₂.

“Substituted amino” includes those groups recited in the definition of “substituted” herein, and particularly refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Aminocarbonyl” or “amido” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalky, or where the R groups are joined to form an alkylene group.

“Arylalkyloxy” refers to an —O-arylalkyl radical where arylalkyl is as defined herein.

“Arylamino” means a radical —NHR where R represents an aryl group as defined herein.

“Aryloxycarbonyl” refers to a radical —C(O)—O-aryl where aryl is as defined herein.

“Arylsulfonyl” refers to a radical —S(O)₂R where R is an aryl or heteroaryl group as defined herein.

“Azido” refers to the radical —N₃.

“Carbamoyl” refers to the radical —C(O)N(R)₂ where each R group is independently hydrogen, alkyl, cycloalkyl or aryl, as defined herein, which may be optionally substituted as defined herein.

“Carboxy” refers to the radical —C(O)OH.

“Carboxyamino” refers to the radical —N(H)C(O)OH.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Substituted cycloalkyl” includes those groups recited in the definition of “substituted” herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Substituted cycloalkenyl” includes those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Fused Cycloalkenyl” refers to a cycloalkenyl having two of its ring carbon atoms in common with a second aliphatic or aromatic ring and having its olefinic unsaturation located to impart aromaticity to the cycloalkenyl ring.

“Cyanato” refers to the radical —OCN.

“Cyano” refers to the radical —CN.

“Dialkylamino” means a radical —NRR′ where R and R′ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Ethenyl” refers to substituted or unsubstituted —(C═C)—.

“Ethylene” refers to substituted or unsubstituted —(C—C)—.

“Ethynyl” refers to —(C≡C)—.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to, —X, —R¹⁴, —O⁻, ═O, —OR¹⁴, —SR¹⁴, —S⁻, ═S, —NR¹⁴R¹⁵, ═NR¹⁴, —CX₃, —CF₃, —CN, —OCN, —SCN, —NO, —NO₂, ═N₂, —N₃, —S(O)₂O⁻, —S(O)₂OH, —S(O)₂R¹⁴, —OS(O₂)O⁻, —OS(O)₂R¹⁴, —P(O)(O⁻)₂, —P(O)(OR¹⁴)(O⁻), —OP(O)(OR¹⁴)(OR¹⁵), —C(O)R¹⁴, —C(S)R¹⁴, —C(O)OR¹⁴, —C(O)NR¹⁴R¹⁵, —C(O)O⁻, —C(S)OR¹⁴, —NR¹⁶C(O)NR¹⁴R¹⁵, —NR¹⁶C(S)NR¹⁴R¹⁵, —NR¹⁷C(NR¹⁶)NR¹⁴R¹⁵ and —C(NR¹⁶)NR¹⁴R¹⁵, where each X is independently a halogen; each R¹⁴, R¹⁵, R¹⁶ and R¹⁷ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR¹⁸R¹⁹, —C(O)R¹⁸ or —S(O)₂R¹⁸ or optionally R¹⁸ and R¹⁹ together with the atom to which they are both attached form a cycloheteroalkyl or substituted cycloheteroalkyl ring; and R¹⁸ and R¹⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

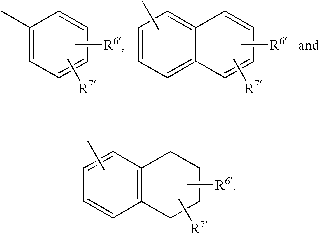

In these formulae one of R^{6′} and R^{7′} may be hydrogen and at least one of R^{6′} and R^{7′} is each independently selected from alkyl, alkenyl, alkynyl, cycloheteroalkyl, alkanoyl, alkoxy, aryloxy, heteroaryloxy, alkylamino, arylamino, heteroarylamino, NR¹⁰COR¹¹, NR¹⁰SOR¹¹, NR¹⁰SO₂R¹⁴, COOalkyl, COOaryl, CONR¹⁰R¹¹, CONR¹⁰OR¹¹, NR¹⁰R¹¹, SO₂NR¹⁰R¹¹, S-alkyl, S-alkyl, SOalkyl, SO₂alkyl, Saryl, SOaryl, SO₂aryl; or R^{6′} and R^{7′} may be joined to form a cyclic ring (saturated or unsaturated) from 5 to 8 atoms, optionally containing one or more heteroatoms selected from the group N, O or S. R¹⁰, R¹¹, and R¹² are independently hydrogen, alkyl, alkenyl, alkynyl, perfluoroalkyl, cycloalkyl, cycloheteroalkyl, aryl, substituted aryl, heteroaryl, substituted or hetero alkyl or the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, tetrahydroisoquinoline, isothiazole, isoxazole, naphthyridine, oxadiazole, oxazole, perimidine, phenanthridine, phenathroline, phenazine, phthalazine, pteridine, purine, pyran, pyrazine, pyrazole, pyridazine, pyridine, pyrimidine, pyrrole, pyrrolizine, quinazoline, quinoline, tetrahydroquinoline, quinolizine, quinoxaline, tetrazole, thiadiazole, thiazole, thiophene, triazole, xanthene, and the like. Particularly, heteroaryl can include other saturated ring systems, and can therefore be derived from indoline, indolizine, tetrahydroquinoline, and tetrahydroisoquinoline. Preferably, the heteroaryl group is between 5-20 membered heteroaryl, with 5-10 membered heteroaryl being particularly preferred. Particular heteroaryl groups are those derived from thiophene, pyrrole, benzothiophene, benzofuran, indole, pyridine, pyrimidine, quinoline, tetrahydroquinoline, isoquinoline, tetrahydroisoquinoline, imidazole, oxazole and pyrazine.

Examples of representative heteroaryls include the following:

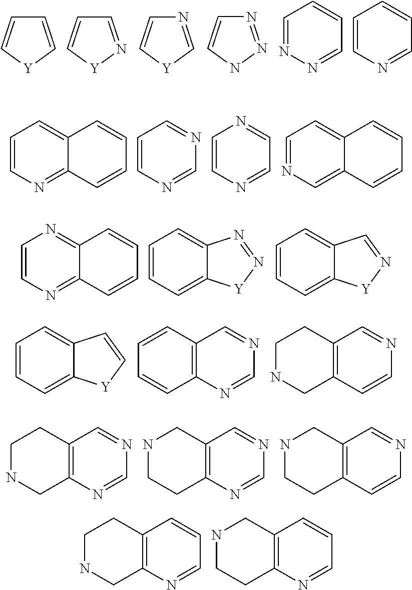

wherein each Y is selected from carbonyl, N, NR⁴, O, and S, where R⁴ is as defined herein.

Examples of representative cycloheteroalkyls include the following

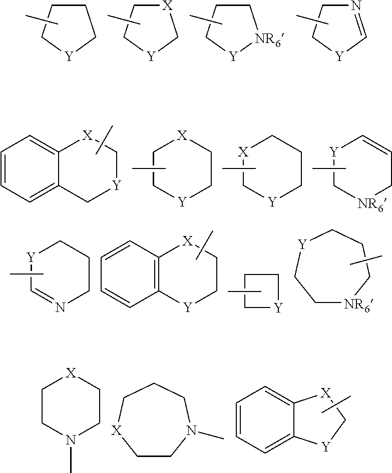

wherein each X is selected from CR⁴₂, NR⁴, O and S; and each Y is selected from NR⁴, O and S, and where R^{6′} is R², R² and R⁴ being as defined herein.

Examples of representative cycloheteroalkenyls include the following:

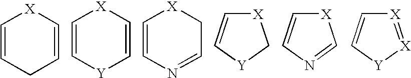

wherein each X is selected from CR⁴, NR⁴, O and S; and each Y is selected from carbonyl, N, NR⁴, O and S, where R⁴ is as defined herein.

Examples of representative aryl having hetero atoms containing substitution include the following:

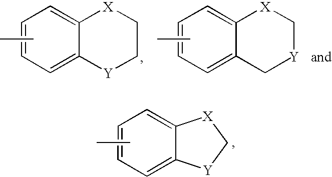

wherein each X is selected from C—R⁴, CR⁴_{2,} NR⁴, O and S; and each Y is selected from carbonyl, NR⁴, O and S, where R⁴ is as defined herein.

“Hetero substituent” refers to a halo, O, S or N atom-containing functionality that may be present as an R⁴ in a CR⁴ group present as substituents directly on W or Z of the compounds of this invention or may be present as a substituent in the “substituted” aryl, heteroaryl and aliphatic groups present in the compounds;

Examples of hetero substituents include:

-halo,

—NO₂, —NH₂, —NHR, —N(R)₂,

—NRCOR, —NRSOR, —NRSO₂R, OH, CN, CO₂R,

—CO₂H,

—O—R,

—CON(R)₂, —CONROR,

—SO₃H, —S—R, —SO₂N(R)₂,

—S(O)R, and —S(O)₂R,

wherein each R is independently an aryl or aliphatic, optionally with substitution. Among hetero substituents containing R groups, preference is given to those materials having aryl and alkyl R groups as defined herein. Where feasible, each R may include hydrogen. Also, where feasible, two R groups when on the same atom, may join to form a heterocyclic ring of 3-8 atoms. For example, two R groups of NR², SO₂NR², and CONR² may join, together with the N atom, to form a N-morpholino, N-pyrrolo, N-piperidino, and N-pyrazolylo ring. Particular hetero substituents are those listed above.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

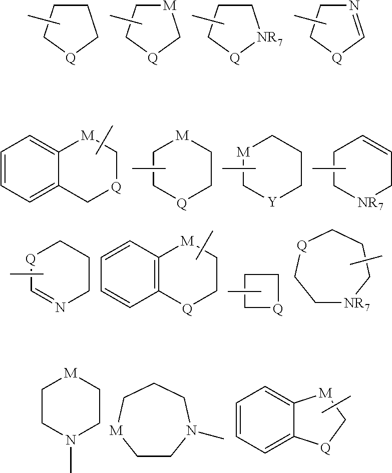

optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR², O, or S; Q is O NR² or S, where R² is as defined herein. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—:

“Dihydroxyphosphoryl” refers to the radical —PO(OH)₂.

“Substituted dihydroxyphosphoryl” includes those groups recited in the definition of “substituted” herein, and particularly refers to a dihydroxyphosphoryl radical wherein one or both of the hydroxyl groups are substituted. Suitable substituents are described in detail below.

“Aminohydroxyphosphoryl” refers to the radical —PO(OH)NH₂.

“Substituted aminohydroxyphosphoryl” includes those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

“Thioalkoxy” refers to the group —SR where R is alkyl.

“Substituted thioalkoxy” includes those groups recited in the definition of “substituted” herein, and particularly refers to a thioalkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as S(O₂)—R wherein R is any substituent described herein. “Aminosulfonyl” or “Sulfonamide” refers to the radical H₂N(O₂)S—, and “substituted aminosulfonyl” “substituted sulfonamide” refers to a radical such as R₂N(O₂)S— wherein each R is independently any substituent described herein.

“Sulfoxide” refers to the divalent radical —S(O)—. “Substituted sulfoxide” refers to a radical such as S(O)—R, wherein R is any substituent described herein.

“Sulfone” refers to the group —SO₂R. In particular embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “human,” “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Representative enol-keto structures and equilibrium are illustrated below:

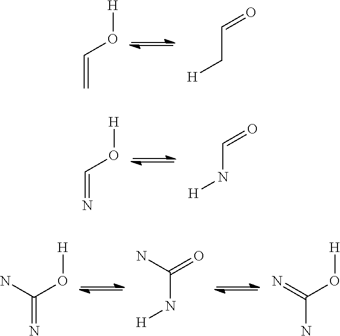

Tautomeric forms may be relevant to the attainment of the optimal-chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

The present invention provides compounds useful for preventing and/or treating a broad range of conditions, associated with abnormalities in the activity of the P2X₇ receptor, among them, rheumatoid arthritis, Parkinson's disease, uveitis, asthma, cardiovascular conditions such as myocardial infarction, the treatment and prophylaxis of pain syndromes (acute and chronic or neuropathic), traumatic brain injury, acute spinal cord injury, neurodegenerative disorders, inflammatory bowel disease and immune dysfunctions such as autoimmune disorders or conditions, in mammals.

In a first embodiment, the present invention, compounds are disclosed that are capable of modulating the activity of the P2X₇ receptor in vivo, having a formula (I):

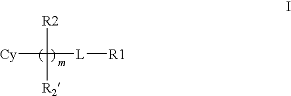

wherein
Cy is a group having a formula:

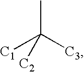

wherein C¹, C² and C³ taken together with the C atom to which they are attached to form a bi or tri cyloalkyl or cycloheteroalkyl ring system of 7-13 atoms; and wherein the ring system is substituted or unsubstituted;
L is

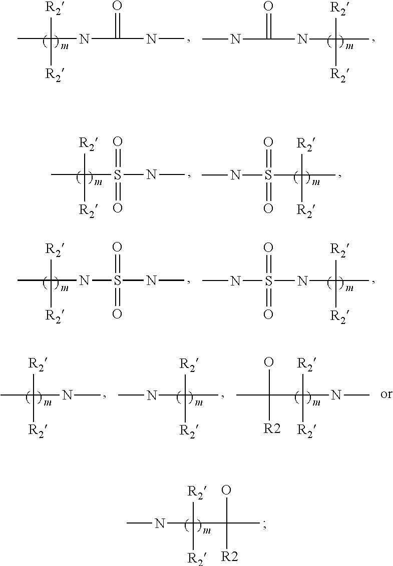

and wherein, when feasible, each —N— and —O atom of the “L” linker is independently substituted with hydrogen or substituted or unsubstituted C₁-C₆ alkyl;
m is 0, 1, 2 or 3;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
n is 0, 1, 2 or 3;
each of R² and R^{2′} is independently selected from hydrogen, substituted or unsubstituted C₁-C₆ alkyl; and
wherein R² and R^{2′} taken together with the C atom to which they are attached may join together to form a cyloalkyl or cycloheteroalkyl ring system of 3-8 atoms; and wherein the ring system is substituted or unsubstituted;
each R⁴ is independently selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;

or a pharmaceutically acceptable salt, solvate or prodrug thereof;
and stereoisomers, isotopic variants and tautomers thereof.

In a second embodiment of the invention, compounds are disclosed that are capable of modulating the activity of the P2X₇ receptor in vivo, having a formula (I):

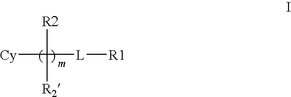

wherein
Cy is

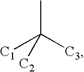

wherein C¹, C² and C³ taken together with the C atom to which they are attached to form a bi- or tri-cyloalkyl or cycloheteroalkyl ring system of 7-13 atoms; and wherein the ring system is substituted or unsubstituted;
L is

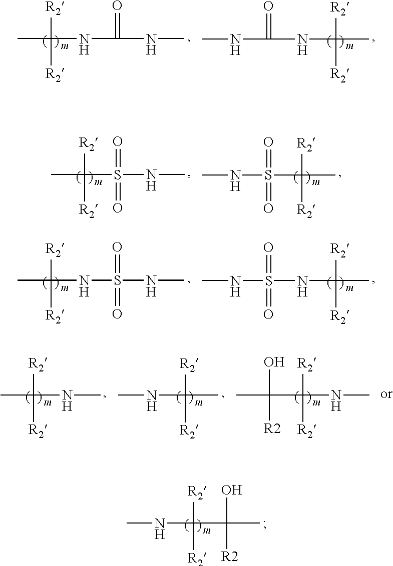

m is 0, 1, 2 or 3;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
n is 0, 1, 2 or 3;
each of R² and R^{2′} is independently selected from hydrogen, substituted or unsubstituted C₁-C₆ alkyl;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
or a pharmaceutically acceptable salt, solvate or prodrug thereof;
and stereoisomers, isotopic variants and tautomers thereof.

Further in accordance with compounds of formula I, and as illustrated in the following non-limiting examples, Cy may be substituted or unsubstituted:

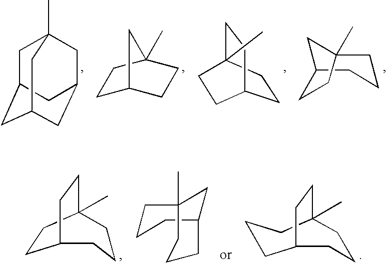

Further in accordance with the compounds of formula I and the following non-limiting examples, Cy may be substituted or unsubstituted:

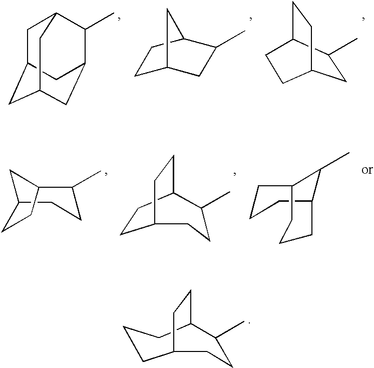

In a further particular embodiment of the compounds of formula I, above, Cy may be

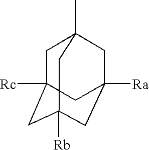

and wherein R^{a}, R^{b} and R^{c} are independently selected from H, halo, hydroxyl, alkyl, amino, and aryl.

Further in accordance with compounds of formulae I, as defined in the preceding paragraph, each of R^{a}, R^{b} and R^{c} are independently selected from H, and halo.

Further in accordance with compounds of formula I, as defined in the preceding paragraph, each of R^{a}, R^{b} and R^{c} are independently selected from H, Br, Cl, OH, Me, NHAc, Ph and F.

Further in accordance with compounds of formula I, as defined in the preceding paragraph, each of R^{a}, R^{b} and R^{c} are independently selected from H and F.

Further in accordance with compounds of formula I, as defined in the preceding paragraph, each of R^{a}, R^{b} and R^{c} is H.

In a further embodiment of the invention, there are provided compounds capable of modulating P2X₇ receptor activity, in vivo, having the following formula II:

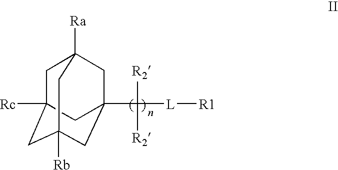

wherein
L is

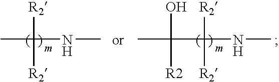

m is 0, 1, 2 or 3;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
n is 0, 1, 2 or 3;
each of R² and R^{2′} is independently selected from hydrogen, substituted or unsubstituted C₁-C₆ alkyl;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio, and
R^{a}, R^{b} and R^{c} are independently selected from H, halo, hydroxyl, alkyl, amino, and aryl;
or a pharmaceutically acceptable salt, solvate or prodrug thereof;
and stereoisomers, isotopic variants and tautomers thereof.

In a further embodiment of the invention, with respect to formula II, each of R¹, R^{b} and R^{c} is H.

In a further embodiment of the invention, with respect to formula II, n is 0.

In a further embodiment of the invention, there are provided compounds capable of modulating P2X₇ receptor activity, in vivo, having the following formula III or IV:

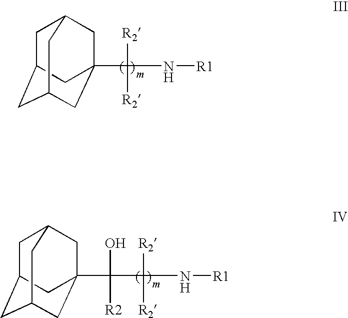

m is 0, 1, 2 or 2;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
each of R² and R^{2′} is independently selected from hydrogen and substituted or unsubstituted C₁-C₆ alkyl;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
or a pharmaceutically acceptable salt, solvate or prodrug thereof;
and stereoisomers, isotopic variants and tautomers thereof.

In a further embodiment of the invention, with respect to formula III, m may be 0.

In a further embodiment of the invention, with respect to formula IV, m may be 0.

In a yet further embodiment of the invention, with respect to formulae III-IV, m may be 1.

In a yet further embodiment of the invention, with respect to formulae III-IV m may be 2.

In a yet further embodiment of the invention, with respect to formulae III-IV, each of R² and R^{2′} is H.

In a yet further embodiment of the invention, with respect to formulae III-IV, each of R² and R^{2′} is Me.

In a yet further embodiment of the invention, with respect to formulae III-IV, one of R² and R^{2′} is H and other is substituted alkyl.

In a yet further embodiment of the invention, with respect to formulae III-IV, one of R² and R^{2′} is H and other is Me.

In a further embodiment of the invention, with respect to formula II, L is

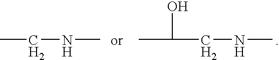

In a further embodiment of the invention, there are provided compounds capable of modulating P2X₇ receptor activity, in vivo, having the following formula V or VI:

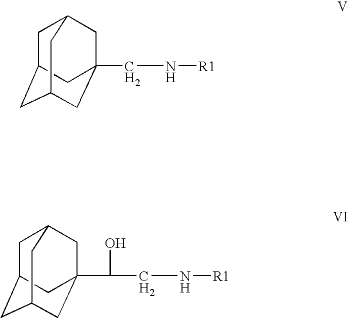

wherein
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, sulfo, sulfanyl, sulfinyl, amido, carboxy, ester, alkyl, substituted alkyl, alkenyl, substituted alkenyl, alkynyl, substituted alkynyl, and sulfonamide;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
or a pharmaceutically acceptable salt, solvate or prodrug thereof;
and stereoisomers, isotopic variants and tautomers thereof.

In a further embodiment of the invention, with respect to formulae I-VI, R¹ is

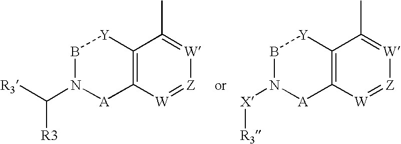

wherein
A is selected from CR^{2′}R^{2″}, CO, and CS;
B is selected from CR^{2′}, CR^{2′}R^{2″}, CO, and CS;
Y is independently selected from CR^{2′} and CR^{2′}R^{2″};
W, W′ and Z are independently selected from CR⁴ and N, provided that all three of W, W′ and Z cannot be N at the same time;
X′ is selected from —CO—, —CO—NH—, —SO—, —SO₂—, and —SO₂NH—;
R³ is hydrogen or a functional group selected from acyl, substituted acyl, substituted or unsubstituted acylamino, substituted or unsubstituted alkyl, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio; or R³ is a 4-9 membered carbocyclic or heterocyclic ring which can be optionally substituted with at least one substituent selected from a R⁴ group;
each of R^{2′}, R^{2″} and R^{3′} is independently selected from hydrogen, substituted or unsubstituted C₁-C₆ alkyl;
R^{3″} is a group selected from alkyl, substituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl, substituted or unsubstituted bicycloaryl and substituted or unsubstituted bicycloheteroalkyl, or R^{3″} is a 4-9 membered carbocyclic or heterocyclic ring which can be optionally substituted with at least one substituent selected from a R⁴ group;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio;
and the dotted bond is a single or a double bond.

In a further embodiment of the invention, with respect to formulae I-VI, R¹ is

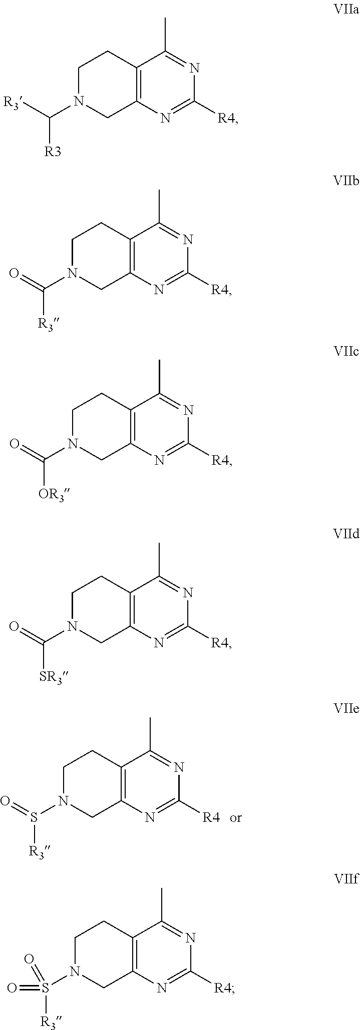

R³ is hydrogen or a group selected from substituted or unsubstituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl; or R³ is a 4-9 membered carbocyclic or heterocyclic ring which can be optionally substituted with at least one substituent selected from a R⁴ group;
R^{3′} is independently selected from hydrogen, substituted or unsubstituted C₁-C₆ alkyl;
R^{3″} is a group selected from alkyl, substituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl; substituted or unsubstituted bicycloaryl and substituted or unsubstituted bicycloheteroalkyl, or R^{3″} is a 4-9 membered carbocyclic or heterocyclic ring which can be optionally substituted with at least one substituent selected from a R⁴ group;
R⁴ is selected from H, alkyl, substituted alkyl, acyl, substituted acyl, substituted or unsubstituted acylamino, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio.

In one embodiment of the invention, with respect to formulae I-VI, R¹ is as defined in the preceding paragraph (VIIa-f) and wherein R^{3′} is H.

In one embodiment of the invention, with respect to formulae I-VI, R¹ is as defined in the preceding paragraph (VIIa-f) and wherein the compound is

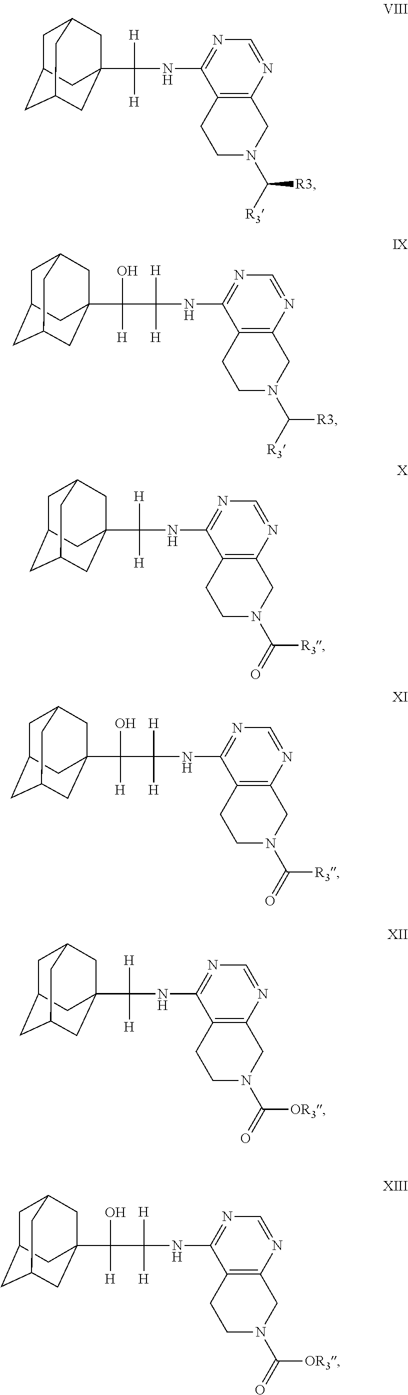

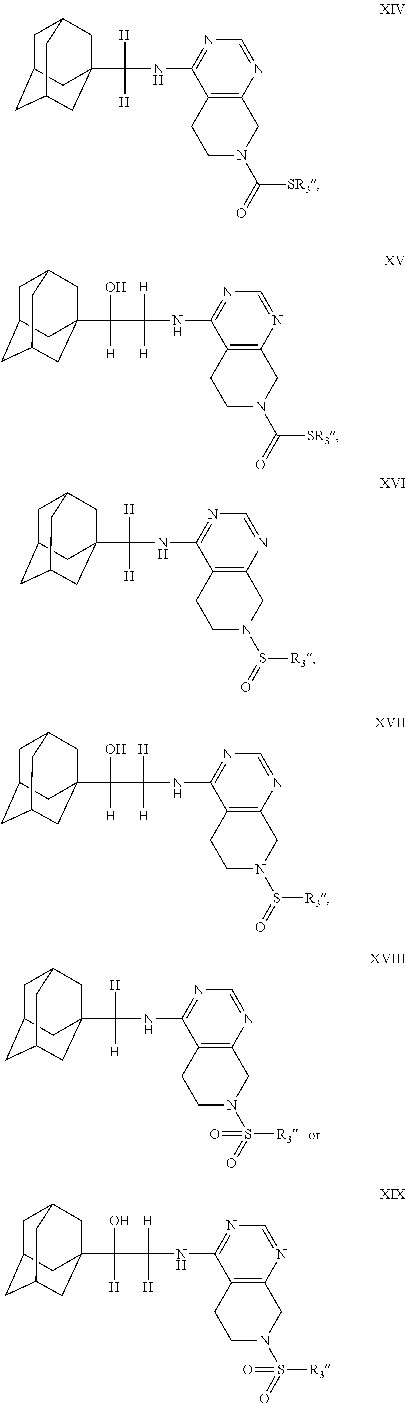

and wherein R³, R^{3′} and R^{3″} are as defined in the preceding paragraphs.

In one embodiment of the invention, with respect to formulae VIII-IX, R^{3′} is H.

In another embodiment of the invention, with respect to formulae VIII-IX, R^{3′} is substituted or unsubstituted alkyl.

In another embodiment of the invention, with respect to formulae VIII-IX, R^{3′} is substituted Me.

In another embodiment of the invention, with respect to formulae VIII-IX, R^{3′} is substituted alkyl and the substitution is selected from aryl, heteroaryl, cycloalkyl, heterocycloalkyl, halo, alkoxy, hydroxy, cyano, and aryloxy.

In another embodiment of the invention, with respect to formulae VIII-IX, R^{3′} is substituted alkyl and the substitution is selected from hydroxyl, amino, substituted amino, alkoxy, carboxy, carbalkoxy, sulfonyl, sulfanyl, sulfinyl, alkyl, halo, cyano, —NHSO₂R^{2′}, —NHCONH₂, —NHCONHSO₃R^{2′}, and wherein R^{2′} is substituted or unsubstituted C₁-C₆ alkyl.

In another embodiment of the invention, with respect to formulae VIII-IX, R^{3′} is substituted alkyl and the substitution is selected from phenyl, OH, NH₂, NHMe, CN, NHEt, NHCONHSO₃Me, SO₂Me, CF₃, SO₂CF₃, NHSO₂CF₃, NHSO₂Me, Me, CO₂H, CO₂Et, pyrrolidinyl, pyrazolyl, piperidinyl, or morpholinyl.

In another embodiment of the invention, with respect to formulae VIII-IX, R^{3′} is substituted alkyl and the substitution is selected from —(CH₂)₃—OH, —(CH₂)₄—NHMe, —(CH₂)₄—OH, —(CH₂)₂—CH(OH)—CH₂OH, —(CH₂)₄—CO₂H, —(CH₂)₄—NHEt, —(CH₂)₃—NHEt, —(CH₂)₂—NH—(CH₂)₂OH, —(CH₂)₃—NH—(CH₂)₃OH, —(CH₂)₄—NH₂, —(CH₂)₃—NHCONHSO₂Me, —(CH₂)₃—NH—(CH₂)₂—Me, —(CH₂)₂CO₂H, and N-Me-piperidinyl.

In another embodiment of the invention, with respect to formulae VIII-IX, R^{3′} is substituted alkyl and the substitution is selected from Ph, Cl, F, Br, CN, OH, OMe, OPh, CF₃, CHF₂, OCF₃, t-Bu, SMe, SOMe, SO₂Me, SO₃H, SO₃Me, pyridyl, cyclopropyl, cyclopentyl and cyclohexyl.

In another embodiment of the invention, with respect to formulae VIII-IX, R^{3′} is CH₂OH.

In one embodiment of the invention, with respect to formulae VIII-IX, R³ is H.

In another embodiment of the invention, with respect to formulae VIII-IX, R³ is substituted or unsubstituted alkyl.

In another embodiment of the invention, with respect to formulae VIII-IX, R³ is substituted Me.

In another embodiment of the invention, with respect to formulae VIII-IX, R³ is substituted alkyl and the substitution is selected from aryl, heteroaryl, cycloalkyl, heterocycloalkyl, halo, alkoxy, hydroxy, cyano, and aryloxy.

In another embodiment of the invention, with respect to formulae VIII-IX, R³ is substituted alkyl and the substitution is selected from hydroxyl, amino, substituted amino, alkoxy, carboxy, carbamoyl, carbalkoxy, sulfonyl, sulfanyl, sulfinyl, alkyl, halo, cyano, —NHSO₂R^{2′}, —NHCONH₂, —NHCONHSO₃R^{2′}, and wherein R^{2′} is substituted or unsubstituted C₁-C₆ alkyl.

In another embodiment of the invention, with respect to formulae VIII-IX, R³ is substituted alkyl and the substitution is selected from phenyl, OH, NH₂, NHMe, CN, NHEt, NHCONHSO₃Me, SO₂Me, CF₃, SO₂CF₃, NHSO₂CF₃, NHSO₂Me, Me, CO₂H, CO₂Et, CONH₂, pyrrolidinyl, pyrazolyl, piperidinyl, or morpholinyl.

In another embodiment of the invention, with respect to formulae VIII-IX, R³ is substituted alkyl and the substitution is selected from —(CH₂)₃—OH, —(CH₂)₄—NHMe, —(CH₂)₄—OH, —(CH₂)₂, CH(OH)—CH₂OH, —(CH₂)₄—CO₂H, —(CH₂)₄—NHEt, —(CH₂)₃—NHEt, —(CH₂)₂—NH—(CH₂)₂OH, —(CH₂)₃—NH—(CH₂)₃OH, —(CH₂)₄—NH₂, —(CH₂)₃—NHCONHSO₂Me, —(CH₂)₃—NH—(CH₂)₂-Me, —(CH₂)₂CO₂H, and N-Me-piperidinyl.

In another embodiment of the invention, with respect to formulae VIII-IX, R³ is substituted alkyl and the substitution is selected from Ph, Cl, F, Br, CN, OH, OMe, OPh, CF₃, CHF₂, OCF₃, t-Bu, SMe, SOMe, SO₂Me, SO₃H, SO₃Me, pyridyl, cyclopropyl, cyclopentyl and cyclohexyl.

In another embodiment of the invention, with respect to formulae VIII-IX, R³ is selected from hydrogen, carbamoyl, carboxy, and carbalkoxy.

In another embodiment of the invention, with respect to formulae VIII-IX, R³ is CONH₂, CO₂H, CO₂Et, CONMe₂, pyrrolidinyl, pyrazolyl, piperidinyl, or morpholinyl.

In another embodiment of the invention, with respect to formulae VIII-IX, R³ is CONH₂.

In another embodiment of the invention, with respect to formulae X-XIX R^{3″} is alkyl. In another particular embodiment, R^{3″} is methyl, iso-Pr, or t-Bu.

In another embodiment of the invention, with respect to formulae X-XIX R^{3″} is substituted alkyl and the substitution is selected from aryl, heteroaryl, cycloalkyl, heterocycloalkyl, halo, alkoxy, hydroxy, cyano, and aryloxy.

In another embodiment of the invention, with respect to formulae X-XIX, R^{3″} is substituted alkyl and the substitution is selected from hydroxyl, amino, substituted amino, alkoxy, carboxy, carbamoyl, carbalkoxy, sulfonyl, sulfanyl, sulfinyl, alkyl, halo, cyano, —NHCO₂R^{2′}, —NHSO₂R^{2′}, —NHCONH₂, —NHCONHSO₃R^{2′}, and wherein R^{2′} is substituted or unsubstituted C₁-C₆ alkyl.

In another embodiment of the invention, with respect to formulae X-XIX, R^{3″} is substituted alkyl and the substitution is selected from phenyl, OH, NH₂, NHMe, CN, NHEt, NHCONHSO₃Me, SO₂Me, CF₃, SO₂CF₃, NHSO₂CF₃, NHSO₂Me, Me, CO₂H, CO₂Et, pyrrolidinyl, pyrazolyl, piperidinyl, or morpholinyl.

In another embodiment of the invention, with respect to formulae X-XIX, R^{3″} is substituted alkyl and the substitution is selected from —(CH₂)₃—OH, —(CH₂)₄—NHMe, —(CH₂)₄—OH, —(CH₂)₂₋CH(OH)—CH₂OH, —(CH₂)₄—CO₂H, —(CH₂)₄—NHEt, —(CH₂)₃—NHEt, —(CH₂)₂—NH—(CH₂)₂OH, —(CH₂)₃—NH—(CH₂)₃OH, —(CH₂)₄—NH₂, —(CH₂)₃—NHCONHSO₂Me, —(CH₂)₃—NH—(CH₂)₂-Me, —(CH₂)₂CO₂H, and N-Me-piperidinyl.

In another embodiment of the invention, with respect to formulae X-XIX, R^{3″} is substituted alkyl and the substitution is selected from Ph, Cl, F, Br, CN, OH, OMe, OPh, CF₃, CHF₂, OCF₃, t-Bu, SMe, SOMe, SO₂Me, SO₃H, SO₃Me, pyridyl, cyclopropyl, cyclopentyl and cyclohexyl.

In another embodiment of the invention, with respect to formulae VIII-XIX, R³ or R^{3″} is independently

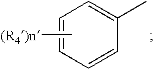

and wherein n′ is selected from 1-5 and each of R^{4′} is independently selected from hydrogen, alkyl, substituted alkyl, acyl, substituted acyl, substituted or unsubstituted acylamino, substituted or unsubstituted alkylamino, substituted or unsubstituted alkylthio, substituted or unsubstituted alkoxy, aryloxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted or unsubstituted sulfone, substituted or unsubstituted sulfanyl, substituted or unsubstituted aminosulfonyl, substituted or unsubstituted arylsulfonyl, sulfuric acid, sulfuric acid ester, substituted or unsubstituted dihydroxyphosphoryl, substituted or unsubstituted aminodihydroxyphosphoryl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio.

In another embodiment of the invention, with respect to formulae VIII-XIX, R³ or R^{3″} is

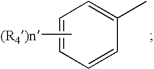

and n′ is 1-3.

In another embodiment of the invention, with respect to formulae VIII-XIX, R³ or R^{3″} is

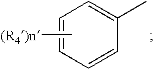

and n′ is 1-2.

In another embodiment of the invention, with respect to formulae VIII-XIX, R³ or R^{3″} is

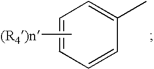

and n′ is 1.

In another embodiment of the invention, with respect to formulae VIII-XIX, R³ or R^{3″} is

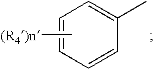

and each of R^{4′} is independently selected from Me, Et, Ph, Cl, F, Br, CN, OH, OMe, OEt, OPh, CF₃, CHF₂, OCHF₂, NMe₂, OCF₃, t-Bu, SMe, NHCOMe, OCH₂Ph, CH═CH—CO₂H, SOMe, SO₂Me, SO₃H, SO₃Me, and pyridyl.

In another embodiment of the invention, with respect to formulae VIII-XIX, R^{4′} is independently selected from aryloxy and heteroaryloxy.

In another embodiment of the invention, with respect to formulae VIII-XIX, R^{4′} is substituted phenyl.

In another embodiment of the invention, with respect to formulae VIII-XIX, R³ or R^{3″} is independently substituted or unsubstituted cycloalkyl, heterocycloalkyl, heteroaryl, bicycloaryl or bicycloheteroaryl.

In another embodiment of the invention, with respect to formulae VIII-XIX, R³ or R^{3″} is independently substituted or unsubstituted naphthalene, furanyl, thiophenyl, pyrrolyl, imidazolyl, pyridyl, pyrimidinyl, quinoline, isoquinolinyl, triazolyl, oxazolyl, pyrazolyl, piperidinyl, piperizinyl, tetrahydrofuranyl, morpholinyl, azepinyl, coumarinyl, tetrahydroquinolinyl, tetrahydroisoquinolinyl, indolyl, benzopyranyl, benzofuranyl, benzodioxolyl, or benzodioxanyl.

In another embodiment of the invention, with respect to formulae VIII-XIX, R³ or R^{3″} is substituted or unsubstituted pyridyl.

In another embodiment of the invention, with respect to formulae VIII-XIX, R³ or R^{3″} is independently substituted or unsubstituted

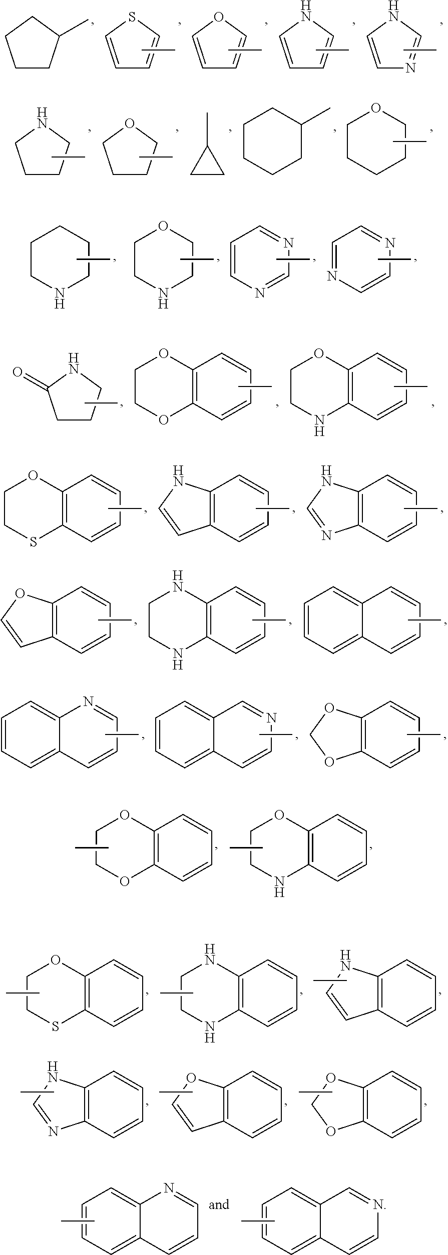

In another embodiment of the invention, with respect to formulae VIII-XIX, R³ or R^{3″} is independently substituted C₁-C₆ alkyl and the substitution is selected from hydroxyl, amino, substituted amino, alkoxy, carboxy, carbamoyl, carbalkoxy, sulfonyl, sulfanyl, sulfinyl, alkyl, halo, cyano, —NHSO₂R^{2′}, —NHCONH₂, —NHCONHSO₃R^{2′} and wherein R^{2′} is substituted or unsubstituted C₁-C₆ alkyl.

In another embodiment of the invention, with respect to formulae VIII-XIX, R³ or R^{3″} is independently substituted C₁-C₆ alkyl and the substitution is selected from phenyl, OH, NH₂, NHMe, CN, NHEt, NHCONHSO₃Me, SO₂Me, CF₃, SO₂CF₃, NHSO₂CF₃, NHSO₂Me, Me, OMe, CO₂H, CO₂Et, pyrrolidinyl, pyrazolyl, piperidinyl, or morpholinyl.

In another embodiment of the invention, with respect to formulae VIII-XIX, R³ or R^{3″} is independently (CH₂)₃—OH, —(CH₂)₄—NHMe, —(CH₂)₄—OH, —(CH₂)₂₋CH(OH)—CH₂OH, —(CH₂)₄—CO₂H, —(CH₂)₄—NHEt, —(CH₂)₃—NHEt, —(CH₂)₂—NH—(CH₂)₂OH, —(CH₂)₃—NH—(CH₂)₃OH, —(CH₂)₄—NH₂, —(CH₂)₃—NHCONHSO₂Me, —(CH₂)₃—NH—(CH₂)₂-Me, —(CH₂)₂CO₂H, or N-Me-piperidinyl.

In another embodiment of the invention, with respect to formulae VIII-XIX, R³ or R^{3″} is independently substituted C₁-C₆ alkyl and the substitution is selected from aryl, heteroaryl, cycloalkyl, heterocycloalkyl, halo, alkoxy, hydroxy, cyano, and aryloxy.

In another embodiment of the invention, with respect to formulae VIII-XIX, R³ or R^{3″} is independently substituted C₁-C₆ allyl and the substitution is selected from aryl, heteroaryl, cycloalkyl, heterocycloalkyl, halo, alkoxy, hydroxy, cyano, and aryloxy.

In another embodiment of the invention, with respect to formulae VIII-XI and XVI-IX, R³ or R^{3″} is independently Ph, CF₃, CHF₂, OCF₃, t-Bu, SMe, pyridyl, cyclopropyl, cyclopentyl and cyclohexyl.

In another embodiment of the invention, with respect to formulae VIII-XI and XVI-IX,

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The compounds of this invention may be modified to enhance their uptake, for example, into tissues or cells, by proteins or other agents, including and not limited to, transporters. Additionally, the compounds may be modified to provide sustained systemic concentrations of the compound. The modification may be direct or indirect. Direct modification may include, and is not limited to, modifying the compounds either alone, by linkage to a conjugate moiety, or a combination thereof to be substrates for active transporters. Indirect modification may include, and is not limited to, administering one or more conjugate moieties to enhance the uptake of the compound. Direct or indirectly modified compounds may be administered, for example, by oral, parenteral, transdermal, topical, injectable or other routes.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

### Formulation 1—Tablets

A compound of the invention is admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

### Formulation 2—Capsules

A compound of the invention is admixed as a dry powder with a starch diluent in an approximate 1:1 weight ratio. The mixture is filled into 250 mg capsules (125 mg of active amide compound per capsule).

### Formulation 3—Liquid

A compound of the invention (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

A compound of the invention is admixed as a dry powder with a dry gelatin binder in an approximate 1:2 weight ratio. A minor amount of magnesium stearate is added as a lubricant. The mixture is formed into 450-900 mg tablets (150-300 mg of active amide compound) in a tablet press.

### Formulation 5—Injection

A compound of the invention is dissolved or suspended in a buffered sterile saline injectable aqueous medium to a concentration of approximately 5 mg/ml.

### Formulation 6—Topical

Stearyl alcohol (250 g) and a white petrolatum (250 g) are melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### Methods of Treatment

The present compounds are used as therapeutic agents for the treatment of conditions in mammals that are causally related or attributable to aberrant activity of the P2X₇ receptor. Accordingly, the compounds and pharmaceutical compositions of this invention find use as therapeutics for preventing and/or treating autoimmune, inflammatory and cardiovascular conditions in mammals including humans.

In a method of treatment aspect, this invention provides a method of treating a mammal susceptible to or afflicted with a condition associated with arthritis, uveitis, asthma, myocardial infarction, traumatic brain injury, acute spinal cord injury, inflammatory bowel disease and autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. The present amines have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

In additional method of treatment aspects, this invention provides methods of treating a mammal susceptible to or afflicted with neurodegenerative diseases and disorders such as, for example Parkinson's disease, multiple sclerosis; diseases and disorders which are mediated by or result in neuroinflammation such as, for example traumatic brain injury, and encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders, uveitis and atherosclerosis; itch/pruritus such as, for example psoriasis; obesity; lipid disorders; cancer; blood pressure; spinal cord injury; and renal disorders method comprises administering an effective condition-treating or condition-preventing amount of one or more of the pharmaceutical compositions just described.

As a further aspect of the invention there is provided the present amine compounds for use as a pharmaceutical especially in the treatment or prevention of the aforementioned conditions and diseases. There is also provided the use of a present amine compound in the manufacture of a medicament for the treatment or prevention of one of the aforementioned conditions and diseases.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a neurodegenerative, autoimmune or inflammatory condition, the compounds of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other compounds that demonstrate the same or a similar therapeutic activity, and that are determined to safe and efficacious for such combined administration.

### General Synthetic Procedures

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following schemes are presented with details as to the preparation of representative compounds of this invention that have been listed herein. The compounds of the invention may be prepared from known or commercially available starting materials and reagents by one skilled in the art of organic synthesis.

### Intermediate 1

### N-Benzyl-4-chloro-5,6,7,8-tetrahydropyrido[3,4-d]pyrimidine

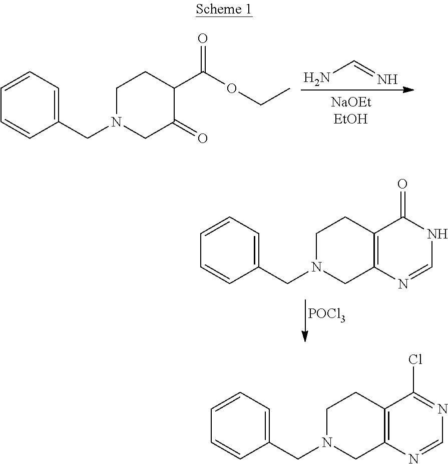

### a) 7-Benzyl-5,6,7,8-tetrahydro-3H-pyrido[3,4-d]pyrimidin-4-one

A 2-necked 1 liter RBF (round bottom flask) was charged with ethyl 1-benzyl-3-oxopiperidine-4-carboxylate (30 g, 101 mmol), formamidine acetic acid salt (10.5 g, 101 mmol) and EtOH (450 mL). The resulting mixture was stirred at 0° C. and treated with NaOEt (21% in EtOH) (112.6 mL, 299 mmol). The reaction mixture was heated at 60° C. overnight and monitored for completion via LCMS and TLC (DCM:MeOH:95:5) (DCM—dichloromethane). Additional starting carboxylate (1 g) was added, followed by another gram of the same after 12 h with continued heating at 60° C. The reaction was complete after 4 h as indicated by LCMS. The cooled reaction was concentrated under vacuum and the residue was treated with conc. HCl (300 mL) and stirred overnight at room temperature. The solvents were removed under vacuum and the resulting solids were treated with EtOH (300 mL), stirred for 15 minutes and then filtered to furnish 44 g of crude 7-benzyl-5,6,7,8-tetrahydro-3H-pyrido[3,4-d]pyrimidin-4-one as the HCl salt, which was used as such for the next step without further purification.

LCMS (0.1% formic acid modifier) calculated. (M+1)⁺ 242.12, observed 242.3.

¹H NMR (CD₃OD) (free base) δ 7.98 (s, 1 H), 7.35-7.32 (m, 5 H), 3.72 (s, 2 H), 3.41-3.40 (m, 2H), 2.75 (t, J=5.7 Hz, 2 H), 2.57 (m, 2 H).

### b) 7-Benzyl-4-chloro-5,6,7,8-tetrahydropyrido[3,4-d]pyrimidine

A RBF (250 mL) equipped with a reflux condenser was charged with 35.3 g of 7-benzyl-5,6,7,8-tetrahydro-3H-pyrido[3,4-d]pyrimidin-4-one HCl followed by the addition of POCl₃ (60 mL). The resulting mixture was refluxed for 4 h under argon, cooled to RT and diluted with DCM (200 mL). The crude reaction was then poured into ice-cold water (200 mL) and stirred overnight at 0° C. The layers were separated and the pH of the aqueous layer was adjusted to 7 via the addition of satd. NaHCO₃. The aqueous layer was extracted with DCM (3×100 mL) and the combined organic layers were washed with satd. NaHCO₃ (50 mL), dried over anhyd. Na₂SO₄, filtered and concentrated to yield 7-benzyl-4-chloro-5,6,7,8-tetrahydropyrido[3,4-d]pyrimidine as a viscous dark brown oil (21 g, 81 mmol, 80%). LCMS (0.1% formic acid modifier) calculated. (M+1)⁺ 260.09, observed 260.1.

¹H NMR (CD₃OD) δ 8.68 (s, 1 H), 7.36-7.29 (m, 5 H), 3.75 (s, 2 H), 3.65 (s, 2H), 2.86(s, 4 H),

### Intermediate 2

### Adamantan-1-yl-methyl-(5,6,7,8-tetra hydro-pyrido[3,4-d]pyrimidin-4-yl)-amine

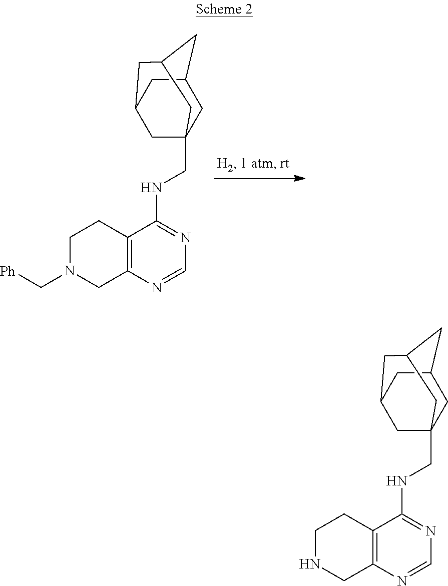

A RBF (50 mL) was charged with adamantan-1-yl-methyl-(7-benzyl-5,6,7,8-tetrahydro-pyrido[3,4-d]pyrimidin-4-yl)-amine (1.1 g, 2.6 mmol), palladium hydroxide (0.3 g, 20%, 0.4 mmol) and methanol (10 mL). The mixture was stirred under a hydrogen atmosphere (balloon) at room temperature for 16 hours. The reaction mixture was filtered through a pad of Celite, and rinsed with MeOH (30 mL). The combined filtrates were concentrated under reduced pressure gave adamantan-1-ylmethyl-(5,6,7,8-tetra hydro-pyrido[3,4-d]pyrimidin-4-yl)-amine as a light yellow solid (0.72 g, 2.2 mmol, 84%).

LCMS (0.1% formic acid modifier) calculated. (M)⁺ 298.43, observed 299.5.

¹H NMR (CDCl₃) δ 8.41 (s, 1 H), 4.51 (bs, 1H), 3.89 (s, 2H), 3.27-3.21 (m, 4H), 2.39 (bs, 2H), 1.99 (bs, 3H), 1.74-1.54 (m, 12H).

### Intermediate 3

### 1-Adamantan-1-yl-2-(7-benzyl-5,6,7,8-tetrahydro-pyrido[3,4-d]pyrimidin-4-ylamino)-ethanol

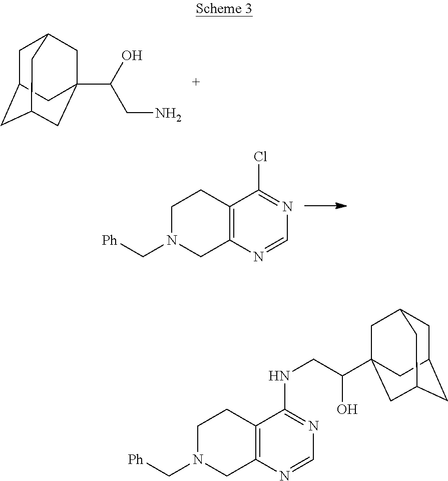

A mixture of 7-benzyl-4-chloro-5,6,7,8-tetrahydropyrido[3,4-d]pyrimidine (1.23 g, 0.00474 mol), 1-adamantan-1-yl-2-amino-ethanol (1.39 g, 0.00710 mol) and N,N-diisopropylethylamine (1.6 mL, 0.0095 mol) in acetonitrile (30 mL) was heated via microwave at 150° C. for one hour. The mixture was cooled to room temperature and poured into saturated sodium bicarbonate solution and extracted with ethyl acetate. The organic layer was washed with brine, dried over sodium sulfate, filtered and concentrated under vacuum to leave a yellow solid. The solid was purified by flash chromatography over silica gel (0-10% methanol in dichloromethane gradient) to give the 1-adamantan-1-yl-2-(7-benzyl-5,6,7,8-tetrahydro-pyrido[3,4-d]pyrimidin-4-ylamino)-ethanol.

LCMS (0.1% formic acid modifier) calculated. (M+1)⁺ 418.57, observed 419.

### Intermediate 4

### Adamantan-1-yl-2-(5,6,7,8-tetrahydro-pyrido[3,4-d]pyrimidin-4-ylamino)-ethanol

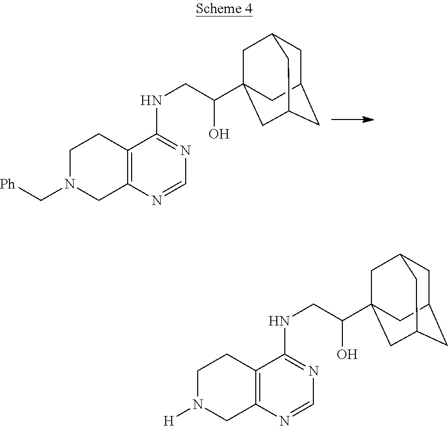

Into a 500 ml Parr flask was combined 1-adamantan-1-yl-2-(7-benzyl-5,6,7,8-tetrahydro-pyrido[3,4-d]pyrimidin-4-ylamino)-ethanol (1.57 g, 0.00371 mol), palladium hydroxide (0.05 g, 0.0004 mol), and methanol (10 mL, 0.4 mol). The flask was pressurized to 40 psi using a Parr shaker apparatus. The mixture was filtered over Celite and the filtrate was reduced in vacuo to yield 1-adamantan-1-yl-2-(5,6,7,8-tetrahydro-pyrido[3,4-d]pyrimidin-4-ylamino)-ethanol as an off white solid.

LCMS (0.1% formic acid modifier) calculated. (M+1)+ 328.45, observed 329.

### Intermediate 5

### 3-[(5,6,7,8-Tetrahydro-pyrido[3,4-d]pyrimidin-4-ylmethyl)-amino]-adamantan-1-ol

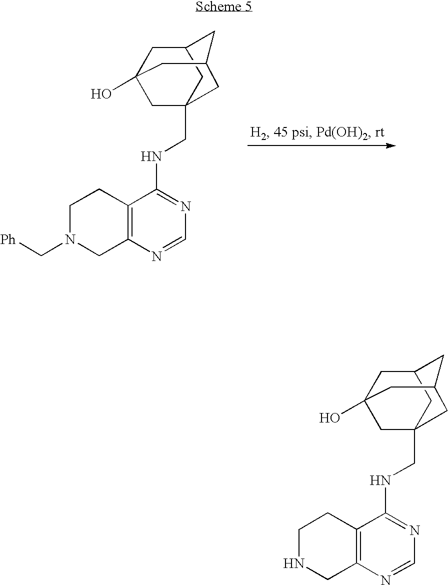

Into a 500 ml Parr apparatus vessel was combined 3-[(7-benzyl-5,6,7,8-tetrahydro-pyrido[3,4-d]pyrimidin-4-ylamino)-methyl]-adamantan-1-ol (prepared using a general method described for the Intermediate 2) (1.0 g, 0.0020 mol), palladium hydroxide (0.2 g, 0.001 mol), and methanol (40 mL). The vessel was pressurized to 45 psi under hydrogen overnight with a parr shaker apparatus. The mixture was filtered through a pad of Celite and the filtrate was reduced in vacuo. The residue was purified via flash chromatography (40 g of silica gel, 0-20% MeOH/CH₂Cl₂ gradient) to yield 3-[(5,6,7,8-tetrahydro-pyrido[3,4-d]pyrimidin-4-ylmethyl)-amino]-adamantan-1-ol as an off white solid (0.50 g, 0.0015 mol, 76%).

LCMS (0.1% formic acid modifier) calculated. (M)+ 314.43, observed 315.3.

¹H NMR (CDCl₃) δ 8.40 (s, 1 H), 4.56 (bs, 1H), 3.38-3.36 (m, 2H), 3.23-3.20 (m, 2H), 2.38-2.36 (m, 2H), 2.23 (bs, 2H), 1.72-1.46 (m, 12H).

### Representative Synthetic Methods for Preparation of Compounds of the Invention

The following methods (Method A-Method J) or some modification thereof, were used or can be used to prepare compounds of this invention. Appropriate reagents and corresponding starting materials were utilized in the preparation of these compounds. Various purification methods known to those skilled in the art were used to purify the final compounds.

The compounds prepared or can be prepared using these methods along with their method of preparation are listed in Table 1 and 2.

### Method A

### Preparation of Representative Examples

### Compound 1

### Adamantan-1-ylmethyl-(7-benzyl-5,6,7,8-tetrahydro-pyrido[3,4-d]pyrimidin-4-yl)-amine

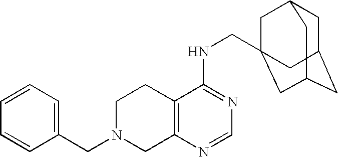

A mixture of 7-benzyl-4-chloro-5,6,7,8-tetrahydropyrido[3,4-d]pyrimidine (Intermediate 1, 1.6 g, 0.0060 mol), 1-adamantanemethylamine (1.0 g, 0.0060 mol) and N,N-diisopropylethylamine (1.44 g, 0.0111 mol) in acetonitrile (30 mL, 0.6 mol) was heated via microwave at 150° C. for 2.5 hours. The mixture was cooled to room temperature and then concentrated under reduced pressure and purified via flash chromatography (40 g of silica gel, 0-100% EtOAc/hexanes gradient) to give adamantan-1-yl-methyl-(7-benzyl-5,6,7,8-tetrahydro-pyrido[3,4-d]pyrimidin-4-yl)-amine as an off-white solid (1.54 g, 3.57 mmol, 59%).

LCMS calculated. (M)⁺ 388.55, observed 388.6.

¹H NMR (CDCl₃) δ 8.39 (s, 1 H), 7.38-7.27 (m, 5 H), 4.56 (bs, 1H), 3.73 (bs, 2 H), 3.57 (bs, 2H), 3.26-3.24 (m, 2H), 2.83 (bs, 2H), 2.45 (bs, 2H), 1.74 (bs, 3H), 1.64-1.24 (m, 12H).

### Compound 326

### 3-[(7-Benzyl-5,6,7,8-tetrahydro-pyrido[3,4-d]pyrimidin-4-ylamino)-methyl]-adamantan-1-ol

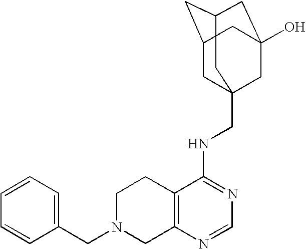

LCMS calculated. (M+1)⁺ 404.55, observed 405.2.

¹H NMR (CD₃OD) δ 8.12 (s, 1H), 7.48-7.32 (m, 5H), 3.78 (s, 2H), 3.38-3.32 (m, 4H), 2.88-2.78 (m, 2H), 2.52-2.48 (m, 2H), 2.18 (s, 2H), 1.68-1.47 (m, 12H).

### Method B

### Preparation of Representative Example

### Compound 4

### Adamantan-1-ylmethyl-(7-benzenesulfonyl-5,6,7,8-tetrahydro-pyrido[3,4-d]pyrimidin-4-yl)-amine

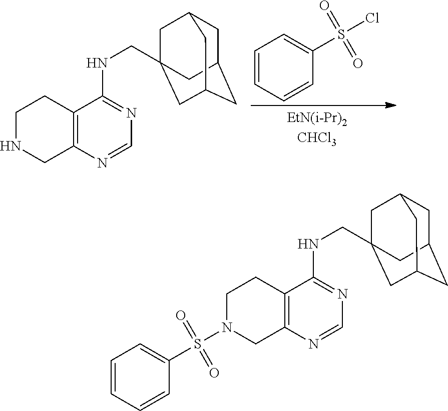

In one well of a 96-well polypropylene reaction plate was added 2-adamantan-1-ylmethyl-(5,6,7,8-tetrahydropyrido[3,4-d]pyrimidin-4-yl)amine (Intermediate 2, 2.9 mg, 10 μmol) in 100 μl of anhydrous chloroform. To the reaction was added benzenesulfonyl chloride (2.64 mg, 15 μmol), followed by di-isopropylethylamine (5.2 mg, 40 μmol). The reaction plate was heated at 50° C. for 15 minutes and the solvent was evaporated. The residue was dissolved in DMSO and purified using LC-MS based purification (50 mm×10 mm Phenomenex Gemini Column using a 30-100% acetonitrile-water gradient). ESI-MS m/z 439 (M+H)⁺.

### Method C

### Preparation of Representative Example

### Compound 77

### {4-[(Adamantan-1-ylmethyl)-amino]-5,8-dihydro-6H-pyrido[3,4-d]pyrimidin-7-yl}-phenyl-methanone

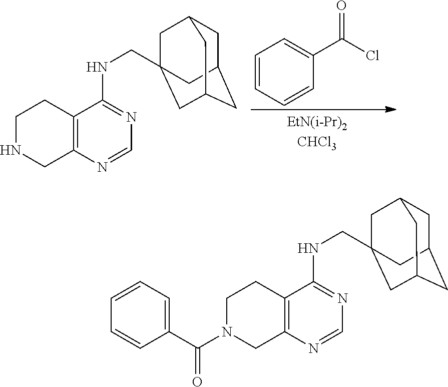

In one well of a 96-well polypropylene reaction plate was added adamantan-1-ylmethyl-(5,6,7,8-tetrahydropyrido[3,4-d]pyrimidin-4-yl)amine (Intermediate 2, 2.9 mg, 10 μmol) in 100 μl of anhydrous chloroform. To the reaction was added benzoyl chloride (2.1 mg, 15 μmol), followed by diisopropylethylamine (5.2 mg, 40 μmol). The reaction plate was heated at 50° C. for 15 minutes and the solvent was evaporated. The residue was dissolved in DMSO and purified using LC-MS based purification (50 mm×10 mm Phenomenex Gemini Column using a 10-100% acetonitrile-water gradient). ESI-MS m/z 403 (M+H)⁺.

### Method C1

### Preparation of Representative Examples

### Compound 248

### 1-{4-[(Adamantan-1-ylmethyl)-amino]-5,8-dihydro-6H-pyrido[3,4-d]pyrimidin-7-yl}-2-(3-chloro-phenoxy)-ethanone

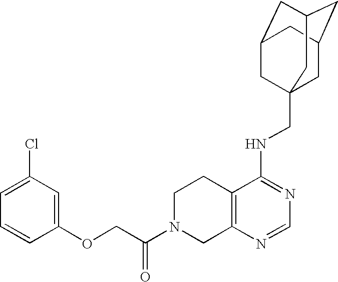

A mixture of 3-chlorophenoxyacetyl chloride (30 mg, 0.0001 mol) and methylene chloride (3 mL, 0.05 mol) was stirred at room temperature for 10 minutes and adamantan-1-ylmethyl-(5,6,7,8-tetrahydro-pyrido[3,4-d]pyrimidin-4-yl)-amine (Intermediate 2, 40 mg, 0.0001 mol) was added. The resulting mixture was stirred at room temperature for 15 hours. The mixture was poured onto a pad of silica gel, flash chromatographed over silica gel (12 g of silica gel, 0-10% methanol in dichloromethane gradient) to give 1-{4-[(adamantan-1-ylmethyl)-amino]-5,8-dihydro-6H-pyrido[3,4-d]pyrimidin-7-yl}-2-(3-chloro-phenoxy)-ethanone a white solid (34 mg, 50% yield).

LCMS (0.1% formic acid modifier) calculated. (M+1)⁺ 467.00, observed 468.8.

¹H NMR (CDCl₃) δ 8.44 (s, 1H), 7.21-7.16 (m, 1H), 6.98-6.92 (m, 3H), 4.74 (d, J=9.0 Hz, 2H), 4.64-4.59 (m, 2H), 3.95-3.96 (m, 2H), 3.27 (d, J=5.96 Hz, 2H), 2.46-2.43 (m, 2H), 2.04-2.00 (m, 3H), 1.75-1.26 (m, 12H).

### Compound 257

### 4-[(Adamantan-1-ylmethyl)-amino]-5,8-dihydro-6H-pyrido[3,4-d]pyrimidin-7-yl-pyridin-3-yl-methanone

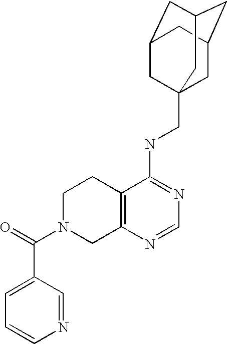

Into a 20 ml reaction vial was combined Niacin (41.2 mg, 0.000335 mol), oxalyl chloride (0.0284 mL, 0.000335 mol), methylene chloride (4 mL, 0.06 mol) and N,N-dimethylformamide (0.002 mL, 0.00003 mol). The mixture was stirred at room temperature for 20 minutes and heated at 50° C. for 10 minutes. The contents were reduced in vacuo and dried on high vacuum for 10 minutes. The resulting oil was dissolved in methylene chloride and adamantan-1-ylmethyl-(5,6,7,8-tetrahydro-pyrido[3,4-d]pyrimidin-4-yl)-amine (Intermediate 2, 80 mg, 0.0003 mol) was added followed by N,N-diisopropylethylamine (0.22 mL, 0.0012 mol). The reaction was stirred at room temperature overnight. The volatiles were removed under reduced pressure and the resulting oil was purified directly by column chromatography using a methanol:methylene chloride (0-10% ) gradient. The combined pure fractions were reduced in vacuo to yield the title compound as an off white solid. (0.079 mg, 70%)

LC-MS (M+H)=404.0

HNMR CDCl3 δ 8.74 (d, 1H), 8.70 (d,1H), 8.38 (brs, 1H), 7.80 (s, 1H), 7.39 (s,1H), 4.88-4.46 (m, 2H), 4.17-3.69 (m, 2H), 3.28 (d, 2H), 2.56 (brs, 2H), 2.00 (brs, 3H), 1.78-1.52 (m, 12H).

### Compound 503

### [4-(2-Adamantan-1-yl-2-hydroxyethylamino)-5,6,7,8-tetrahydropyrido[3,4-d]pyrimidin-7-yl]phenylmethanone

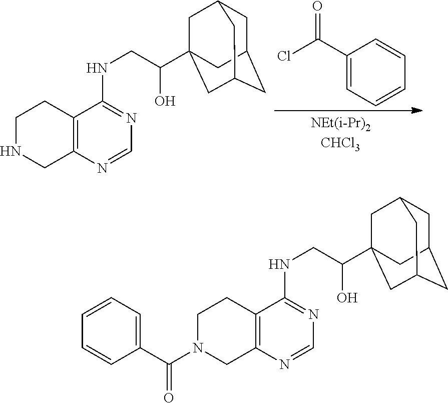

In one well of a 96-well polypropylene reaction plate was added 1-adamantan-1-yl-2-(5,6,7,8-tetrahydro-pyrido[3,4-d]pyrimidin-4-ylamino)-ethanol (Intermediate 5, 10 μmol) in 100 μl of anhydrous chloroform. The reaction was added benzoyl chloride (2.1 mg, 15 μmol), followed by diisopropylethylamine (5.2 mg, 40 μmol). The reaction plate was heated at 50° C. for 15 minutes and the solvent was evaporated. The residue was dissolved in DMSO and purified using LC-MS based purification (50 mm×10 mm Phenomenex Gemini Column using a 10-100% acetonitrile-water gradient) to give the pure [4-(2-adamantan-1-yl-2-hydroxy-ethylamino)-5,8-dihydro-6H-pyrido[3,4-d]pyrimidin-7-yl]-phenyl-methanone.

ESI-MS m/z 433 (M+H)⁺.

### Compound 687

### [4-(2-Adamantan-1-yl-2-hydroxy-ethylamino)-5,8-dihydro-6H-pyrido[3,4-d]pyrimidin-7-yl]-morpholin-4-yl-methanone

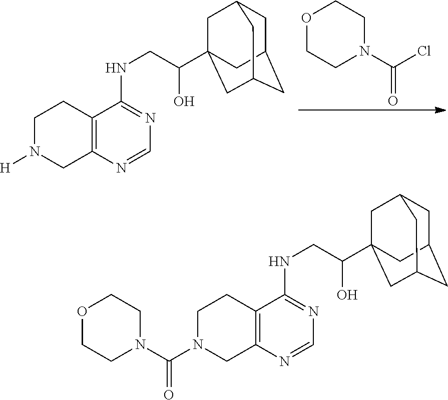

A mixture of 1-adamantan-1-yl-2-(5,6,7,8-tetrahydro-pyrido[3,4-d]pyrimidin-4-ylamino)-ethanol (Intermediate 5, 30 mg, 0.00009 mol), 4-morpholinecarbonyl chloride (16 mg, 0.00011 mol) and N,N-diisopropylethylamine (29 mg, 0.00022 mol) in methylene chloride (1 mL) was stirred at room temperature for 2 hours. The mixture was poured onto a pad of silica gel, purified via flash chromatography (4 g of silica gel, 0-10% methanol in dichloromethane gradient) to give [4-(2-Adamantan-1-yl-2-hydroxy-ethylamino)-5,8-dihydro-6H-pyrido[3,4-d]pyrimidin-7-yl]-morpholin-4-yl-methanone as a white solid (18 mg, 40% yield).

LCMS (0.1% formic acid modifier) calculated. (M+1)⁺ 441.57, observed 442.5.

¹H NMR (CDCl₃) δ 8.48 (s, 1H), 5.12 (br, 1H), 4.32 (s, 2H), 3.82-3.78 (m, 1H), 3.72-3.68 (m, 4H), 3.32-3.25 (m, 4H), 3.10-3.02 (m, 4H), 2.50 (s, 2H), 2.00 (s, 3H), 1.78-1.52 (m, 12H).

### Compound 700

### 1-[4-(2-Adamantan-1-yl-2-hydroxy-ethylamino)-5,8-dihydro-6H-pyrido[3,4-d]pyrimidin-7-yl]-2-(4-trifluoromethoxy-phenoxy)-ethanone

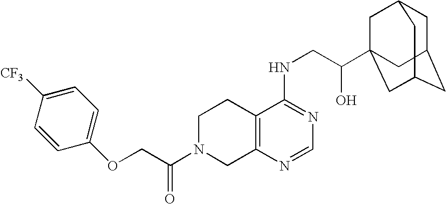

A mixture of 4-(trifluoromethoxy)phenoxyacetyl chloride (37 mg, 0.00015 mol), triethyl amine (40 mg, 0.0004 mol), and 1-adamantan-1-yl-2-(5,6,7,8-tetrahydro-pyrido[3,4-d]pyrimidin-4-ylamino)-ethanol (Intermediate 5, 40 mg, 0.0001 mol) in methylene chloride (3 mL) was stirred at room temperature for 15 hours. The mixture was poured onto a pad of silica gel, and flash chromatography (12 g of silica gel, 0-10% methanol in dichloromethane gradient) gave the product as a white solid (31 mg, 50% yield).

LCMS (0.1% formic acid modifier) calculated. (M+1)⁺ 546.58, observed 546.7.

¹H NMR (CDCl₃) δ 8.48 (s, 1H), 7.20-7.16 (m, 2H), 6.96-6.88 (m, 2H), 5.18 (br, 1H), 4.70 (s, 2H), 4.56-4.50 (m, 2H), 3.98-3.88 (m, 3H), 3.38-3.22 (m, 2H), 2.51-2.46 (m, 2H), 2.02 (s, 3H), 1.88-1.56 (m, 12H).

### Compound 701

### 1-[4-(2-Adamantan-1-yl-2-hydroxy-ethylamino)-5,8-dihydro-6H-pyrido[3,4-d]pyrimidin-7-yl]-2-(3-chloro-phenoxy)-ethanone

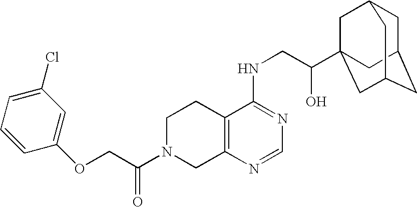

A mixture of 3-chlorophenoxyacetyl chloride (30 mg, 0.0001 mol), triethyl amine (40 mg, 0.0004 mol) and 1-adamantan-1-yl-2-(5,6,7,8-tetrahydro-pyrido[3,4-d]pyrimidin-4-ylamino)-ethanol (Intermediate 5, 40 mg, 0.0001 mol) in methylene chloride (3 mL) was stirred at room temperature for 15 hours. The mixture was poured onto a pad of silica gel, and flash chromatography (12 g of silica gel, 0-10% methanol in dichloromethane gradient) gave the product as a white solid (29 mg, 40% yield).

LCMS (0.1% formic acid modifier) calculated. (M+1)⁺ 497.03, observed 498.3.

¹H NMR (CDCl₃) δ 8.48 (s, 1H), 7.20-6.88 (m, 4H), 4.66-4.52 (m, 3H), 3.90-3.82 (m, 2H), 3.34-3.22 (m, 2H), 2.54-2.48 (m, 2H), 2.00 (s, 3H), 1.76-1.45 (m, 14H).

### Compound 703

### [4-(2-Adamantan-1-yl-2-hydroxy-ethylamino)-5,8-dihydro-6H-pyrido[3,4-d]pyrimidine-7-yl]-(S)-pyrrolidin-3-yl-methanone

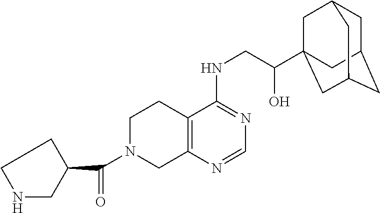

A mixture of 1 M HCl ethyl ether solution (3 mL, 0.003 mol) and (S)-3-[4-(2-adamantan-1-yl-2-hydroxy-ethylamino)-5,8-dihydro-6H-pyrido[3,4-d]pyrimidine-7-carbonyl]-pyrrolidine-1-carboxylic acid tert-butyl ester (23 mg, 0.000044 mol) was stirred at room temperature for 15 hours. The mixture was poured onto a pad of silica gel and flash chromatography (4 g of silica gel, 0-10% methanol in dichloromethane gradient) gave the product as a white solid (18 mg, 93% yield).

LCMS (0.1% formic acid modifier) calculated. (M+1)⁺ 425.57, observed 426.0.

¹H NMR (CDCl₃) δ 8.48 (s, 1H), 5.21 (br, 1H), 4.52-4.48 (m, 1H), 4.04-3.68 (m, 4H), 3.32-3.08 (m, 3H), 2.88-2.76 (m, 1H), 2.56-2.46 (m, 6H), 2.18-2.10 (m, 1H), 2.00 (s, 3H), 1.88-1.56 (m, 12H).

### Method C2

### Preparation of Representative Examples

### Compound 115

### 4-[(Adamantan-1-ylmethyl)-amino]-5,8-dihydro-6H-pyrido[3,4-d]pyrimidin-7-yl-cyclopropyl-methanone

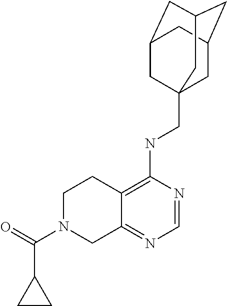

Into a 20 ml reaction vial was combined adamantan-2-ylmethyl-(5,6,7,8-tetra hydro-pyrido[3,4-d]pyrimidin-4-yl)-amine (Intermediate 2, 0.02 g, 0.00009 mol) 1-hydroxybenzotriazole hydrate (0.020 g, 0.00013 mol), N-(3-dimethylaminopropyl)-N′-ethylcarbodiimide hydrochloride (0.025 g, 0.00013 mol), methylene chloride (2 mL, 0.03 mol) N,N-diisopropylethylamine (0.11 mL, 0.00061 mol) and cyclopropanecarboxylic acid (1.0El uL, 0.00013 mol), The mixture was stirred at room temperature overnight. The volatiles were removed under reduced pressure and the resulting oil was taken up in methylene chloride (20 ml) and washed with brine (1×20 ml) and satd. sodium bicarbonate (1×20 ml). The organic was dried over sodium sulfate and reduced in vacuo. The mixture was chromatographed using a methanol: methylene chloride (0-10%) gradient on an Isco flash chromatography system. The combined pure fractions were reduced in vacuo to yield the title compound as an off white solid. (18.1 mg, 60%)

LC-MS (M+H)=366.9

¹H NMR CDCl3 δ 8.45 (s, 1H), 4.75-4.51(m,3H), 4.01-3.89(m,2H), 3.27(d,2H), 2.59-2.39(m,2H), 2.03(brs,3H), 1.86-1.77(m,1H), 1.76-1.52 (m,12H), 1.07-0.98(m,2H), 0.85-0.79(m,2H)

### Compound 249

### {4-[(Adamantan-1-ylmethyl)-amino]-5,8-dihydro-6H-pyrido[3,4-d]pyrimidin-7-yl}-(tetrahydro-furan-3-yl)-methanone

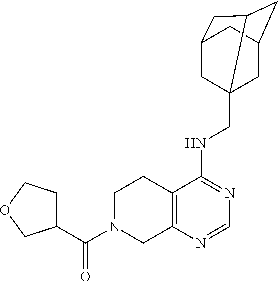

A mixture of tetrahydro-3-furoic acid (17 mg, 0.00015 mol), N-(3-dimethylaminopropyl)-N′-ethylcarbodiimide hydrochloride (28 mg, 0.00015 mol), 1-hydroxybenzotriazole hydrate (22 mg, 0.00015 mol) and N,N-diisopropylethylamine (80 mg, 0.0006 mol) in methylene chloride (3 mL, 0.05 mol) was stirred at room temperature for 10 minutes and adamantan-1-ylmethyl-(5,6,7,8-tetrahydro-pyrido[3,4-d]pyrimidin-4-yl)-amine (Intermediate 2, 40 mg, 0.0001 mol) was added. The resulting mixture was stirred at room temperature for 15 hours. The mixture was poured onto a pad of silica gel and flash chromatography over silica gel (12 g of silica gel, 0-10% methanol in dichloromethane gradient) gave {4-[(adamantan-1-ylmethyl)-amino]-5,8-dihydro-6H-pyrido[3,4-d]pyrimidin-7-yl}-(tetrahydro-furan-3-yl)-methanone as a white solid (40 mg, 80% yield).

LCMS (0.1% formic acid modifier) calculated. (M+1)⁺ 396.53, observed 396.6.

¹H NMR (CDCl₃) δ 8.49 (s, 1H), 4.54-4.51 (m, 3H), 3.99-3.82 (m, 6H), 3.32-3.20 (m, 3H), 2.51-2.48 (m, 2H), 2.23-2.10 (m, 1H), 2.01-1.98 (m, 3H), 1.87-1.47 (m, 12H).

### Compound 251

### 1-{4-[(Adamantan-1-ylmethyl)-amino]-5,8-dihydro-6H-pyrido[3,4-d]pyrimidin-7-yl}-2-(4-trifluoromethoxy-phenoxy)-ethanone

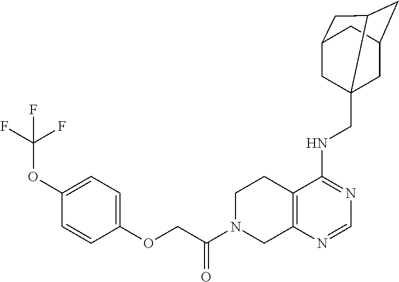

The title compounds was prepared similarly to 1-{4-[(adamantan-1-ylmethyl)-amino]-5,8-dihydro-6H-pyrido[3,4-d]pyrimidin-7-yl}-2-(3-chloro-phenoxy-ethanone using the appropriate reagents.

LCMS calculated. (M+1)⁺ 516.56, observed 516.7.

¹H NMR (CDCl₃) δ 8.44 (s, 1H), 7.15-7.11 (m, 2H), 6.99-6.91 (m, 2H), 4.77-4.75 (m, 2H), 4.64-4.59 (m, 2H), 3.95-3.89 (m, 2H), 3.27 (d, J=6.0 Hz, 2H), 2.46-2.43 (m, 2H), 2.00 (s, 3H), 1.75-1.53 (m, 12H).

### Compound 350

### {4-[(3-Hydroxy-adamantan-1-ylmethyl)-amino]-5,8-dihydro-6H-pyrido[3,4-d]pyrimidin-7-yl}-pyridin-3-yl-methanone

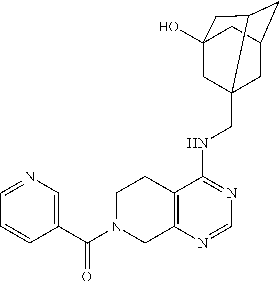

3-[(5,6,7,8-Tetrahydro-pyrido[3,4-d]pyrimidin-4-ylamino)-methyl]-adamantan-1-ol (Intermediate 5, 100 mg, 0.0003 mol), Niacin (59 mg, 0.00048 mol), N-(3-dimethylaminopropyl)-N′-ethylcarbodiimide hydrochloride (91 mg, 0.00048 mol), 1-hydroxybenzotriazole hydrate (73 mg, 0.00048 mol) and N,N-diisopropylethylamine (200 mg, 0.0016 mol) were stirred in methylene chloride (4 mL, 0.06 mol) at room temperature for 16 hours. The mixture was purified via flash chromatography (1.2 g of silica gel, 0-10% MeOH/CH₂Cl₂ gradient) to give {4-[(3-hydroxy-adamantan-1-ylmethyl)-amino]-5,8-dihydro-6H-pyrido[3,4-d]pyrimidin-7-yl}-pyridin-3-yl-methanone as a white solid (96 mg, 70% yield).

LCMS calculated. (M+1)⁺ 419.52, observed 419.7.

¹H NMR (CDCl₃) δ 8.67-8.62 (m, 2H), 8.44-8.41 (m, 1H), 7.88-7.70 (m, 1H), 7.41-7.37 (m, 1H), 4.88-4.61 (m, 3H), 4.21-4.15 (m, 1H), 3.41-3.32 (m, 2H), 2.58-2.51 (m, 2H), 2.21 (s, 2H), 1.66-1.41 (m, 12H).

### Compound 686

### 1-[4-(2-Adamantan-1-yl-2-hydroxy-ethylamino)-5,8-dihydro-6H-pyrido[3,4-d]pyrimidin-7-yl]-3,3,3-trifluoro-propan-1-one

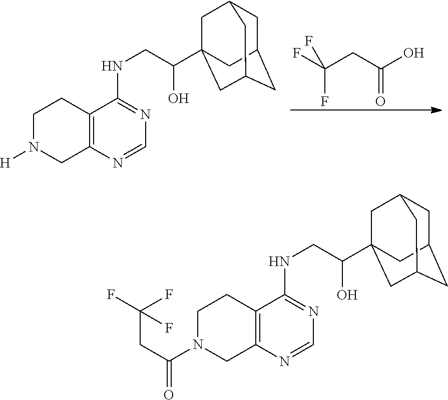

A mixture of 3,3,3-trifluoropropionic acid (13.0 mg, 0.000102 mol), N-(3-dimethylaminopropyl)-N′-ethylcarbodiimide hydrochloride (21.0 mg, 0.000110 mol), 1-hydroxybenzotriazole hydrate (17.0 mg, 0.000111 mol) and N,N-diisopropylethylamine (23.6 mg, 0.000183 mol) in methylene chloride (3 mL) was stirred at room temperature for 10 minutes and 1-adamantan-1-yl-2-(5,6,7,8-tetrahydro-pyrido[3,4-d]pyrimidin-4-ylamino)-ethanol (Intermediate 5, 30.0 mg, 0.0000913 mol) was added. The resulting mixture was stirred at room temperature for 15 hours. The mixture was poured onto a pad of silica gel and flash chromatographed (4 g of silica gel, 0-10% methanol in dichloromethane gradient) to give the product as a white solid (16 mg, 38% yield).

LCMS (0.1% formic acid modifier) calculated. (M+1)⁺ 438.49, observed 439.3.

¹H NMR (CDCl₃) δ 8.48 (s, 1H), 4.67 (s, 1H), 4.50 (s, 2H), 4.01-3.68 (m, 2H), 3.46-3.23 (m, 4H), 2.49-2.46 (m, 2H), 2.01 (s, 3H), 1.78-1.48 (m, 12 H).

### Compound 690

### (R)-2-[4-(2-Adamantan-1-yl-2-hydroxy-ethylamino)-5,8-dihydro-6H-pyrido[3,4-d]pyrimidine-7-carbonyl]-pyrrolidine-1-carboxylic acid tert-butyl ester

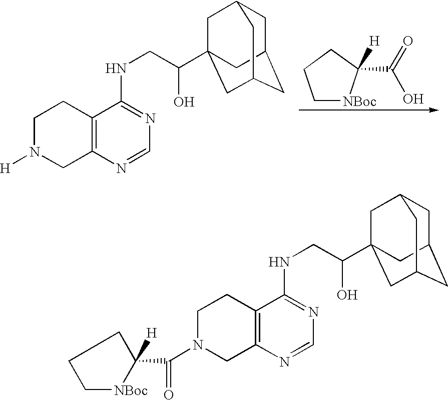

A mixture of N-(tert-butoxycarbonyl)-D-proline (21.8 mg, 0.000102 mol), N-(3-dimethylaminopropyl)-N′-ethylcarbodiimide hydrochloride (21.0 mg, 0.000110 mol), 1-hydroxybenzotriazole hydrate (17.0 mg, 0.000111 mol) and N,N-diisopropylethylamine (23.6 mg, 0.000183 mol) in methylene chloride (3 mL) was stirred at room temperature for 10 minutes and 1-adamantan-1-yl-2-(5,6,7,8-tetrahydro-pyrido[3,4-d]pyrimidin-4-ylamino)-ethanol (Intermediate 5, 30.0 mg, 0.0000913 mol) was added. The resulting mixture was stirred at room temperature for 15 hours. The mixture was poured onto a pad of silica gel, flash chromatography over silica gel (4 g of silica gel, 0-10% methanol in dichloromethane gradient) to give (R)-2-[4-(2-Adamantan-1-yl-2-hydroxy-ethylamino)-5,8-dihydro-6H-pyrido[3,4-d]pyrimidine-7-carbonyl]-pyrrolidine-1-carboxylic acid tert-butyl ester as a white solid (40 mg, 80% yield).

LCMS (0.1% formic acid modifier) calculated. (M−1)⁻ 525.68, observed 524.5.

¹H NMR (CDCl₃) δ 8.49 (s, 1H), 5.12 (br, 1H), 4.63-4.51 (m, 3H), 4.05-3.32 (m, 6H), 2.52 (s, 2H), 2.00 (s, 3H), 1.78-1.52 (m, 16H), 1.50 (s, 9H).

### Compound 695

### [4-(2-Adamantan-1-yl-2-hydroxy-ethylamino)-5,8-dihydro-6H-pyrido[3,4-d]pyrimidin-7-yl]-(tetrahydro-pyran-4-yl)-methanone

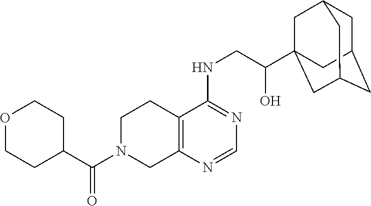

A mixture of tetrahydro-pyran-4-carboxylic acid (19 mg, 0.00015 mol), N-(3-Dimethylaminopropyl)-N′-ethylcarbodiimide hydrochloride (28 mg, 0.00015 mol), 1-Hydroxybenzotriazole-hydrate (22 mg, 0.00015 mol) and N,N-Diisopropylethylamine (80 mg, 0.0006 mol) in methylene chloride (3 mL) was stirred at room temperature for 10 minutes and 1-Adamantan-1-yl-2-(5,6,7,8-tetrahydro-pyrido[3,4-d]pyrimidin-4-ylamino)-ethanol (40 mg, 0.0001 mol) was added. The resulting mixture was stirred at room temperature for 15 hours. The mixture was poured onto a pad of silica gel, flash chromatographed (12 g of silica gel, 0-10% methanol in dichloromethane gradient) to give the product as a white solid (33 mg, 60% yield).

LCMS (0.1% formic acid modifier) calculated. (M+1)⁺ 440.50, observed 441.6.

¹H NMR (CDCl₃) δ 8.48 (s, 1H), 5.13 (br, 1H), 4.50 (s, 2H), 4.01-3.88 (m, 5H), 3.51-3.22 (m, 4H), 2.92-2.76 (m, 2H), 2.53-2.48 (m, 2H), 2.00-1.51 (m, 17H).

### Compound 699

### [4-(2-Adamantan-1-yl-2-hydroxy-ethylamino)-5,8-dihydro-6H-pyrido[3,4-d]pyrimidin-7-yl]-(tetrahydrofuran-3-yl)-methanone

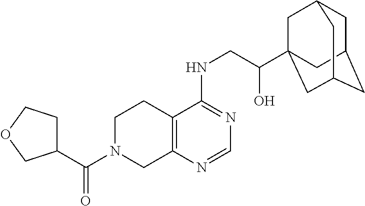

A mixture of Tetrahydro-3-furoic acid (17 mg, 0.00015 mol), N-(3-Dimethylaminopropyl)-N′-ethylcarbodiimide hydrochloride (28 mg, 0.00015 mol), 1-Hydroxybenzotriazole hydrate (22 mg, 0.00015 mol) and N,N-Diisopropylethylamine (80 mg, 0.0006 mol) in methylene chloride (3 mL) was stirred at room temperature for 10 minutes and 1-adamantan-1-yl-2-(5,6,7,8-tetrahydro-pyrido[3,4-d]pyrimidin-4-ylamino)-ethanol (Intermediate 5, 40 mg, 0.0001 mol) was added. The resulting mixture was stirred at room temperature for 15 hours. The mixture was poured onto a pad of silica gel and flash chromatographed (12 g of silica gel, 0-10% methanol in dichloromethane gradient) to give the product as a white solid (30 mg, 60% yield).

LCMS (0.1% formic acid modifier) calculated. (M+1)⁺ 426.55, observed 426.5.

### Compound 702

### (R)-1-{2-[4-(2-Adamantan-1-yl-2-hydroxy-ethylamino)-5,8-dihydro-6H-pyrido[3,4-d]pyrimidin-7-yl]-2-oxo-ethyl}-4-hydroxy-pyrrolidin-2-one

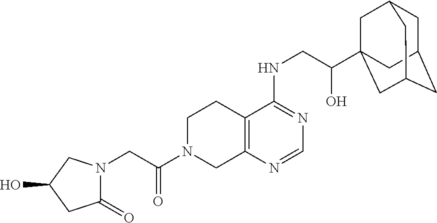

A mixture of (R)-(4-hydroxy-2-oxo-pyrrolidin-1-yl)-acetic acid (20 mg, 0.0001 mol), N-(3-Dimethylaminopropyl)-N′-ethylcarbodiimide hydrochloride (28 mg, 0.00015 mol), 1-Hydroxybenzotriazole hydrate (22 mg, 0.00015 mol) and N,N-Diisopropylethylamine (80 mg, 0.0006 mol) in methylene chloride (3 mL) was stirred at room temperature for 10 minutes and 1-adamantan-1-yl-2-(5,6,7,8-tetrahydro-pyrido[3,4-d]pyrimidin-4-ylamino)-ethanol (Intermediate 5, 40 mg, 0.0001 mol) was added. The resulted mixture was stirred at room temperature for 15 hours. The mixture was poured onto a pad of silica gel and flash chromatographed (12 g of silica gel, 0-10% methanol in dichloromethane gradient), followed by preparative TLC to yield the product as a white solid (16 mg, 20% yield).

LCMS (0.1% formic acid modifier) calculated. (M+1)⁺ 469.58, observed 469.9.

¹H NMR (CD₃OD) δ 8.32 (s, 1H), 4.53-4.34 (m, 4H), 4.21-4.14 (m, 1H), 3.89-3.72 (m, 4H), 3.32-3.22 (m, 4H), 2.86-2.23 (m, 4H), 2.00 (s, 3H), 1.76-1.56 (m, 12H).

### Compound 704

### [4-(2-Adamantan-1-yl-2-hydroxy-ethylamino)-5,8-dihydro-6H)-pyrido[3,4-d]pyrimidin-7-yl]-(R)-2,3-dihydro-benzo[1,4]dioxin-2-yl-methanone

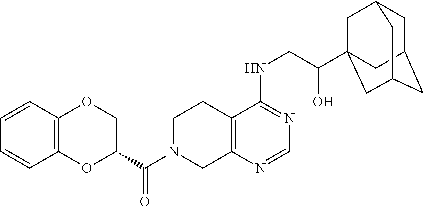

1-Adamantan-1-yl-2-(5,6,7,8-tetrahydro-pyrido[3,4-d]pyrimidin-4-ylamino)-ethanol (Intermediate 5, 40 mg, 0.0001 mol), (R)-1,4-benzodioxane-2-carboxylic acid (26 mg, 0.00015 mol), N-(3-dimethylaminopropyl)-N′-ethylcarbodiimide hydrochloride (28 mg, 0.00015 mol), 1-hydroxybenzotriazole hydrate (22 mg, 0.00015 mol) and N,N-diisopropylethylamine (63 mg, 0.00049 mol) were stirred in methylene chloride (3 mL) at room temperature for 15 hours. The mixture was purified by flash chromatography (12 g of silica gel, 0-10% methanol in dichloromethane gradient) to give the desired product as a white solid (25 mg, 40% yield).

LCMS (0.1% formic acid modifier) calculated. (M+1)⁺ 490.59, observed 491.3.

¹H NMR (CDCl₃) δ 8.45 (s, 1H), 6.91-6.86 (m, 4H), 5.09-4.11 (m, 6 H), 3.88-3.75 (m, 2H), 3.36-3.29 (m, 2H), 3.04-3.01 (m, 2H), 2.00 (s, 3H), 1.76-1.58 (m, 12H).

### Compound 705

### [4-(2-Adamantan-1-yl-2-hydroxy-ethylamino)-5,8-dihydro-6H-pyrido[3,4-d]pyrimidin-7-yl]-(S)-2,3-dihydro-benzo[1,4]dioxin-2-yl-methanone

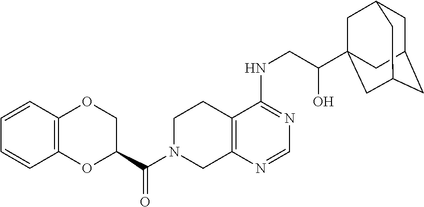

1-Adamantan-1-yl-2-(5,6,7,8-tetrahydro-pyrido[3,4-d]pyrimidin-4-ylamino)-ethanol (Intermediate 5, 45 mg, 0.00014 mol), (S)-2,3-dihydro-benzo[1,4]dioxine-2-carboxylic acid (26 mg, 0.00015 mol), N-(3-dimethylaminopropyl)-N′-ethylcarbodiimide hydrochloride (28 mg, 0.00015 mol), 1-hydroxybenzotriazole hydrate (22 mg, 0.00015 mol) and N,N-diisopropylethylamine (60 mg, 0.0005 mol) were stirred in methylene chloride (3 mL) at room temperature for 15 hours. The mixture was purified by flash chromatography (12 g of silica gel, 0-10% methanol in dichlorohaethane gradient) to give the desired product as a white solid (65 mg, 96% yield).

LCMS (0.1% formic acid modifier) calculated. (M+1)⁺ 490.59, observed 491.3.

¹H NMR (CDCl₃) δ 8.45 (s, 1H), 6.91-6.86 (m, 4H), 5.09-4.11 (m, 6H), 3.88-3.75 (m, 2H), 3.36-3.29 (m, 2H), 3.04-3.01 (m, 2H), 2.00 (s, 3H), 1.76-1.58 (m, 12H).

### Compound 706

### (S)-1-{2-[4-(2-Adamantan-1-yl-2-hydroxy-ethylamino)-5,8-dihydro-6H-pyrido[3,4-d]pyrimidin-7-yl]-2-oxo-ethyl}-4-hydroxy-pyrrolidin-2-one

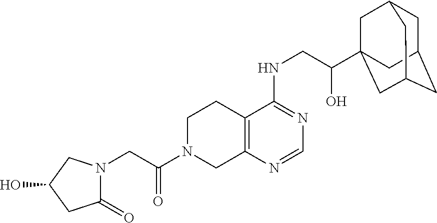

1-Adamantan-1-yl-2-(5,6,7,8-tetrahydro-pyrido[3,4-d]pyrimidin-4-ylamino)-ethanol (Intermediate 5, 34 mg, 0.00010 mol), (s)-(4-hydroxypyrrolidin-1-yl)-acetic acid (20 mg, 0.00012 mol), N-(3-Dimethylaminopropyl)-N′-ethylcarbodiimide hydrochloride (30 mg, 0.00016 mol), 1-Hydroxybenzotriazole hydrate (24 mg, 0.00016 mol) and N,N-Diisopropylethylamine (100 mg, 0.0008 mol) were stirred in methylene chloride (3 mL) at room temperature for 15 hours. The reaction mixture was purified via flash chromatography (12 g of silica gel, 0-10% methanol in dichloromethane gradient), followed by preparative TLC to give the desired product as a white solid (5 mg, 10% yield).

LCMS (0.1% formic acid modifier) calculated. (M+1)⁺ 469.58, observed 469.8.

¹H NMR (CDCl₃) δ 8.31 (s, 1H), 4.56-4.41 (m, 4H), 4.29-4.23 (m, 1H), 3.90-3.80 (m, 4H), 3.38-3.27 (m, 4H), 2.81-2.23 (m, 4H), 2.01 (s, 3H), 1.81-1.65 (m, 12H).

### Compound 707

### {2-[4-(2-Adamantan-1-yl-2-hydroxy-ethylamino)-5,8-dihydro-6H-pyrido[3,4d]pyrimidin-7-yl]-2-oxo-ethyl}-carbamic acid tert-butyl ester

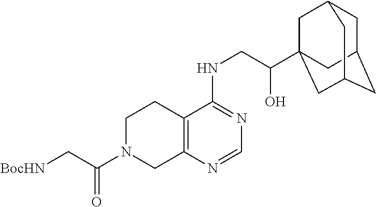

1-Adamantan-1-yl-2-(5,6,7,8-tetrahydro-pyrido[3,4-d]pyrimidin-4-ylamino)-ethanol (Intermediate 5, 60 mg, 0.0002 mol), N-α-(tert-Butoxycarbonyl)glycine (38 mg, 0.00022 mol), 1-Hydroxybenzotriazole hydrate (34 mg, 0.00022 mol), N-(3-Dimethylaminopropyl)-N′-ethylcarbodiimide hydrochloride (42 mg, 0.00022 mol) and N,N-Diisopropylethylamine (200 mg, 0.001 mol) were dissolved in methylene chloride (5 mL). The mixture was stirred at room temperature for 16 hours. The mixture was purified via flash chromatography (12 g of silica gel, 0-10% methanol in dichloromethane gradient) to give the desired product as a white solid 71 mg, 80% yield).

LCMS (0.1% formic acid modifier) calculated. (M+1)⁺ 485.62, observed 486.3.

¹H NMR (CDCl₃) δ 8.42 (s, 1H), 5.52-5.48 (m, 1H), 5.06-5.00 (m, 1H), 4.00-3.86 (m, 2H), 3.22-3.12 (m, 2H), 3.08-3.00 (m, 2H), 2.58-2.51 (m, 4H), 2.00 (s, 3H), 1.76-1.52 (m, 12H), 1.48 (s, 9H).

### Compound 708

### {(S)-2-[4-(2-Adamantan-1-yl-2-hydroxy-ethylamino)-5,8-dihydro-6H-pyrido[3,4-d]pyrimidin-7-yl]-1-methyl-2-oxo-ethyl}-carbamic acid tert-butyl ester

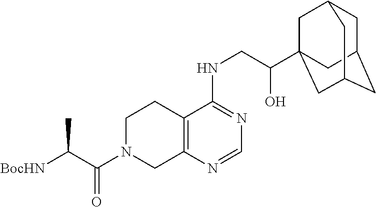

1-Adamantan-1-yl-2-(5,6,7,8-tetrahydro-pyrido[3,4-d]pyrimidin-4-ylamino)-ethanol (Intermediate 5, 60 mg, 0.00018 mol), N-(tert-Butoxycarbonyl)-L-alanine (52 mg, 0.00027 mol), 1-Hydroxybenzotriazole hydrate (42 mg, 0.00027 mol), N-(3-Dimethylaminopropyl)-N′-ethylcarbodiimide hydrochloride (52 mg, 0.00027 mol) and N,N-Diisopropylethylamine (120 mg, 0.00091 mol) were dissolved in methylene chloride (5 mL). The mixture was stirred at room temperature for 16 hours and then purified via flash chromatography (12 g of silica gel, 0-10% methanol in dichloromethane gradient) to give the desired product as a white solid (76 mg, 83% yield).

LCMS (0.1% formic acid modifier) calculated. (M+1)⁺ 499.65, observed 500.7.

¹H NMR (CDCl₃) δ 8.43 (s, 1H), 5.52-5.50 (m, 1H), 5.08-5.02 (m, 1H), 4.68-4.46 (m, 2H), 3.88-3.79 (m, 2H), 3.38-3.25 (m, 2H), 2.51-2.46 (m, 2H), 2.00 (s, 3H), 1.68-1.56 (m, 12H), 1.48 (s, 9H), 1.47-1.38 (m, 3H).

### Compound 709

### {(S)-2-[4-(2-Adamantan-1-yl-2-hydroxy-ethylamino)-5,8-dihydro-6H-pyrido[3,4-d]pyrimidin-7-yl]-1-benzyl-2-oxo-ethyl}-carbamic acid tert-butyl ester

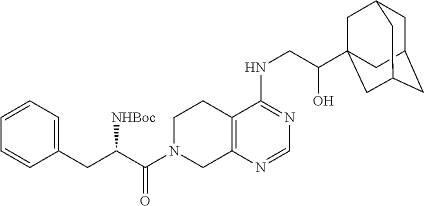

1-Adamantan-1-yl-2-(5,6,7,8-tetrahydro-pyrido[3,4-d]pyrimidin-4-ylamino)-ethanol (Intermediate 5, 60 mg, 0.0002 mol), N-(tert-Butoxycarbonyl)-L-Phenylalanine (73 mg, 0.00027 mol), 1-Hydroxybenzotriazole hydrate (42 mg, 0.00027 mol), N-(3-Dimethylaminopropyl)-N′-ethylcarbodiimide hydrochloride (52 mg, 0.00027 mol) and N,N-Diisopropylethylamine (120 mg, 0.00091 mol) were dissolved in methylene chloride (5 mL). The mixture was stirred at room temperature for 18 hours, purified via chromatography (12 g of silica gel, 0-10% methanol in dichloromethane gradient) to give the desired product as a white solid (82 mg, 80% yield).

LCMS (0.1% formic acid modifier) calculated. (M+1)⁺ 575.74, observed 576.8.

¹H NMR (CDCl₃) δ 8.43 (s, 0.5H), 8.36 (s, 0.5H), 7.24-7.08 (m, 5H), 5.02-4.82 (m, 1H), 4.00-3.92 (m, 1H), 3.32-3.22 (m, 2H), 3.06-2.98 (m, 6H), 2.52-2.46 (m, 2H), 2.02-1.96 (m, 3H), 1.76-1.58 (m, 12H), 1.46 (s, 9H).

### Compound 710

### {(R)-2-[4-(2-Adamantan-1-yl-2-hydroxy-ethylamino)-5,8-dihydro-6H-pyrido[3,4-d]pyrimidin-7-yl]-1-methyl-2-oxo-ethyl}-carbamic acid tert-butyl ester

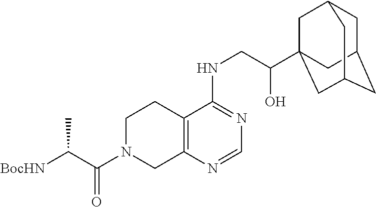

1-Adamantan-1-yl-2-(5,6,7,8-tetrahydro-pyrido[3,4-d]pyrimidin-4-ylamino)-ethanol (Intermediate 5, 60 mg, 0.0002 mol), N-(tert-Butoxycarbonyl)-D-alanine (52 mg, 0.00027 mol), 1-Hydroxybenzotriazole hydrate (42 mg, 0.00027 mol), N-(3-Dimethylaminopropyl)-N′-ethylcarbodiimide hydrochloride (52 mg, 0.00027 mol) and N,N-Diisopropylethylamine (120 mg, 0.00091 mol) were dissolved in methylene chloride (5 mL). The mixture was stirred at room temperature for 18 hours. Purification via flash chromatography (12 g of silica gel, 0-10% methanol in dichlorometlane gradient) gave the desired product as a white solid (73 mg, 80% yield).

LCMS (0.1% formic acid modifier) calculated. (M+1)⁺ 499.65, observed 500.0.

¹H NMR (CDCl₃) δ 8.43 (s, 1H), 5.52-5.50 (m, 1H), 5.08-5.02 (m, 1H), 4.68-4.46 (m, 2H), 3.88-3.79 (m, 2H), 3.38-3.25 (m, 2H), 2.51-2.46 (m, 2H), 2.00 (s, 3H), 1.68-1.56 (m, 12H), 1.48 (s, 9H), 1.47-1.38 (m, 3H).

### Compound 711

### {(R)-2-[4-(2-Adamantan-1-yl-2-hydroxy-ethylamino)-5,8-dihydro-6H-pyrido[3,4-d]pyrimidin-7-yl]-1-benzyl-2-oxo-ethyl}-carbamic acid tert-butyl ester

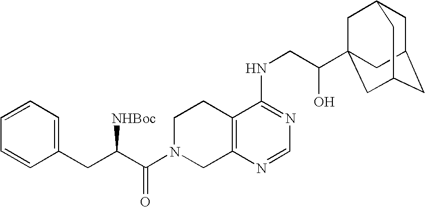

1-Adamantan-1-yl-2-(5,6,7,8-tetrahydro-pyrido[3,4-d]pyrimidin-4-ylamino)-ethanol (Intermediate 5, 60 mg, 0.0002 mol), N-(tert-Butoxycarbonyl)-D-phenylalanine (73 mg, 0.00027 mol), 1-Hydroxybenzotriazole hydrate (42 mg, 0.00027 mol), N-(3-Dimethylaminopropyl)-N′-ethylcarbodiimide hydrochloride (52 mg, 0.00027 mol) and N,N-Diisopropylethylamine (35 mg, 0.00027 mol) were dissolved in methylene chloride (5 mL). The mixture was stirred at room temperature for 18 hours. Purification via flash chromatography (12 g of silica gel, 0-10% MeOH/CH₂Cl₂ gradient) to give the desired product as a white solid (64 mg, 60% yield).

LCMS (0.1% formic acid modifier) calculated. (M+1)⁺ 575.74, observed 576.5.

¹H NMR (CDCl₃) δ 8.43 (s, 0.5H), 8.36 (s, 0.5H), 7.24-7.08 (m, 5H), 5.02-4.82 (m, 1H), 4.00-3.92 (m, 1H), 3.32-3.22 (m, 2H), 3.06-2.98 (m, 6H), 2.52-2.46 (m, 2H), 2.02-1.96 (m, 3H), 1.76-1.58 (m, 12H), 1.46 (s, 9H).

### Compound 716

### 1-[4-(2-Adamantan-1-yl-2-hydroxy-ethylamino)-5,8-dihydro-6H-pyrido[3,4-d]pyrimidin-7-yl]-2-morpholin-4-yl-ethanone

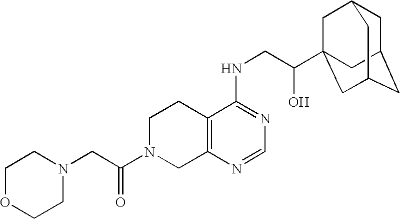

Morpholin-4-yl-acetic acid (26 mg, 0.00018 mol), 1-Adamantan-1-yl-2-(5,6,7,8-tetrahydro-pyrido[3,4-d]pyrimidin-4-ylamino)-ethanol (Intermediate 5, 40 mg, 0.0001 mol), N-3-Dimethylaminopropyl)-N′-ethylcarbodiimide hydrochloride (35 mg, 0.00018 mol), 1-Hydroxybenzotriazole hydrate (28 mg, 0.00018 mol) and N,N-Diisopropylethylamine (120 mg, 0.00091 mol) were stirred in methylene chloride (3 mL) at room temperature over night. The mixture was purified via flash chromatography (12 g of silica gel, 0-10% MeOH/CH₂Cl₂ gradient) to yield a white solid (54 mg, 90% yield).

LCMS (0.1% formic acid modifier) calculated. (M+1)⁺ 455.59, observed 456.1.

¹H NMR (CDCl₃) δ8.48 (s, 1H), 5.12 (br, 1H), 4.62-4.56 (m, 2H), 3.96-3.88 (m, 3H), 3.69-3.58 (m, 4H), 3.48-3.26 (m, 4H), 2.53-2.48 (m, 6H), 2.00 (s, 3H), 1.76-1.62 (m, 12H).

### Compound 718

### 1-[4-(2-Adamantan-1-yl-2-hydroxy-ethylamino)-5,8-dihydro-6H-pyrido[3,4-d]pyrimidin-7-yl]-2-dimethylamino-ethanone

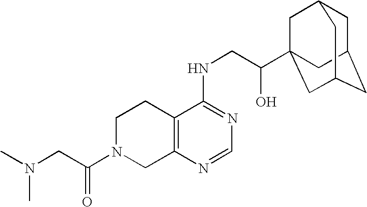

N,N-dimethyl glycine (19 mg, 0.00018 mol), 1-adamantan-1-yl-2-(5,6,7,8-tetrahydro-pyrido[3,4-d]pyrimidin-4-ylamino)-ethanol (Intermediate 5, 50 mg, 0.0002 mol), N-(3-dimethylaminopropyl)-N′-ethylcarbodiimide hydrochloride (35 mg, 0.00018 mol), 1-hydroxybenzotriazole hydrate (28 mg, 0.00018 mol) and N,N-diisopropylethylamine (100 mg, 0.0008 mol) were stirred in Methylene chloride (3 mL) at room temperature for 18 hours. The mixture was purified via flash chromatography (12 g of silica gel, 0-10% MeOH/CH2Cl2 gradient) to give a white solid (56 mg, 74% yield).

LCMS (0.1% formic acid modifier) calculated. (M+1)⁺ 413.56, observed 414.5.

¹H NMR (CDCl₃) δ 8.46 (s, 1H), 5.42 (br, 1H), 4.52-4.48 (m, 2H), 3.90-3.42 (m, 8H), 2.52-2.38 (m, 7H), 2.00 (s, 3H), 1.75-1.61 (m, 12H).

### Compound 719

### 1-[4-(2-Adamantan-1-yl-2-hydroxy-ethylamino)-5,8-dihydro-6H-pyrido[3,4-d]pyrimidin-7-yl]-2-(4-isopropyl-piperazin-1-yl)-ethanone

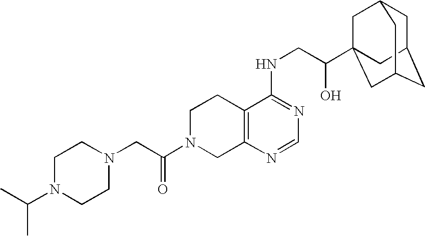

1-Adamantan-1-yl-2-(5,6,7,8-tetrahydro-pyrido[3,4-d]pyrimidin-4-ylamino)-ethanol (Intermediate 5, 50 mg, 0.0002 mol), (4-isopropyl-piperazin-1-yl)-acetic acid (34 mg, 0.00018 mol), N-(3-dimethylaminopropyl)-N′-ethylcarbodiimide hydrochloride (35 mg, 0.00018 mol), 1-hydroxybenzotriazole hydrate (28 mg, 0.00018 mol) and N,N-diisopropylethylamine (98 mg, 0.00076 mol) were stirred in Methylene chloride (3 mL) at room temperature for 18 hours. The mixture was purified via flash chromatography (12 g of silica gel, 15% MeOH/CH₂Cl₂) to give a white solid (42 mg, 44% yield).

LCMS (0.1% formic acid modifier) calculated. (M+1)⁺ 496.69, observed 497.6.

¹H NMR (CDCl₃) δ 8.49 (s, 1H), 5.06 (br, 1H), 4.56-4.52 (m, 2H), 3.88-3.68 (m, 3H), 3.38-3.26 (m, 4H), 2.61-2.48 (m, 11H), 2.00 (s, 3H), 1.78-1.56 (m, 12H), 1.02-0.98 (m, 6H).

### Method C3

### Preparation of Representative Examples

### Compound 692

### (S)-2-[4-(2-Adamantan-1-yl-2-hydroxy-ethylamino)-5,8-dihydro-6H-pyrido[3,4-d]pyrimidin-7-yl]-(R)-pyrrolidin-2-yl-methanone

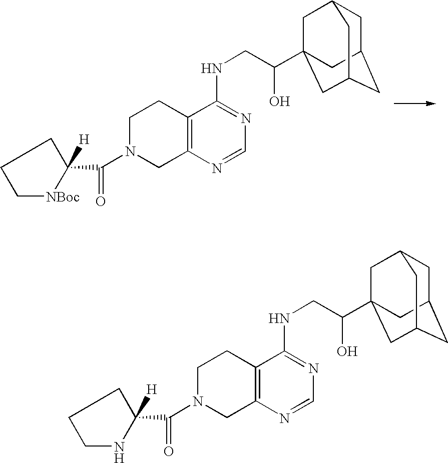

A mixture of 1 M HCl ethyl ether solution (3 mL, 0.003 mol) and (R)-2-[4-(2-Adamantan-1-yl-2-hydroxy-ethylamino)-5,8-dihydro-6H-pyrido[3,4-d]pyrimidine-7-carbonyl]-pyrrolidine-1-carboxylic acid tert-butyl ester (20 mg, 0.00004 mol) was stirred at room temperature for 15 hours. The mixture was poured onto a pad of silica gel and flash chromatographed (4 g of silica gel, 0-10% methanol in dichloromethane gradient) to give [4-(2-adamantan-1-yl-2-hydroxy-ethylamino)-5,8-dihydro-6H-pyrido[3,4-d]pyrimidin-7-yl]-(R)-pyrrolidin-2-yl-methanone as a white solid (4.5 mg, 30% yield).

LCMS (0.1% formic acid modifier) calculated. (M+1)⁺ 425.57, observed 426.4.

¹H NMR (CDCl₃) δ 8.48 (s, 1H), 5.21 (br, 1H), 4.52-4.48 (m, 1H), 4.04-3.68 (m, 4H), 3.32-3.08 (m, 3H), 2.88-2.76 (m, 1H), 2.56-2.46 (m, 6H), 2.18-2.10 (m, 1H), 2.00 (s, 3H), 1.88-1.56 (m, 12H).

### Compound 712

### (R)-1-[4-(2-Adamantan-1-yl-2-hydroxy-ethylamino)-5,8-dihydro-6H-pyrido[3,4-d]pyrimidin-7-yl]-2-amino-propan-1-one

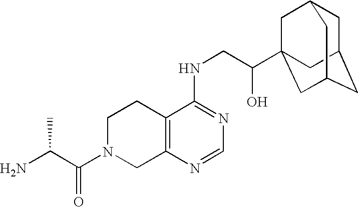

Trifluoroacetic acid (1 mL, 0.01 mol) was added to a solution of {(R)-2-[4-(2-Adamantan-1-yl-2-hydroxy-ethylamino)-5,8-dihydro-6H-pyrido[3,4-d]pyrimidin-7-yl]-1-methyl-2-oxo-ethyl}-carbamic acid tert-butyl ester (60 mg, 0.0001 mol) in methylene chloride (2 mL). The mixture was stirred at room temperature for 2 hours, concentrated and treated with triethyl amine (1 mL), followed by re-concentration. Purification via flash chromatography (12 g of silica gel, 0-20% MeOH/CH₂Cl₂ gradient) gave the desired product as a white solid (48 mg, 100% yield).

LCMS (0.1% formic acid modifier) calculated. (M+1)⁺ 399.53, observed 400.3.

¹H NMR (CDCl₃) δ 8.48 (s, 1H), 5.06-5.02 (m, 1H), 4.56-4.49 (m, 1H), 3.99-3.86 (m, 4H), 3.32-3.28 (m, 2H), 3.02-2.96 (m, 1H), 2.51-2.46 (m, 1H), 2.00 (s, 3H), 1.76-1.55 (m, 12H), 1.32-1.26 (m, 3H).

### Compound 713

### (S)-1-[4-(2-Adamantan-1-yl-2-hydroxy-ethylamino)-5,8-dihydro-6H-pyrido[3,4-d]pyrimidin-7-yl]-2-amino-propan-1-one

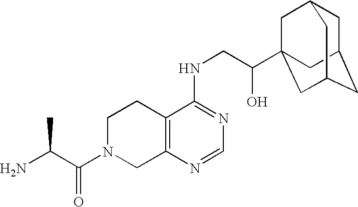

Trifluoroacetic Acid (1 mL, 0.01 mol) was added to a solution of {(S)-2-[4-(2-Adamantan-1-yl-2-hydroxy-ethylamino-5,8-dihydro-6H-pyrido[3,4-d]pyrimidin-7-yl]-1-methyl-2-oxo-ethyl}-carbamic acid tert-butyl ester (65 mg, 0.00013 mol) in methylene chloride (2 mL). The mixture was stirred at room temperature for 2 hours. The volatiles were removed under vacuum and the residue was treated with triethyl amine (2 mL), concentrated, purified via flash chromatography (12 g of silica gel, 0-20% MeOH/CH₂Cl₂ gradient) to give the desired product as a white solid (49 mg, 91% yield).

LCMS (0.1% formic acid modifier) calculated. (M+1)⁺ 399.53, observed 400.4.

¹H NMR (CDCl₃) δ 8.48 (s, 1H), 5.06-5.02 (m, 1H), 4.56-4.49 (m, 1H), 3.99-3.86 (m, 4H), 3.32-3.28 (m, 2H), 3.02-2.96 (m, 1H), 2.51-2.46 (m, 1H), 2.00 (s, 3H), 1.76-1.55 (m, 12H), 1.32-1.26 (m, 3H).

### Compound 714

### (S)-1-[4-(2-Adamantan-1-yl-2-hydroxy-ethylamino)-5,8-dihydro-6H-pyrido[3,4-d]pyrimidin-7-yl]-2-amino-3-phenyl-propan-1-one

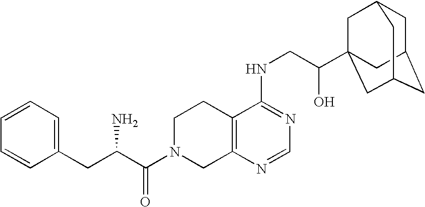

Trifluoroacetic acid (1 mL, 0.01 mol) was added to a solution of {(S)-2-[4-(2-Adamantan-1-yl-2-hydroxy-ethylamino)-5,8-dihydro-6H-pyrido[3,4-d]pyrimidin-7-yl]-1-benzyl-2-oxo-ethyl}-carbamic acid tert-butyl ester (70 mg, 0.0001 mol) in methylene chloride (2 mL). The mixture was stirred at room temperature for 2 hours. Concentrated to dryness and treated with Triethyl amine (1 mL). Concentration, followed by purification via flash chromatography (12 g of silica gel, 0-20% MeOH/CH₂Cl₂ gradient) gave the desired product as a white solid (58 mg, 100% yield).

LCMS (0.1% formic acid modifier) calculated. (M+1)⁺ 475.63, observed 476.4.

¹H NMR (CDCl₃) δ 8.36 (s, 1H), 7.32-6.98 (m, 5H), 5.03-4.96 (m, 1H), 4.65-4.55 (m, 1H), 4.03-3.96 (m, 2H), 3.88-3.65 (m, 2H), 3.36-3.24 (m, 2H), 3.02-2.86 (m, 4H), 2.00 (s, 3H), 1.76-1.64 (m, 12H).

### Compound 715

### 1-[4-(2-Adamantan-1-yl-2-hydroxy-ethylamino)-5,8-dihydro-6H-pyrido[3,4-d]pyrimidin-7-yl]-2-amino-ethanone

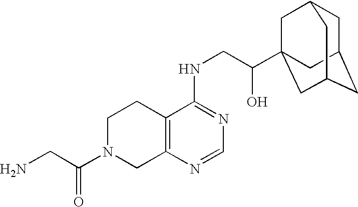

Trifluoroacetic acid (1 mL, 0.01 mol) was added to a solution of {2-[4-(2-Adamantan-1-yl-2-hydroxy-ethylamino)-5,8-dihydro-6H-pyrido[3,4-d]pyrimidin-7-yl]-2-oxo-ethyl}-carbamic acid tert-butyl ester (60 mg, 0.0001 mol) in methylene chloride (3 mL). The mixture was stirred at room temperature for 2 hours. Concentrated, treated with triethyl amine (3 mL) and then concentrated again. Purified with flash chromatography (12 g of silica gel, 0-20% MeOH/CH₂Cl₂ gradient) to give a white solid (31 mg, 60% yield).

LCMS (0.1% formic acid modifier) calculated. (M+1)⁺ 385.50, observed 386.3.

### Compound 720

### 1-[4-(2-Adamantan-1-yl-2-hydroxy-ethylamino)-5,8-dihydro-6H-pyrido[3,4-d]pyrimidin-7-yl]-2-piperazin-1-yl-ethanone

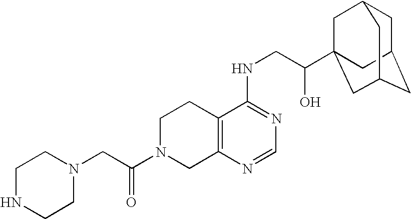

4-{2-[4-(2-Adamantan-1-yl-2-hydroxy-ethylamino)-5,8-dihydro-6H-pyrido[3,4-d]pyrimidin-7-yl]-2-oxo-ethyl}-piperazine-1-carboxylic acid tert-butyl ester (160 mg, 0.00029 mol) was stirred with 2 N HCl diethyl ether solution (2 mL) at room temperature for 3 days. The resulting white solid was collected via filtration and rinsed with diethyl ether (110 mg, 78% yield).

LCMS (0.1% formic acid modifier) calculated. (M+1)⁺ 490.06, observed (M-HCl)⁺ 455.4.

¹H NMR (DMSO-d₆) δ 9.75 (br, 2H), 8.68 (s, 1H), 4.65-3.36 (m, 18H), 2.66 (s, 2H), 2.00 (s, 3H), 1.68-1.51 (m, 12H).

### Compound 723

### (R)-1-[4-(2-Adamantan-1-yl-2-hydroxy-ethylamino)-5,8-dihydro-6H-pyrido[3,4-d]pyrimidin-7-yl]-2-amino-3-phenyl-propan-1-one

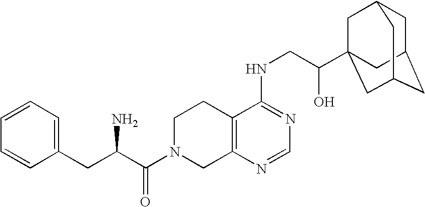

Trifluoroacetic acid (1 mL, 0.01 mol) was added to a solution of {(R)-2-[4-(2-adamantan-1-yl-2-hydroxy-ethylamino)-5,8-dihydro-6H-pyrido[3,4-d]pyrimidin-7-yl]-1-benzyl-2-oxo-ethyl}-carbamic acid tert-butyl ester (60 mg, 0.0001 mol) in methylene chloride (3 mL). The mixture was stirred at room temperature for 2 hours. The volatiles were removed via evaporation and purified via flash chromatography (12 g of silica gel, 0-10% MeOH/CH₂Cl₂ gradient) to give a white solid (36 mg, 70% yield).

LCMS (0.1% formic acid modifier) calculated. (M+1)⁺ 475.63, observed 476.4.

¹H NMR (CDCl₃) δ 8.36 (s, 1H), 7.32-6.98 (m, 5H), 5.03-4.96 (m, 1H), 4.65-4.55 (m, 1H), 4.03-3.96 (m, 2H), 3.88-3.65 (m, 2H), 3.36-3.24 (m, 2H), 3.02-2.86 (m, 4H), 2.00 (s, 3H), 1.76-1.64 (m, 12H).

### Compound 724

### R)-1-[4-(2-Adamantan-1-yl-2-hydroxy-ethylamino)-5,8-dihydro-6H-pyrido[3,4-d]pyrimidin-7-yl]-2,3-dimethyl-butan-1-one

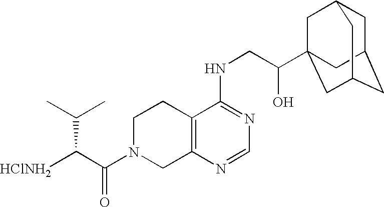

{(R)-1-[4-(2-Adamantan-1-yl-2-hydroxy-ethylamino)-5,8-dihydro-6H-pyrido[3,4-d]pyrimidine-7-carbonyl]-2-methyl-propyl}-carbamic acid tert-butyl ester (90 mg, 0.0002 mol) was stirred in 1 M HCl in ethyl ether solution (4 mL) at room temperature over 24 hours. The resulting white solid was collected via filtration (54 mg, 70% yield).

LCMS (0.1% formic acid modifier) calculated. (M+1)⁺ 464.04, observed (m-HCl)⁺ 428.2.

¹H NMR (DMSO-d6) δ 9.42 (br 0.5H), 8.98 (br, 0.5H), 8.88 (s, 1H), 8.32 (br, 2H), 5.00-4.89 (m, 1H), 4.56-3.08 (m, 9H), 2.61-2.52 (m, 2H), 2.03-1.96 (m, 3H), 1.76-1.56 (m, 12H), 1.22-1.12 (m, 6H).

### Compound 725

### (S)-1-[4-(2-Adamantan-1-yl-2-hydroxy-ethylamino)-5,8-dihydro-6H-pyrido[3,4-d]pyrimidin-7-yl]-2,3-dimethyl-butan-1-one

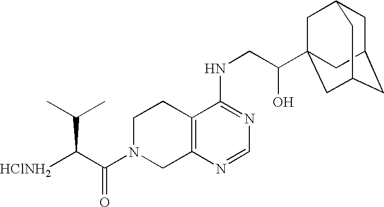

{(S)-1-[4-(2-Adamantan-1-yl-2-hydroxy-ethylamino)-5,8-dihydro-6H-pyrido[3,4-d]pyrimidine-7-carbonyl]-2-methyl-propyl}-carbamic acid tert-butyl ester (97 mg, 0.00018 mol) was stirred in 1 M HCl in ethyl ether solution (4 mL) at room temperature over 24 hours. The resulting white solid was collected via filtration (25 mg, 29% yield).

LCMS (0.1% formic acid modifier) calculated. (M+1)⁺ 464.04, observed (M-HCl)⁺ 428.2.

¹H NMR (DMSO-d6) δ 9.42 (br 0.5H), 8.98 (br, 0.5H), 8.88 (s, 1H), 8.32 (br, 2H), 5.00-4.89 (m, 1H), 4.56-3.08 (m, 9H), 2.61-2.52 (m, 2H), 2.03-1.96 (m, 3H), 1.76-1.56 (m, 12H), 1.22-1.12 (m, 6H).

### Compound 726

### (S)-1-[4-(2-Adamantan-1-yl-2-hydroxy-ethylamino)-5,8-dihydro-6H-pyrido[3,4-d]pyrimidin-7-yl]-2-amino-3-hydroxy-propan-1-one

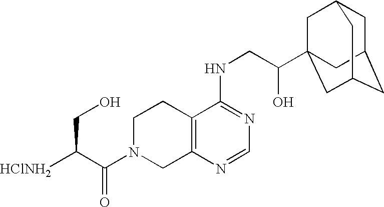

{(S)-2-[4-(2-Adamantan-1-yl-2-hydroxy-ethylamino)-5,8-dihydro-6H-pyrido[3,4-d]pyrimidin-7-yl]-1-hydroxymethyl-2-oxo-ethyl}-carbamic acid tert-butyl ester (80 mg, 0.0002 mol) was stirred in 1M HCl in ethyl ether solution at room temperature for 18 hours. The resulting white solid was collected via filtration (52 mg, 70% yield):

LCMS (0.1% formic acid modifier) calcd. (M+1)⁺ 451.99, observed (M-HCl)⁺ 416.2.

¹H NMR (DMSO-d6) δ 9.56 (br, 1H), 8.60 (s, 1H), 8.32 (br, 2H), 5.56 (br, 1H), 4.62-4.48 (m, 2H), 3.98-3.00 (m, 9H), 2.61(s, 2H), 2.00 (s, 3H), 1.76-1.48 (m, 12H).

### Compound 727

### (R)-1-[4-(2-Adamantan-1-yl-2-hydroxy-ethylamino)-5,8-dihydro-6H-pyrido[3,4-d]pyrimidin-7-yl]-2-amino-3-hydroxy-propan-1-one

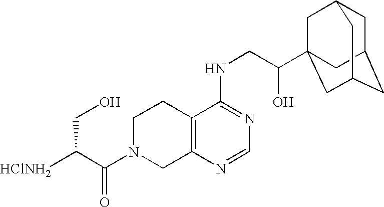

{(R)-2-[4-(2-Adamantan-1-yl-2-hydroxy-ethylamino)-5,8-dihydro-6H-pyrido[3,4-d]pyrimidin-7-yl]-1-hydroxymethyl-2-oxo-ethyl}-carbamic acid tert-butyl ester (80 mg, 0.0002 mol) was stirred in 1M HCl in ethyl ether solution at room temperature for 18 hours. The resulting white solid was collected via filtration (56 mg, 80% yield).

LCMS (0.1% formic acid modifier) calculated. (M+1)⁺ 451.99, observed (M-HCl)⁺ 416.2.

¹H NMR (DMSO-d6) δ 9.56 (br, 1H), 8.60 (s, 1H), 8.32 (br, 2H), 5.56 (br, 1H), 4.62-4.48 (m, 2H), 3.98-3.00 (m, 9H), 2.61(s, 2H), 2.00 (s, 3H), 1.76-1.48 (m, 12H).

### Method C4

### Compound 717

### 1-[4-(2-Adamantan-1-yl-2-hydroxy-ethylamino)-5,8-dihydro-6H-pyrido[3,4-d]pyrimidin-7-yl]-2-pyrrolidin-1-yl-ethanone

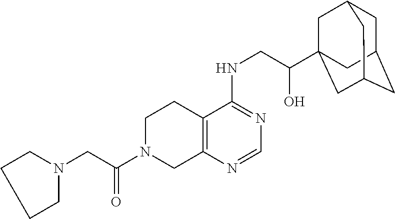

Pyrrolidin-1-yl-acetic acid hydrochloride (24 mg, 0.00015 mol), 1-adamantan-1-yl-2-(5,6,7,8-tetrahydro-pyrido[3,4-d]pyrimidin-4-ylamino)-ethanol (Intermediate 5, 40 mg, 0.0001 mol), N,N,N′,N′-Tetramethyl-O-(7-azabenzotriazol-1-yl)uronium Hexafluorophosphate (56 mg, 0.00015 mol) and N,N-Diisopropylethylamine (60 mg, 0.0005 mol) in N,N-Dimethylformamide (3 mL) were stirred in at room temperature for 2 hours. The mixture was diluted with CH2Cl2 (50 mL), washed with water (10 mL×3), dried over Na2SO4, filtered and concentrated. Purification via flash chromatography (12 g of silica gel, 0-10% MeOH/CH₂Cl₂ gradient) gave a white solid (21 mg, 40% yield).

LCMS (0.1% formic acid modifier) calculated. (M+1)⁺ 439.59, observed 440.3.

¹H NMR (CDCl₃) δ 8.40 (s, 1H), 5.48 (br, 0.6 H), 5.30 (br, 0.4H), 4.63-4.59 (m, 2H), 3.90-3.81 (m, 4H), 3.50-3.22 (m, 6H), 2.61-2.42 (m, 6H), 2.01 (s, 3H), 1.80-1.59 (m, 12H).

### Method D

### Preparation of Representative Examples

### Compound 171

### Adamantan-1-ylmethyl-(7-pyridin-4-ylmethyl-5,6,7,8-tetrahydro-pyrido[3,4-d]pyrimidin-4-yl)-amine

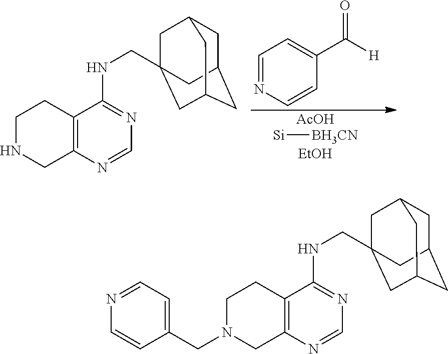

In a 2.0 ml sealed vessel was added 2-adamantan-1-ylmethyl-(5,6,7,8-tetrahydropyrido[3,4-d]pyrimidin-4-yl)amine (Intermediate 2, 2.9 mg, 10 μmol) in 600 μl of absolute ethanol. To the reaction was added nicotinaldehyde (1.6 mg, 15 μmol), followed by acetic acid (60 μl) and silica bound sodium cyanoborohydride (15 mg, 15 μmol). The reaction was heated at 120° C. for 5 minutes via microwave and the solvents were evaporated. The residue was dissolved in DMSO and purified using LC-MS based purification (50 mm×10 mm Phenomenex Gemini Column using a 10-100% acetonitrile-water gradient). ESI-MS m/z 389 (M+H)⁺.

### Method E

### Preparation of Representative Examples

### Compound 266

### {1-{4-[(Adamantan-1-ylmethyl)-amino]-5,8-dihydro-6H-pyrido[3,4-d]pyrimidin-7-yl}-2-piperidin-1-yl-ethanone

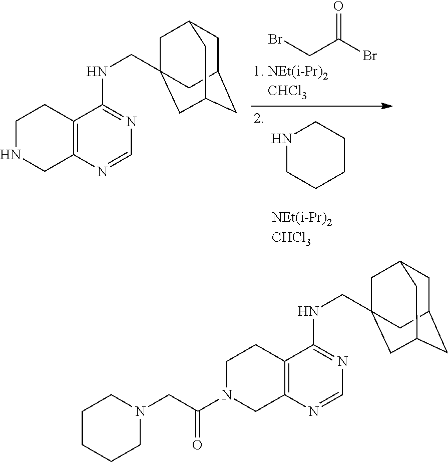

In one well of a 96-well polypropylene reaction plate was added adamantan-1-ylmethyl-(5,6,7,8-tetrahydropyrido[3,4-d]pyrimidin-4-yl)amine (Intermediate 2, 2.9 mg, 10 μmol) in 100 μl of anhydrous chloroform. To the reaction was added bromoacetyl bromide (2.0 mg, 10 μmol), followed by diisopropylethylamine (1.95 mg, 15 μmol). The reaction plate was agitated for 15 minutes. Piperidine was added (1.75 mg, 20 μmol), followed by diisopropylethylamine (2.6 mg, 20 μmol). After reaction completion, the solvents were evaporated. The residue was dissolved in DMSO and purified using LC-MS based purification (50 mm×10 mm Phenomenex Gemini Column using a 10-100% acetonitrile-water gradient). ESI-MS m/z 424 (M+H)⁺.

### Method F

### Preparation of the Intermediate Nitrophenyl Ester

### 4-[(Adamantan-1-ylmethyl)-amino]-5,8-dihydro-6H-pyrido[3,4-d]pyrimidine-7-carboxylic acid 4-nitro-phenyl ester

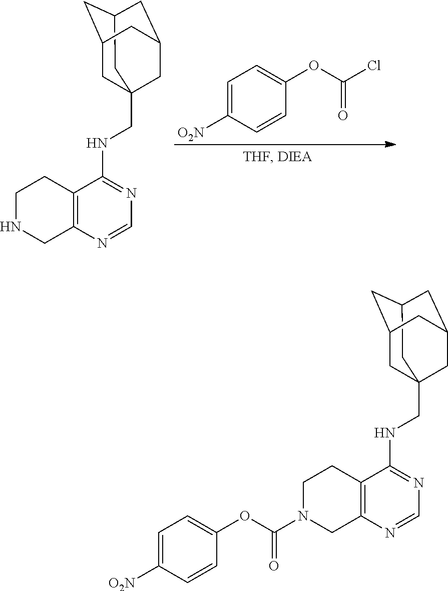

Adamantan-1-ylmethyl-(5,6,7,8-tetrahydro-pyrido[3,4-d]pyrimidin-4-yl)-amine (Intermediate 2, 1.2 g, 4.03 mmol) was dissolved in 60 mL of THF and placed in a 2-necked, round-bottom flask, equipped with an argon inlet and a stir bar. Diisopropylethylamine (0.92 mL, 5.24 mmol) and p-nitrophenylchloroformate (0.97 g, 4.83 mmol) were added to the flask. The mixture was stirred under a gentle argon flow at room temperature for 2 hours. The reaction progress was monitored by LC/MS analysis. The solvents were removed under vacuum and the product was purified by flash chromatography over silica gel to give 4-[(adamantan-1-ylmethyl)-amino]-5,8-dihydro-6H-pyrido[3,4-d]pyrimidine-7-carboxylic acid 4-nitro-phenyl ester (0.59 g, 1.2 mmol, 32%) as light yellow crystals.

LCMS (M+1)⁺ 464.22, observed 464.1.

### Representative Parallel Synthesis of Carbamate Derivatives

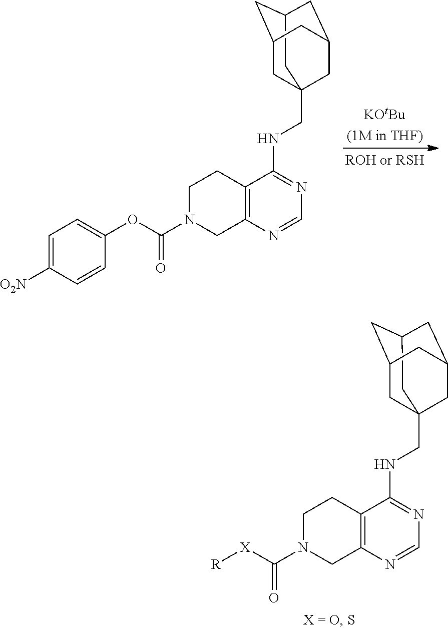

The 4-[(Adamantan-1-ylmethyl)-amino]-5,8-dihydro-6H-pyrido[3,4-d]pyrimidine-7-carboxylic acid 4-nitro-phenyl ester was distributed into 8-mL vials (0.025 g, 0.0539 mmol). To each vial, 2 mL of anhydrous THF was added, followed by 3 equivalents of corresponding alcohols or thiols. Each vial was gently shaken to ensure complete dissolving of reagents. To each vial, 1 M KOtBu in THF solution (0.162 mL, 0.162 mmol) was added and the vials were shaken at room temperature for 3-4 h or overnight. The reaction mixtures were then treated with 0.5 mL of methanol, evaporated to dryness and re-dissolved in 1,2-dichloroethane/methanol 1:1 mixture (in some cases, DMSO was used) for purification by HPLC.

### Preparation of Representative Examples Using Method F

### Compound 250

### 4-[(Adamantan-1-ylmethyl)-amino]-5,8-dihydro-6H-pyrido[3,4-d]pyrimidine-7-carboxylic acid (R)-(tetrahydro-furan-3-yl)ester

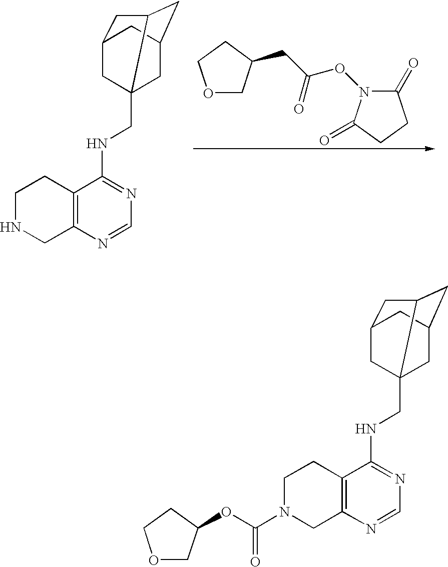

A mixture of (R)-2,5-dioxopyrrolidin-1-yl tetrahydrofuran-3-yl carbonate (33 mg, 0.00015 mol), and triethylamine (40 mg, 0.0004 mol) in methylene chloride (3 mL, 0.05 mol) was stirred at room temperature for 10 minutes and then adamantan-1-ylmethyl-(5,6,7,8-tetra hydro-pyrido[3,4-d]pyrimidin-4-yl)-amine (Intermediate 2, 40 mg, 0.0001 mol) was added. The resulting mixture was stirred at room temperature for 15 hours. The mixture was poured onto a pad of silica gel and flash chromatographed over silica gel (12 g of silica gel, 0-10% methanol in dichloromethane gradient) to give the product as a white solid (40 mg, 80% yield).

LCMS calculated. (M+1)⁺ 412.53, observed 412.7.

¹H NMR (CDCl₃) δ 8.48 (s, 1H), 5.47-5.32 (m, 1H), 4.67-4.46 (m, 3H), 3.98-3.72 (m, 6H), 3.32-3.28 (m, 2H), 2.49 (s, 2H), 2.06-2.01 (m, 4H), 1.88-1.51 (m, 12H).

### Method G

### Preparation of Representative Example

### Compound 501

### 1-Adamantan-1-yl-2-(7-benzyl-5,6,7,8-tetrahydro-pyrido[3,4-d]pyrimidin-4-ylamino)-ethanol

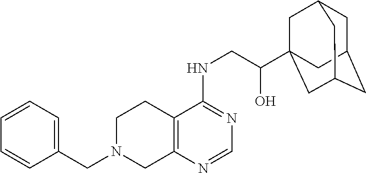

A mixture of 7-benzyl-4-chloro-5,6,7,8-tetrahydropyrido[3,4-d]pyrimidine (Intermediate 1, 1.23 g, 0.00474 mol), 1-adamantan-1-yl-2-amino-ethanol (1.39 g, 0.00710 mol) and N,N-diisopropylethylamine (1.6 mL, 0.0095 mol) in acetonitrile (30 mL, 0.6 mol) was heated via microwave at at 150° C. for one hour. LC/MS analysis showed only product and unreacted 7-benzyl-4-chloro-5,6,7,8-tetrahydropyrido[3,4-d]pyrimidine. The mixture was cooled to room temperature and poured into sat. sodium bicarbonate. The aqueous layer was extracted with ethyl acetate and the organic layer was washed with brine and dried over sodium sulfate. The organic layer was concentrated to give a yellow solid. The residue was purified by flash chromatography over silica gel (0-10% methanol in dichloromethane gradient) to give 1-adamantan-1-yl-2-(7-benzyl-5,6,7,8-tetrahydro-pyrido[3,4-d]pyrimidin-4-ylamino)-ethanol as a yellow powder after drying.

LCMS calculated. (M+1)⁺ 418.57, observed 419.

### Method H

### Preparation of Representative Example

### Compound 696

### 4-(2-Adamantan-1-yl-2-hydroxy-ethylamino)-5,8-dihydro-6H-pyrido[3,4-d]pyrimidine-7-carboxylic acid (S)-(tetrahydro-furan-3-yl) ester

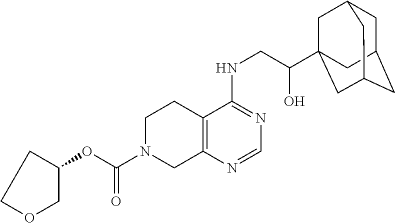

A mixture of (S)-2,5-dioxopyrrolidin-1-yl tetrahydrofuran-3-yl carbonate (33 mg, 0.00015 mol), triethyl amine (40 mg, 0.0004 mol), and 1-adamantan-1-yl-2-(5,6,7,8-tetrahydro-pyrido[3,4d]pyrimidin-4-ylamino)-ethanol (Intermediate 5, 40 mg, 0.0001 mol) in methylene chloride (3 mL) was stirred at room temperature for 15 hours. The mixture was poured onto a pad of silica gel and flash chromatographed (12 g of silica gel, 0-10% methanol in dichloromethane gradient) to give the product as a white solid (32 mg, 60% yield).

LCMS (0.1% formic acid modifier) calculated. (M+1)⁺ 442.55, observed 443.5.

¹H NMR (CDCl₃) δ 8.49 (s, 1H), 5.32-5.01 (m, 2H), 4.50 (s, 2H), 4.00-3.66 (m, 8H), 3.32-3.22 (m, 2H), 2.48 (s, 2H), 2.00 (s, 3H), 1.76-1.52 (m, 12H).

### Method I

### Compound 502

### 1-Adamantan-1-yl-2-[7-(2,4-difluorobenzyl)-5,6,7,8-tetrahydro-pyrido[3,4-d]pyrimidin-4-ylamino]-ethanol

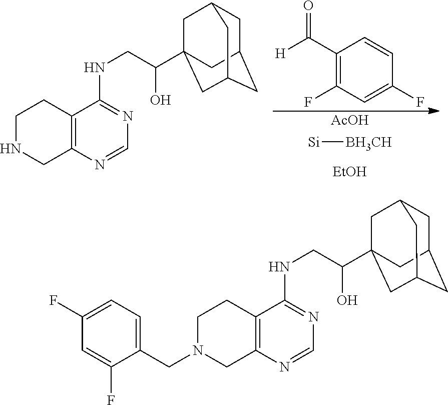

In a 2.0 ml μ-wave vessel was added 1-adamantan-1-yl-2-(5,6,7,8-tetrahydropyrido[3,4-d]pyrimidin-4-ylamino)ethanol (Intermediate 5, 3.27 mg, 10 μmol) in 600 μl of absolute ethanol. To the reaction was added 2,4-difluorobenzaldehyde (2.1 mg, 15 μmol), followed by acetic acid (60 μl) and silica bound sodium cyanoborohydride (15 mg, 15 μmol). The reaction was heated at 120° C. for 5 minutes and the solvent was evaporated. The residue was dissolved in DMSO and purified using LC-MS based purification (50 mm×10 mm Phenomenex Gemini Column using a 10-100% acetonitrile-water gradient).

### Method J

### Compound 685

### 4-(2-Adamantan-1-yl-2-hydroxy-ethylamino)-5,8-dihydro-6H-pyrido[3,4-d]pyrimidine-7-carboxylic acid-tert-butyl ester

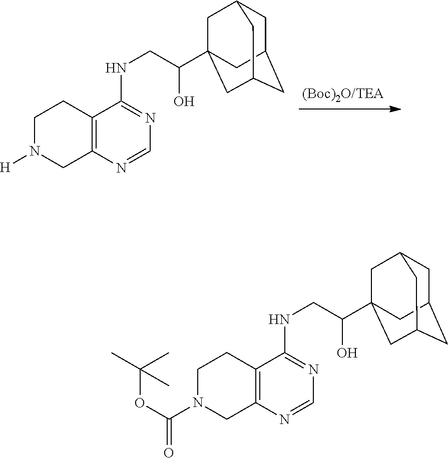

A mixture of 1-Adamantan-1-yl-2-(5,6,7,8-tetrahydro-pyrido[3,4-d]pyrimidin-4-ylamino)-ethanol (Intermediate 5, 30.0 mg, 0.0000913 mol), di-tert-Butyldicarbonate (22.0 mg, 0.000101 mol) and triethyl amine (14.0 mg, 0.000138 mol) in methylene chloride (3 mL) was stirred at rt for 2 hours. The mixture was poured onto a pad of silica gel and then purified by flash chromatography (4 g of silica gel, 60% EtOAc/Hexanes gradient) to give 4-(2-Adamantan-1-yl-2-hydroxy-ethylamino)-5,8-dihydro-6H-pyrido[3,4-d]pyrimidine-7-carboxylic acid-tert-butyl ester as a white solid (22 mg, 55% yield).

LCMS (0.1% formic acid modifier) calculated. (M+1)⁺ 428.57, observed 429.2.

¹H NMR (CDCl₃) δ 8.48 (s, 1H), 5.18 (br, 1H), 4.49 (s, 2H), 3.88-3.82 (m, 1H), 3.78-3.70 (m, 2H), 3.38-3.29 (m, 2H), 2.49-2.46 (m, 2H), 2.01 (s, 3H), 1.76-1.51 (m, 12H), 1.49 (s, 9H).

Additional compounds of the invention, which are prepared or can be prepared using the procedures and synthetic methods described above, or some modification thereof, and using the corresponding starting materials and appropriate reagents, are listed in Tables 1 and 2. For the sake of convenience, the closest method of synthesis for a compound is depicted in Tables 1 and 2. It will be obvious to one of skill in the art that the method may require minor modifications to obtain acceptable yields. It will also be obvious to one of skill in the art that the compound may be prepared by other methods known in the art. Furthermore, the final compounds of this invention are or can be purified using various purification methods known to those skilled in the art.

The following biological examples, Examples 1-13, are offered to illustrate the present invention and are not to be construed in any way as limiting its scope. In the examples below, all temperatures are in degrees Celsius (unless otherwise indicated).

### In Vitro Assay Methods

### Example 1

The P2X₇ receptor is strongly expressed in macrophage-derived cell lines, including, but not limited to, J774 (mouse macrophage line, American Type Culture Collection (ATCC), Rockville, Md., ATCC TIB-67), P388 (mouse cell line, ATCC CCL-46), P815 (mouse mast cell mastocytoma-derived line, ATCC TIB-64), THP-1 (Human monocyte-derived cell line, ATCC TIB202) and U937 (human cell line derived from histiocytic lymphoma, induceable to monocyte differentiation, ATCC CRL-1593.2) and in isolated macrophage cultures. Human or non-human animal macrophages are isolated using the procedure noted below.

The P2Z/P2X₇ receptor can be characterized by measuring channel opening, for instance ion flux, and/or by assessing pore formation, including by monitoring dye uptake or cell lysis in cells naturally expressing this receptor. Compounds such as ATP, 2′ and 3′-(O)-(4-benzoyl benzoyl) ATP (BzATP) effect the formation of pores in the plasma membrane of these cells, particularly at low extracellular divalent ion concentrations (Buisman et al, Proc. Natl. Acad. Sci. USA 85:7988 (1988); Zambon et al, Cell. Immunol 156:458 (1994); Hickman et al Blood 84:2452 (1994)). Large molecular size dyes, including propidium dye YO-PRO-1, can be seen entering macrophage-derived cell lines during cell recordings (Hickman et al, Blood 84:2452 (1994); Wiley et al, Br J Pharmacol 112:946 (1994); Steinberg et al, J Biol Chem 262:8884 (1987)). Ethidium bromide (a fluorescent DNA probe) can also be monitored, where an increase in the fluorescence of intracellular DNA-bound ethidium bromide is observed. Expression of recombinant rat or human rP2X₇ in cells, including HEK293 cells, and in Xenopus oocytes demonstrates influx and pore formation by whole cell recordings and YO-PRO-1 fluorescence (Suprenant et al, Science 272:735 (1996); Rassendren et al, J Biol Chem 272:5482 (1997)).

The compounds of the invention may be tested for antagonist activity at the P2X₇ receptor. Tests that may be performed include and are selected from: (i) electrophysiological experiments; (ii) YO-PRO1 fluorescence; (iii) ethidium bromide fluorescence; and (iv) IL-1β release from stimulated macrophages, including as described below. Compounds can be tested in vivo in animal models including for inflammation models (e.g. paw edema model, collagen-induced arthritis, EAE model of MS).

### Isolation of Human Macrophages

Monocyte-derived human or non-human animal macrophage cultures are prepared as described by Blanchard et al (Blanchard et al, J Cell Biochem 57:452 (1995); Blanchard et al, J Immunol 147:2579 (1991)). Briefly, monocytes are isolated from leukocyte concentrates obtained from a healthy volunteer. Leukocytes are suspended in RPMI 1460 medium (Life Techologies, Inc.) with 20% serum (human for human cells), 2 mM glutamine, 5 mM HEPES, and 100 μg/ml streptomycin. Cells are allowed to adhere to culture flasks for 1-2 h, after which nonadherent cells are washed away. Adherent cells are cultured for 7-14 d in this medium plus interferon-γ (human for human cells) (1000 units/ml). Macrophages are recovered from the culture flask by pipetting with cold phosphate-buffered saline and plated onto glass coverslips for electrophysiological or other experiments carried out 12-24 h later.

### Example 2

### Electrophysiological Experiments

Whole cell recordings are made using the EPC9 patch-clamp amplifier and Pulse acquisition programs (HEKA, Lambrecht, Germany). Whole-cell recordings are obtained from cells, e.g. J774A.1 cells (American Type Culture Collection, Rockville, Md., ATCC TIB-67)); agonists are applied for periods of 1 to 3 s by a fast-flow U-tube delivery system [E. M. Fenwick, A. Marty, E. Neher, J. Physiol, (London) 331, 577 (1982)]. The internal pipette solution is 140 mM cesium-aspartate or potassium-aspartate, 20 mM NaCl, 110 mM EGTA, and 5 mM Hepes; normal external solution is 145 mM NaCl, 2 mM KCl, 2 mM CaCl₂, 1 mM MgCl_{2,} 10 mM Hepes, and 12 mM glucose. Low divalent external solution is nominally magnesium-free with 0.3 mM CaCl₂. Concentration-response curves are constructed in low divalent solution by recording currents in response to 1 s applications of agonist at 8 min intervals with normal external solution present for 6 min before each application. This protocol is necessary to prevent the development of sustained inward currents.

Reversal potentials (Eᵣₑᵥ) are obtained by application of ATP (300 μM) or BzATP (30 μM) (controls), or the compound being tested, while the membrane is held at various potentials or by application of voltage ramps from −120 to 30 or 50 mV. Permeability ratios are calculated from Fᵣₑᵥ by first computing α (=P_{Na}/P_{K′} where P is permeability) for internal (i) and external (o) concentrations [Na]_{I}=20 mM, [Na]ₒ=145 mM, [K]ₒ=0 mM, and [K]_{I}=140 mM from α=([145/exp(EᵣₑᵥFIRT)]−20)/140 (where F is the Faraday, R is the gas constant, and T is the absolute temperature). Other Pₓ/P_{Na} values, when [X]ₒ=145 mM, [Na]_{I}=20 mM, [K]_{I}=140 mM, and [Na]ₒ=[K]ₒ=[X]_{I}=0 mM, are computed from Pₓ/P_{Na}=[(exp)EᵣₑᵥF/RT)] (20+140α))/145. In order of size, X is cesium, methylamine, tris(hydroxymethyl)-aminomethane, tetraethylammonium, and N-methyl-D-glucamine. The internal solution also contains 10 mM EGTA and 5 mM Hepes, External solutions also contain 10 mM glucose and normal or low concentrations of divalent cations; pH is maintained at 7.3 with HCl, histidine, or Hepes as required, and the osmolarity of all solutions is 295 to 315.

### Example 3

### YO-PRO1 Fluorescence

The Photonics Imaging (IDEA) system for microscopic fluorescence measurements (Photonics, Planegg, Germany) is used. Coverslips are placed at the stage of a Zeiss Axiovert 100 or equivalent inverted microscope and viewed under oil immersion with a 40× Fluor objective. YO-PRO-1 (10 μM; Molecular Probes, Eugene, Oreg.) is added to the superfusion fluid during electrophysiological recordings 3 to 6 min before switching to low divalent solution and washed out upon switching back to normal divalent solution, after which the fluorescent lamp is turned on and cells are examined with a fluorescein isothiocyanate filter. YO-PRO1 fluorescence is measured using 491/509 nm excitation/emission wavelengths. Images are obtained at 5-20 s intervals during continuous superfusion (2 ml/min) with YO-PRO1 and varying concentrations of control ATP, BzATP or compound to be tested. For each experiment, the time course of YO-PRO1 fluorescence obtained for 10-20 individual cells and then averaged to obtain the mean fluorescence signal. Results are expressed as mean signal at 3 min for rP2X_{7,} and the signal at 10 min is used for P2X₇ and human macrophage cells. All experiments are carried out at room temperature.

### Example 4

### Ethidium Bromide

Compounds of the invention are tested for antagonist activity at the P2X₇ receptor by monitoring Ethidium Bromide entering P2X₇ receptor-expressing cells on pore formation. The test is performed in 96-well flat bottomed microtitre plates, the wells being filled with 250 μl of test solution comprising 200 μl of a suspension of P2X₇-expressing cells (e.g. THP-1 cells, J774 cells, etc.) (2.5×10⁶ cells/ml) containing 10⁻⁴M ethidium bromide, 25 μl of a high potassium buffer solution containing 10⁻⁵M BzATP, and 25 μl of a high potassium buffer solution containing test compound. The plate is covered with a plastic sheet and incubated at 37° C. for one hour. The plate is then read in a Perkin-Elmer fluorescent plate reader, excitation 520 nm, emission 595 nm, slit widths: Ex 15 nm, EM 20 nm. For the purposes of comparison, BzATP (a P2X₇ receptor agonist) and pyridoxal 5-phosphate (a P2X₇ receptor agonist) are used separately in the test as controls. From the readings obtained, a pIC₅₀ figure is calculated for each test compound. This figure is the negative logarithm of the concentration of test compound necessary to reduce the BzATP agonist activity by 50%.

### Example 5

### Pore Formation

THP-1 cells (ATCC Cat #285-IF-100) are plated in 96 well plates at a concentration of 200,000 cells per well and allowed to differentiate in RPMI-1640 media (ATCC Cat #30-2001) containing 10% FBS, 100 IU/mL penicillin, 100 ug/mL streptomycin, 100 ng/mL LPS and 100 ng/mL IFN-gamma for 16 hours. Following differentiation, the cells are pretreated with the compound of interest at the appropriate concentration for 30 minutes in RPMI-1640 media containing 100 IU/mL penicillin, 100 ug/mL streptomycin. The pretreatment media is then replaced with assay buffer (20 mM HEPES, 10 mM d-glucose, 118 mM NMDG, 5 mM KCl, 0.4 mM CaCl2) containing 5 uM Yo-Pro 1 (Molecular Probes Cat # Y3603) and the compound of interest at the appropriate concentration and the cells are incubated for an additional 10 minutes. 2′,3′-O-(4-benzoylbenzoyl)-adenosine 5′-triphosphate (Sigma Aldrich Cat# B6396) is then added to a final concentration of 40 uM and fluoroscence readings measured at 491/509 excitation/emission every minute for 50 minutes using a Tecan Safire plate reader. During this time temperature is maintained at of 37° C. Background adjusted fluorescence levels between drug treated and non-treated cells are used to calculate the percent inhibition.

### Example 6

### IL-1β Release Assay

This Example demonstrates the testing of the compounds of this invention for efficacy as inhibitors of P2X₇-mediated release of IL-1β from human macrophages activated by the Alzheimer's beta amyloid peptide 1-42.

### Cell Isolation

Monocytes are isolated from peripheral blood mononuclear cells (PBMCs) as follows. Whole blood is layered directly onto Histopak 1077-1 columns (Sigma Biochemicals) and centrifuged at 800×g for 15 minutes. The PBMC band of cells is removed to a fresh 50 ml culture tube and diluted 1:1 with wash buffer (Phosphate buffered saline, pH 7.4 containing 2 mM EDTA and 5 mg/ml BSA) followed by centrifugation at 800×g for 5 minutes. Cells are then washed by sequential resuspension of the cell pellet in wash buffer and centrifugation at 600×g for 5 minutes. The wash process is repeated until the supernatent is clear of contaminating platelets (generally, 5 to 6 washes). Monocytes are then purified from the PBMCs by negative selection using a monocyte isolation kit (Miltenyi Biotec, Inc.) that contains antibodies to non-monocytic cells, running the cells over a magnetic column to remove antibody-bound cells, and collecting the flow through volume of monocytes. Monocytes are washed once with wash buffer and seeded at 10E5 cells per well in 100 μl serum-free RPMI 1640 in 96-well plates and incubated for 1 hour at 37° C. in a 5% CO₂/95% humidified tissue culture incubator. After 1 hour, the medium is replaced with 100 μl complete culture medium (RPMI 1640, 10% human serum-type AB (heat inactivated), 25 mM HEPES, 2 mM glutamine, 50 U/ml each of penicillin and streptomycin) and incubated overnight (16 hours).

### Dosing Regimen

The next day, the culture medium is replaced with 100 μl fresh complete culture medium in the absence or presence of human beta amyloid 142 peptide (5 μM) and incubated at 37° C. in a 5% CO₂/95% humidified tissue culture incubator for 5 hours. Medium is then removed and discarded. Each well is washed once with Hanks buffered saline (HBSS) containing 1 mM CaCl₂ followed by the addition of 80 μl of HBSS/CaCl₂-inhibiting compound of the present invention (10× stock in HBSS/CaCl₂ for a final concentration of 23 nM and 206 nM) and incubated 15 minutes in the tissue culture incubator followed by the addition of either 10 μl of HBSS/CaCl₂ or 10 μl of benzoyl ATP (BzATP; 3 mM stock in HBSS/CaCl₂ for a 300 μM final concentration) and incubated for a further 30 minutes in the tissue culture incubator. Medium is then removed to new 96-well plates for storage at −70° C. until the IL-1β content was quantitated by ELISA (from R&D Systems). The cells are washed once with HBSS/CaCl₂ followed by lysing the cells with 100 μl ice cold lysis buffer (100 mM Tris, pH 7.6, 1% Triton X-100, and 1 tablet per 30 ml Complete™ protease inhibitor from Roche Biochemicals, Inc). Cell lysates are stored at −70° C. until the IL-1β is quantitated by ELISA.

### Example 7

### Alternate Method for IL-1β Release Assay

THP-1 cells (ATCC Cat #285-IF-100) are plated in 96 well plates at a concentration of 200,000 cells per well and allowed to differentiate in RPMI-1640 media (ATCC Cat #30-2001) containing 10% FBS, 100 IU/mL penicillin, 100 ug/mL streptomycin, 100 ng/mL LPS and 100 ng/mL IFN-γ for 16 hours. Following differentiation, the cells are treated for an additional 2 hours in RPMI-1640 media containing 100 IU/mL penicillin, 100 ug/mL streptomycin and fresh LPS at 100 ng/mL. The cells are then pretreated for 30 minutes with the compound of interest at the appropriate concentration in RPMI media containing 100 IU/mL penicillin, 100 ug/mL streptomycin. Following the pretreatment 2′,3′-O-(4-benzoylbenzoyl)-adenosine 5′-triphosphate (Sigma Aldrich Cat# B6396) is added to a final concentration of 250 uM and the cells incubated for an additional 45 minutes. 30 uL of cell supernatant is then collected and IL-1β levels determined via ELISA (R&D systems Cat. # HSLB50) according to manufacturers recommendations using the Tecan Safire plate reader. Background adjusted IL-1β levels of drug treated and non-treated cells are used to calculate the percent inhibition.

### In Vivo Assay Methods

### Example 8

### EAE Model

This example illustrates the efficacy of the compounds of this invention in the treatment of multiple sclerosis. As described herein, experimental autoimmune encephalomyelitis (EAE) model is used to show such an efficacy. The following procedures are employed in this model.

### Animals

SJL/J female mice, 8 wks. old, are obtained from Jackson Laboratories.

### Antigens

Myelin Proteolipid Protein (PLP 139-151) (HSLGKWLGHPDKF) (Cat # H-2478) is obtained from BACHEM, Bioscience, Inc., 3700 Horizon Dr., King of Prussia, Pa. 19406, 1-610-239-0300 (phone), 1-610-239-0800 (fax).

Complete Freund's Adjuvant H37 Ra [1 mg/ml Mycobacterium Tuberculosis H37 Ra] is obtained from Difco 1-800-521-0851 (Cat #3114-60-5, 6×10 ml).

Mycobacterium Tuberculosis is also obtained from Difco, 1-800-521-0851 (Cat #3114-33-8, 6.times.100 mg).

### Pertussis Toxin

Bordetella Pertussis, (Lyophilized powder containing PBS and lactose) is obtained from List Biological Laboratories, 1-408-866-6363 (Product #180, 50 ug).

### Induction of EAE in Mice

PLP139-151 peptide is dissolved in H₂O:PBS (1:1) solution to a concentration 7.5 mg/10 ml (for 75 μg PLP per group) and emulsified with an equal volume of CFA supplemented with 40 mg/10 ml heated-killed mycobacterium tuberculosis H37Ra. Mice are injected s.c. with 0.2 ml of peptide emulsion in the abdominal flank (0.1 ml on each side). On the same day and 72 hours later, mice are injected i.v. with 100% of 35 ng and 50 ng of Bordetella Pertussis toxin in saline respectively.

Clinical Assessment
STAGE 0: Normal
STAGE 0.5: Partial limp tail
STAGE 1: Complete Limp Tail
STAGE 2: Impaired righting reflex
STAGE 2.5: Righting reflex is delayed (Not weak enough to be stage 3).
STAGE 3: Partial hind limb paralysis
STAGE 3.5: One leg is completely paralyzed, and one leg is partially paralyzed,
STAGE 4: Complete hind limb paralysis.
STAGE 4.5: Legs are completely paralyzed and Moribund
STAGE 5: Death due to EAE

Clinical Courses of EAE
Acute phase: First clinical episode (Day 10-18)
Remission: Phase of clinical improvement following a clinical episode; characterized by a reduction (>=one grade) in clinical score for at least two days after the peak score of acute phase or a disease relapse.

Relapse: Increase of at least one grade in clinical score for at least two days after remission has been attained.

The animals treated with the compounds of this invention generally would be expected to show improvements in clinical scores.

### Example 9

### Stroke Model

This Example illustrates a protocol for determining the efficacy of the compounds of the present invention for the treatment of stroke using an animal model.

Male Sprague Dawley rats (Charles River) weighing 280-320 g are given free access to food and water and acclimatized for a minimum of 4 days before use in experiments. All rats for use in studies are to be fasted beginning at 3:00 pm the day prior to surgery but given free access to water. Prior to surgery each rat is weighed. The rat is initially induced with 5% isoflurane (Aerrane, Fort Dodge), combined with 30% O₂, 70% N₂O for 2-5 minutes. The rat is then placed on a circulating water-heating pad and into a nose cone for spontaneous respiration of anesthetic gases. The isoflurane is reduced to 2%. A rectal probe is inserted and body temperature maintained at 36.5-37.5° C. The hair is clipped at all surgical sites and these regions will then be scrubbed with Betadine.

### Surgical Procedure

A temporalis muscle probe is placed into the right temporalis muscle and “brain” temperature” is monitored. A midline neck incision is made in the upper thorax of the rat. Careful dissection, isolation and retraction of the sternomastoideus, digastricus, and sternohyoideus muscles is made to expose the right common, internal and external carotid arteries. The right common carotid artery is isolated with a 5-0 silk suture. During surgery the suture is released allowing reperfusion every 2-4 minutes. The right external carotid and superior thyroid arteries are also isolated and the superior thyroid is cauterized, while the external carotid is ligated distally with a 5-0 silk suture. Another 5-0 silk suture is loosely tied around the external carotid artery. The occipital artery is isolated, ligated and incised. The internal carotid is isolated.

With the common and external carotid arteries immobilized, an aneurysm clip is placed onto the internal carotid artery. A small incision is made at the distal end of the external carotid. A 3-0 nylon suture coated with poly-L-lysine is then inserted into the external carotid and up into the common carotid artery. The loosely tied 5-0 silk suture around the external carotid is now gently tightened around the filament. The external carotid artery is then incised and the remaining piece of the external carotid artery with the filament is rotated so that the filament may be inserted into the internal carotid artery the length of insertion depending on the weight and rat strain. In Sprague Dawley rats the monofilament is inserted 18-19 mm (18 mm for rats weighing <300 gm, 19 mm for rats weighing .gtoreq.300 gm) effectively blocking blood flow to the middle cerebral artery.

The external jugular vein will be cannulated with PE 50 tubing for I.V. administration of compounds. The cannula will be exteriorized at the previously shaven, scruff of the neck and sutured in place. The wound will be closed by means of suture. The right femoral artery is catheterized for blood gas and glucose determination during surgery.

Two hours after the insertion of the monofilament suture the rats are re-anesthetized with the same anesthetic combination used initially and placed back into the nose cone with the reduction of isoflurane concentration to 2%. The neck incision is reopened to expose the external carotid artery. The restoration of blood flow is accomplished by completely withdrawing the intraluminal suture from the carotid arteries. The incision is then closed with 3-0 silk in an interrupted stitch.

Compound Administration

Five groups of 15 animals are subjected to the above methodology. Compounds are infused (I.V.) at various doses (dose response) over different time period's post MCAo. A pre-determined concentration is infused over a pre-selected time period beginning at various intervals post MCAo. Vehicle-treated controls receive an infusion of normally 0.9 ml/hr. A positive control compound is run at the same time.

### Neurological Tests

Prior to surgery, 2 hours following the onset of ischaemia and 24 hours after ischaemia a battery of neurological tests is performed. The postural reflex test, which is designed to examine upper body posture, when the rat is suspended by the tail above a flat surface. A normal rat will extend the entire body and both forelimbs towards the surface. Rats with an infarction will consistently flex the contralateral limb and show signs of body rotation.

The rats respond to a gentle lateral push with a finger behind the shoulders. A normal rat would resist such a push, whereas a rat with an infarction will not. The elicited forelimb placing in response to visual and tactile stimuli. The animal is held by the body so that the lateral or dorsal forepaw surface is placed against a bench. This test is repeated but on this occasion obstructing the view of the rat. Upon completion of each experiment, all animals are deeply anaesthetized with isoflurane (5%), euthanized by decapitation, and the brains removed, the extent and location of the ischaemic damage is verified histologically by means of tetrazolium chloride.

### Example 10

### DNBS Model

This Example illustrates the anti-inflammatory activity of the compounds of this invention using a model of 2,4-dinitrobenzenesulfonic acid (DNBS) induced distal colitis (a model of inflammatory bowel disease).

### Test Substance and Dosing Pattern

A compound of this invention is dissolved in vehicle of 2% Tween 80 in distilled water for oral administration at a dose of 50 mg/kg or dissolved in vehicle of 2% Tween 80 and 0.9% NaCl for intraperitoneal injection at 30 mg/kg. The dose is given once daily for 7 consecutive days. Dosing volume is 10 ml/kg. DNBS is challenged 2 hours after dosing on the second day.

### Animals

In these studies, male Wistar, Long Evans rats provided by animal breeding center of MDS Panlabs Taiwan, Ltd. and Balb/cByJ derived male mice (weighing 20±2 gms), provided by National Laboratory Animals Breeding Research center (NALBRC, Taiwan), may be used. Space allocation of 6 animals may be 45×23×15 cm. Animals are housed in APEC® cages (Allentown Caging, Allentown, N.J. 08501, USA) in a positive pressure isolator (NuAire®, Mode: Nu-605, airflow velocity 50±5 ft/min, HEPA Filter) and maintained in a controlled temperature (22° C.-24° C.) and humidity (60%-80%) environment with 12 hours light dark cycles for at least one week in MDS Panlabs Taiwan laboratory prior to being used. Free access to standard lab chow for rats (Fwusow Industry Co., Limited, Taiwan) and tap water was granted. All aspects of this work including housing, experimentation and disposal of animals would be performed in general accordance with the International Guiding Principles for Biomedical Research Involving Animals (CIOMS Publication No. ISBN 92 90360194, 1985).

### Chemicals

DNBS is obtained from TCI, Tokyo, Japan, ethanol is from Merck, Germany and Sulfasalazine is purchased from Sigma, USA.

### Equipment

Electriconic scale (Tanita, model 1140, Japan), Electriconic scale (Sartorius, R160P, Germany), Glass syringe (2 ml, Mitsuba, Japan), Rat oral needle, Hypodermic needle (25G.times.1″TOP Corporation, Japan), Stainless Scissors (Klappenclear, Germany), Stainless Forceps (Klappenclear, Germany).

### Method

Groups of 3 Wistar derived male rats weighing 180±20 gms are used. Distal colitis is induced by intra-colonic instillation of DNBS (2,4-dinitrobenzene sulfonic acid, 30 mg in 0.5 ml ethanol 30%) after which, 2 ml of air is gently injected through the cannula to ensure that the solution remains in the colon. Test substance is administered orally (PO) at a dose of 50 mg/kg or intraperitoneally (IP) at 30 mg/kg once daily for 7 consecutive days. DNBS is instillated into the distal colon of each animal 2 hours after dosing on the second day. The control group is similarly treated with vehicle alone and sulfasalazine (300 mg/kg, PO) is used as reference agent. Animals are fasted 24 hours before DNBS challenge and 24 hours after the final treatment when they are sacrificed and each colon is removed and weighed. During the experiments, presence of diarrhea is recorded daily. When the abdominal cavity is opened before removal of the colon, adhesions between the colon and other organs are noted. After weighing the colon, the extent of colonic ulceration is observed and noted as well. Colon-to-body weight ratio is then calculated for each animal according to the formula: Colon (g)/BW×100%. The “Net” increase in ratio of Vehicle-control+DNBS group relative to Vehicle-control group is used as a base value for comparison With test substance treated groups and expressed as % decrease in inflammation. A 30 percent or more (30%) decrease in “Net” colon-to-body weight ratio for each test substance treated group relative to the “Net” vehicle+DNBS treated group is considered significant.

### Example 11

### Carrageenan Model

This Example illustrates the anti-inflammatory activity of the present compounds using a model of carrageenan induced paw edema (a model of inflammation, carrageenan).

### Test Substance and Dosing Pattern

A compound of this invention is dissolved in vehicle of 2% Tween 80/0.9% NaCl and administered intraperitoneally at a dose of 30 mg/kg 30 minutes before carrageenan (1% 0.1 ml/paw) challenge. Dosing volume is 10 ml/kg.

### Animals

Animals are conditioned in accordance with the procedures set forth in the previous Example.

### Chemicals

Carrageenan is obtained from TCI, Japan; Pyrogen free saline is from Astar, Taiwan; and Aspirin is purchased from ICN BioMedicals, USA.

### Equipment

Glass syringe (1 ml and 2 ml Mitsuba, Japan), Hypodermic needle 24Gx1″ (Top Corporation, Japan), Plethysmometer #7150 (UGO Basile, Italy), and Water cell 25 mm Diameter, #7157 (UGO Basile, Italy).

### Method

Test substance (Example) is administered IP (30 mg/kg) to groups of 3 Long Evans derived male overnight fasted rats weighing 150±20 gms 30 minutes before right hind paw injection of carrageenan (0.1 ml of 1% suspension intraplantar). Hind paw edema, as a measure of inflammation, is recorded 3 hours after carrageenan administration using a plethysmometer (Ugo Basile Cat. #7150) with water cell (25 mm diameter, Cat. #7157). Reduction of hind paw edema by 30 percent or more (30%) indicated significant acute anti-inflammatory activity.

### Example 12

### Collagen Model

This Example illustrates the anti-inflammatory activity of the present compounds using a model of Balb/c mice subjected to monoclonal antibody (mAb) type II collagen induced arthritis.

### Test Substance and Dosing Pattern

A compound of this invention is dissolved in vehicle of 2% Tween 80/0.9% NaCl, at doses of 50 or 30 and administered orally (50 mg/kg) or intraperitoneally at 30 mg/kg once daily for 3 consecutive days after monoclonal antibody of collagen was injected. Dosing volume is 20 ml/kg.

### Animals

Animals are conditioned in accordance with the procedures set forth in the previous Example.

### Chemicals

Lipopolysaccharide is obtained from Sigma, USA; Indomethacin is from Sigma, USA; Arthrogen-CIA™. Monoclonal Antibodies D8, F10, DI-2G and A2 are obtained from IBL, Japan; Phosphated-Buffer Saline is purchased from Sigma, USA; and Tween 80 is from Wako, Japan.

### Equipment

Plethysmometer (Ugo Basile, Italy) and Water Cell (Ugo Basile, Italy).

### Method

Groups of 5 Balb/cByJ mice strain, 6-8 weeks of age, are used for the induction of arthritis by monoclonal antibodies (mAbs) responding to type II collagen, plus lipopolysaccharide (LPS). The animals are administered intravenously with a combination of 4 different mabs in a total of 4 mg/mouse at day 0, and followed by intravenous 25 μg of LPS 72 hours later (day 3). From day 3, one hour after LPS administration, ML-659 at 50 mg/kg (PO) or 30 mg/kg (IP) and vehicle (2% Tween 80/0.9% NaCl, PO) as well as the positive control indomethacin, 3 mg/kg (PO) are administrated once daily for 3 consecutive days. A plethysmometer (Ugo Basile Cat #7150) with water cell (12 mm diameter) is used for the measurement of increase in volume of the two hind paws at day 0, 5, 7, 10, 14, and 17. The percent inhibition of increase in volume is calculated by the following formula:

Inhibition (%): [1−(Tn−To)/(Cn−Co)]×100

### Where:

Co (Cn): volume of day 0 (day n) in vehicle control
To (Tn): volume of day 0 (day n) in test compound-treated group
The reduction of both of two hind paws edema by more than 30% is considered significant.

### Example 13

### Neuropathic Pain Model

This example illustrates the analgesic activity of the compounds of this invention using a Sciatic Nerve ligation model of mononeuropathic pain

### Test System

Adult male Sprague Dawley (SD) rats weighing 250-300 gm (Charles River Laboratories, San Diego, Calif.) were used. The animal room was lighted artificially at a 12-hr light-dark cycle (from 7:00 A.M. to 7:00 P.M) with water and food supply ad libitum. Animals were allocated randomly into groups.

### Model Induction

Sciatic nerve ligation (SNL, Seltzer's model):

Under anesthesia with pentobarbital (50 mg/kg, i.p.) and aseptic techniques, the selective nerve injury is created by tightly ligating the selective portion of the common sciatic nerve according to the method of Seltzer (1990). Briefly, the high-thigh level of the left sciatic nerve is exposed after skin incision and blunt separation of muscles at a site near the trochanter just distal to the point at which the posterior biceps semitendious nerve nerve branches from the common sciatic nerve. The nerve is then fixed in this position with fine forceps by pinching the epineurium on its dorsal aspect, taking care not to press the nerve against underlying structures. An 8-0 silicon-treated silk suture is inserted into the nerve with a ⅜ curved, reversed-cutting mini-needle, and tightly ligated so that the dorsal ⅓-½ of the nerve is trapped in the ligature. The muscles are sutured in layers, and the skin closed with wound clips. Animals are then returned to their home cages. Rats exhibiting postoperative neurological deficits or poor grooming are excluded from the experiments.

### Equipment

The following equipment was used in the current studies: von Frey filament set (Touch-test Sensory Evaluator, North Coast Medical Inc., Morgan Hill, Calif.).

### Statistical Methods:

Within each experiment mean, standard error of the mean (SEM) and statistical significance are calculated using the average, standard error of the mean and unpaired, two-tailed t-Test functions, respectively, using Microsoft Excel®. Statistical significance of effects observed between individual experiments is determined, using Prism (GraphPad Software Inc., San Diego, Calif.) for the one-way or two-way analysis of variance (ANOVA) function. Statistical analyses are performed with a confidence limit of 0.95 and a significance level of 0.05.

The synthetic and biological examples described in this application are offered to illustrate this invention and are not to be construed in any way as limiting the scope of this invention. In the examples, all temperatures are in degrees Celsius (unless otherwise indicated). The compounds that have been prepared in accordance with the invention along with their biological activity data are presented in following Tables. The syntheses of these representative compounds are or may be carried out in accordance with the methods set forth above.

### Exemplary Compounds of the Invention

The following compounds have been or can be prepared according to the methods of the invention. For the purposes of Table 1, activity of each compound is expressed as follows:

TABLE 1
Compounds

| | | | | IL-1β % | |
|---|---|---|---|---|---|
| | | | | inhib @ | |
| | | | | 0.1 μM | Method |
| | | MW | MS | or | of |
| ID | Structure | (calc) | (obs) | 0.3 μM | Synthesis |
| 1 | 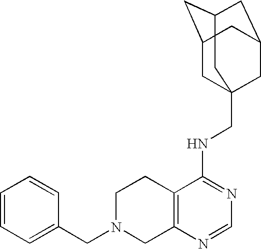 | 388.56 | 389.83 | ++++ | A |
| 2 | 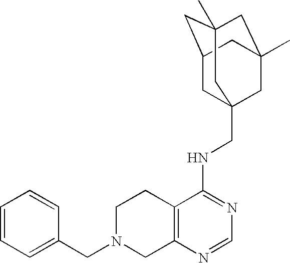 | 416.61 | 417.82 | * | A |
| 3 | 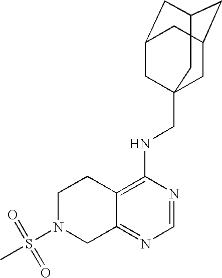 | 376.52 | 377.31 | ** | B |
| 4 | 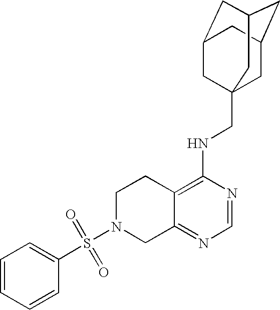 | 438.59 | 439.30 | *** | B |
| 5 | 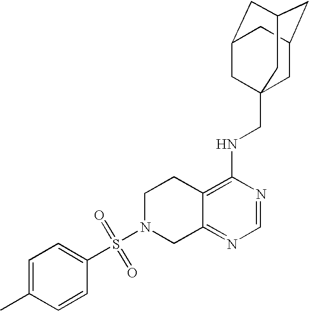 | 452.62 | 453.09 | *** | B |
| 6 | 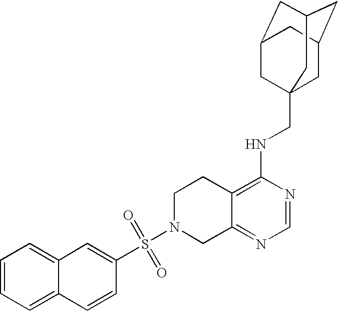 | 488.65 | 489.31 | * | B |
| 7 | 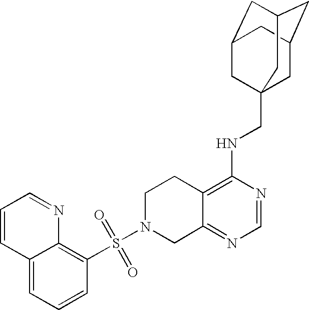 | 489.64 | 490.24 | **** | B |
| 8 | 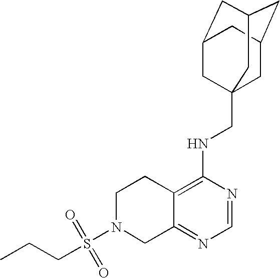 | 404.58 | 405.36 | *** | B |
| 9 | 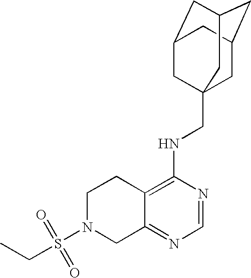 | 390.55 | 391.33 | ++++ | B |
| 10 | 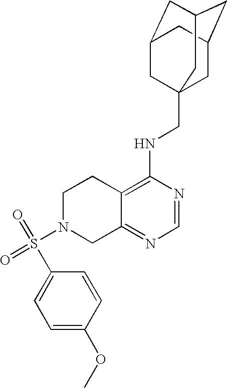 | 468.62 | 469.32 | ** | B |
| 11 | 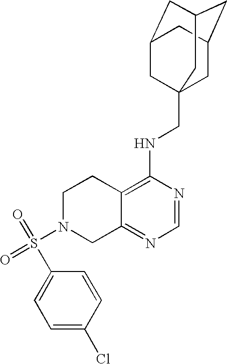 | 473.04 | 473.16 | * | B |
| 12 | 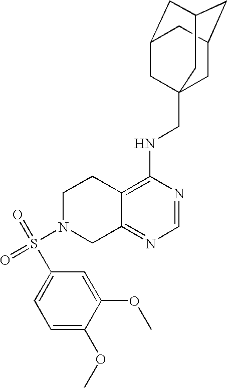 | 498.64 | 499.28 | ** | B |
| 13 | 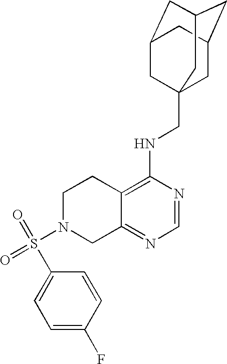 | 456.58 | 457.31 | ** | B |
| 14 | 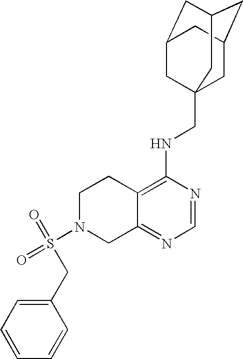 | 452.62 | 453.09 | **** | B |
| 15 | 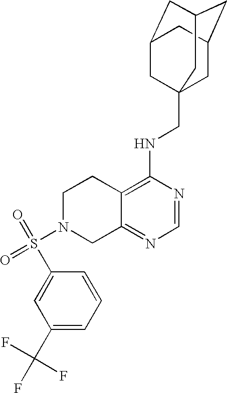 | 506.59 | 507.22 | * | B |
| 16 | 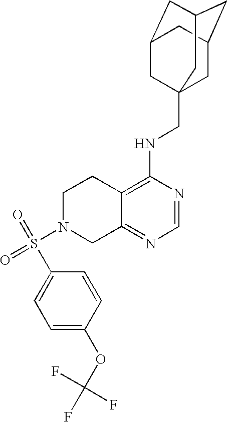 | 522.59 | 523.36 | * | B |
| 17 | 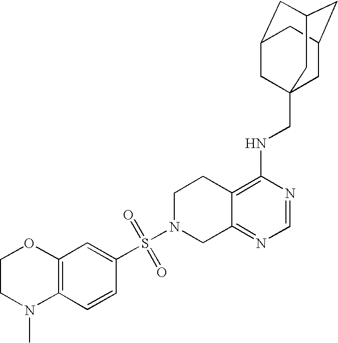 | 509.67 | 510.41 | * | B |
| 18 | 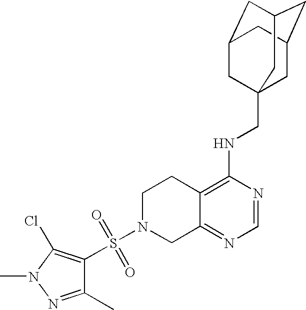 | 491.06 | 491.20 | * | B |
| 19 | 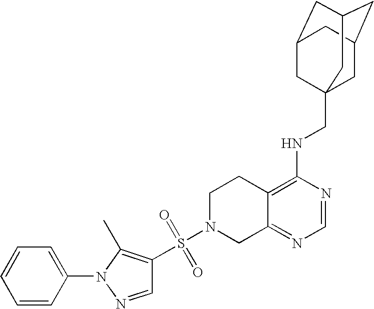 | 518.68 | 519.32 | *** | B |
| 20 | 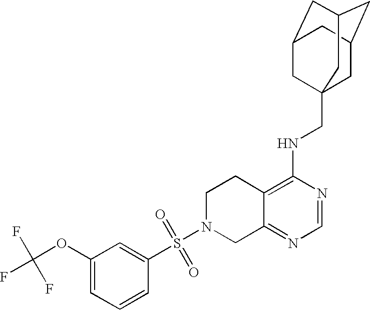 | 522.59 | 523.36 | ** | B |
| 21 | 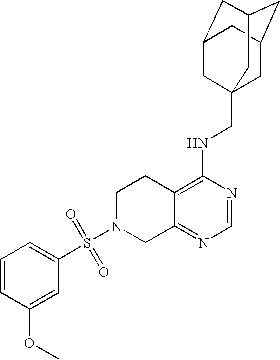 | 468.62 | 469.32 | *** | B |
| 22 | 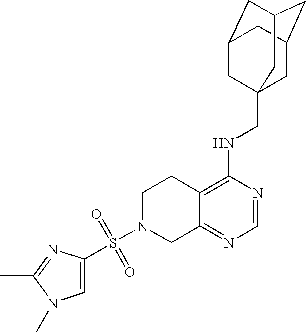 | 456.61 | 457.33 | * | B |
| 23 | 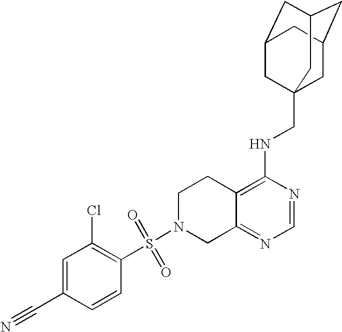 | 498.05 | 498.37 | * | B |
| 24 | 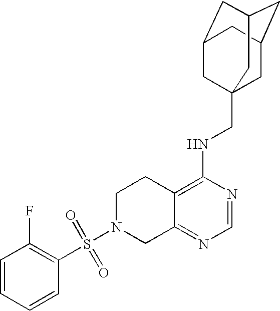 | 456.58 | 457.31 | ++++ | B |
| 25 | 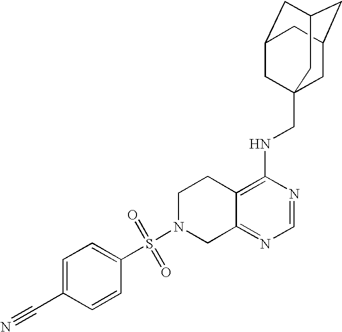 | 463.60 | 464.13 | ** | B |
| 26 | 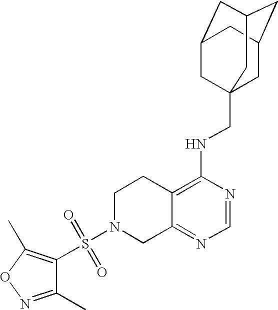 | 457.60 | 458.37 | *** | B |
| 27 | 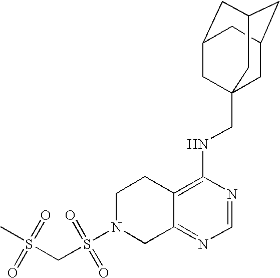 | 454.61 | 455.18 | | B |
| 28 | 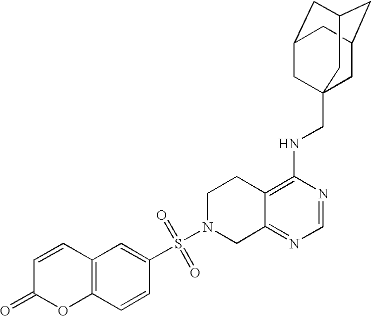 | 506.62 | 507.24 | ** | B |
| 29 | 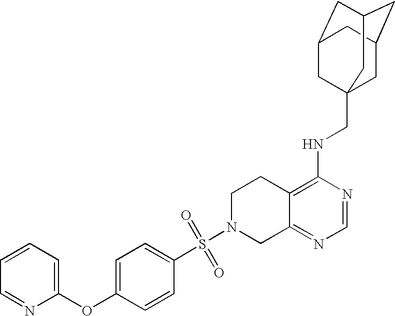 | 531.68 | 532.15 | **** | B |
| 30 | 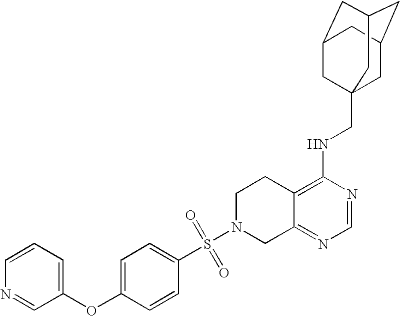 | 531.68 | 530.74 | | B |
| 31 | 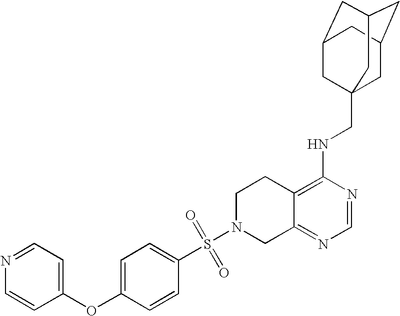 | 531.68 | 532.14 | ** | B |
| 32 | 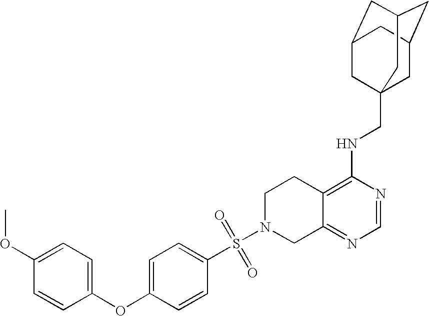 | 560.72 | 561.12 | * | B |
| 33 | 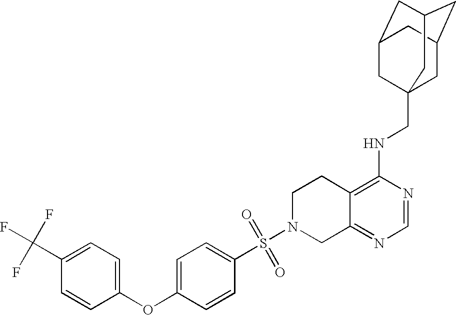 | 598.69 | 599.28 | * | B |
| 34 | 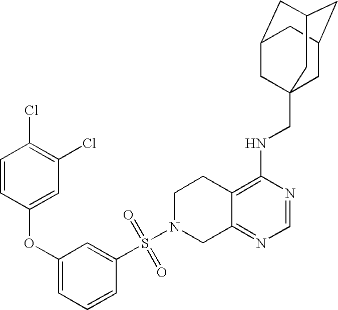 | 599.58 | 599.13 | * | B |
| 35 | 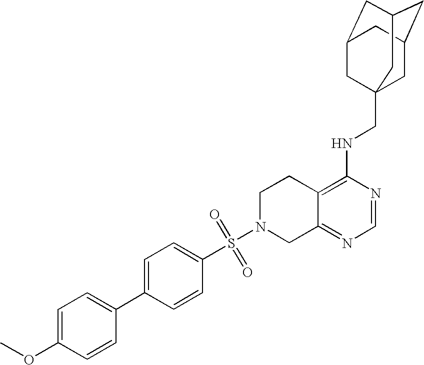 | 544.72 | 545.28 | * | B |
| 36 | 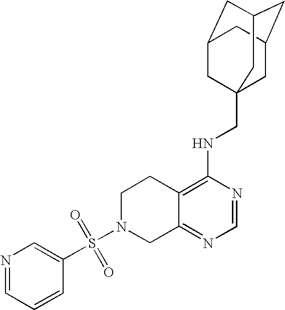 | 439.58 | 440.28 | | B |
| 37 | 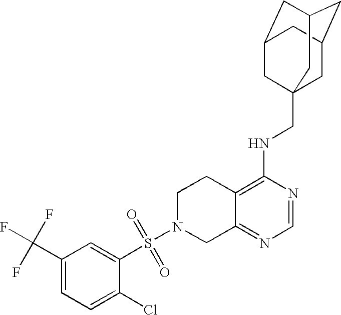 | 541.04 | 541.23 | ** | B |
| 38 | 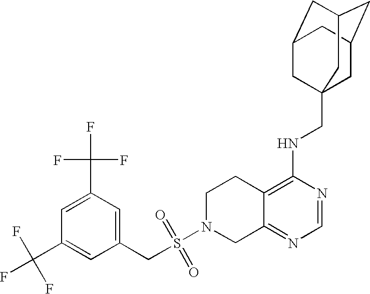 | 588.61 | 589.26 | * | B |
| 39 | 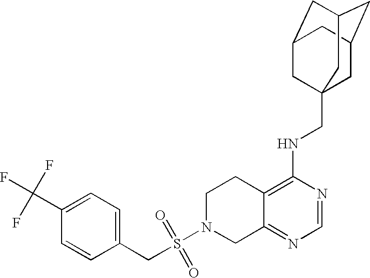 | 520.62 | 521.27 | * | B |
| 40 | 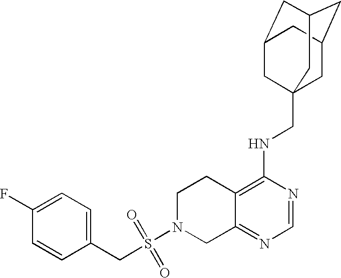 | 470.61 | 471.38 | * | B |
| 41 | 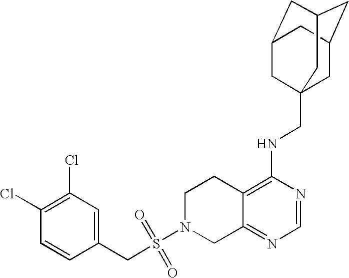 | 521.51 | 521.18 | * | B |
| 42 | 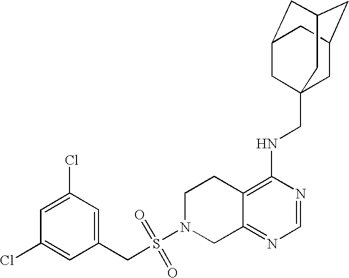 | 521.51 | 521.19 | ** | B |
| 43 | 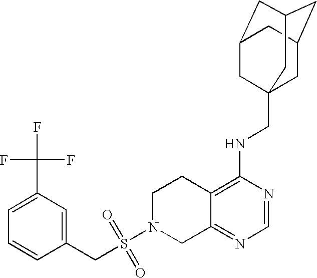 | 520.62 | 521.27 | ** | B |
| 44 | 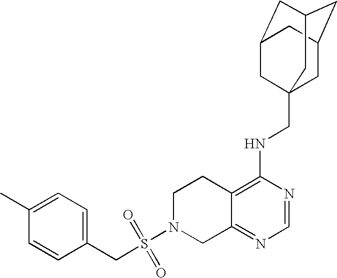 | 466.65 | | | B |
| 45 | 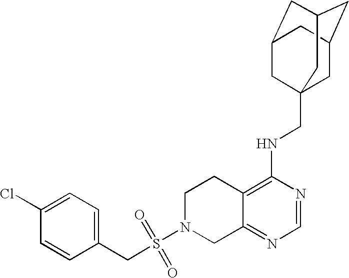 | 487.06 | 487.27 | ** | B |
| 46 | 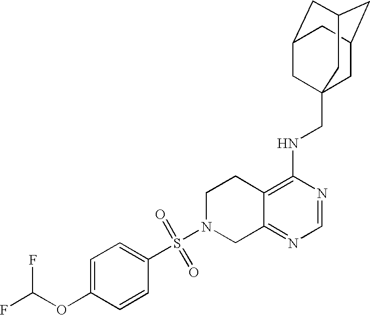 | 504.60 | 505.14 | ** | B |
| 47 | 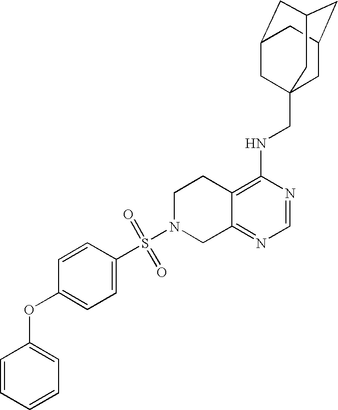 | 530.69 | 531.20 | +++ | B |
| 48 | 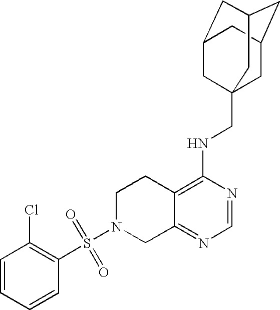 | 473.04 | 473.16 | **** | B |
| 49 | 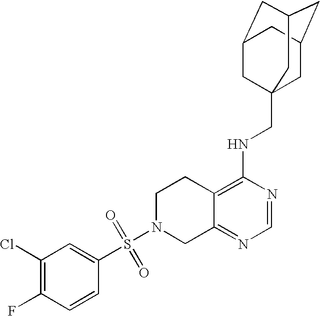 | 491.03 | 491.15 | *** | B |
| 50 | 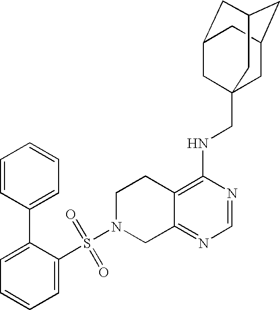 | 514.69 | 515.31 | ++ | B |
| 51 | 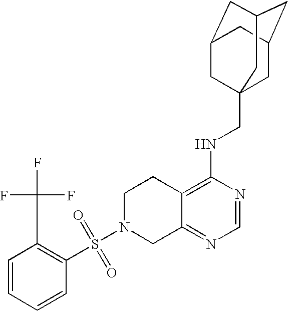 | 506.59 | 507.22 | * | B |
| 52 | 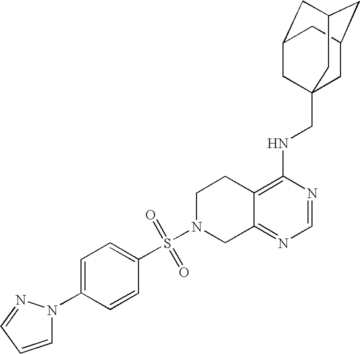 | 504.66 | 505.22 | * | B |
| 53 | 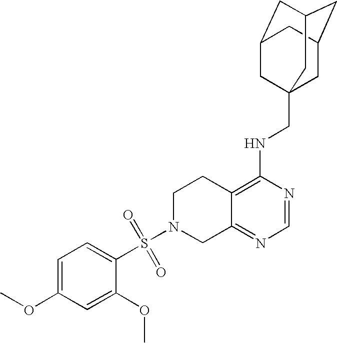 | 498.64 | 499.26 | *** | B |
| 54 | 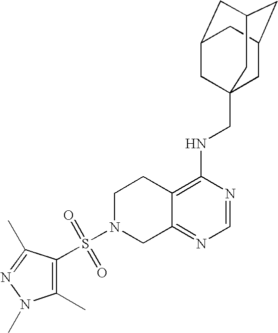 | 470.64 | 471.41 | * | B |
| 55 | 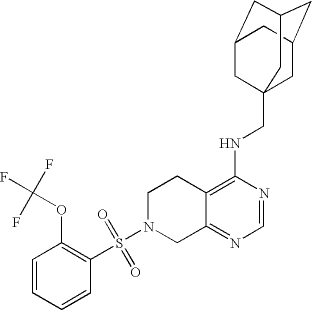 | 522.59 | 523.35 | * | B |
| 56 | 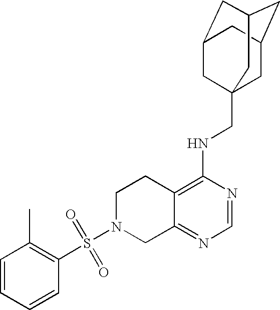 | 452.62 | 453.06 | ** | B |
| 57 | 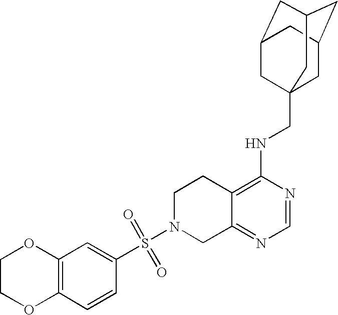 | 496.63 | 497.37 | *** | B |
| 58 | 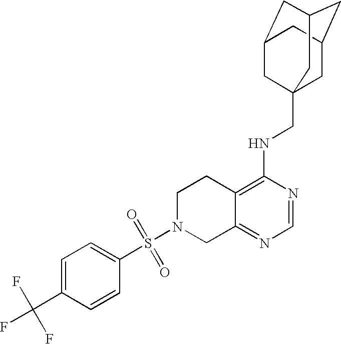 | 506.59 | 507.23 | * | B |
| 59 | 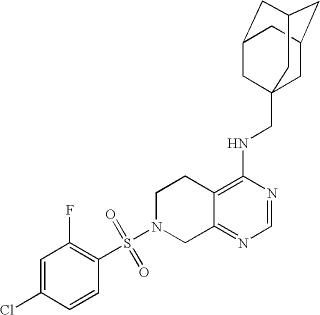 | 491.03 | 491.14 | ** | B |
| 60 | 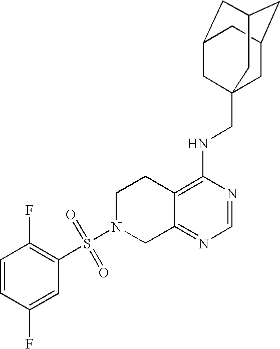 | 474.57 | 475.09 | ** | B |
| 61 | 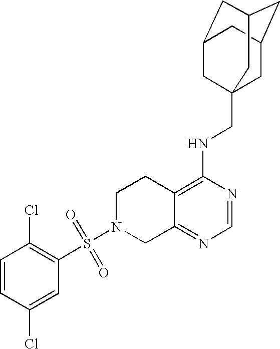 | 507.48 | 507.10 | *** | B |
| 62 | 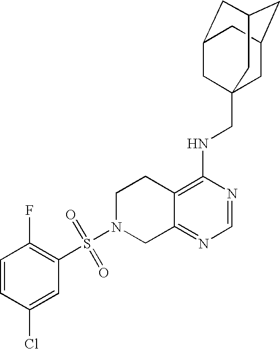 | 491.03 | 491.14 | ** | B |
| 63 | 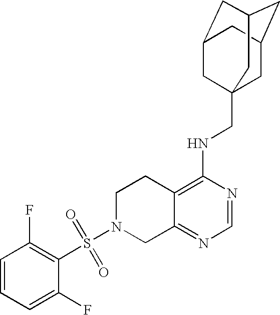 | 474.57 | 475.11 | **** | B |
| 64 | 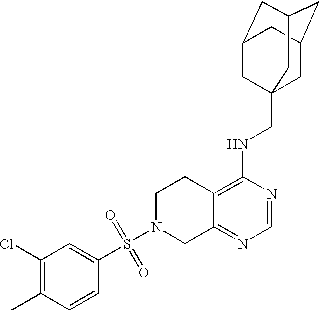 | 487.06 | 487.27 | ** | B |
| 65 | 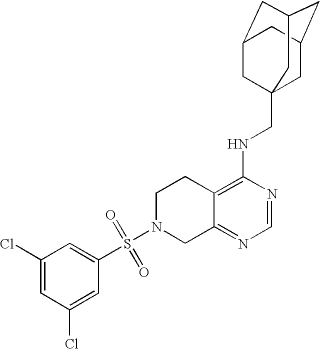 | 507.48 | 507.09 | * | B |
| 66 | 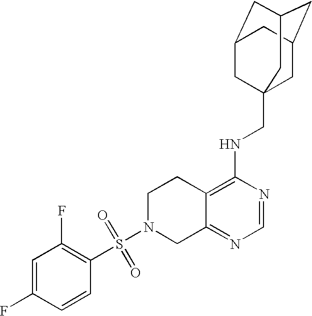 | 474.57 | 475.10 | ++ | B |
| 67 | 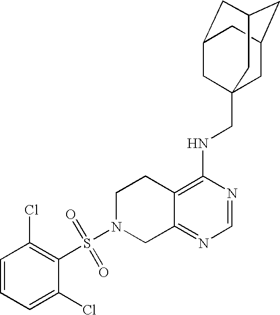 | 507.48 | 507.08 | **** | B |
| 68 | 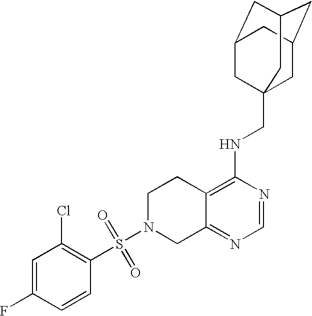 | 491.03 | 491.14 | * | B |
| 69 | 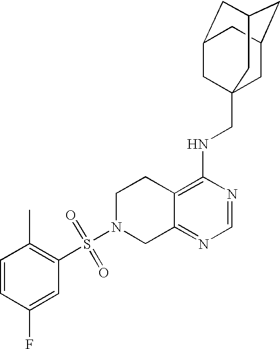 | 470.61 | 471.37 | * | B |
| 70 | 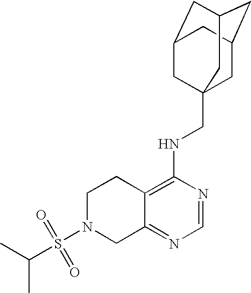 | 404.58 | 405.37 | *** | B |
| 71 | 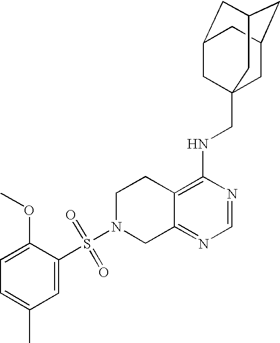 | 482.65 | 483.31 | ** | B |
| 72 | 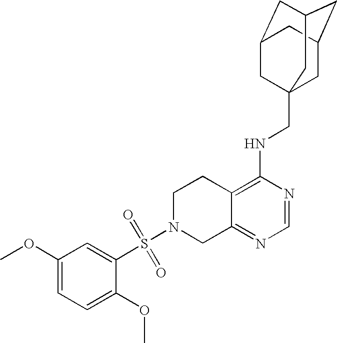 | 498.64 | 499.26 | ** | B |
| 73 | 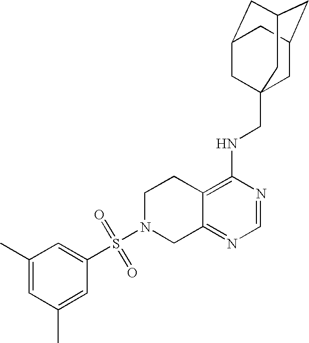 | 466.65 | 467.27 | * | B |
| 74 | 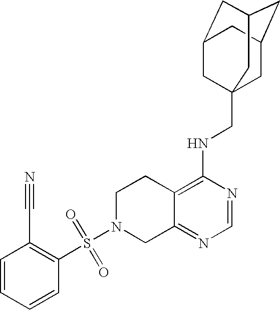 | 463.60 | 464.13 | ** | B |
| 75 | 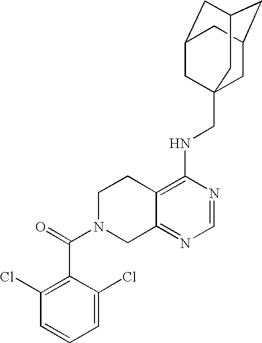 | 471.43 | 471.35 | ++++ | C |
| 76 | 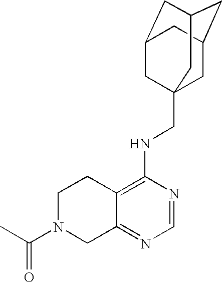 | 340.47 | 341.19 | | C |
| 77 | 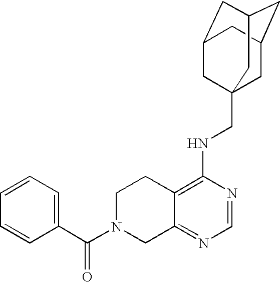 | 402.54 | 403.37 | ++++ | C |
| 78 | 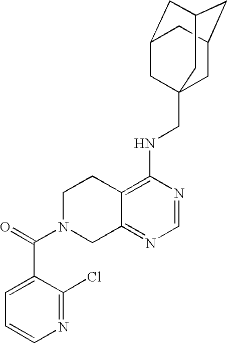 | 437.97 | 438.15 | | C |
| 79 | 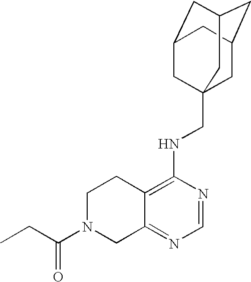 | 354.50 | 355.30 | | C |
| 80 | 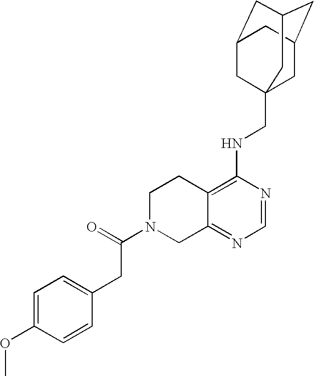 | 446.59 | 447.25 | **** | C |
| 81 | 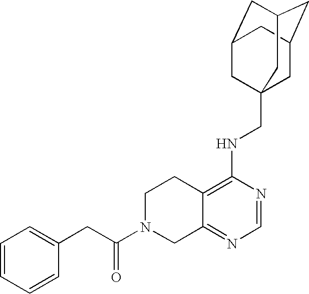 | 416.57 | 417.39 | *** | C |
| 82 | 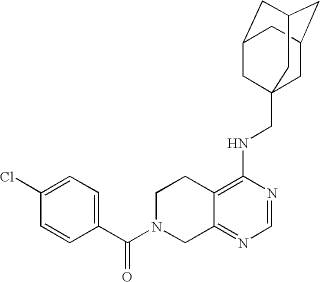 | 436.98 | 437.12 | ** | C |
| 83 | 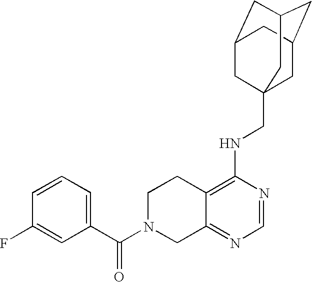 | 420.53 | 421.20 | ++++ | C |
| 84 | 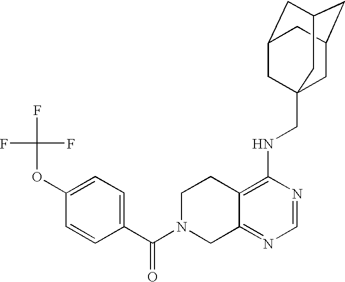 | 486.54 | 487.30 | * | C |
| 85 | 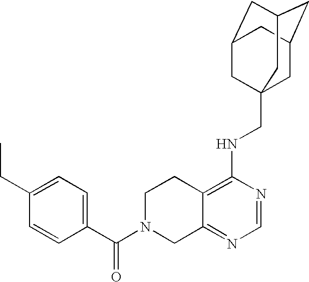 | 430.59 | 431.35 | *** | C |
| 86 | 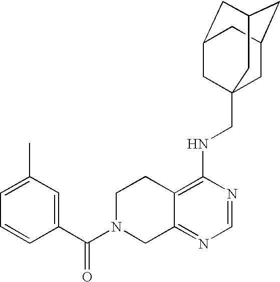 | 416.57 | 417.39 | **** | C |
| 87 | 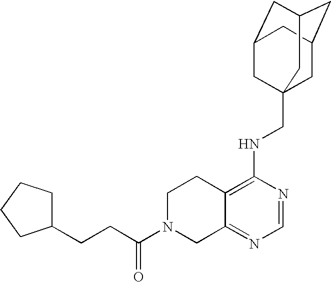 | 422.61 | 423.29 | | C |
| 88 | 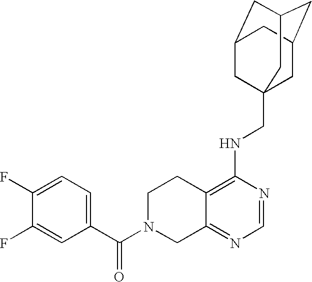 | 438.52 | 439.28 | **** | C |
| 89 | 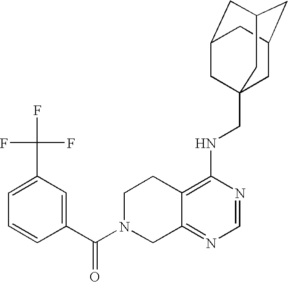 | 470.54 | 471.39 | ** | C |
| 90 | 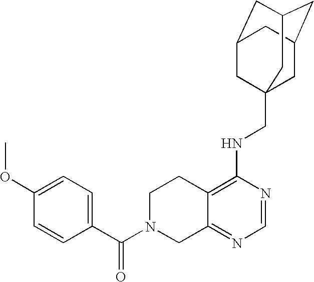 | 432.56 | 433.30 | * | C |
| 91 | 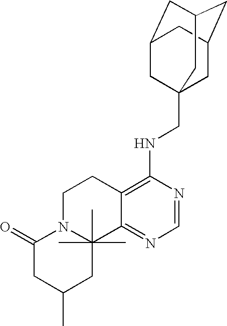 | 438.66 | 439.47 | | C |
| 92 | 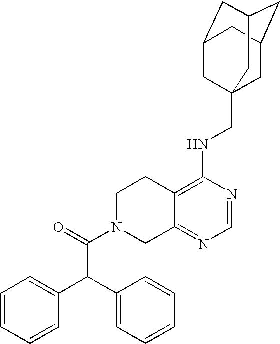 | 492.66 | 493.36 | * | C |
| 93 | 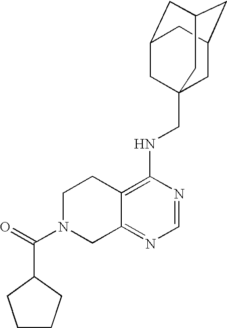 | 394.56 | 395.19 | | C |
| 94 | 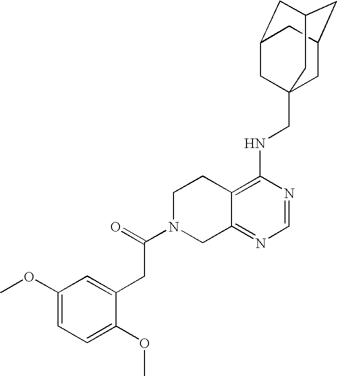 | 476.62 | 477.30 | | C |
| 95 | 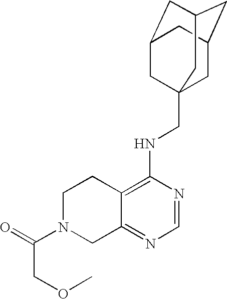 | 370.49 | 371.21 | | C |
| 96 | 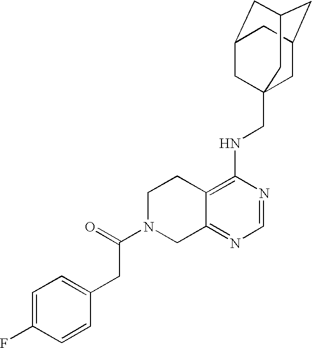 | 434.56 | 435.36 | *** | C |
| 97 | 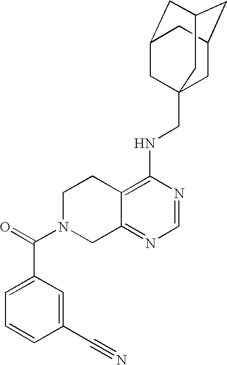 | 427.55 | 428.18 | ++++ | C |
| 98 | 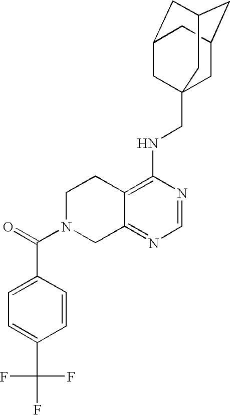 | 470.54 | 471.39 | ** | C |
| 99 | 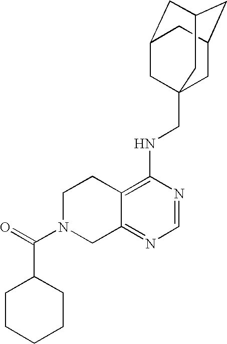 | 408.59 | 409.38 | | C |
| 100 | 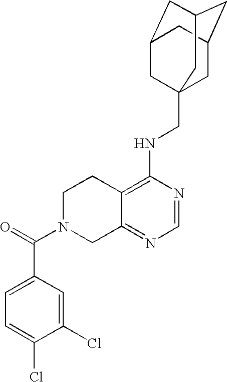 | 471.43 | 471.34 | ** | C |
| 101 | 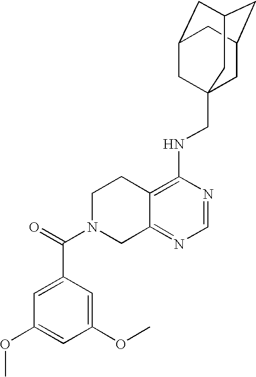 | 462.59 | 463.29 | * | C |
| 102 | 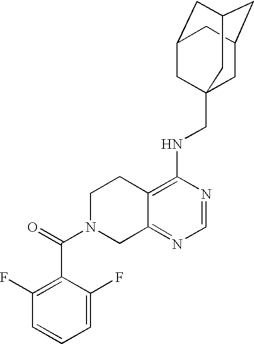 | 438.52 | 439.30 | *** | C |
| 103 | 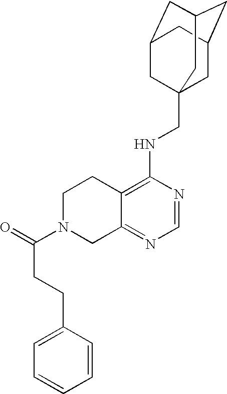 | 430.59 | 431.37 | **** | C |
| 104 | 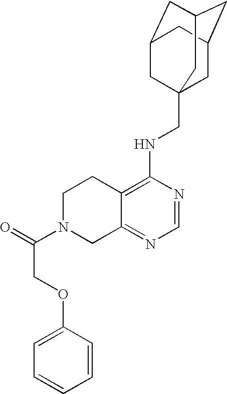 | 432.56 | 433.31 | +++ | C |
| 105 | 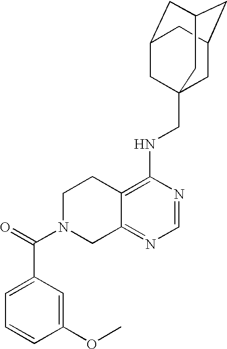 | 432.56 | 433.31 | ++ | C |
| 106 | 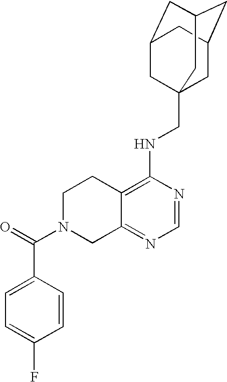 | 420.53 | 421.22 | ++++ | C |
| 107 | 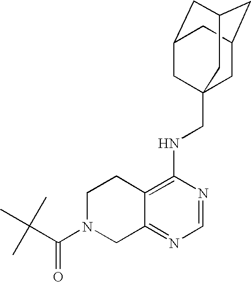 | 382.55 | 383.32 | *** | C |
| 108 | 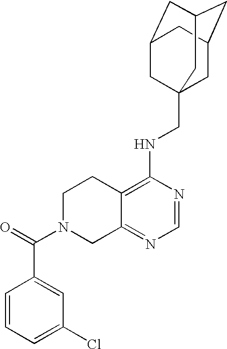 | 436.98 | 437.21 | **** | C |
| 109 | 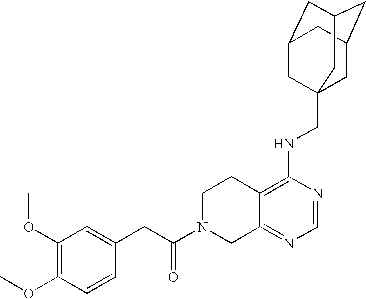 | 476.62 | 477.47 | | C |
| 110 | 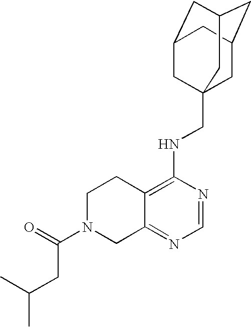 | 382.55 | 383.32 | | C |
| 111 | 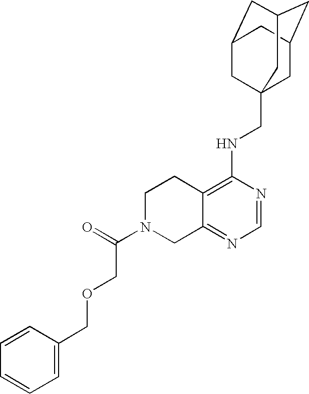 | 446.59 | 447.24 | **** | C |
| 112 | 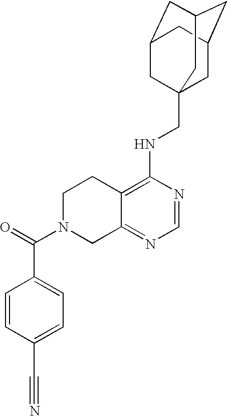 | 427.55 | 428.19 | | C |
| 113 | 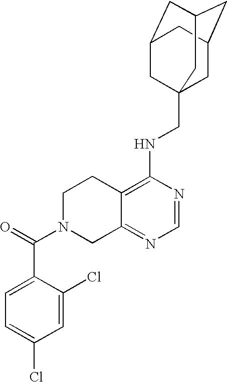 | 471.43 | 471.35 | **** | C |
| 114 | 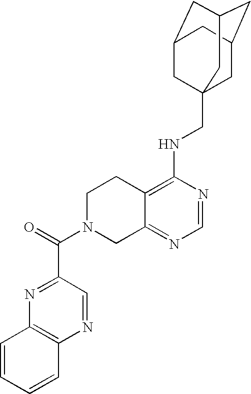 | 454.58 | 455.32 | | C |
| 115 | 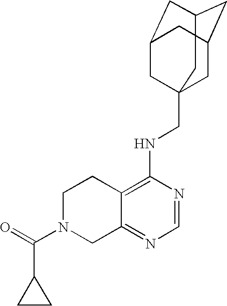 | 366.51 | 367.65 | ++++ | C2 |
| 116 | 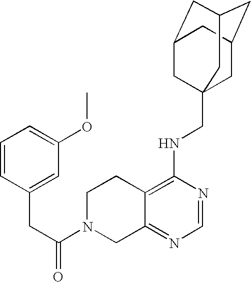 | 446.59 | 447.24 | ** | C |
| 117 | 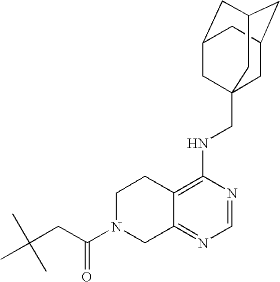 | 396.58 | 397.30 | | C |
| 118 | 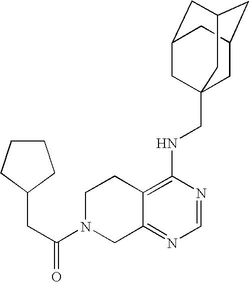 | 408.59 | 409.38 | | C |
| 119 | 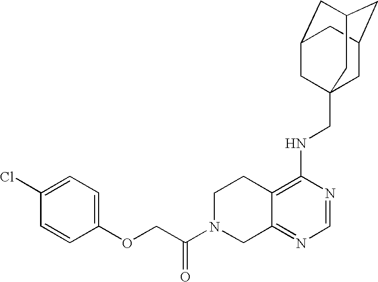 | 467.01 | 467.26 | ** | C |
| 120 | 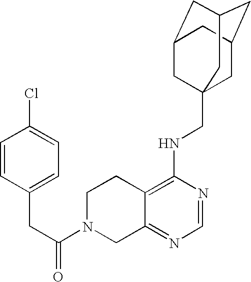 | 451.01 | 451.19 | ** | C |
| 121 | 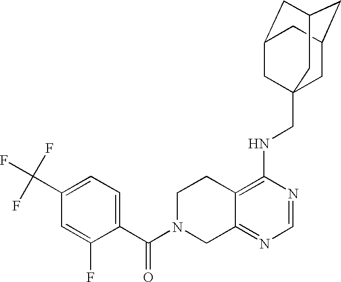 | 488.53 | 489.29 | ** | C |
| 122 | 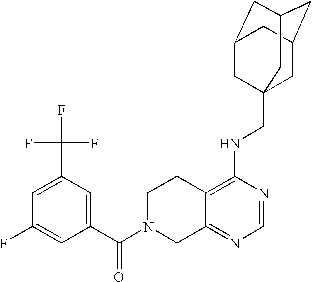 | 488.53 | 489.29 | * | C |
| 123 | 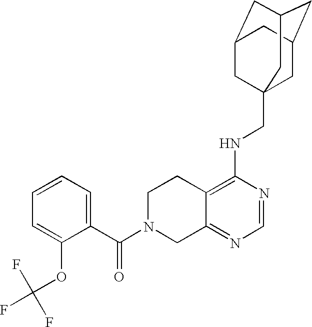 | 486.54 | | | C |
| 124 | 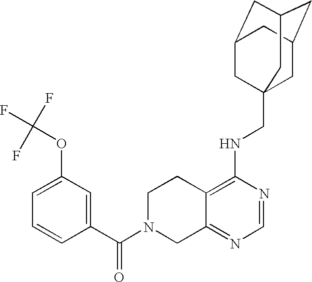 | 486.54 | | | C |
| 125 | 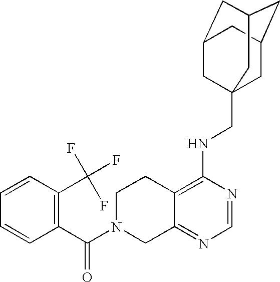 | 470.54 | 471.41 | | C |
| 126 | 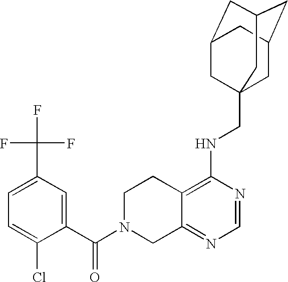 | 504.98 | 505.15 | * | C |
| 127 | 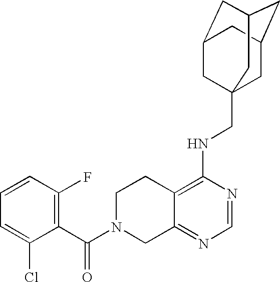 | 454.97 | 455.22 | **** | C |
| 128 | 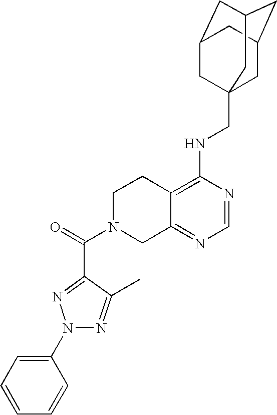 | 483.62 | 484.49 | **** | C |
| 129 | 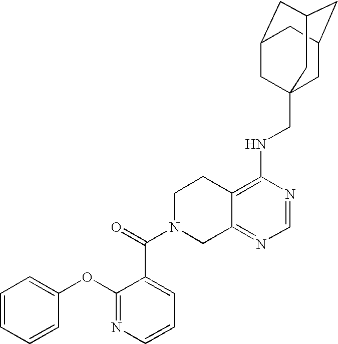 | 495.62 | 496.49 | **** | C |
| 130 | 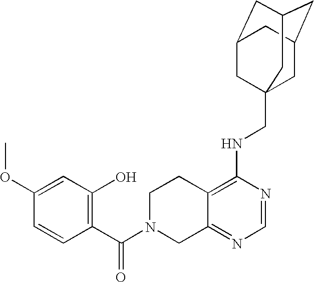 | 462.59 | | | C |
| 131 | 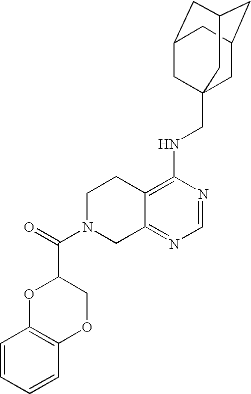 | 460.57 | 461.27 | | C |
| 132 | 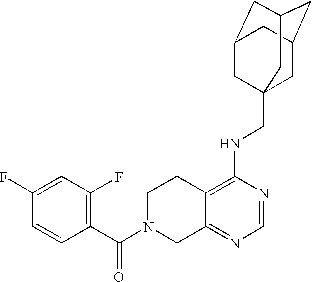 | 438.52 | 439.31 | *** | C |
| 133 | 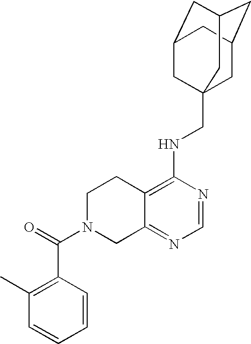 | 416.57 | 417.41 | ** | C |
| 134 | 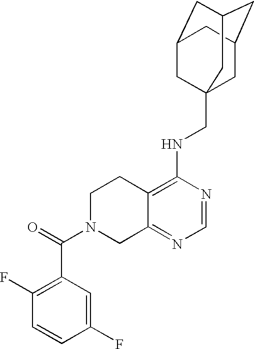 | 438.52 | 439.30 | **** | C |
| 135 | 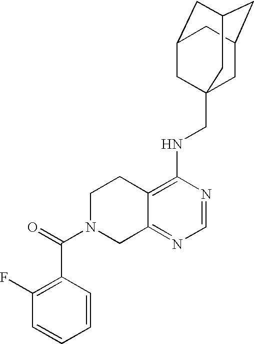 | 420.53 | 421.21 | +++ | C |
| 136 | 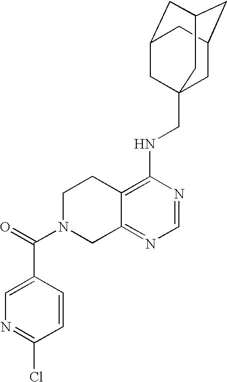 | 437.97 | 438.16 | * | C |
| 137 | 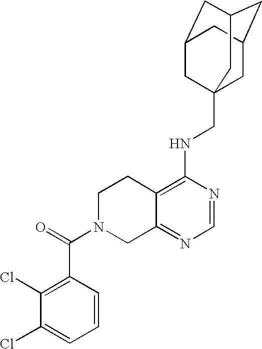 | 471.43 | 471.34 | **** | C |
| 138 | 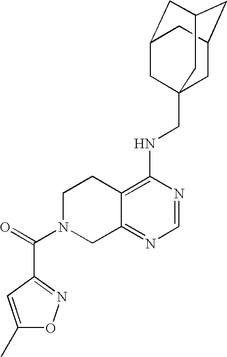 | 407.52 | 408.24 | | C |
| 139 | 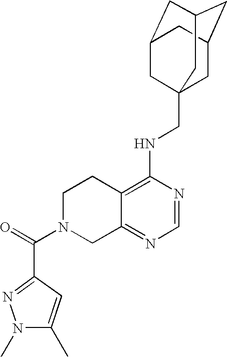 | 420.56 | 421.24 | * | C |
| 140 | 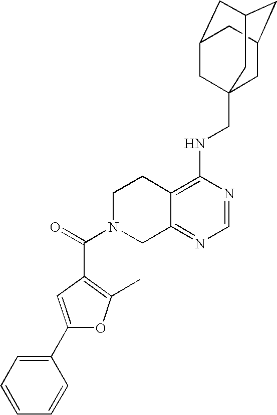 | 482.62 | 483.41 | | C |
| 141 | 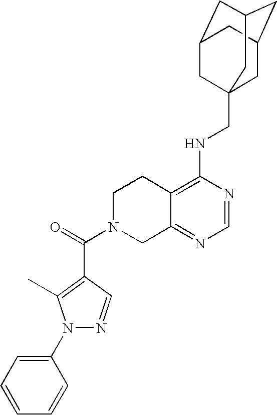 | 482.63 | 483.35 | | C |
| 142 | 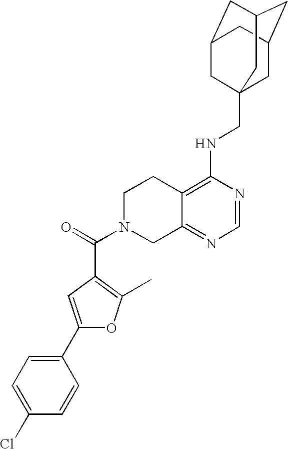 | 517.07 | 517.28 | | C |
| 143 | 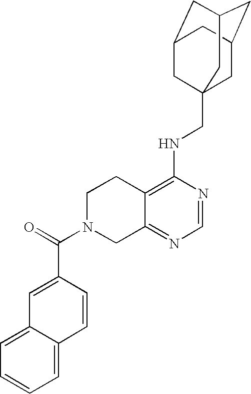 | 452.60 | 453.10 | *** | C |
| 144 | 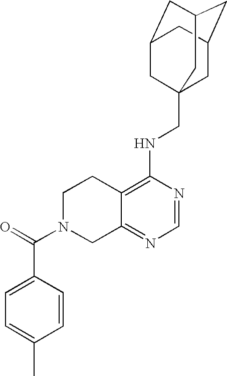 | 416.57 | 417.40 | *** | C |
| 145 | 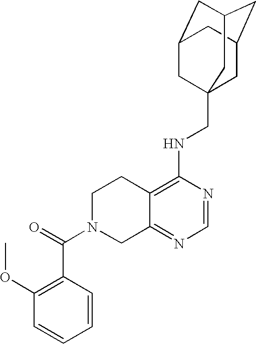 | 432.56 | 433.31 | | C |
| 146 | 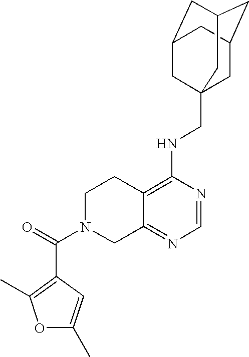 | 420.55 | 421.23 | **** | C |
| 147 | 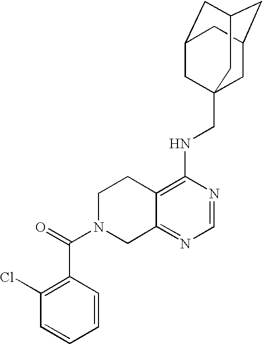 | 436.98 | 437.12 | * | C |
| 148 | 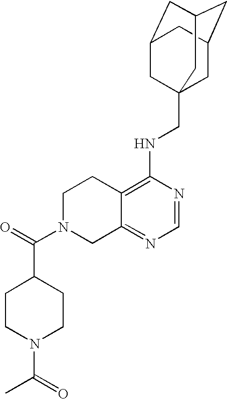 | 451.61 | 452.17 | * | C |
| 149 | 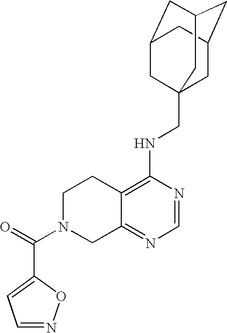 | 393.49 | 394.18 | ++++ | C |
| 151 | 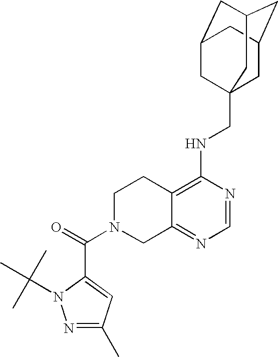 | 462.64 | 463.31 | ** | C |
| 152 | 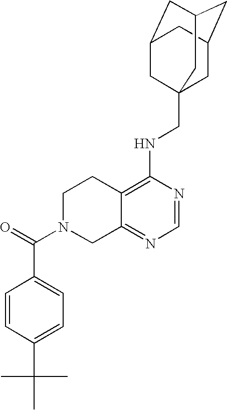 | 458.65 | 459.38 | **** | C |
| 153 | 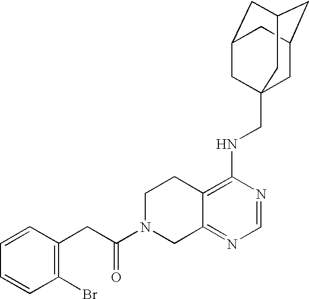 | 495.46 | 495.30 | * | C |
| 154 | 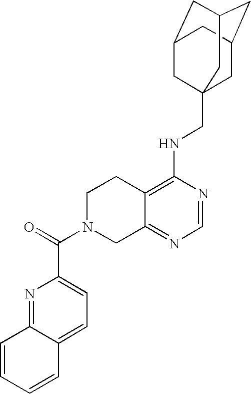 | 453.59 | 454.16 | ** | C |
| 155 | 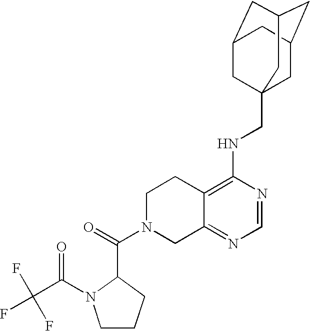 | 491.55 | 492.26 | | C |
| 156 | 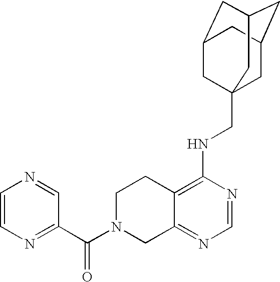 | 404.52 | 405.38 | | C |
| 157 | 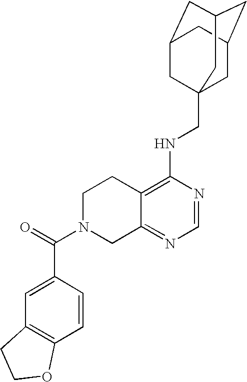 | 444.58 | 445.36 | | C |
| 158 | 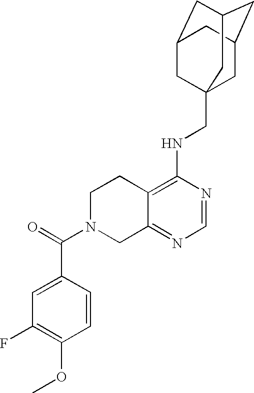 | 450.55 | 451.21 | **** | C |
| 159 | 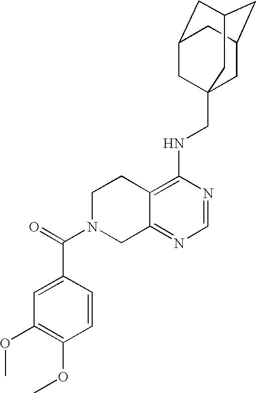 | 462.59 | 463.30 | **** | C |
| 160 | 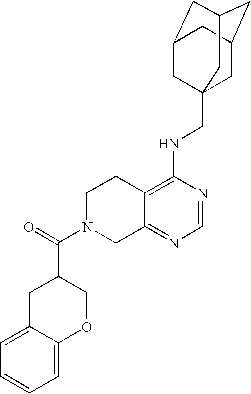 | 458.60 | 459.36 | **** | C |
| 161 | 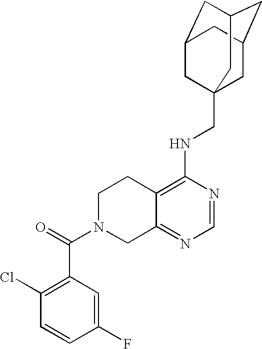 | 454.97 | | | C |
| 162 | 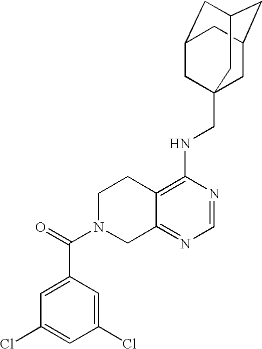 | 471.43 | 471.37 | | C |
| 163 | 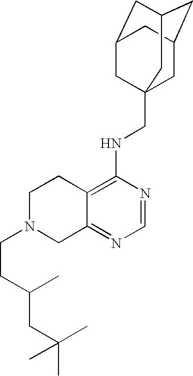 | 424.67 | 425.30 | **** | D |
| 164 | 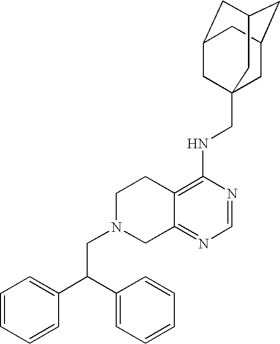 | 478.68 | 479.22 | **** | D |
| 165 | 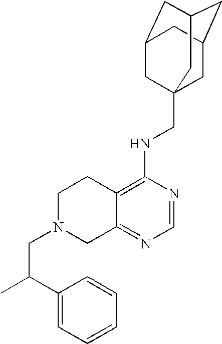 | 416.61 | 417.43 | * | D |
| 166 | 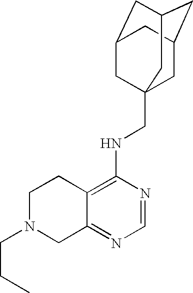 | 340.51 | 341.24 | * | D |
| 167 | 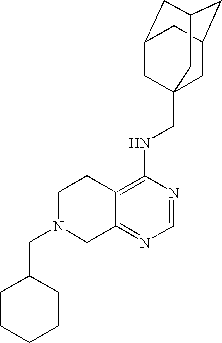 | 394.60 | 395.37 | **** | D |
| 168 | 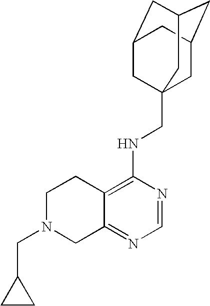 | 352.52 | 353.30 | * | D |
| 169 | 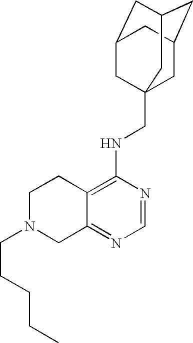 | 368.57 | 369.27 | **** | D |
| 170 | 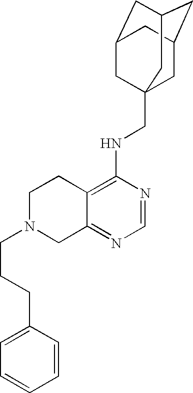 | 416.61 | 417.43 | * | D |
| 171 | 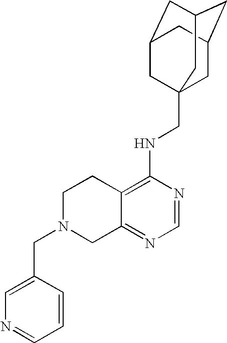 | 389.54 | 390.47 | ++++ | D |
| 172 | 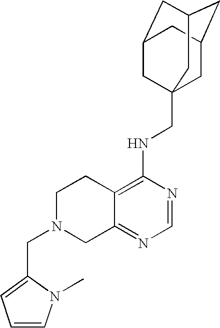 | 391.56 | | | D |
| 173 | 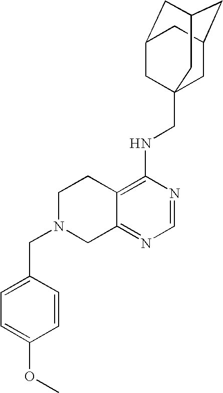 | 418.58 | 419.31 | * | D |
| 174 | 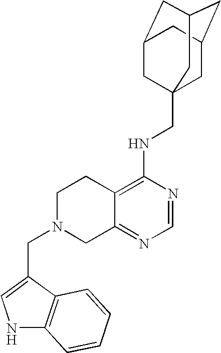 | 427.59 | 428.62 | | D |
| 175 | 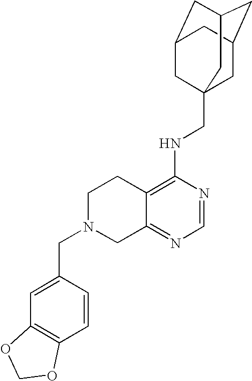 | 432.56 | 433.31 | ** | D |
| 176 | 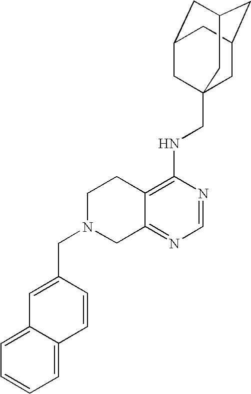 | 438.62 | 439.36 | * | D |
| 177 | 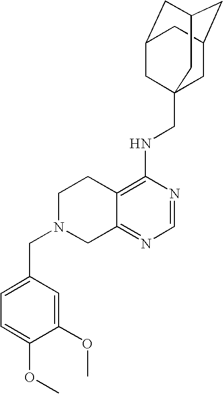 | 448.61 | 449.19 | * | D |
| 178 | 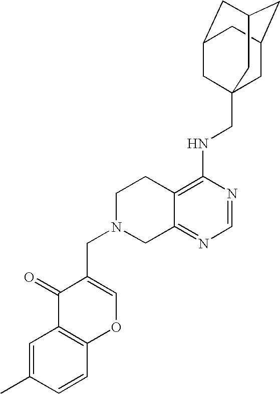 | 470.61 | 471.42 | ** | D |
| 179 | 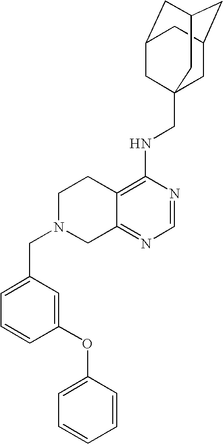 | 480.65 | 481.30 | ** | D |
| 180 | 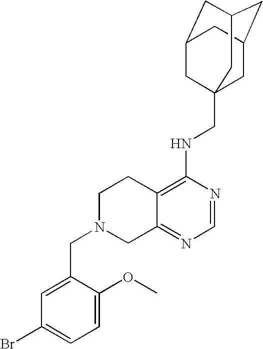 | 497.48 | 497.34 | | D |
| 181 | 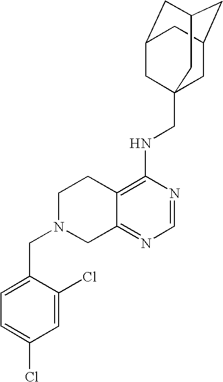 | 457.45 | 457.29 | ** | D |
| 182 | 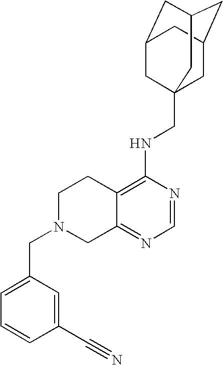 | 413.57 | 414.23 | **** | D |
| 183 | 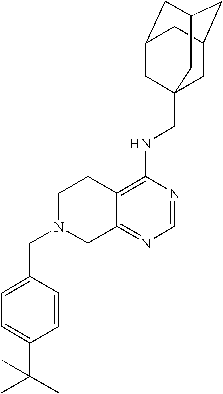 | 444.66 | 445.46 | | D |
| 184 | 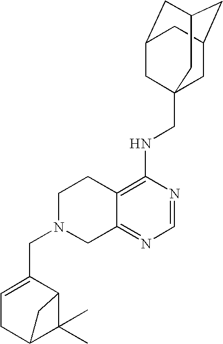 | 432.65 | 433.44 | **** | D |
| 185 | 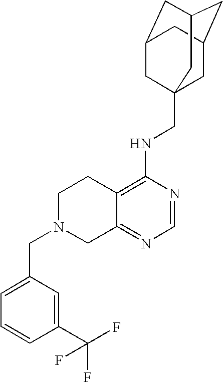 | 456.55 | 457.42 | | D |
| 186 | 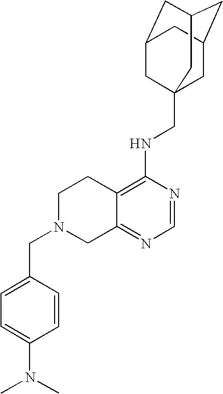 | 431.62 | 432.47 | | D |
| 187 | 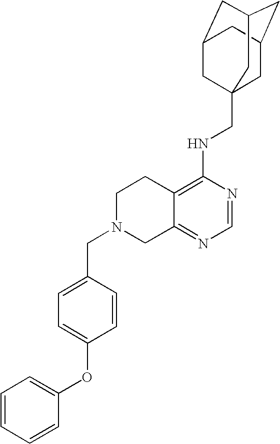 | 480.65 | 481.30 | *** | D |
| 188 | 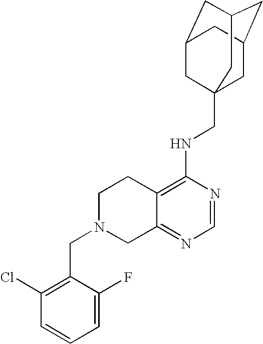 | 440.99 | 441.28 | | D |
| 189 | 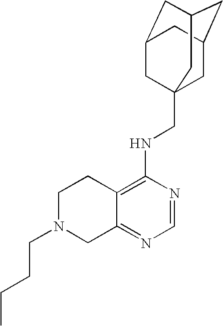 | 354.54 | 355.29 | * | D |
| 190 | 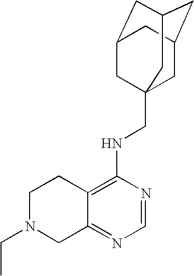 | 326.49 | 327.27 | ** | D |
| 191 | 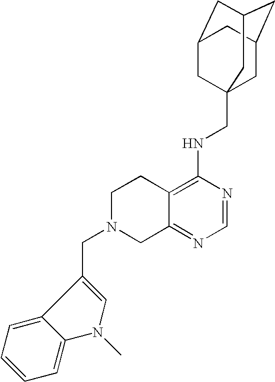 | 441.62 | 442.72 | | D |
| 192 | 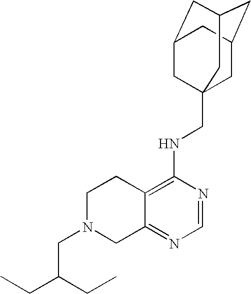 | 382.59 | 383.43 | **** | D |
| 193 | 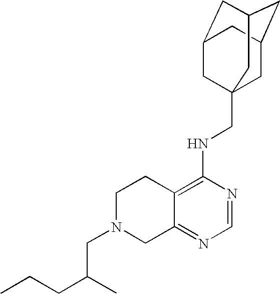 | 382.59 | 383.48 | **** | D |
| 194 | 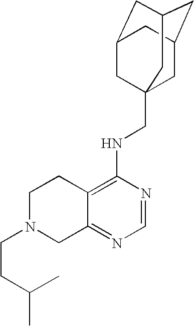 | 368.57 | 369.27 | ** | D |
| 195 | 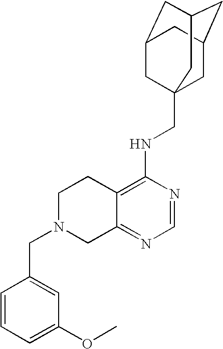 | 418.58 | 419.30 | **** | D |
| 196 | 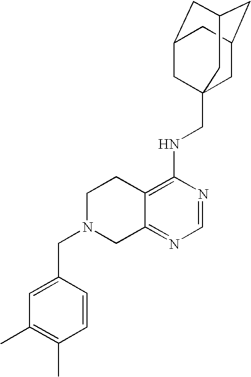 | 416.61 | 417.42 | * | D |
| 197 | 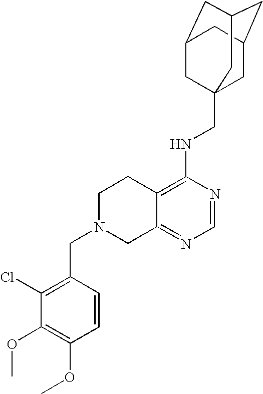 | 483.05 | 483.31 | * | D |
| 198 | 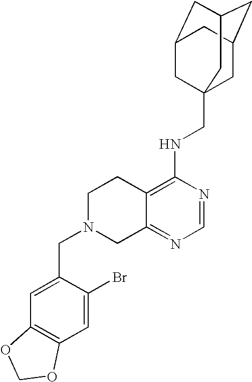 | 511.46 | 511.33 | * | D |
| 199 | 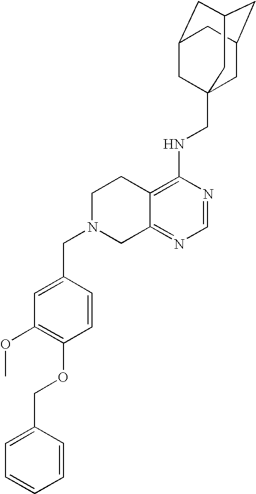 | 524.71 | 525.38 | * | D |
| 200 | 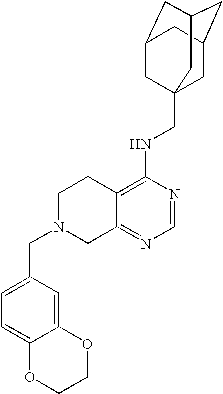 | 446.59 | 447.23 | * | D |
| 201 | 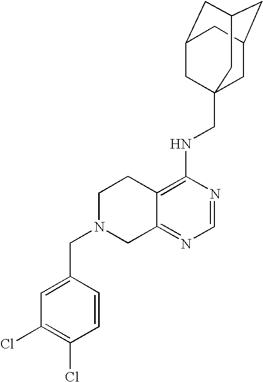 | 457.45 | 457.29 | * | D |
| 202 | 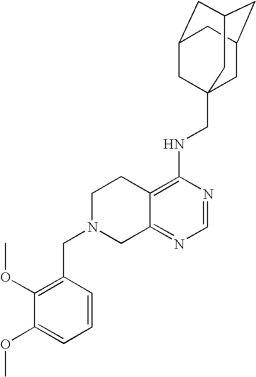 | 448.61 | 449.17 | **** | D |
| 203 | 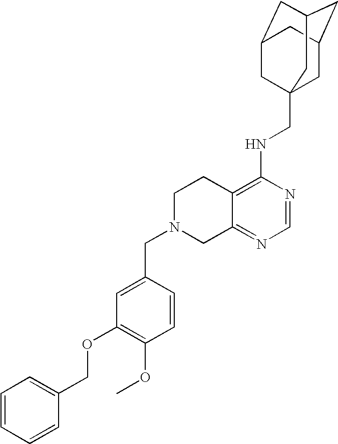 | 524.71 | 525.38 | * | D |
| 204 | 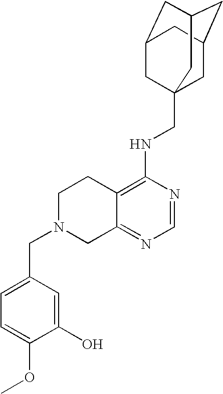 | 434.58 | 435.35 | * | D |
| 205 | 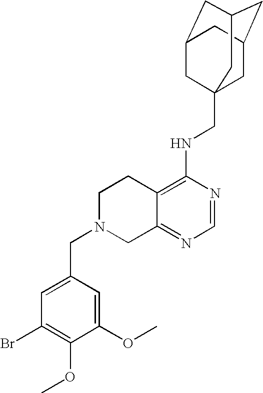 | 527.50 | 527.21 | * | D |
| 206 | 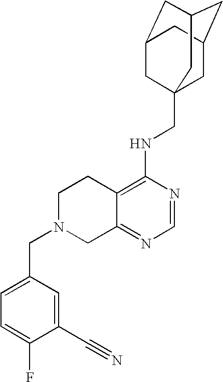 | 431.56 | 432.39 | **** | D |
| 207 | 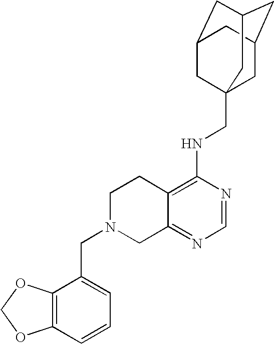 | 432.56 | 433.31 | ++++ | D |
| 208 | 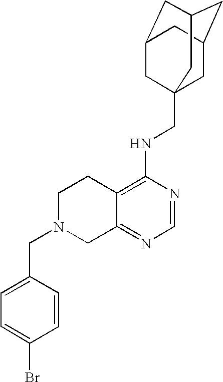 | 467.45 | 467.24 | **** | D |
| 209 | 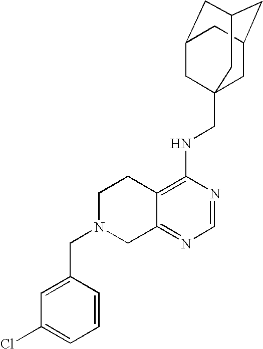 | 423.00 | 423.13 | * | D |
| 210 | 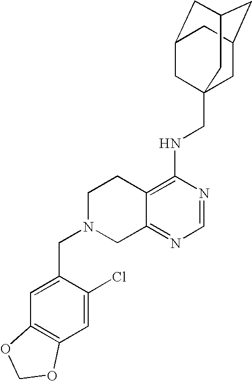 | 467.01 | 467.25 | * | D |
| 211 | 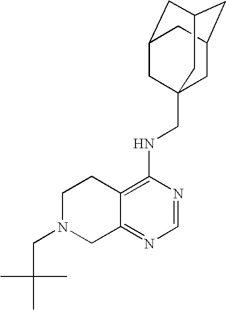 | 368.57 | 369.38 | **** | D |
| 212 | 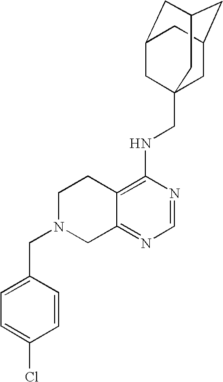 | 423.00 | 423.14 | **** | D |
| 213 | 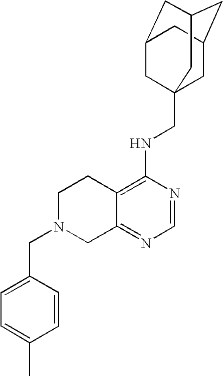 | 402.58 | 403.46 | | D |
| 214 | 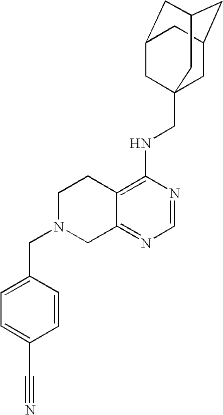 | 413.57 | 414.24 | ** | D |
| 215 | 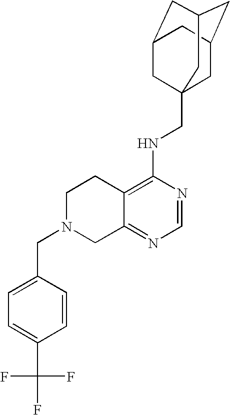 | 456.55 | 457.33 | * | D |
| 216 | 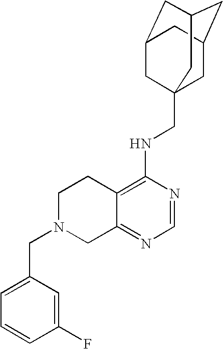 | 406.55 | 407.31 | **** | D |
| 217 | 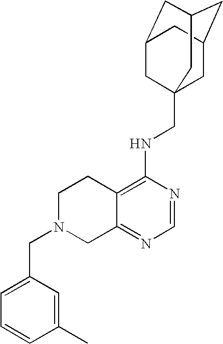 | 402.58 | 403.39 | **** | D |
| 218 | 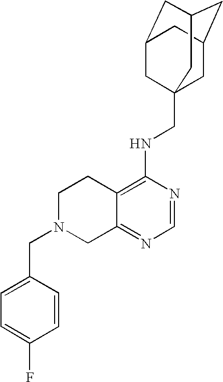 | 406.55 | 407.31 | **** | D |
| 219 | 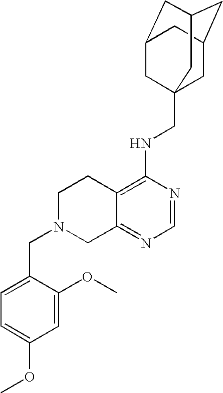 | 448.61 | 449.18 | * | D |
| 220 | 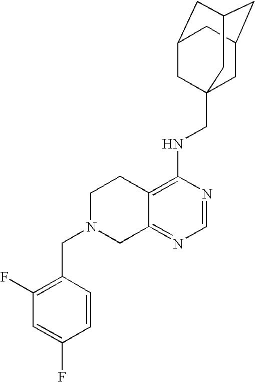 | 424.54 | 425.55 | **** | D |
| 221 | 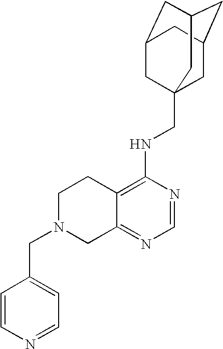 | 389.54 | 390.46 | **** | D |
| 222 | 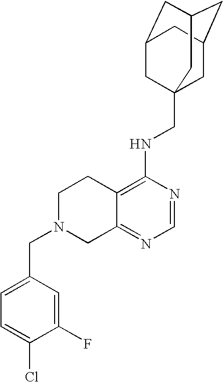 | 440.99 | 441.34 | **** | D |
| 223 | 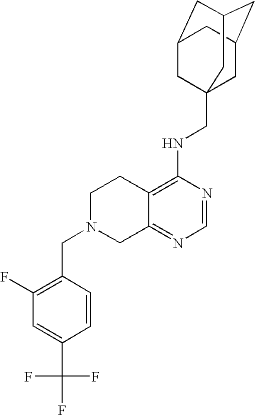 | 474.54 | 475.26 | **** | D |
| 224 | 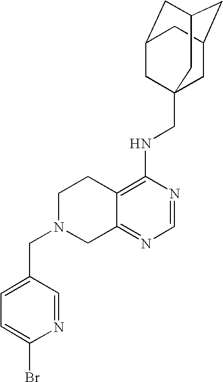 | 468.44 | 468.13 | * | D |
| 225 | 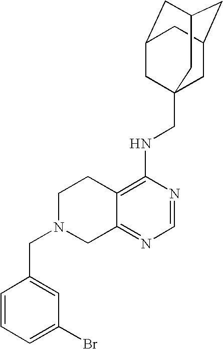 | 467.45 | 467.22 | **** | D |
| 226 | 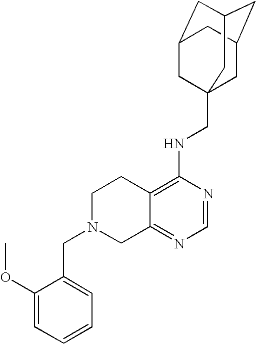 | 418.58 | 419.30 | **** | D |
| 227 | 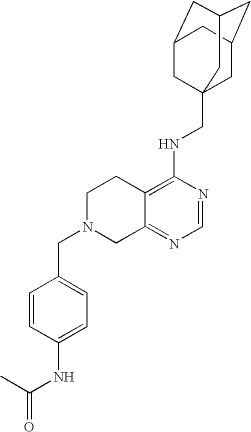 | 445.61 | 446.20 | * | D |
| 228 | 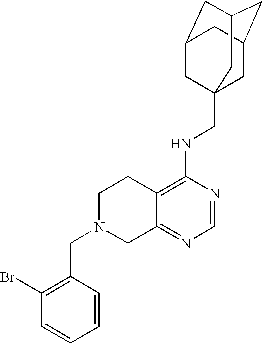 | 467.45 | 467.21 | * | D |
| 229 | 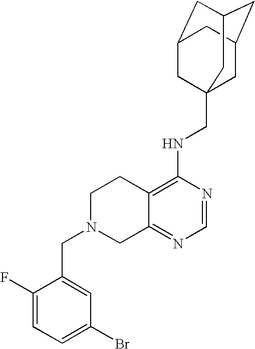 | 485.44 | 485.29 | * | D |
| 230 | 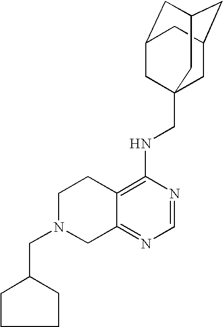 | 380.58 | 381.36 | **** | D |
| 231 | 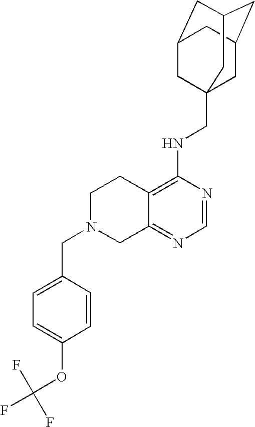 | 472.55 | 473.24 | * | D |
| 232 | 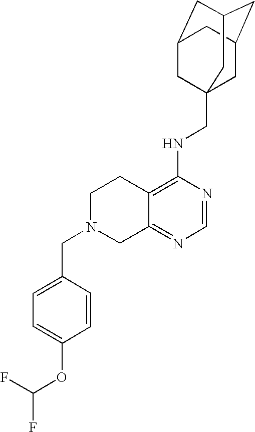 | 454.56 | 455.27 | * | D |
| 233 | 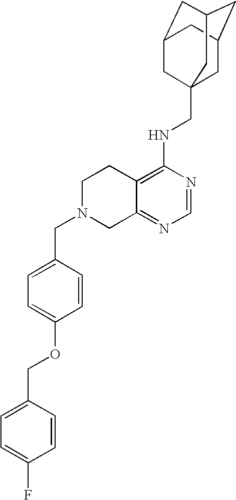 | 512.67 | 513.39 | * | D |
| 234 | 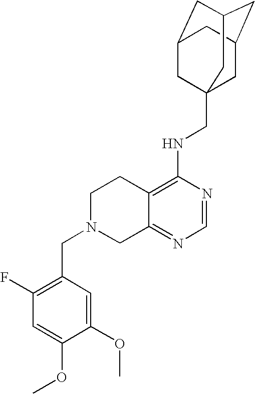 | 466.60 | 467.29 | * | D |
| 235 | 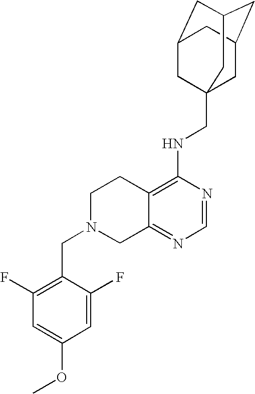 | 454.56 | 455.27 | * | D |
| 236 | 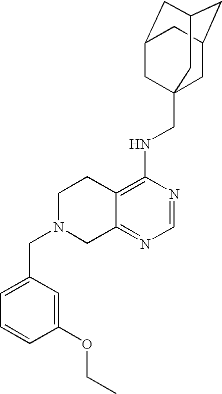 | 432.61 | 433.32 | *** | D |
| 237 | 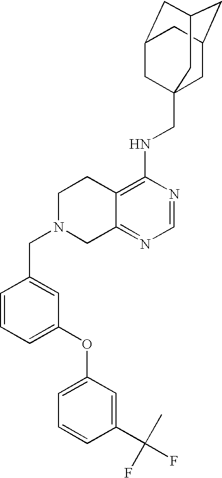 | 548.65 | 549.35 | * | D |
| 238 | 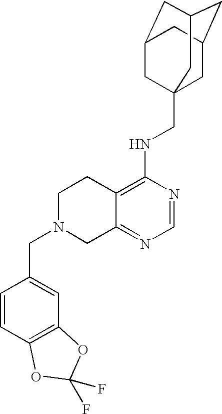 | 468.55 | 469.32 | * | D |
| 239 | 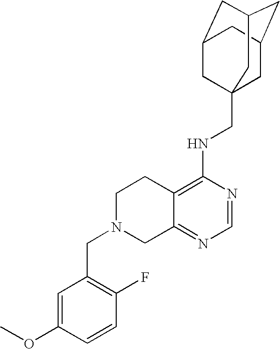 | 436.57 | 437.20 | * | D |
| 240 | 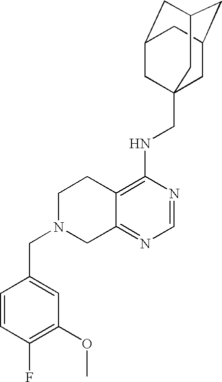 | 436.57 | 437.33 | **** | D |
| 241 | 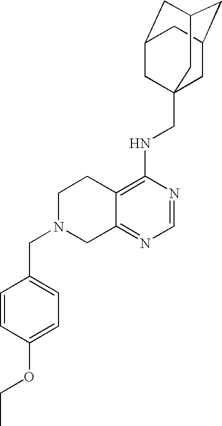 | 432.61 | 433.38 | **** | D |
| 242 | 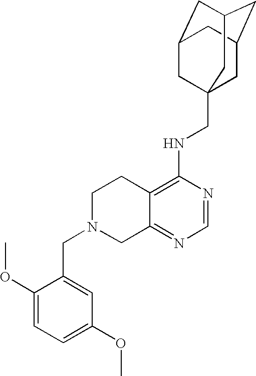 | 448.61 | 449.22 | **** | D |
| 243 | 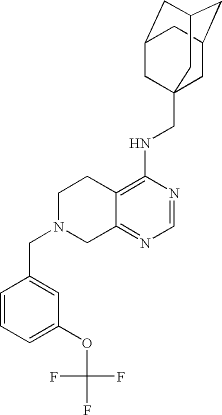 | 472.55 | 473.30 | **** | D |
| 244 | 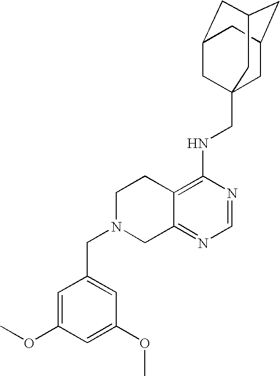 | 448.61 | 449.21 | **** | D |
| 245 | 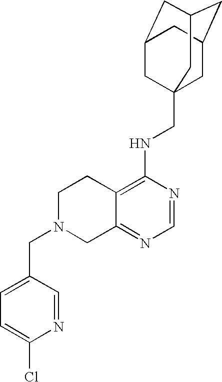 | 423.99 | 424.25 | **** | D |
| 246 | 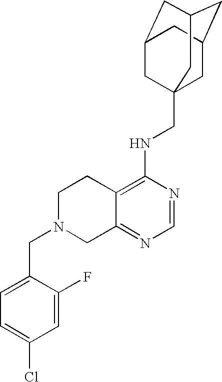 | 440.99 | 441.28 | **** | D |
| 247 | 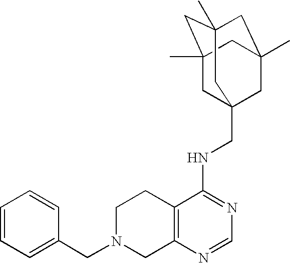 | 430.64 | 431.82 | ** | A |
| 248 | 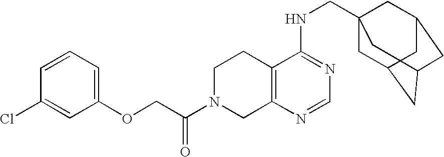 | 467.01 | 466.90 | ++++ | C1 |
| 249 | 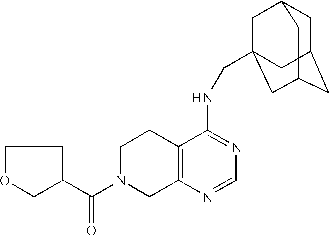 | 396.53 | 396.90 | + | C2 |
| 250 | 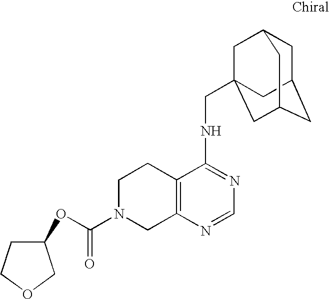 | 412.53 | 412.70 | + | F |
| 251 | 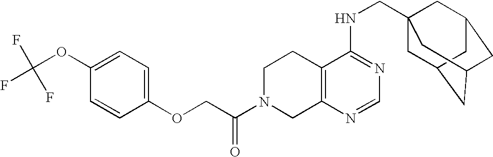 | 516.56 | 516.90 | + | C2 |
| 252 | 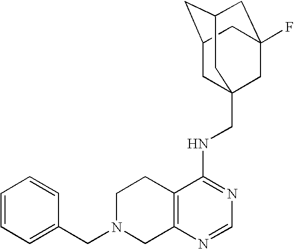 | 406.55 | 407.34 | ++++ | A |
| 253 | 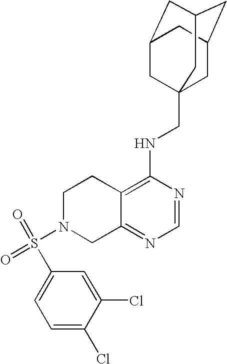 | 507.48 | | | B |
| 254 | 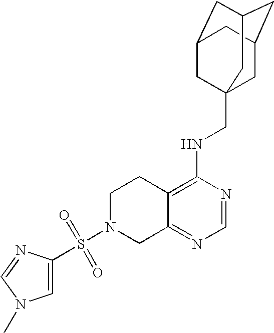 | 442.59 | | | B |
| 255 | 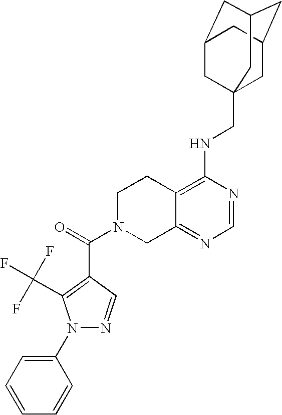 | 536.60 | 537.34 | **** | C |
| 256 | 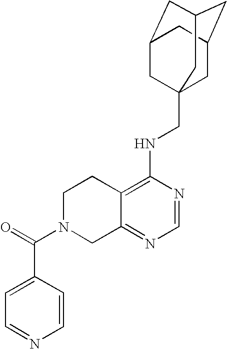 | 403.53 | 404.46 | | C |
| 257 | 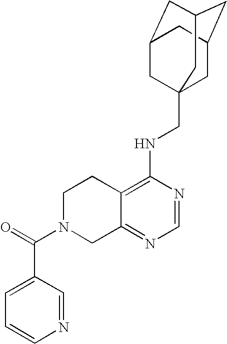 | 403.53 | | ++++ | C1 |
| 258 | 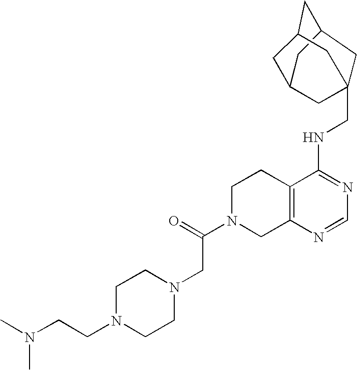 | 495.71 | 496.68 | * | E |
| 259 | 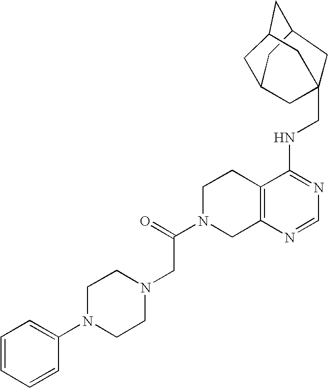 | 500.69 | 501.44 | *** | E |
| 260 | 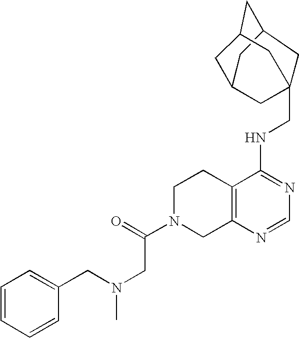 | 459.63 | 460.44 | ++++ | E |
| 261 | 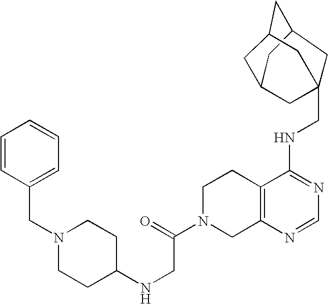 | 528.74 | 529.35 | ** | E |
| 264 | 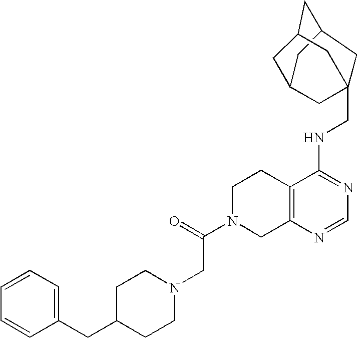 | 513.73 | 514.54 | ** | E |
| 265 | 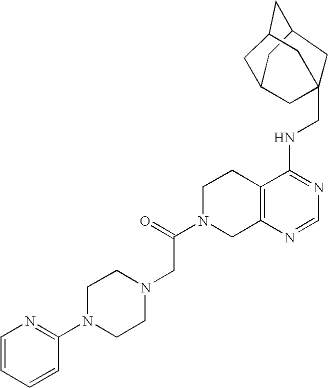 | 501.68 | 502.39 | | E |
| 266 | 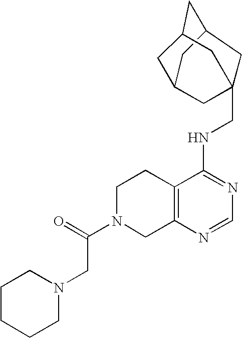 | 423.60 | 424.31 | * | E |
| 267 | 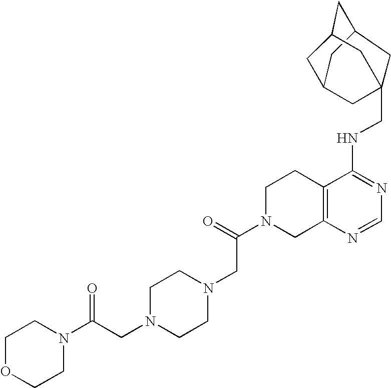 | 551.73 | 552.58 | * | E |
| 268 | 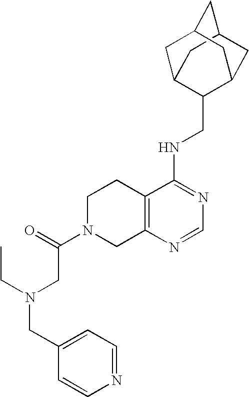 | 474.65 | 475.35 | *** | E |
| 269 | 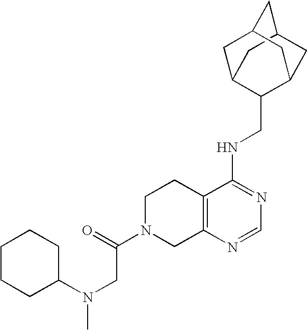 | 451.65 | 452.29 | *** | E |
| 270 | 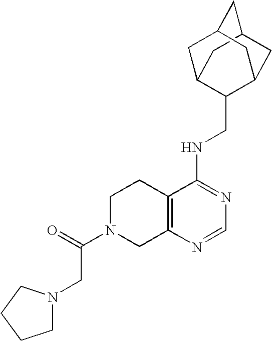 | 409.57 | | | E |
| 271 | 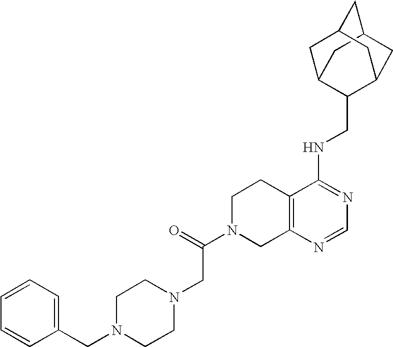 | 514.71 | 515.46 | * | E |
| 272 | 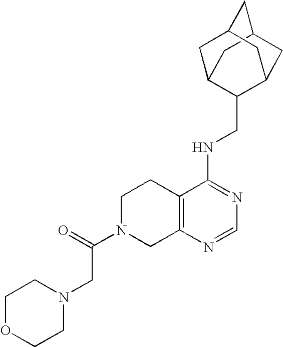 | 425.57 | 426.25 | * | E |
| 273 | 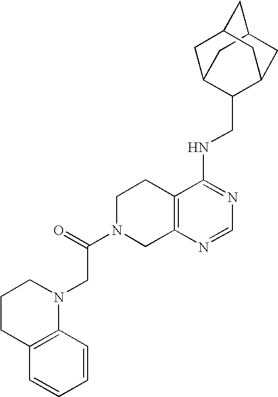 | 471.65 | 472.34 | | E |
| 274 | 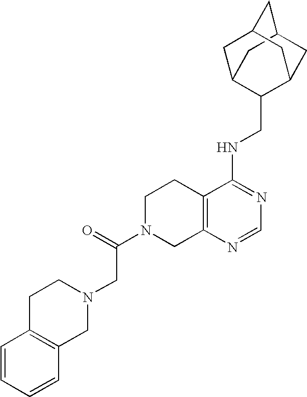 | 471.65 | 472.33 | ** | E |
| 275 | 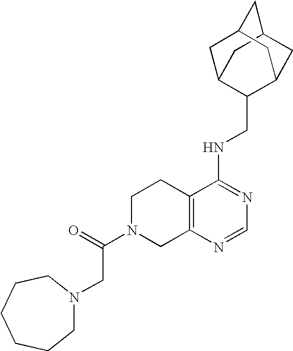 | 437.63 | 438.36 | * | E |
| 276 | 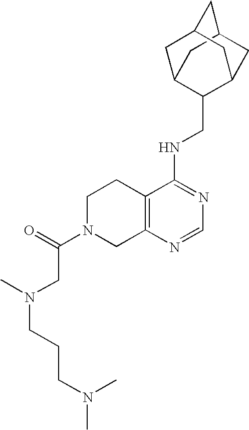 | 454.66 | 455.44 | * | E |
| 277 | 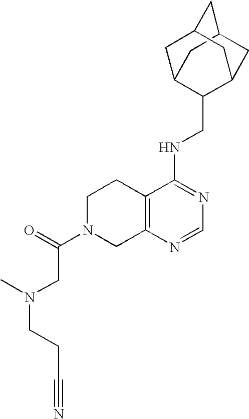 | 422.57 | 423.35 | * | E |
| 278 | 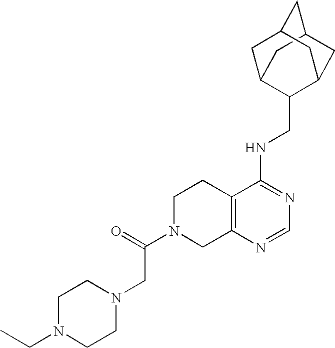 | 452.64 | 453.36 | ** | E |
| 279 | 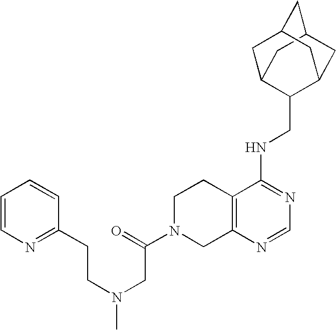 | 474.65 | 475.38 | *** | E |
| 280 | 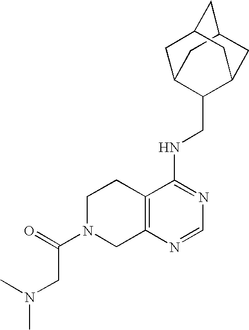 | 383.54 | | | E |
| 281 | 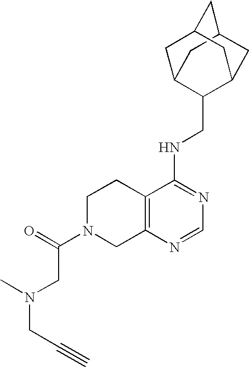 | 407.56 | 408.41 | | E |
| 282 | 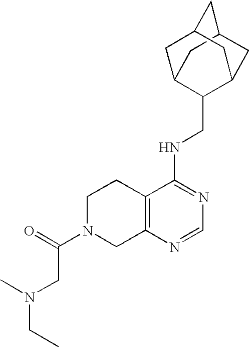 | 397.56 | 398.30 | * | E |
| 283 | 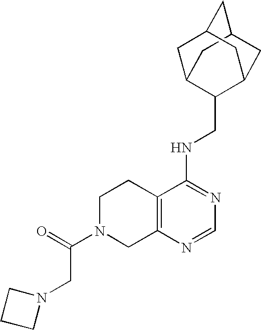 | 395.55 | 396.22 | * | E |
| 284 | 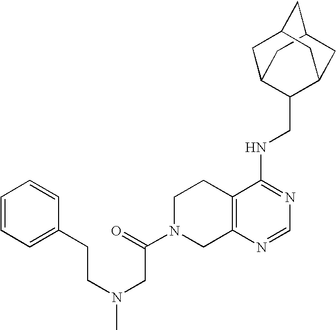 | 473.66 | 474.33 | **** | E |
| 285 | 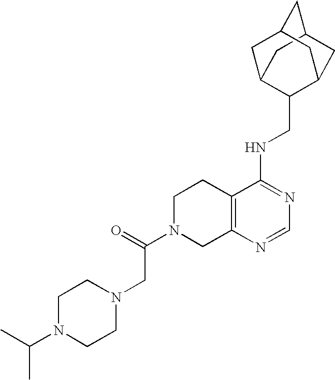 | 466.67 | 467.43 | * | E |
| 286 | 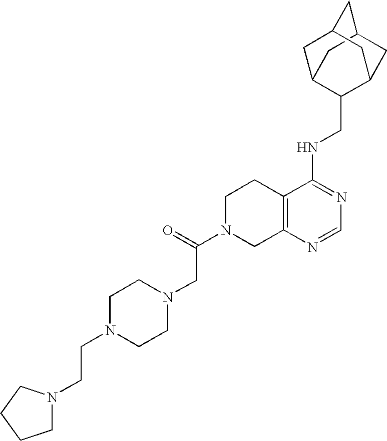 | 521.75 | 522.60 | | E |
| 287 | 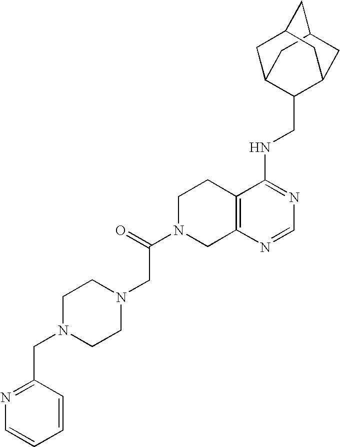 | 515.70 | 516.49 | | E |
| 288 | 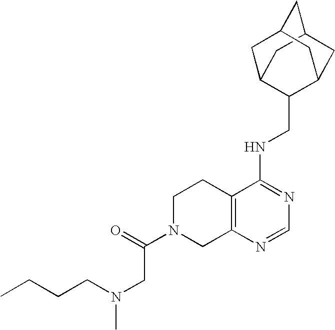 | 425.62 | 426.29 | *** | E |
| 289 | 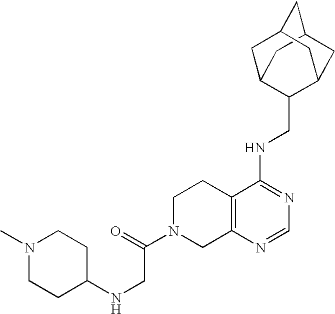 | 452.64 | 453.37 | * | E |
| 290 | 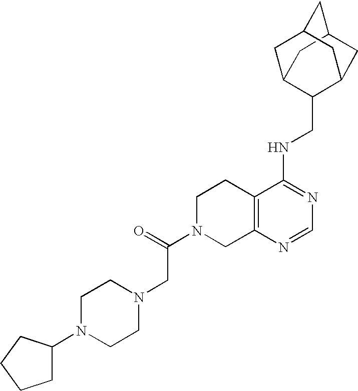 | 492.71 | 493.53 | | E |
| 291 | 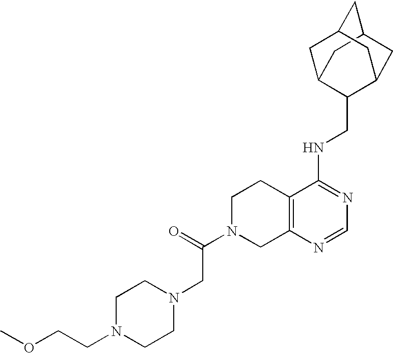 | 482.67 | | | E |
| 292 | 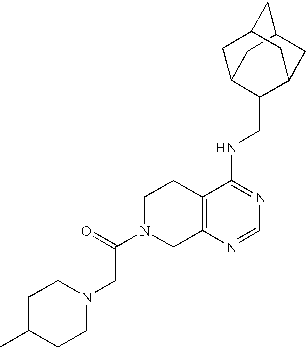 | 437.63 | 438.40 | ** | E |
| 293 | 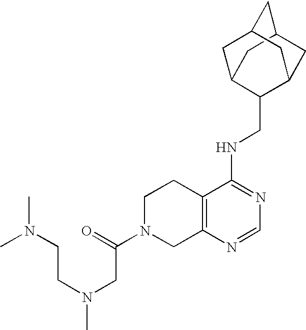 | 440.63 | 441.43 | | E |
| 294 | 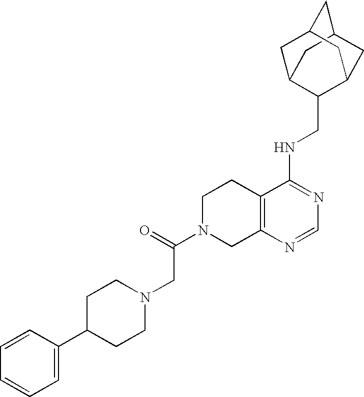 | 499.70 | 500.46 | * | E |
| 295 | 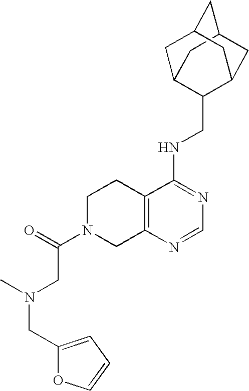 | 449.60 | 450.32 | * | E |
| 296 | 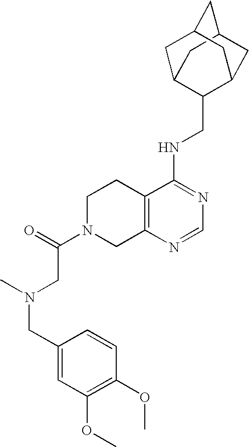 | 519.69 | 520.52 | * | E |
| 297 | 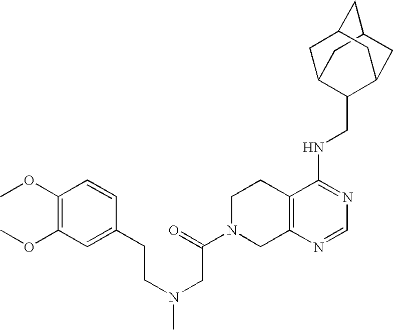 | 533.71 | 534.34 | * | E |
| 298 | 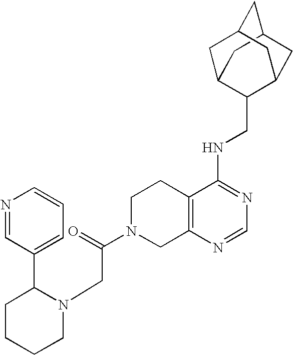 | 500.69 | 501.43 | | E |
| 299 | 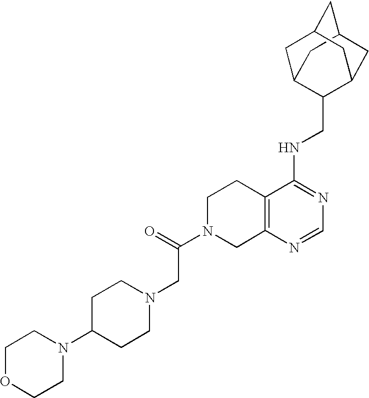 | 508.71 | 509.49 | ** | E |
| 300 | 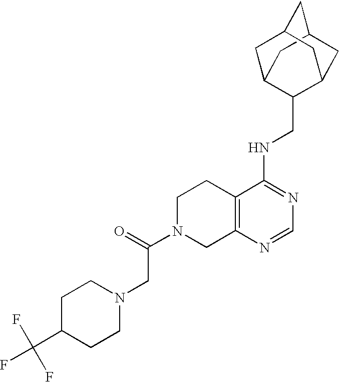 | 491.60 | 492.43 | * | E |
| 301 | 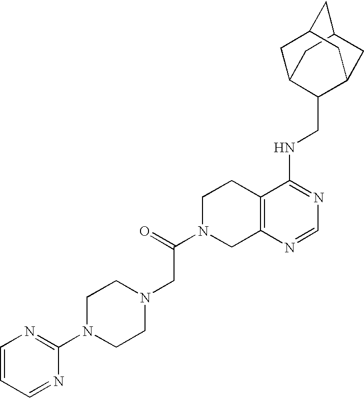 | 502.66 | 503.39 | * | E |
| 302 | 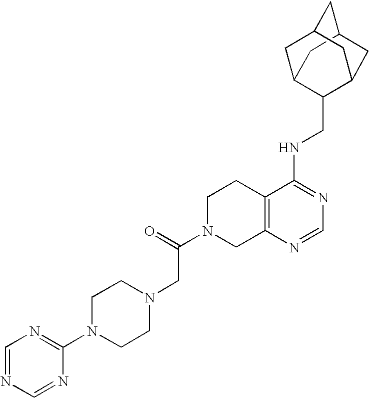 | 503.65 | 504.40 | ** | E |
| 303 | 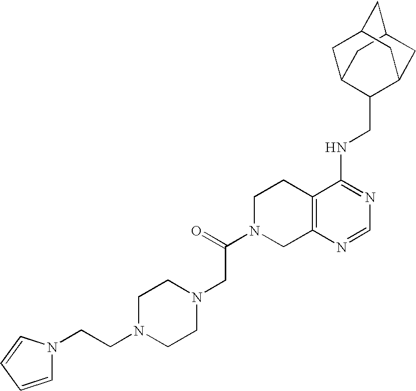 | 517.72 | 518.47 | ** | E |
| 304 | 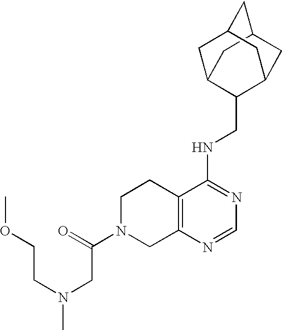 | 427.59 | 428.41 | ** | E |
| 305 | 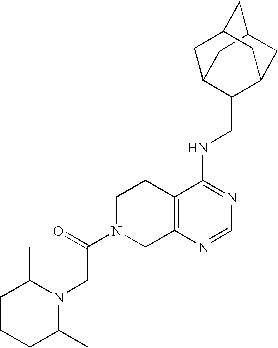 | 451.65 | | | E |
| 306 | 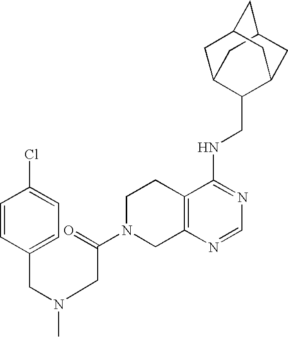 | 494.08 | 494.34 | ** | E |
| 307 | 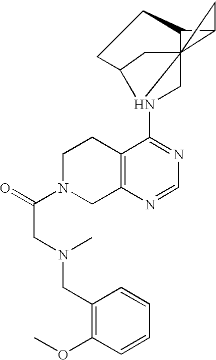 | 489.66 | 490.45 | + | E |
| 308 | 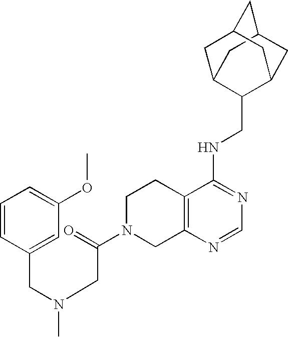 | 489.66 | 490.42 | * | E |
| 309 | 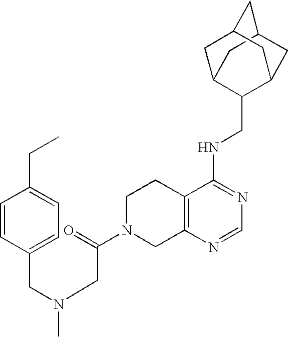 | 487.69 | 488.51 | ** | E |
| 310 | 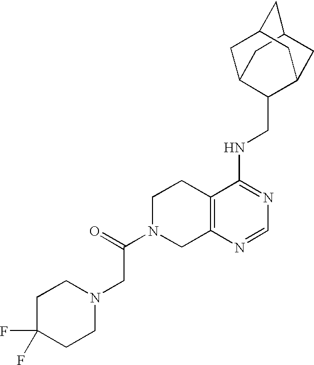 | 459.58 | 460.43 | * | E |
| 311 | 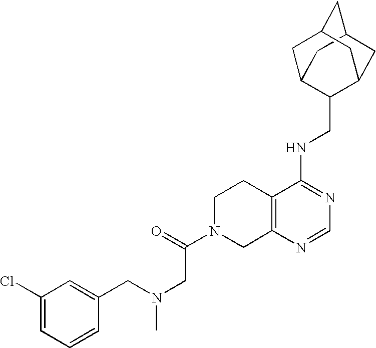 | 494.08 | 494.34 | ** | E |
| 312 | 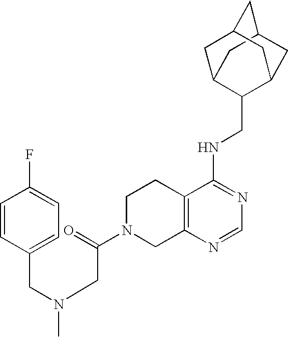 | 477.62 | 478.33 | * | E |
| 313 | 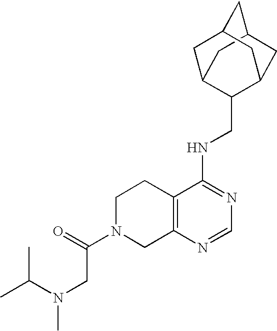 | 411.59 | 412.49 | * | E |
| 314 | 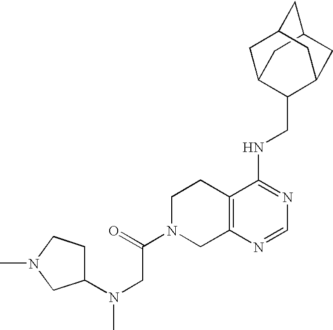 | 452.64 | 453.35 | * | E |
| 315 | 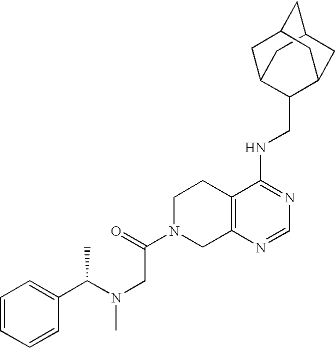 | 473.66 | 474.33 | **** | E |
| 316 | 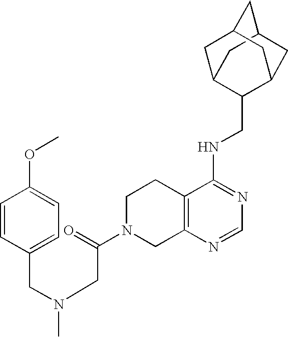 | 489.66 | 490.43 | ** | E |
| 317 | 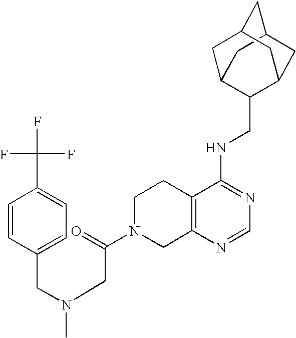 | 527.63 | 528.34 | ** | E |
| 318 | 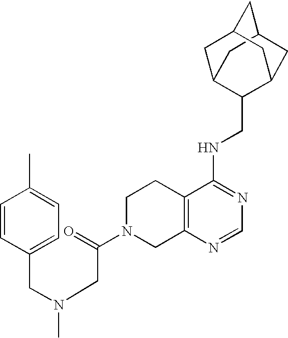 | 473.66 | 474.33 | ** | E |
| 319 | 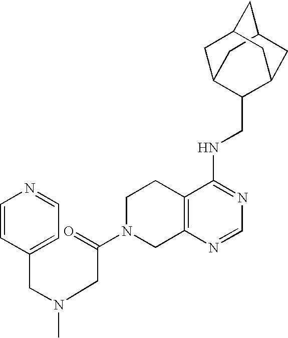 | 460.62 | 461.40 | ** | E |
| 320 | 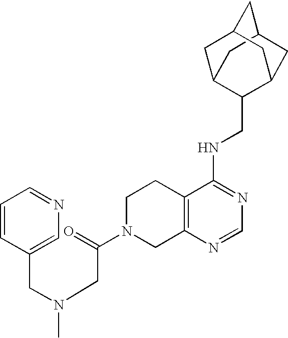 | 460.62 | 461.40 | ** | E |
| 321 | 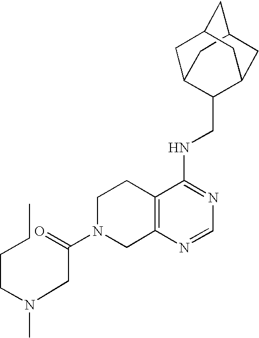 | 439.64 | 440.47 | *** | E |
| 322 | 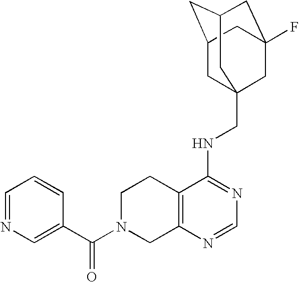 | 421.52 | 422.14 | ++ | C1 |
| 323 | 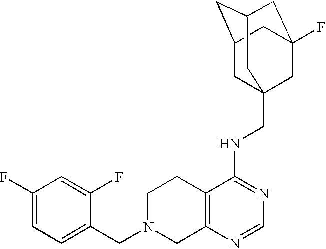 | 442.53 | 443.10 | +++ | D |
| 324 | 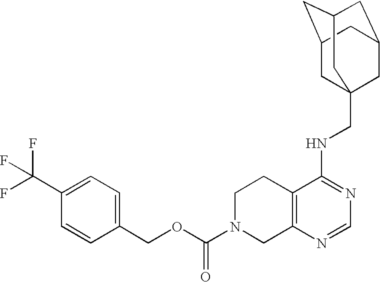 | 500.56 | 498.94 | +++ | F |
| 325 | 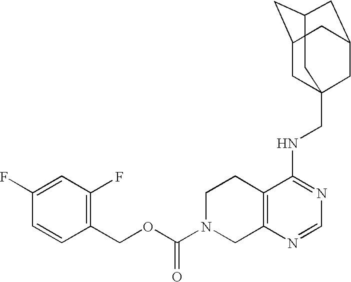 | 468.55 | 469.12 | ++++ | F |
| 326 | 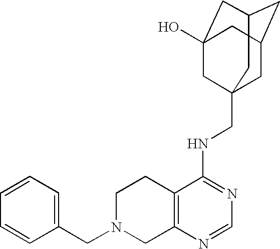 | 404.55 | 405.20 | ++++ | A |
| 327 | 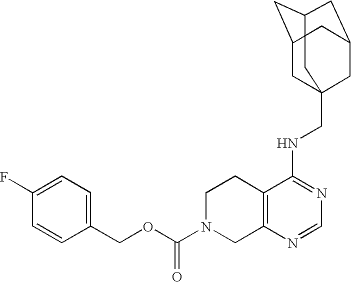 | 450.55 | 451.02 | +++ | F |
| 328 | 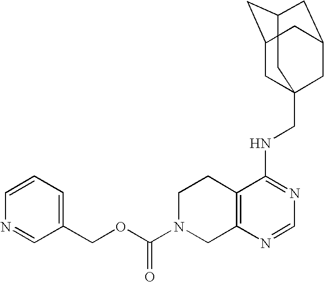 | 433.55 | 434.41 | +++ | F |
| 329 | 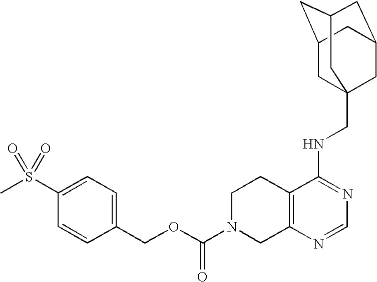 | 510.66 | 511.31 | +++ | F |
| 330 | 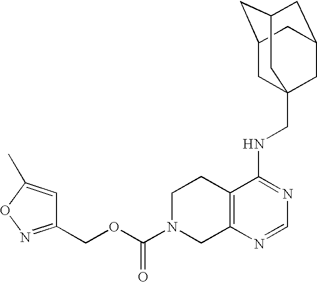 | 437.54 | 438.36 | + | F |
| 331 | 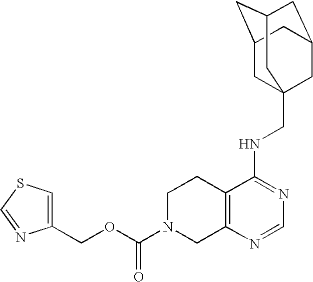 | 439.58 | 440.17 | + | F |
| 332 | 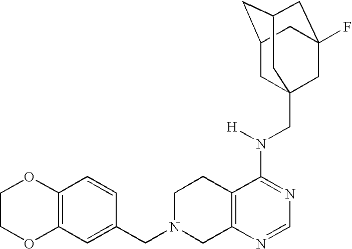 | 464.58 | 465.36 | + | D |
| 333 | 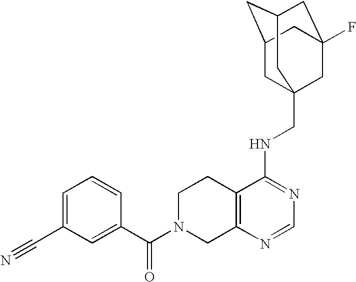 | 445.54 | 446.29 | ++ | C1 |
| 334 | 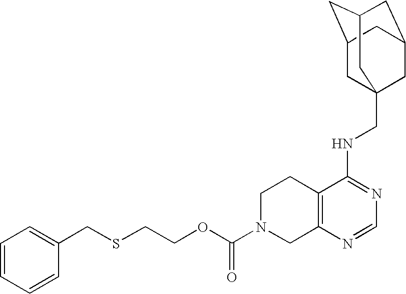 | 492.68 | 493.31 | ++ | F |
| 335 | 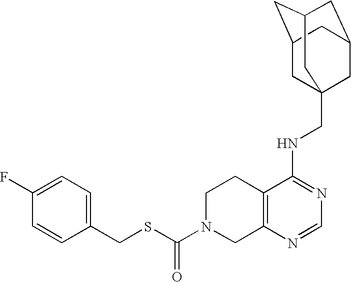 | 466.62 | 467.15 | + | F |
| 336 | 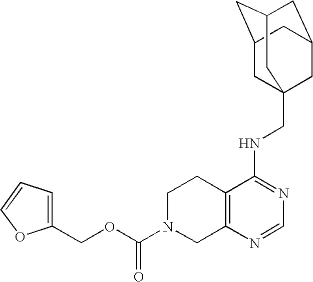 | 422.53 | 423.12 | ++++ | F |
| 337 | 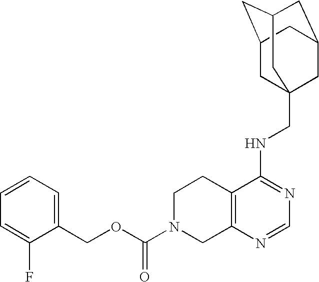 | 450.55 | 451.01 | + | F |
| 338 | 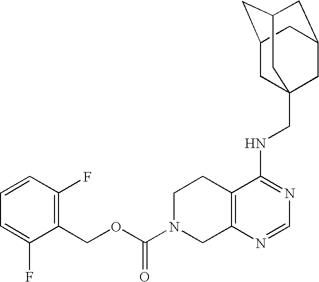 | 468.55 | 469.10 | ++++ | F |
| 339 | 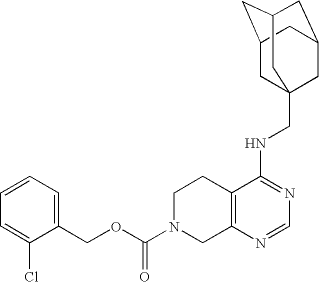 | 467.01 | 467.11 | ++++ | F |
| 340 | 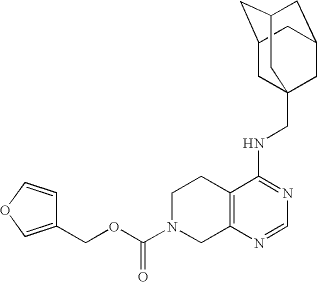 | 422.53 | 423.11 | +++ | F |
| 341 | 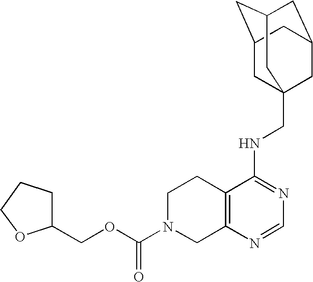 | 426.56 | 427.18 | +++ | F |
| 342 | 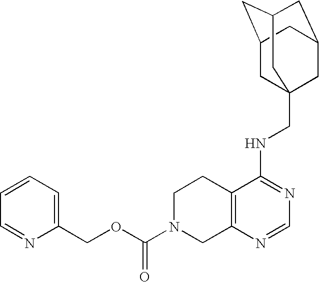 | 433.55 | 434.40 | + | F |
| 343 | 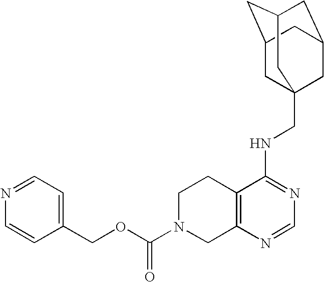 | 433.55 | 434.41 | + | F |
| 344 | 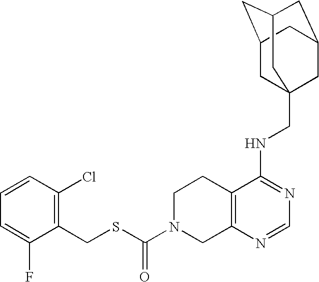 | 501.07 | 501.12 | ++ | F |
| 345 | 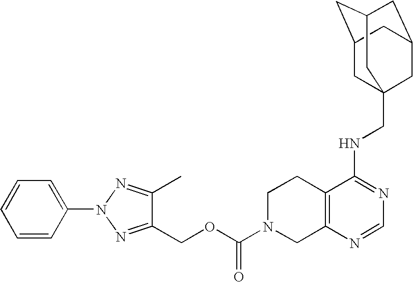 | 513.64 | 514.40 | ++ | F |
| 346 | 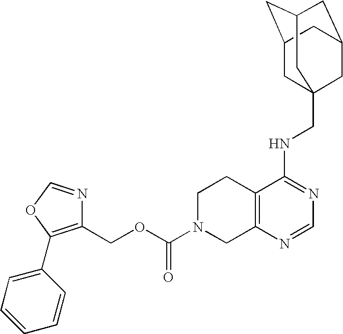 | 499.61 | 500.18 | + | F |
| 347 | 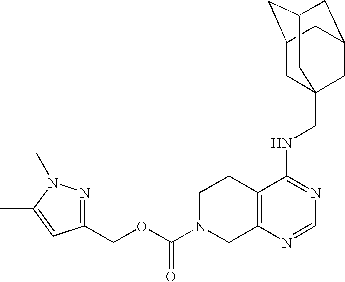 | 450.58 | 451.06 | +++ | F |
| 348 | 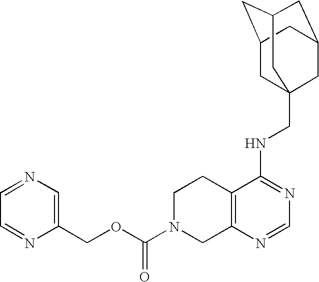 | 434.54 | 435.34 | ++ | F |
| 349 | 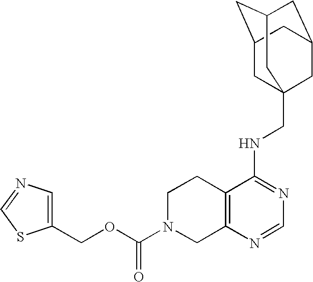 | 439.58 | 440.16 | + | F |
| 350 | 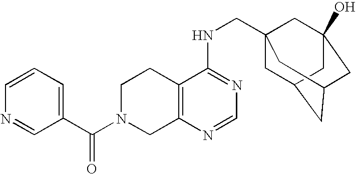 | 419.53 | 419.70 | ++ | C2 |
| 351 | 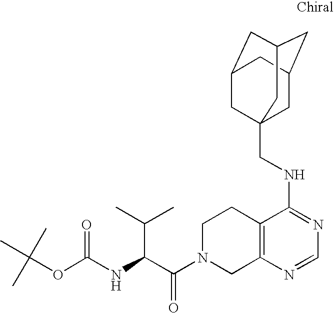 | 497.68 | 498.20 | ++++ | C |

“+” compound exhibited 0-25% inhibition at 0.1 μM
“++” compound exhibited 25-50% inhibition at 0.1 μM
“+++” compound exhibited 50-75% inhibition at 0.1 μM
“++++” compound exhibited 75% or greater inhibition at 0.1 μM
“*” compound exhibited 0-25% inhibition at 0.3 μM
“**” compound exhibited 25-50% inhibition at 0.3 μM
“***” compound exhibited 50-75% inhibition at 0.3 μM
“****” compound exhibited 75% or greater inhibition at 0.3 μM

The following compounds have been or can be prepared according to the methods of the invention. For the purposes of Table 2, activity of each compound is expressed as follows:

TABLE 2
Compounds

| | | | | IL-1β % | |
|---|---|---|---|---|---|
| | | MW | MS | inhib @ 0.3 μM | Method of |
| ID | Structure | (calc) | (obs) | or @ 0.03 μM | Synthesis |
| 501 | 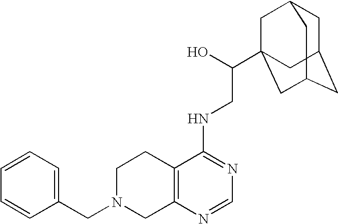 | 418.58 | 419.32 | ++ | G |
| 502 | 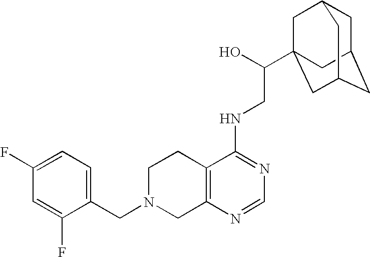 | 454.56 | 455.29 | +++ | I |
| 503 | 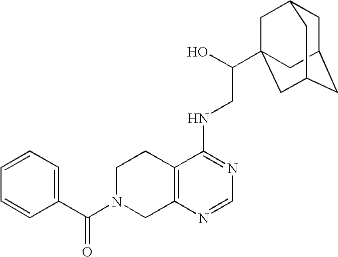 | 432.56 | 433.43 | ++ | C1 |
| 504 | 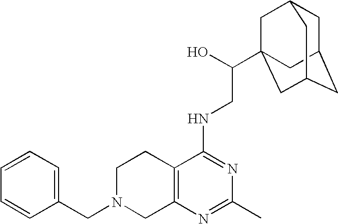 | 432.61 | 433.40 | + | G |
| 505 | 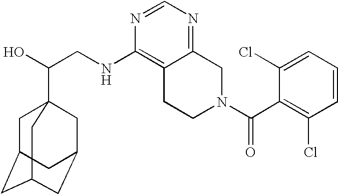 | 501.46 | 501.23 | ++ | C1 |
| 506 | 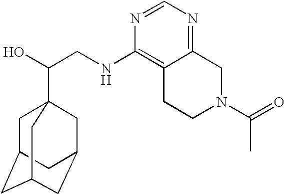 | 370.49 | 371.19 | + | C1 |
| 507 | 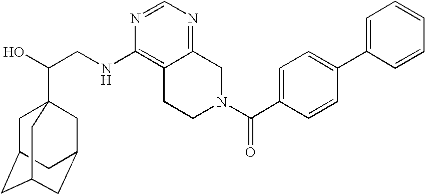 | 508.66 | 509.33 | ++ | C1 |
| 508 | 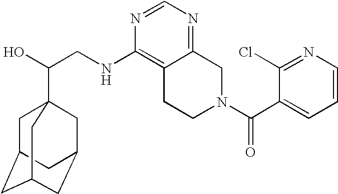 | 468.00 | 468.20 | + | C1 |
| 509 | 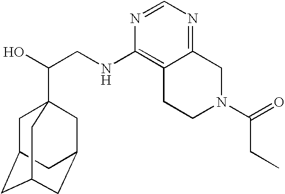 | 384.52 | 385.30 | + | C1 |
| 510 | 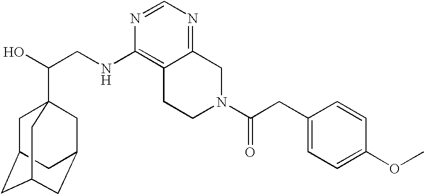 | 476.62 | 477.28 | +++ | C1 |
| 511 | 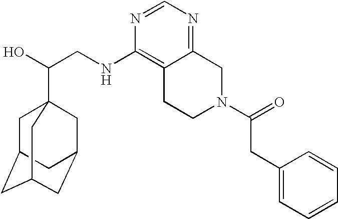 | 446.59 | 447.24 | ++ | C1 |
| 512 | 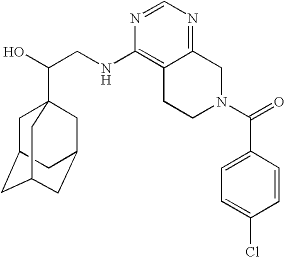 | 467.01 | 467.25 | | C1 |
| 513 | 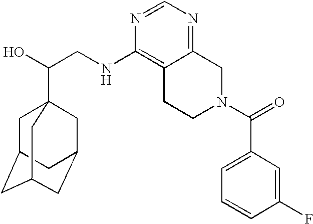 | 450.55 | 451.21 | | C1 |
| 514 | 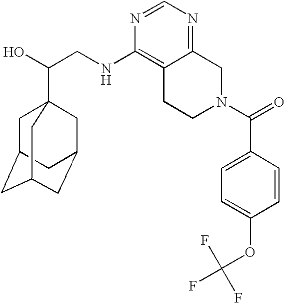 | 516.56 | 517.29 | | C1 |
| 515 | 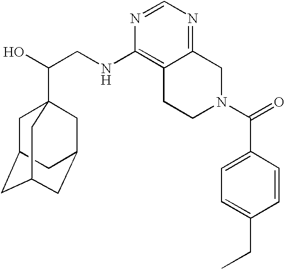 | 460.62 | 461.31 | | C1 |
| 516 | 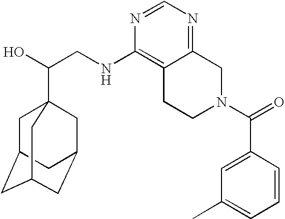 | 446.59 | 447.22 | | C1 |
| 517 | 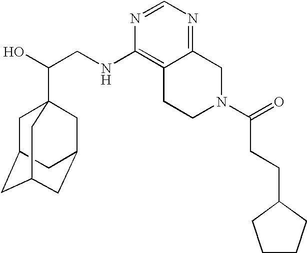 | 452.64 | 453.24 | ++++ | C1 |
| 518 | 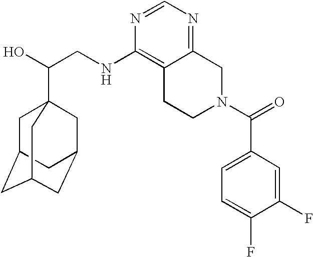 | 468.55 | 469.32 | | C1 |
| 519 | 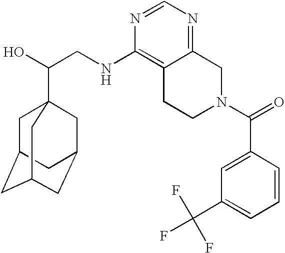 | 500.56 | 501.29 | +++ | C1 |
| 520 | 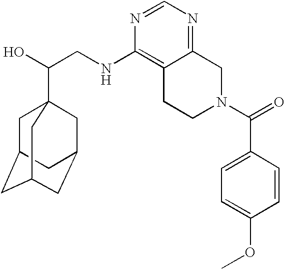 | 462.59 | 463.31 | | C1 |
| 521 | 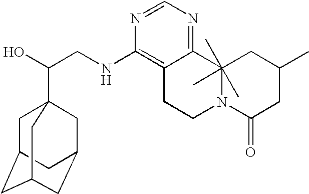 | 468.68 | 469.40 | ++++ | C1 |
| 522 | 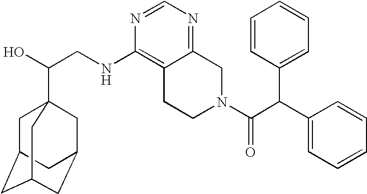 | 522.69 | 523.46 | + | C1 |
| 523 | 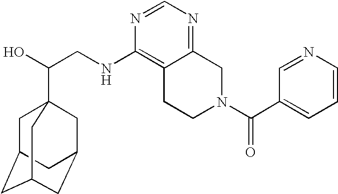 | 433.55 | 434.31 | + | C1 |
| 524 | 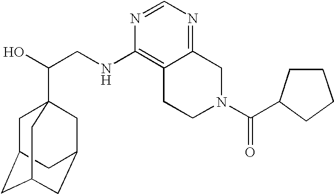 | 424.59 | 425.27 | + | C1 |
| 525 | 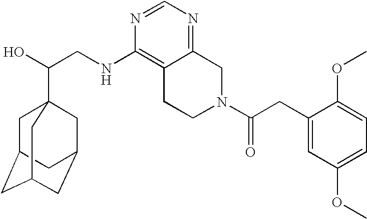 | 506.64 | 507.32 | ++ | C1 |
| 526 | 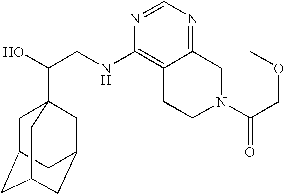 | 400.52 | 401.29 | + | C1 |
| 527 | 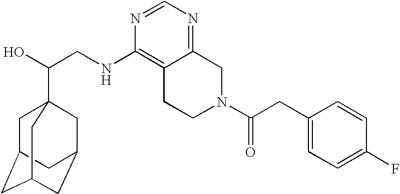 | 464.58 | 465.27 | + | C1 |
| 528 | 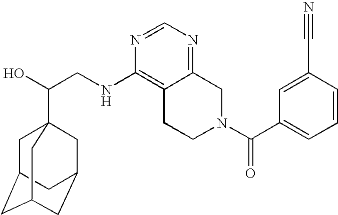 | 457.57 | 458.39 | + | C1 |
| 529 | 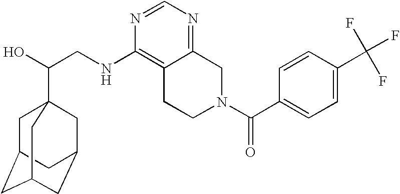 | 500.56 | 501.29 | ++ | C1 |
| 530 | 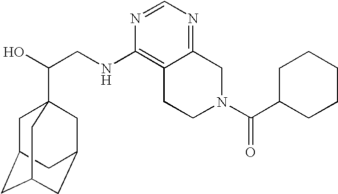 | 438.61 | 439.45 | ++++ | C1 |
| 531 | 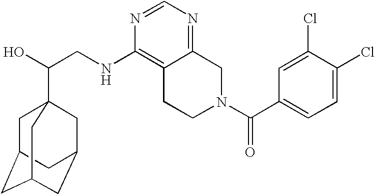 | 501.46 | 501.23 | | C1 |
| 532 | 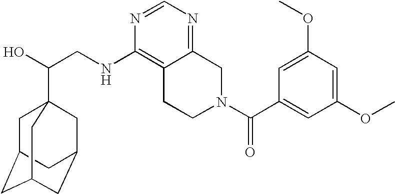 | 492.62 | 493.38 | + | C1 |
| 533 | 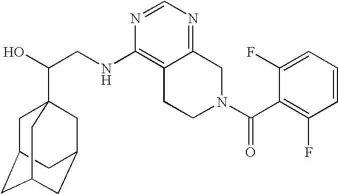 | 468.55 | 469.34 | + | C1 |
| 534 | 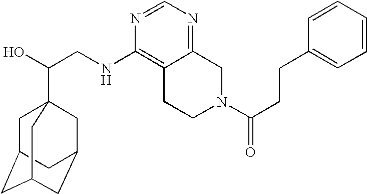 | 460.62 | 461.31 | ++ | C1 |
| 535 | 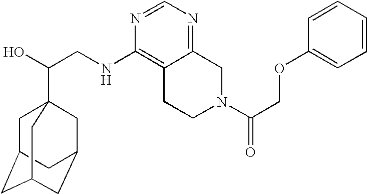 | 462.59 | 463.31 | | C1 |
| 536 | 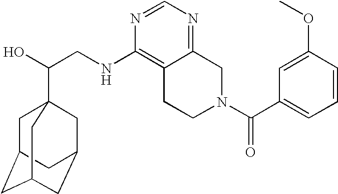 | 462.59 | 463.31 | + | C1 |
| 537 | 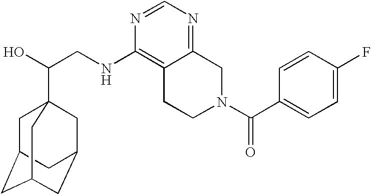 | 450.55 | 451.22 | | C1 |
| 538 | 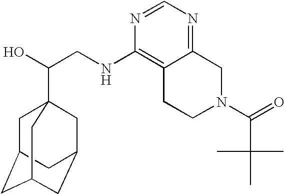 | 412.57 | 413.35 | + | C1 |
| 539 | 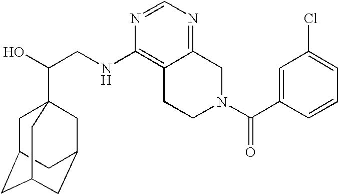 | 467.01 | 467.26 | | C1 |
| 540 | 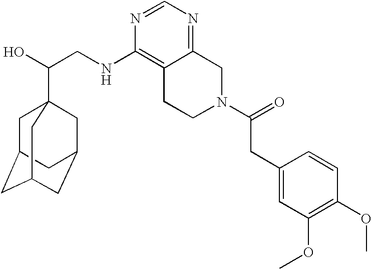 | 506.64 | 507.32 | + | C1 |
| 541 | 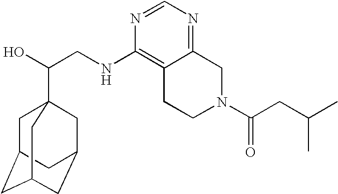 | 412.57 | 413.35 | +++ | C1 |
| 542 | 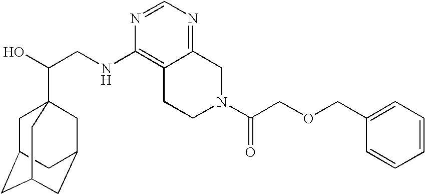 | 476.62 | 477.29 | +++ | C1 |
| 543 | 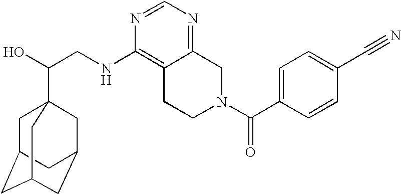 | 457.57 | 458.40 | | C1 |
| 544 | 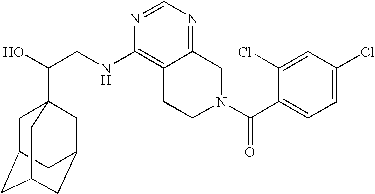 | 501.46 | 501.23 | + | C1 |
| 545 | 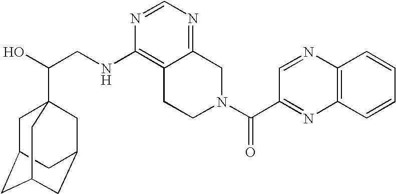 | 484.60 | | | C1 |
| 546 | 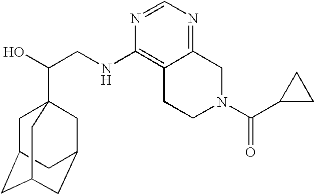 | 396.53 | 397.29 | + | C1 |
| 547 | 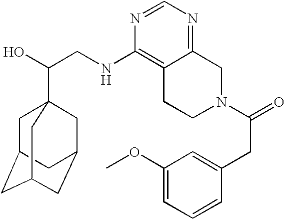 | 476.62 | 477.29 | | C1 |
| 548 | 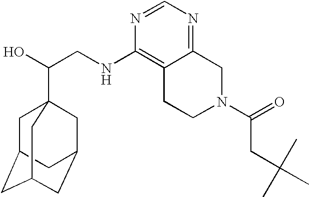 | 426.60 | 427.29 | ++++ | C1 |
| 549 | 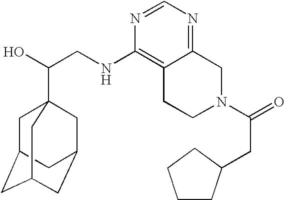 | 438.61 | 439.46 | + | C1 |
| 550 | 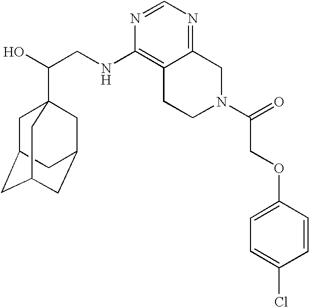 | 497.04 | 497.41 | + | C1 |
| 551 | 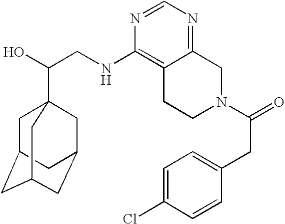 | 481.04 | 481.25 | | C1 |
| 552 | 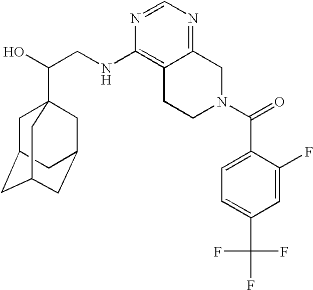 | 518.55 | 519.32 | + | C1 |
| 553 | 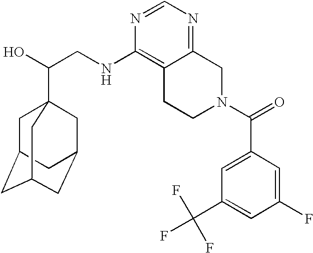 | 518.55 | 519.32 | + | C1 |
| 554 | 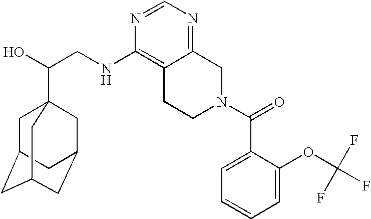 | 516.56 | | | C1 |
| 555 | 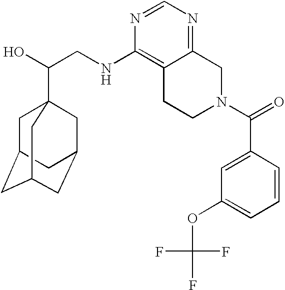 | 516.56 | | | C1 |
| 556 | 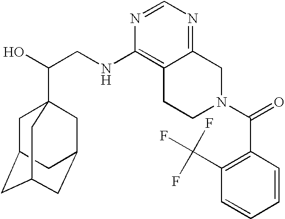 | 500.56 | 501.30 | + | C1 |
| 557 | 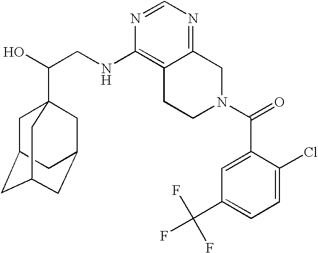 | 535.01 | 535.24 | + | C1 |
| 558 | 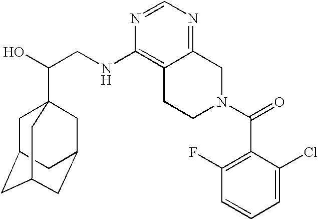 | 485.00 | 485.35 | + | C1 |
| 559 | 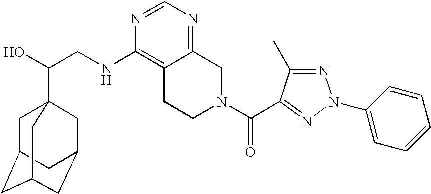 | 513.64 | 514.36 | + | C1 |
| 560 | 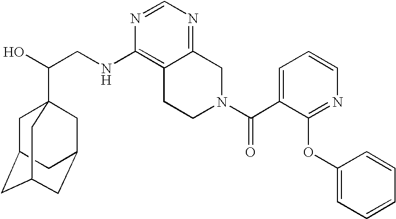 | 525.65 | 526.34 | + | C1 |
| 561 | 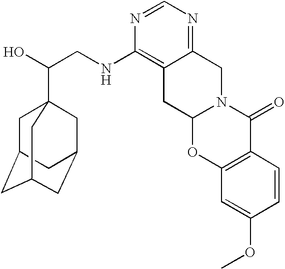 | 492.62 | 493.37 | ++ | C1 |
| 562 | 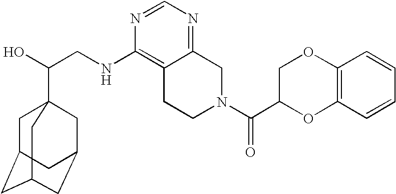 | 490.60 | 491.29 | ++ | C1 |
| 563 | 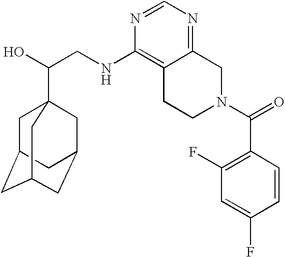 | 468.55 | 469.33 | + | C1 |
| 564 | 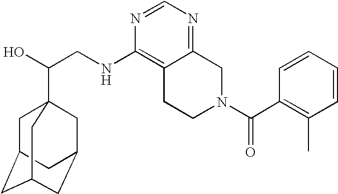 | 446.59 | 447.25 | | C1 |
| 565 | 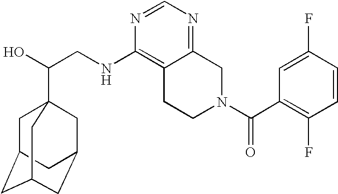 | 468.55 | 469.33 | + | C1 |
| 566 | 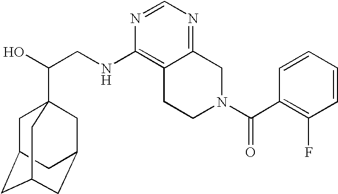 | 450.55 | 451.22 | + | C1 |
| 567 | 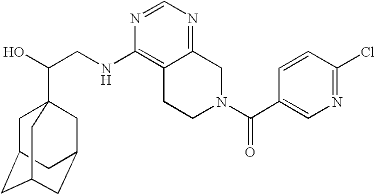 | 468.00 | 468.21 | ++ | C1 |
| 568 | 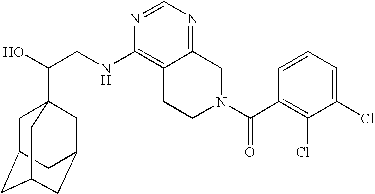 | 501.46 | 501.23 | + | C1 |
| 569 | 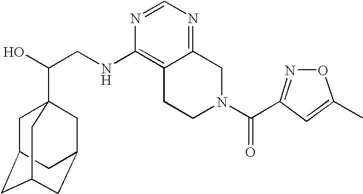 | 437.54 | 438.21 | + | C1 |
| 570 | 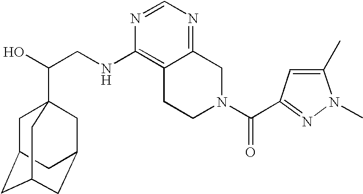 | 450.58 | 451.24 | ++ | C1 |
| 571 | 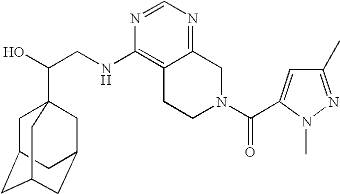 | 450.58 | 451.24 | + | C1 |
| 572 | 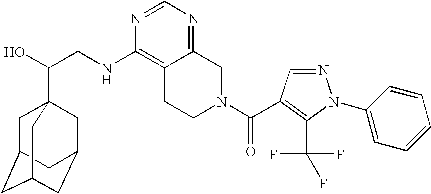 | 566.62 | 567.33 | + | C1 |
| 573 | 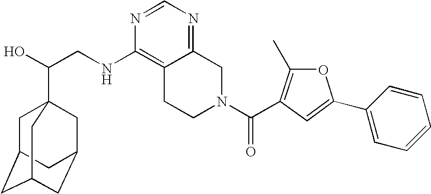 | 512.65 | | | C1 |
| 574 | 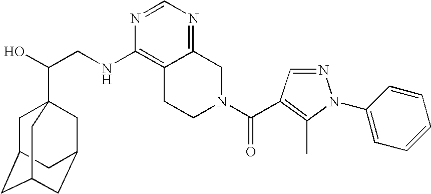 | 512.65 | 513.40 | | C1 |
| 575 | 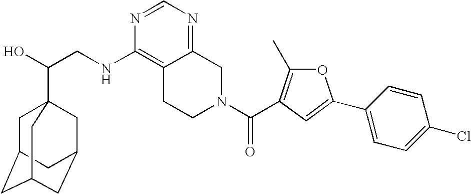 | 547.10 | 547.30 | | C1 |
| 576 | 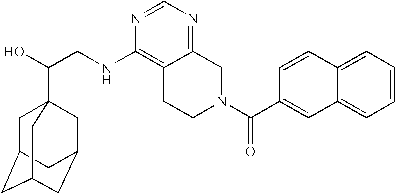 | 482.62 | 483.33 | | C1 |
| 577 | 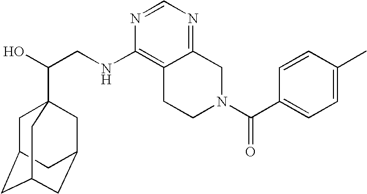 | 446.59 | 447.25 | | C1 |
| 578 | 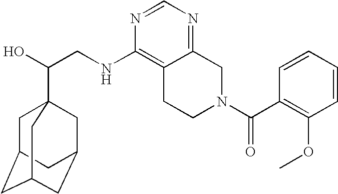 | 462.59 | 463.30 | +++ | C1 |
| 579 | 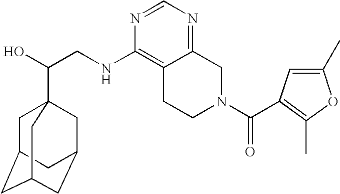 | 450.58 | 451.23 | + | C1 |
| 580 | 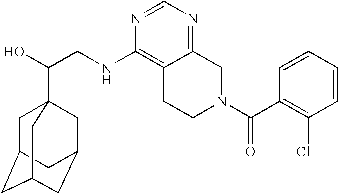 | 467.01 | 467.27 | ++ | C1 |
| 581 | 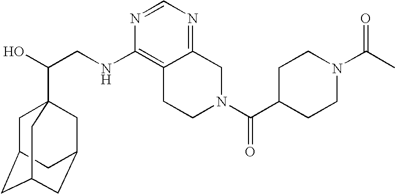 | 481.64 | 482.28 | + | C1 |
| 582 | 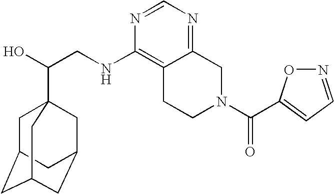 | 423.51 | 424.21 | + | C1 |
| 583 | 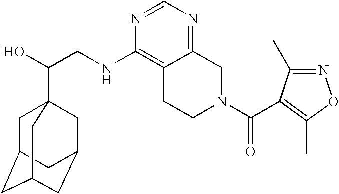 | 451.57 | 452.18 | + | C1 |
| 585 | 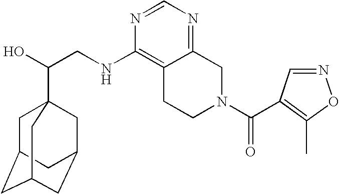 | 437.54 | 438.22 | + | C1 |
| 586 | 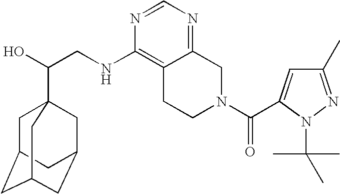 | 492.66 | 493.39 | + | C1 |
| 587 | 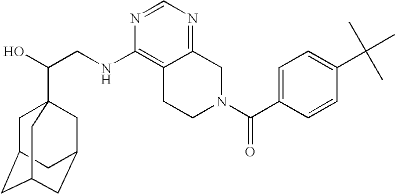 | 488.67 | 489.36 | ++++ | C1 |
| 588 | 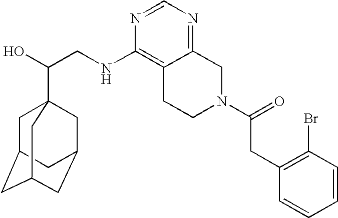 | 525.49 | 527.20 | | C1 |
| 589 | 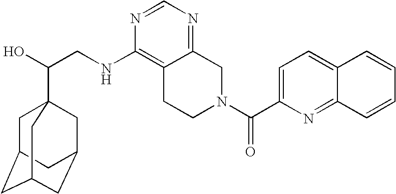 | 483.61 | 484.44 | +++ | C1 |
| 590 | 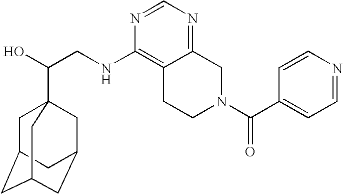 | 433.55 | 434.31 | + | C1 |
| 591 | 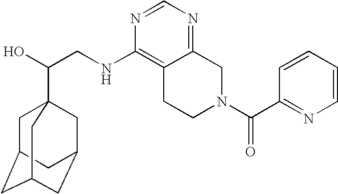 | 433.55 | 434.31 | + | C1 |
| 593 | 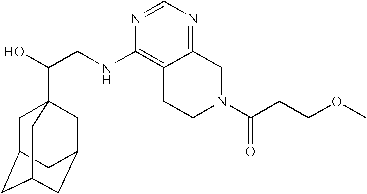 | 414.55 | 415.39 | + | C1 |
| 594 | 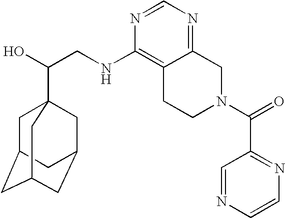 | 434.54 | 435.35 | | C1 |
| 595 | 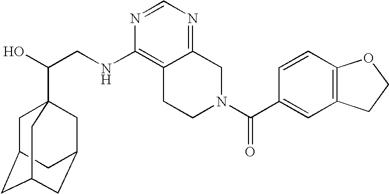 | 474.60 | 475.26 | + | C1 |
| 596 | 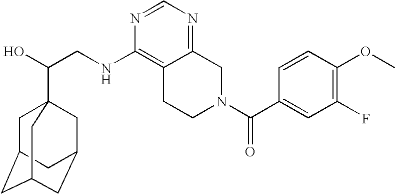 | 480.58 | 481.27 | + | C1 |
| 597 | 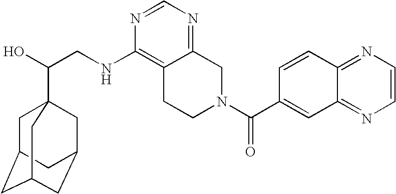 | 484.60 | | | C1 |
| 598 | 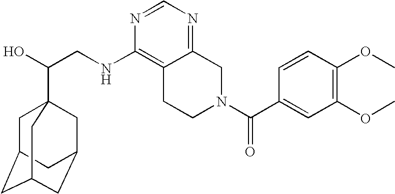 | 492.62 | 493.36 | | C1 |
| 599 | 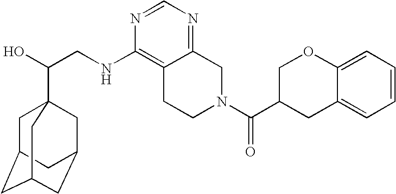 | 488.63 | 489.34 | ++++ | C1 |
| 600 | 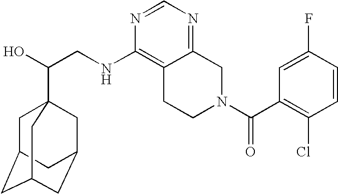 | 485.00 | | | C1 |
| 601 | 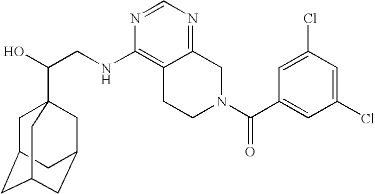 | 501.46 | 501.21 | + | C1 |
| 602 | 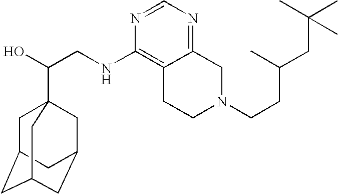 | 454.70 | 455.36 | +++ | I |
| 603 | 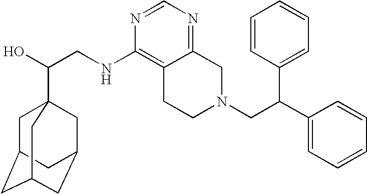 | 508.71 | 509.36 | + | I |
| 604 | 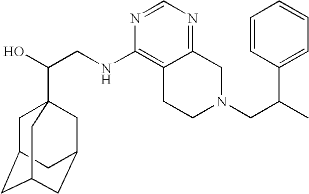 | 446.64 | 447.27 | + | I |
| 605 | 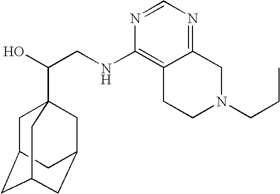 | 370.54 | 371.25 | + | I |
| 606 | 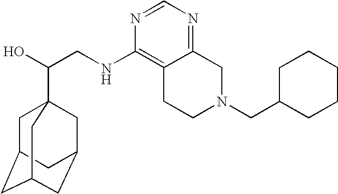 | 424.63 | 425.28 | | I |
| 607 | 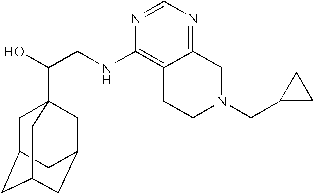 | 382.55 | 383.34 | + | I |
| 608 | 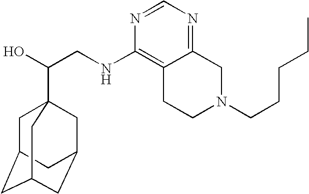 | 398.59 | 399.30 | ++ | I |
| 609 | 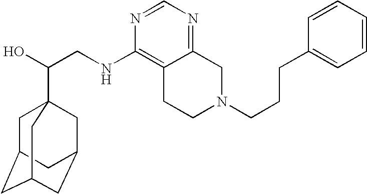 | 446.64 | 447.28 | ++ | I |
| 610 | 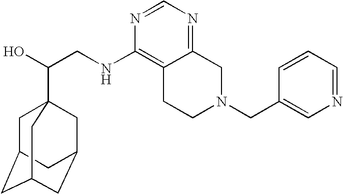 | 419.57 | 420.26 | + | I |
| 611 | 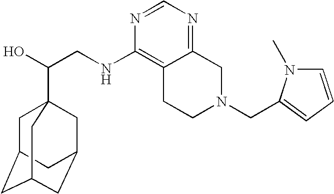 | 421.59 | 420.15 | | I |
| 612 | 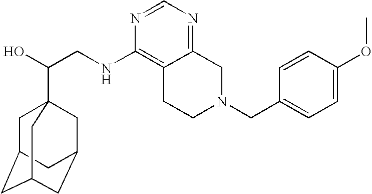 | 448.61 | 449.24 | + | I |
| 613 | 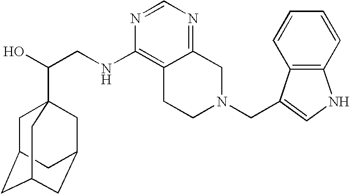 | 457.62 | 458.32 | | I |
| 614 | 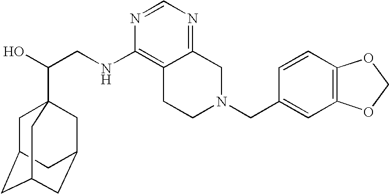 | 462.59 | 463.31 | + | I |
| 615 | 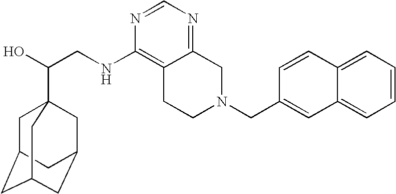 | 468.64 | 469.35 | +++ | I |
| 616 | 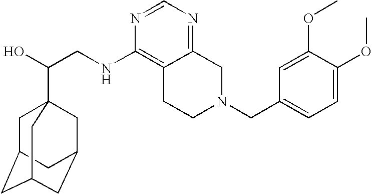 | 478.63 | 479.18 | + | I |
| 617 | 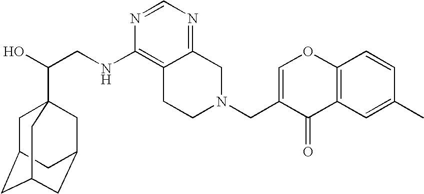 | 500.64 | 501.32 | + | I |
| 618 | 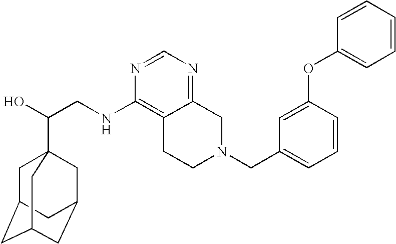 | 510.68 | 511.47 | +++ | I |
| 619 | 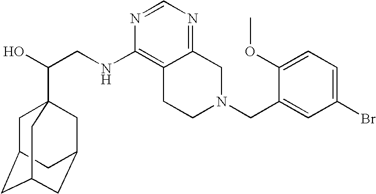 | 527.50 | 529.14 | ++ | I |
| 620 | 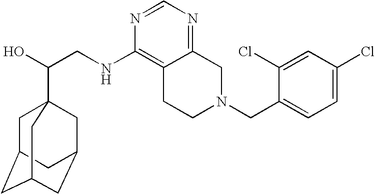 | 487.47 | 487.29 | | I |
| 621 | 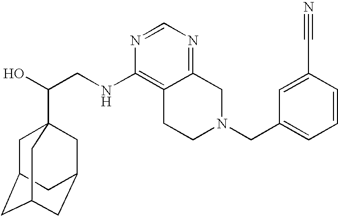 | 443.59 | 444.46 | + | I |
| 622 | 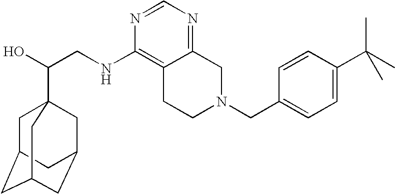 | 474.69 | 475.34 | | I |
| 623 | 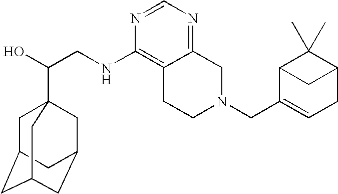 | 462.68 | 463.34 | + | I |
| 624 | 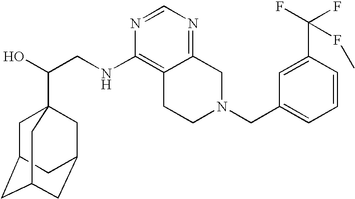 | 486.58 | 487.34 | | I |
| 625 | 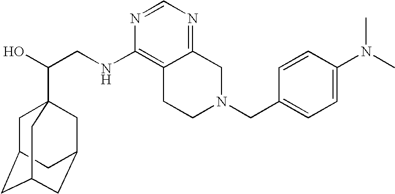 | 461.65 | 462.40 | | I |
| 626 | 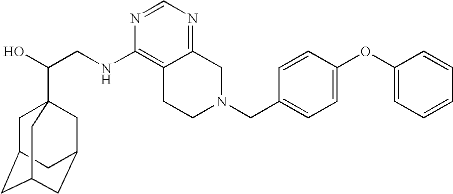 | 510.68 | 511.47 | ++ | I |
| 627 | 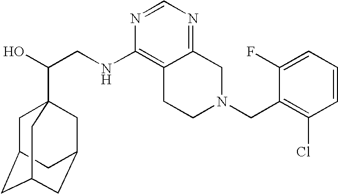 | 471.02 | 471.43 | ++ | I |
| 628 | 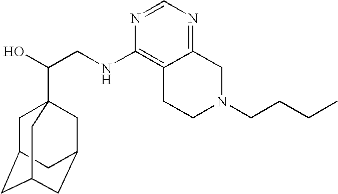 | 384.56 | 385.32 | + | I |
| 629 | 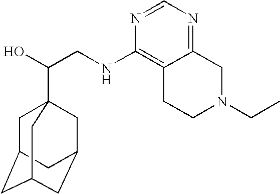 | 356.51 | 357.24 | + | I |
| 630 | 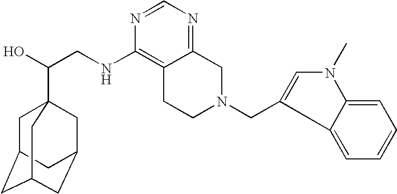 | 471.65 | 472.33 | | I |
| 631 | 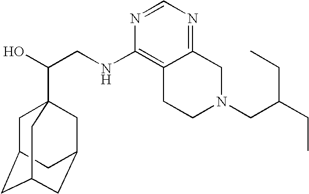 | 412.62 | 413.37 | | I |
| 632 | 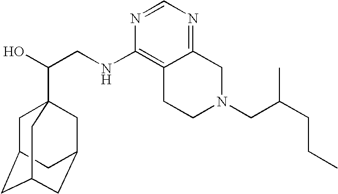 | 412.62 | 413.39 | | I |
| 633 | 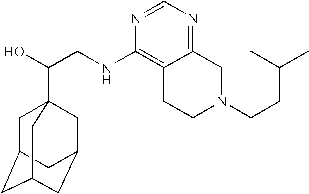 | 398.59 | 399.30 | | I |
| 634 | 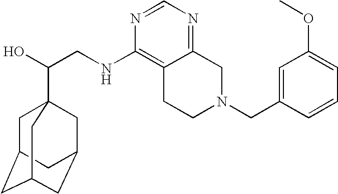 | 448.61 | 449.24 | ++ | I |
| 635 | 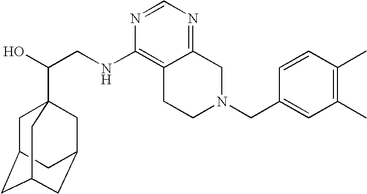 | 446.64 | 447.29 | ++ | I |
| 636 | 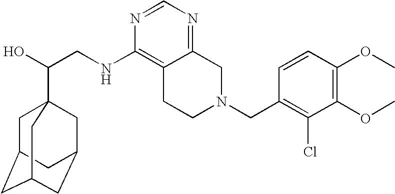 | 513.08 | 513.38 | + | I |
| 637 | 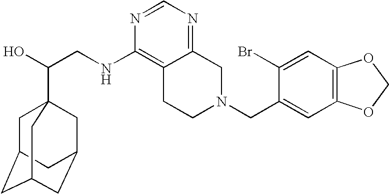 | 541.49 | 541.24 | + | I |
| 638 | 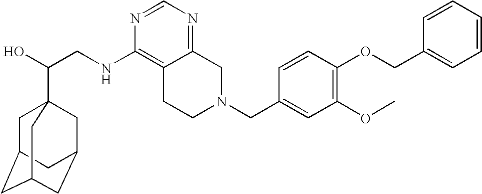 | 554.73 | 555.32 | + | I |
| 639 | 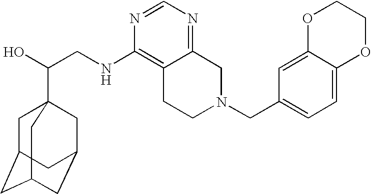 | 476.62 | 477.28 | + | I |
| 640 | 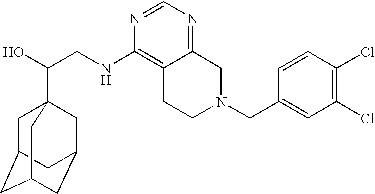 | 487.47 | 487.29 | + | I |
| 641 | 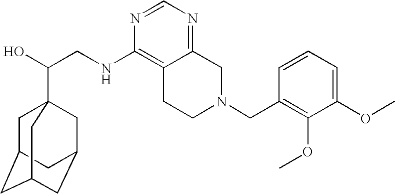 | 478.63 | 479.19 | ++ | I |
| 642 | 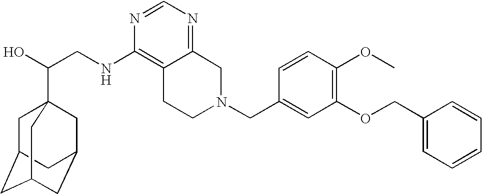 | 554.73 | 555.30 | + | I |
| 643 | 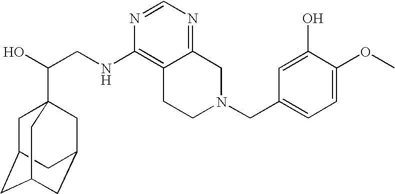 | 464.61 | 465.28 | + | I |
| 644 | 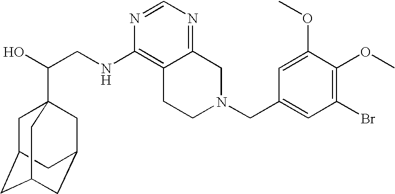 | 557.53 | 557.04 | + | I |
| 645 | 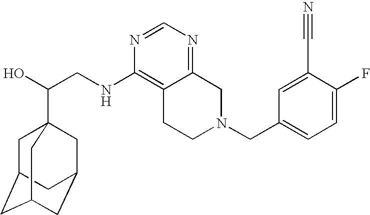 | 461.58 | 462.37 | + | I |
| 646 | 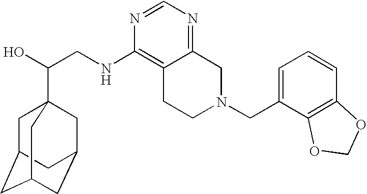 | 462.59 | 463.30 | ++ | I |
| 647 | 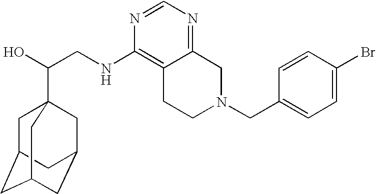 | 497.48 | 497.38 | + | I |
| 648 | 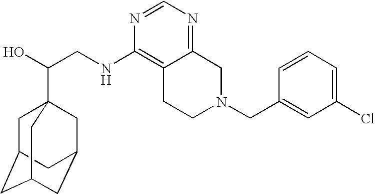 | 453.03 | 453.12 | + | I |
| 649 | 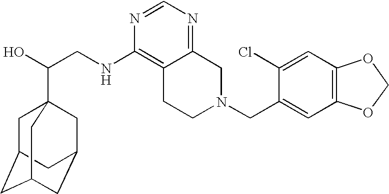 | 497.04 | 497.40 | ++ | I |
| 650 | 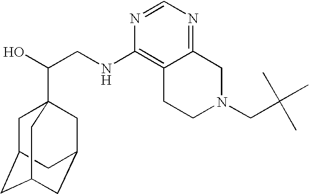 | 398.59 | 399.30 | | I |
| 651 | 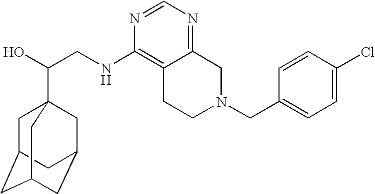 | 453.03 | 453.12 | + | I |
| 652 | 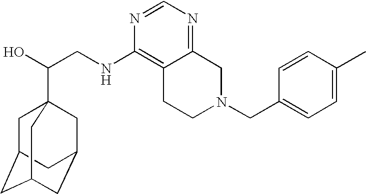 | 432.61 | 433.35 | +++ | I |
| 653 | 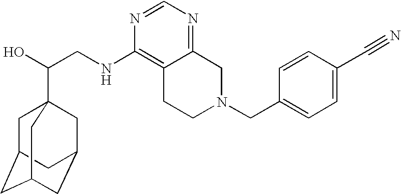 | 443.59 | 444.46 | + | I |
| 654 | 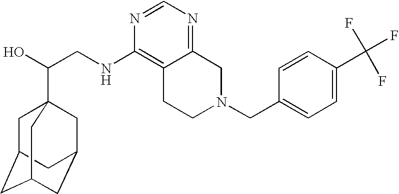 | 486.58 | 487.33 | | I |
| 655 | 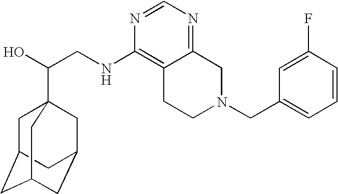 | 436.57 | 437.23 | + | I |
| 656 | 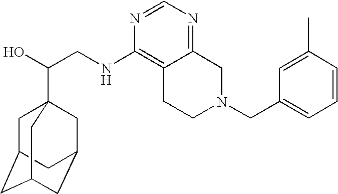 | 432.61 | 433.35 | + | I |
| 657 | 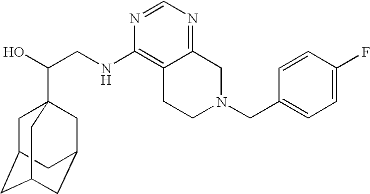 | 436.57 | 437.24 | ++ | I |
| 658 | 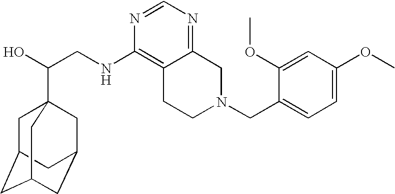 | 478.63 | 479.21 | | I |
| 659 | 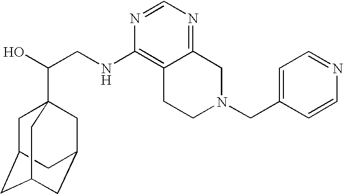 | 419.57 | 420.27 | + | I |
| 660 | 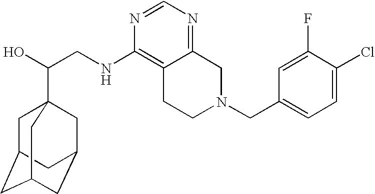 | 471.02 | 471.45 | | I |
| 661 | 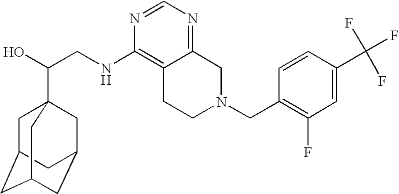 | 504.57 | 505.25 | + | I |
| 662 | 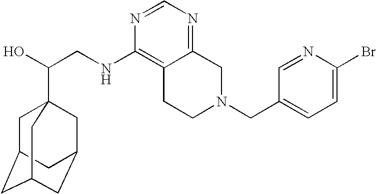 | 498.47 | 498.38 | + | I |
| 663 | 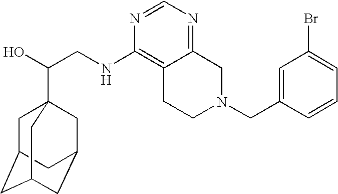 | 497.48 | 497.37 | ++ | I |
| 664 | 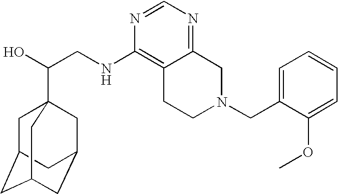 | 448.61 | 449.24 | ++ | I |
| 665 | 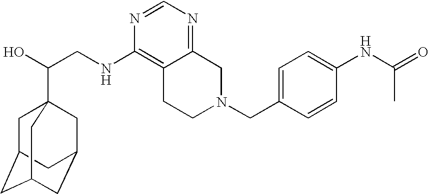 | 475.63 | 476.25 | + | I |
| 666 | 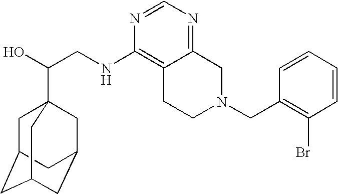 | 497.48 | 499.19 | | I |
| 667 | 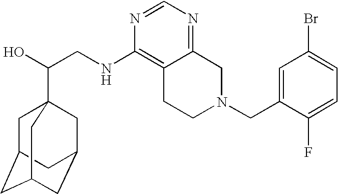 | 515.47 | 517.22 | ++ | I |
| 668 | 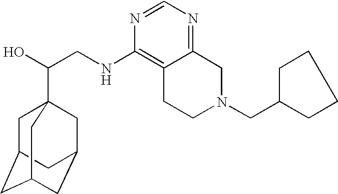 | 410.60 | 411.31 | + | I |
| 669 | 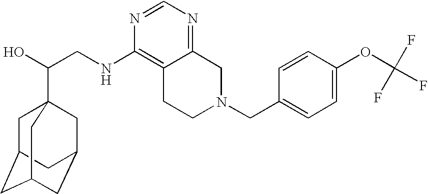 | 502.58 | 503.30 | + | I |
| 670 | 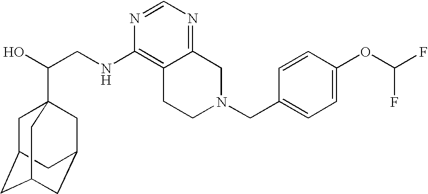 | 484.59 | 485.38 | | I |
| 671 | 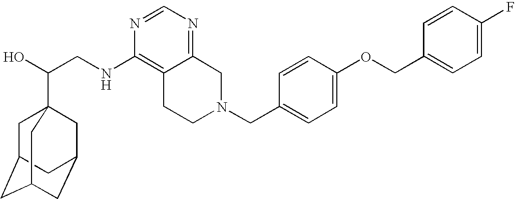 | 542.70 | 543.35 | | I |
| 672 | 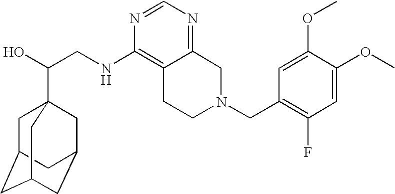 | 496.62 | 497.52 | | I |
| 673 | 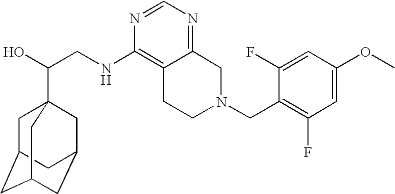 | 484.59 | 485.38 | ++ | I |
| 674 | 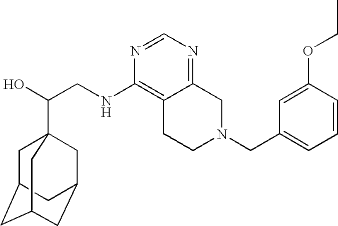 | 462.63 | 463.33 | + | I |
| 675 | 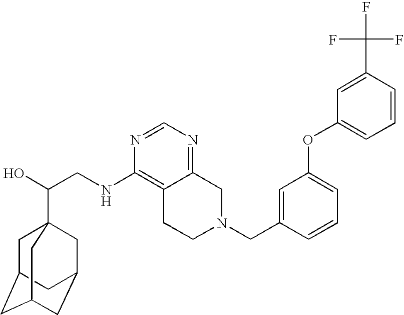 | 578.68 | 579.40 | | I |
| 676 | 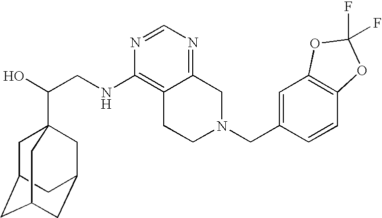 | 498.57 | 499.31 | | I |
| 677 | 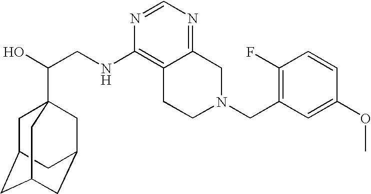 | 466.60 | 467.30 | ++ | I |
| 678 | 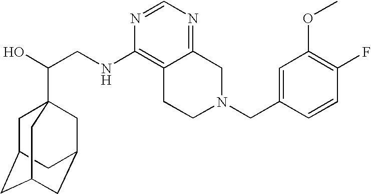 | 466.60 | 467.30 | + | I |
| 679 | 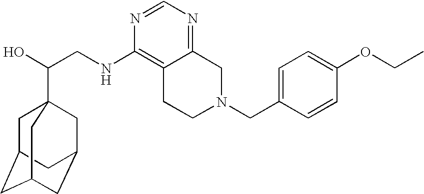 | 462.63 | 463.33 | | I |
| 680 | 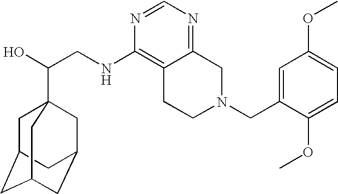 | 478.63 | 479.21 | + | I |
| 681 | 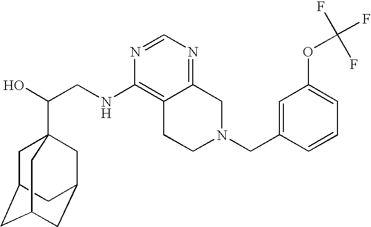 | 502.58 | 503.30 | | I |
| 682 | 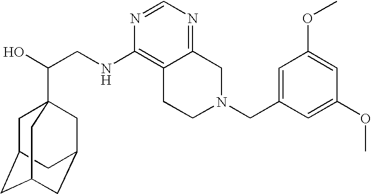 | 478.63 | 479.19 | ++ | I |
| 683 | 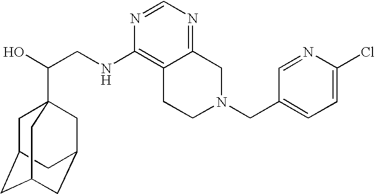 | 454.01 | 454.17 | + | I |
| 684 | 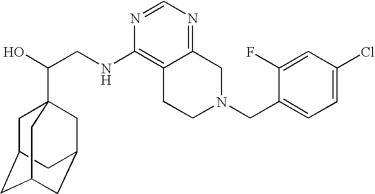 | 471.02 | 471.42 | +++ | I |
| 685 | 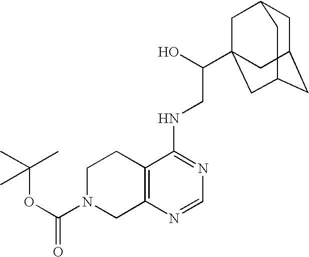 | 428.57 | 429.20 | +++ | I |
| 686 | 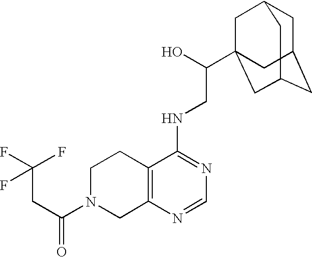 | 438.49 | 439.30 | + | C2 |
| 687 | 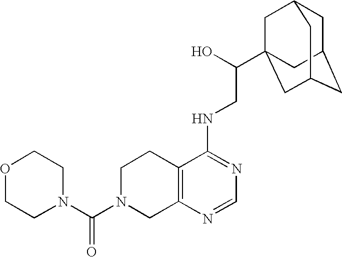 | 441.57 | 442.50 | + | C1 |
| 688 | 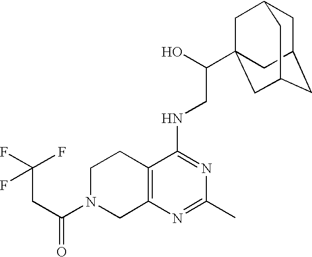 | 452.52 | 452.70 | + | C2 |
| 689 | 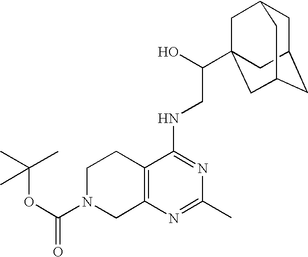 | 442.60 | 443.20 | + | J |
| 690 | 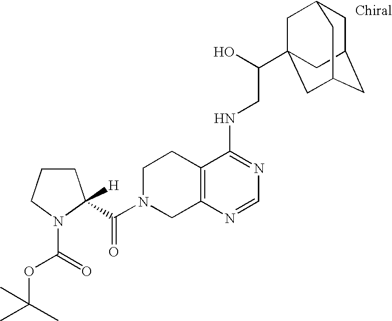 | 525.69 | 524.50 | + | C2 |
| 691 | 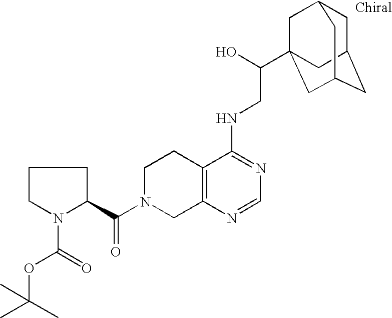 | 525.69 | 524.50 | + | C2 |
| 692 | 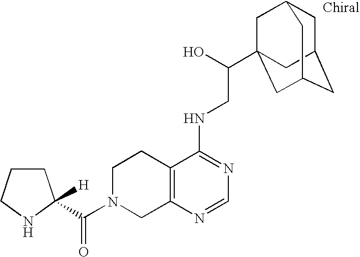 | 425.57 | 426.40 | + | C3 |
| 693 | 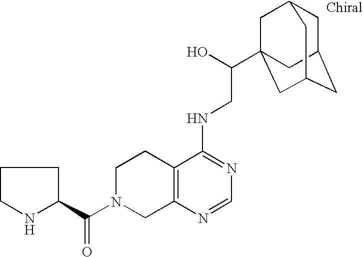 | 425.57 | 426.50 | + | C3 |
| 694 | 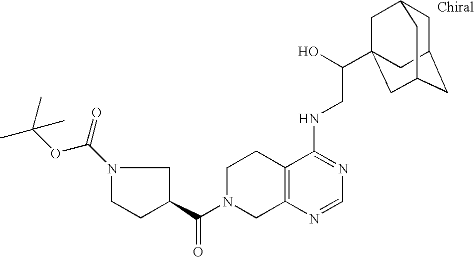 | 525.69 | 526.70 | + | C2 |
| 695 | 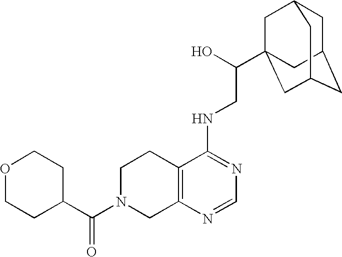 | 440.58 | 441.60 | + | C2 |
| 696 | 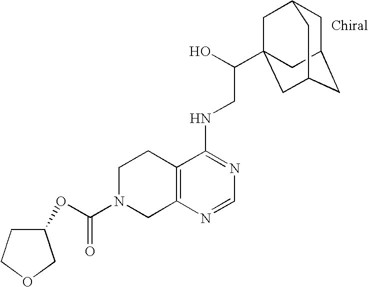 | 442.56 | 443.50 | ++ | H |
| 697 | 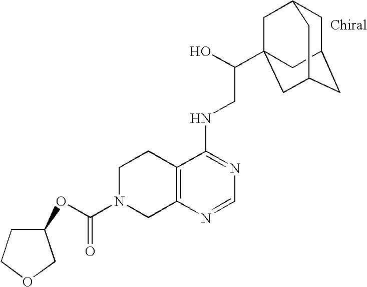 | 442.56 | 443.50 | +++ | H |
| 698 | 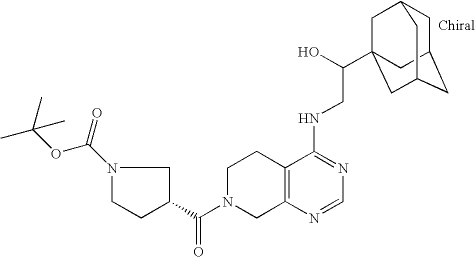 | 525.69 | 526.70 | ++ | C2 |
| 699 | 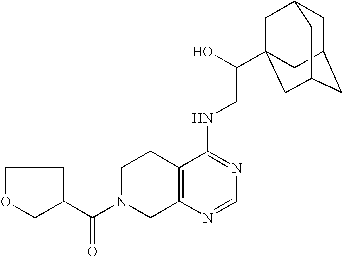 | 426.56 | 426.60 | + | C2 |
| 700 | 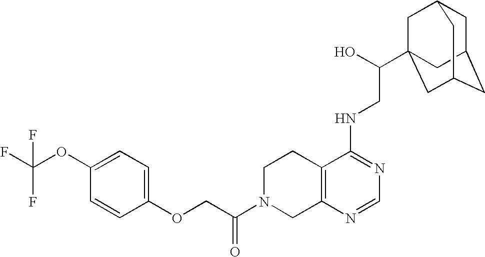 | 546.59 | 546.70 | + | C1 |
| 701 | 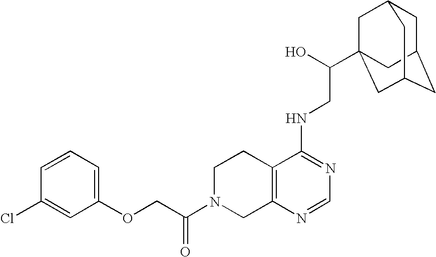 | 497.04 | 498.30 | ++ | C1 |
| 702 | 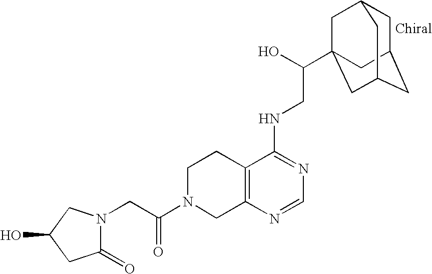 | 469.58 | 469.90 | + | C2 |
| 703 | 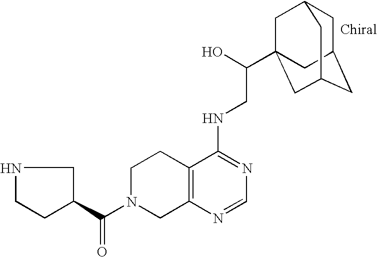 | 425.57 | 426.00 | + | C1 |
| 704 | 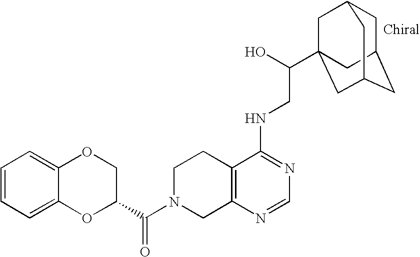 | 490.60 | 491.30 | ++++ | C2 |
| 705 | 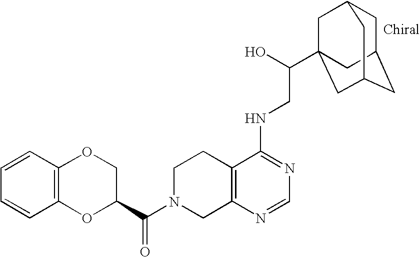 | 490.60 | 491.30 | ++++ | C2 |
| 706 | 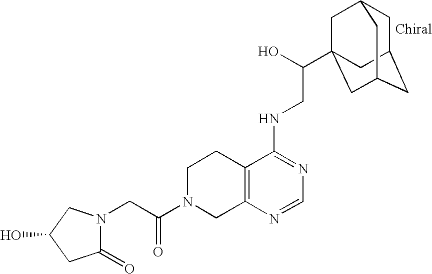 | 469.58 | 469.80 | ++ | C2 |
| 707 | 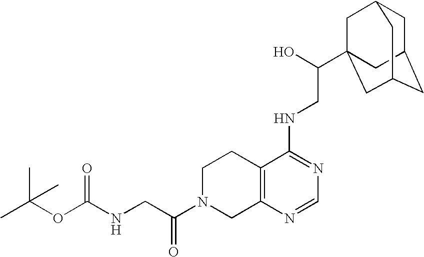 | 485.63 | 486.30 | *** | C2 |
| 708 | 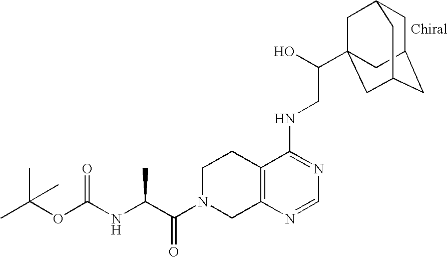 | 499.65 | 500.70 | *** | C2 |
| 709 | 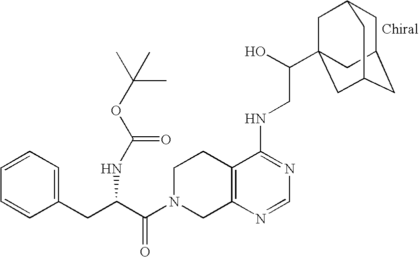 | 575.75 | 576.80 | * | C2 |
| 710 | 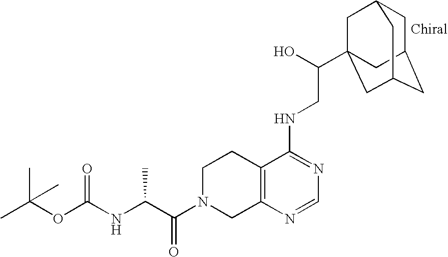 | 499.65 | 500.00 | * | C2 |
| 711 | 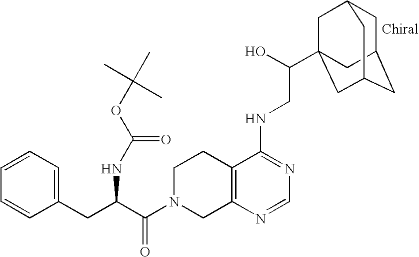 | 575.75 | 576.50 | * | C2 |
| 712 | 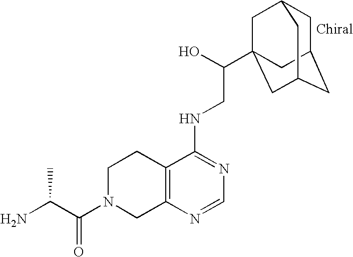 | 399.54 | 400.30 | ++++ | C3 |
| 713 | 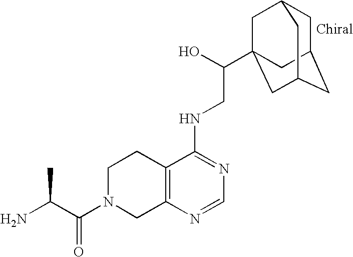 | 399.54 | 400.40 | * | C3 |
| 714 | 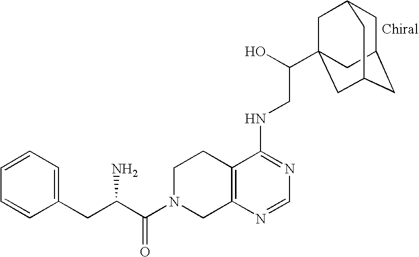 | 475.63 | 476.40 | ++++ | C3 |
| 715 | 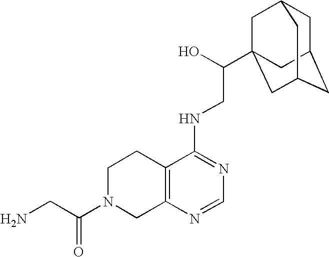 | 385.51 | 386.30 | ++++ | C3 |
| 716 | 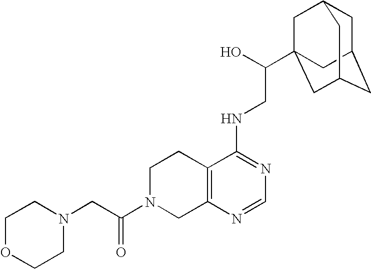 | 455.60 | 456.10 | ++++ | C2 |
| 717 | 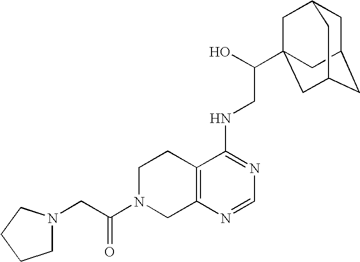 | 439.60 | 440.30 | ++++ | C4 |
| 718 | 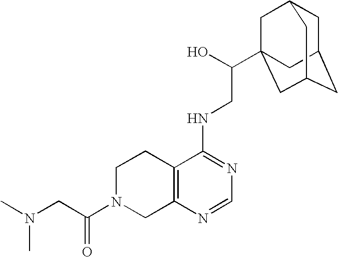 | 413.56 | 414.50 | ++++ | C2 |
| 719 | 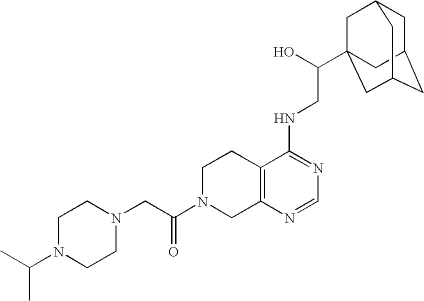 | 496.70 | 497.60 | ++++ | C2 |
| 720 | 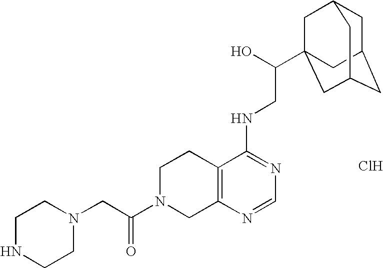 | 491.08 | 455.40 | +++ | C3 |
| 723 | 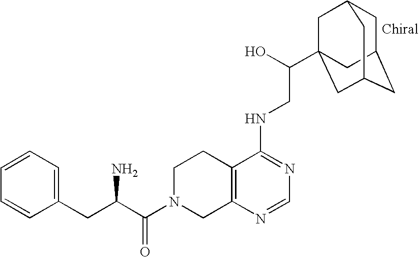 | 475.63 | 476.20 | ++++ | C3 |
| 724 | 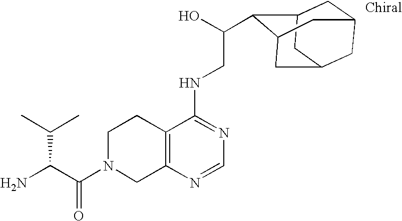 | 427.59 | 428.20 | ++ | C3 |
| 725 | 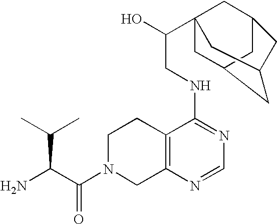 | 427.59 | 428.40 | ++++ | C3 |
| 726 | 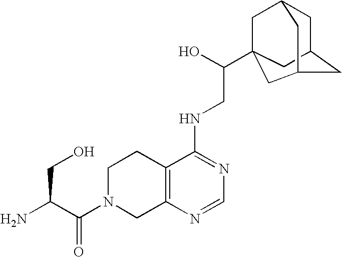 | 415.53 | 416.20 | ++ | C3 |
| 727 | 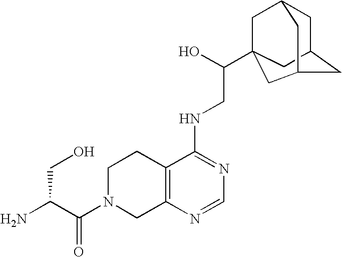 | 415.53 | 415.90 | ++ | C3 |

“+” compound exhibited 0-25% inhibition at 0.3 μM
“++” compound exhibited 25-50% inhibition at 0.3 μM
“+++” compound exhibited 50-75% inhibition at 0.3 μM
“++++” compound exhibited 75% or greater inhibition at 0.3 μM
“*” compound exhibited 0-25% inhibition at 0.03 μM
“**” compound exhibited 25-50% inhibition at 0.03 μM
“***” compound exhibited 50-75% inhibition at 0.03 μM
“****” compound exhibited 75% or greater inhibition at 0.03 μM

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

The chemical names of compounds of invention given in this application are generated using Open Eye Software's Lexichem naming tool or MDL's ISIS Draw Autonom Software tool and not verified.

1. (canceled)

2. (canceled)

3. A compound having a formula: 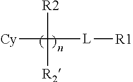
wherein
Cy is 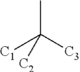
wherein C¹, C² and C³ taken together with the C atom to which they are attached, form a bi- or tri-cyloalkyl or cycloheteroalkyl ring system of 7-13 atoms; and wherein the ring system is substituted or unsubstituted;
L is 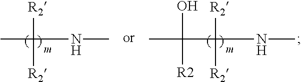
m is 0, 1, 2 or 3;
R¹ is selected from 3-13 membered cycloalkyl, heterocycloalkyl, aryl, heteroaryl, bicycloaryl and bicycloheteroaryl ring systems, which can be optionally substituted with R⁴ or one or more substituents independently selected from halo, hydroxyl, amino, cyano, substituted or unsubstituted sulfo, substituted sulfanyl, substituted sulfinyl, amido, carboxy, ester, alkyl, substituted alkyl, alkenyl, substituted alkenyl, alkynyl, substituted alkynyl, and sulfonamide;
n is 0, 1, 2 or 3;
each of R^{2′} is independently selected from hydrogen and C₁-C₆ alkyl;
R⁴ is selected from H, alkyl, acyl, acylamino, alkylamino, alkythio, alkoxy, alkoxycarbonyl, alkoxycarbonyl, alkylarylamino, arylalkyloxy, arylalkyloxy, amino, aryl, aryl, arylalkyl, substituted or unsubstituted sulfoxide, substituted sulfonyl, substituted sulfanyl, aminosulfonyl, arylsulfonyl, azido, carboxy carbamoyl, cyano, cycloalkyl, cycloheteroalkyl, dialkylamino, halo, heteroaryloxy, heteroaryl, heteroalkyl, hydroxy, nitro, and thiol;
or a pharmaceutically acceptable salt, solvate or prodrug thereof;
and stereoisomers, isotopic variants and tautomers thereof.

4. A compound of claim 3 wherein Cy is substituted or unsubstituted

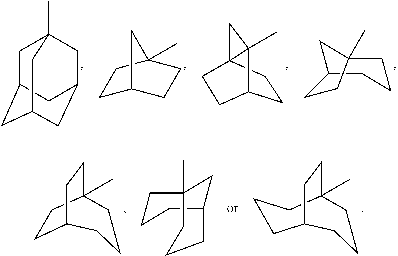

5. (canceled)

6. (canceled)

7. (canceled)

8. (canceled)

9. (canceled)

10. (canceled)

11. (canceled)

12. (canceled)

13. A compound according to claim 3 wherein R¹ is 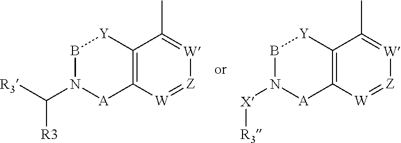
wherein
A is selected from CR^{2′}R^{2″}, CO, and CS;
B is selected from CR^{2′}, CR^{2′}R^{2″}, CO, and CS;
Y is independently selected from CR^{2′} and CR^{2′}R^{2″};
W, W′ and Z are independently selected from CR⁴ and N, provided that all three of W, W′ and Z cannot be N at the same time;
X′ is selected from —CO—, —C(═O)O—, —C(═O)—S—, —C(═O)—NH—, —SO—, —SO₂—, and —SO₂NH—;
R³ is hydrogen or a functional group selected from acyl, substituted acyl, substituted or unsubstituted acylamino, substituted or unsubstituted alkyl, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol; or R³ is a 4-9 membered carbocyclic or heterocyclic ring which can be optionally substituted with at least one substituent selected from a R⁴ group;
each of R², R^{2′}, R^{2″} and R^{3′} is independently selected from hydrogen and substituted and unsubstituted C₁-C₆ alkyl;
R^{3″} is a group selected from alkyl, substituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl, substituted or unsubstituted bicycloaryl and substituted or unsubstituted bicycloheteroalkyl, or R^{3″} is a 4-9 membered carbocyclic or heterocyclic ring which can be optionally substituted with at least one substituent selected from a R⁴ group;
R⁴ is selected from H, alkyl, acyl, acylamino, alkylamino, alkythio, alkoxy, alkoxycarbonyl, alkoxycarbonyl, alkylarylamino, arylalkyloxy, arylalkyloxy, amino, aryl, aryl, arylalkyl, substituted or unsubstituted sulfoxide, substituted sulfonyl, substituted sulfanyl, aminosulfonyl, arylsulfonyl, azido, carboxy, carbamoyl, cyano, cycloalkyl, cycloheteroalkyl, dialkylamino, halo, heteroaryloxy, heteroaryl, heteroalkyl, hydroxy, nitro, and thiol;
and the dotted bond is a single or a double bond.

14. (canceled)

15. (canceled)

16. (canceled)

17. A compound according to claim 3 wherein the compound is depicted by a formula 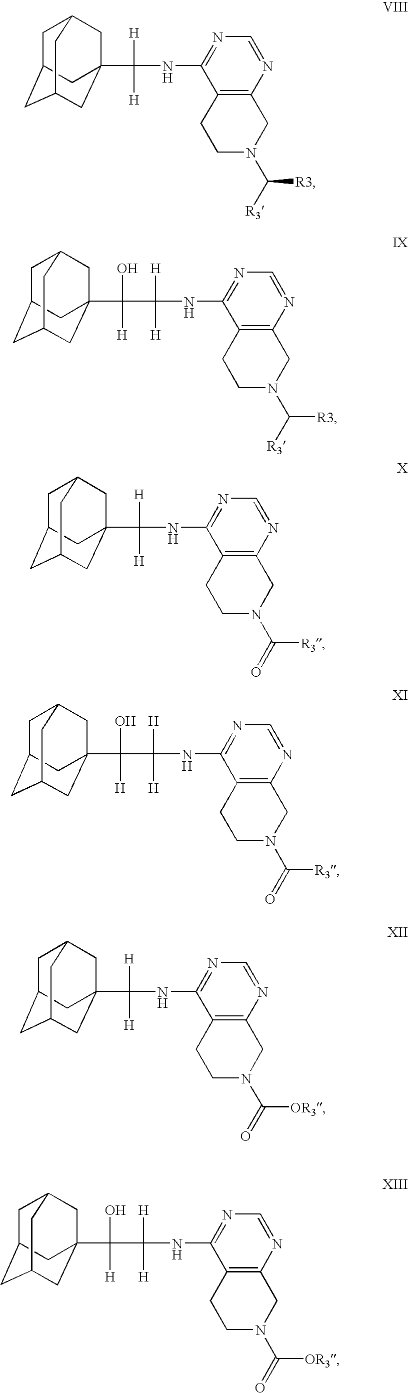 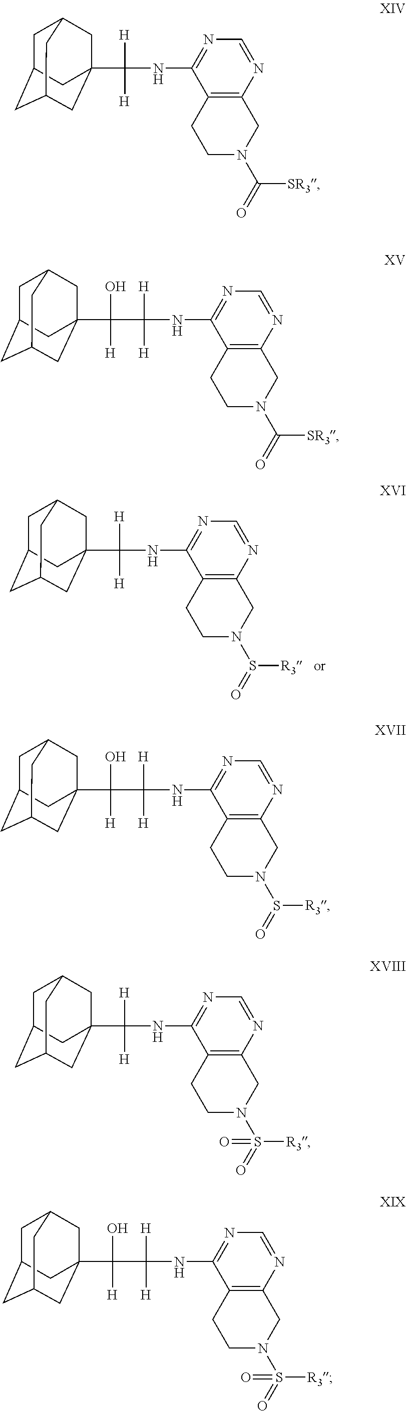
and wherein R³ is hydrogen or a functional group selected from acyl, substituted acyl, substituted or unsubstituted acylamino, substituted or unsubstituted alkyl, substituted or unsubstituted alkylamino, substituted or unsubstituted alkythio, substituted or unsubstituted alkoxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thio; or
R³ is a 4-9 membered carbocyclic or heterocyclic ring which can be optionally substituted with at least one substituent selected from a R⁴ group;
R^{3″} is a group selected from alkyl, substituted alkyl, aryl, substituted aryl, arylalkyl, substituted or unsubstituted cycloalkyl, substituted or unsubstituted cycloheteroalkyl, substituted or unsubstituted heteroaryl, substituted or unsubstituted heteroalkyl, substituted or unsubstituted bicycloaryl and substituted or unsubstituted bicycloheteroalkyl, or R^{3″} is a 4-9 membered carbocyclic or heterocyclic ring which can be optionally substituted with at least one substituent selected from a R⁴ group;
R⁴ is selected from H, alkyl, acyl, acylamino, alkylamino, alkythio, alkoxy, alkoxycarbonyl, alkoxycarbonyl, alkylarylamino, arylalkyloxy, arylalkyloxy, amino, aryl, aryl, arylalkyl, substituted or unsubstituted sulfoxide, substituted sulfonyl, substituted sulfanyl, aminosulfonyl, arylsulfonylazido, carboxy, carbamoyl, cyano, cycloalkyl, cycloheteroalkyl, dialkylamino, halo, heteroaryloxy, heteroaryl, heteroalkyl, hydroxy, nitro, and thiol;
and R^{3′} is selected from hydrogen, methyl or hydroxymethyl.

18. A compound according to claim 17 wherein R³ or R^{3″} is independently 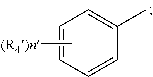
and wherein n′ is selected from 1-5 and each of R^{4′} is independently selected from hydrogen, alkyl, substituted alkyl, acyl, substituted acyl, substituted or unsubstituted acylamino, substituted or unsubstituted alkylamino, substituted or unsubstituted alkythio, substituted or unsubstituted alkoxy, aryloxy, alkoxycarbonyl, substituted alkoxycarbonyl, substituted or unsubstituted alkylarylamino, arylalkyloxy, substituted arylalkyloxy, amino, aryl, substituted aryl, arylalkyl, substituted or unsubstituted sulfoxide, substituted sulfonyl, substituted sulfanyl, substituted or unsubstituted aminosulfonyl, substituted or unsubstituted arylsulfonyl, azido, carboxy, substituted or unsubstituted carbamoyl, cyano, substituted or unsubstituted cycloalkyl, substituted or unsubstituted cycloheteroalkyl, substituted or unsubstituted dialkylamino, halo, heteroaryloxy, substituted or unsubstituted heteroaryl, substituted or unsubstituted heteroalkyl, hydroxy, nitro, and thiol.

19. A compound according to claim 18 wherein n′ is 1, 2 or 3.

20. A compound according to claim 18 wherein each R^{4′} is independently selected from Me, Et, Ph, Cl, F, Br, CN, OH, OMe, OEt, OPh, CF₃, CHF₂, OCHF₂, NMe₂, OCF₃, t-Bu, SMe, NHCOMe, OCH₂Ph, CH═CH—CO₂H, SOMe, SO₂Me, SO₃H, SO₃Me, and pyridyl.

21. A compound according to claim 17 wherein R³ or R^{3″} is independently substituted or unsubstituted cycloalkyl, heterocycloalkyl, heteroaryl, bicycloaryl or bicycloheteroaryl.

22. A compound according to claim 17 wherein R³ or R^{3″} is independently substituted or unsubstituted naphthalene, furanyl, thiophenyl, pyrrolyl, imidazolyl, pyridyl, pyrimidinyl, quinoline, isoquinolinyl, triazolyl, oxazolyl, pyrazolyl, piperidinyl, piperizinyl, tetrahydrofuranyl, morpholinyl, azepinyl, coumarinyl, tetrahydroquinolinyl, tetrahydroisoquinolinyl, indolyl, benzopyranyl, benzofuranyl, benzodioxolyl, or benzodioxanyl.

23. A compound according to claim 17 wherein R³ or R^{3″} is independently selected from

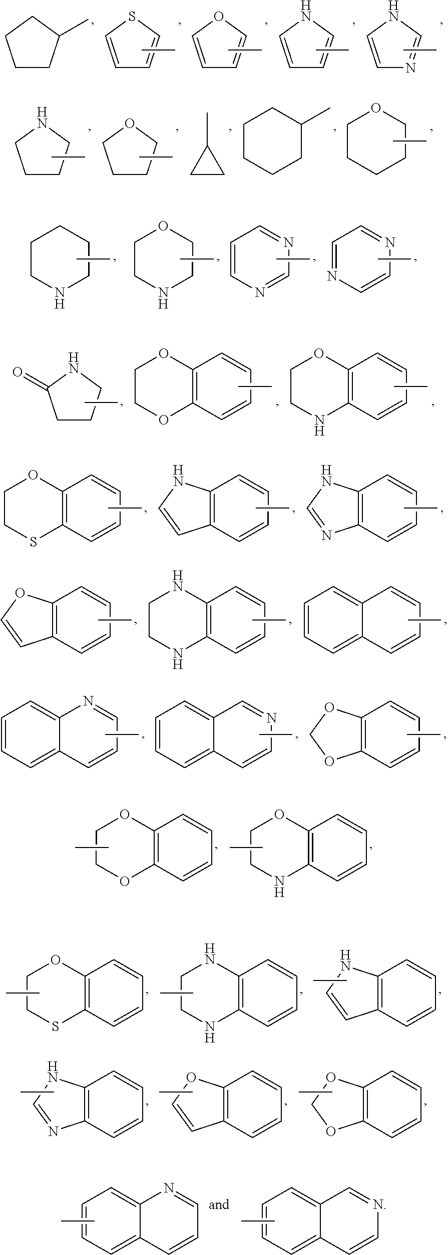

24. A compound according to claim 17 wherein R³ or R^{3″} is independently substituted C₁-C₆ alkyl and the substitution is selected from hydroxyl, amino, substituted amino, alkoxy, carbamoyl, carboxy, carbalkoxy, sulfonyl, sulfanyl, sulfinyl, alkyl, halo, cyano, —NHCO₂R^{2′}, —NHSO₂R^{2′}, —NHCONH₂, —NHCONHSO₃R^{2′} and wherein R^{2′} is substituted or unsubstituted C₁-C₆ alkyl.

25. A compound according to claim 17 wherein R³ or R^{3″} is independently substituted C₁-C₆ alkyl and the substitution is selected from phenyl, OH, NH₂, NHMe, CN, NHEt, NHCONHSO₃Me, SO₂Me, CF₃, SO₂CF₃, NHSO₂CF₃, NHSO₂Me, Me, CO₂H, CO₂Et, CONH₂, pyrrolidinyl, pyrazolyl, piperidinyl, or morpholinyl.

26. A compound according to claim 17 wherein R³ or R^{3″} is independently selected from (CH₂)₃—OH, —(CH₂)₄—NHMe, —(CH₂)₄—OH, —(CH₂)₂₋CH(OH)—CH₂OH, —(CH₂)₄—CO₂H, —(CH₂)₄—NHEt, —(CH₂)₃—NHEt, —(CH₂)₂—NH—(CH₂)₂OH, —(CH₂)₃—NH—(CH₂)₃OH, —(CH₂)₄—NH₂, —(CH₂)₃—NHCONHSO₂Me, —(CH₂)₃—NH—(CH₂)₂-Me, —(CH₂)₂CO₂H, and N-Me-piperidinyl.

27. A compound according to claim 17 wherein R³ or R^{3″} is independently substituted C₁-C₆ alkyl and the substitution is selected from aryl, heteroaryl, cycloalkyl, heterocycloalkyl, halo, alkoxy, hydroxy, cyano, and aryloxy.

28. A compound according to claim 17 wherein R³ or R^{3″} is independently substituted C₁-C₆ alkyl and the substitution is selected Ph, Cl, F, Br, CN, OH, OMe, OPh, CF₃, CHF₂, OCF₃, t-Bu, SMe, SOMe, SO₂Me, SO₃H, SO₃Me, CONH₂, pyridyl, cyclopropyl, cyclopentyl and cyclohexyl.

29. A compound according to claim 17 wherein R³ is selected from hydrogen, carbamoyl, carboxy, and carbalkoxy.

30. A compound according to claim 17 wherein R³ is CONH₂, CO₂H, CO₂Et, CONMe₂, pyrrolidinyl, pyrazolyl, piperidinyl, or morpholinyl.

31. A compound according to claim 1 wherein the compound is selected from compound ID numbers 1-149, 150-261, 264-351, 501-583, 585-591, 593, 720, and 723-727.

32. A pharmaceutical composition comprising a pharmaceutically acceptable carrier and a pharmaceutically effective amount of a compound of claim 3.

33. The pharmaceutical composition of claim 32, wherein the carrier is a parenteral, oral, or topical carrier.

34. (canceled)

35. (canceled)

36. (canceled)

37. A method for preventing, treating or ameliorating in a mammal a disease or condition that is causally related to the aberrant activity of the P2X₇ receptor in vivo, which comprises administering to the mammal an effective disease-treating or condition-treating amount of a pharmaceutical composition according to claim 32.

38. The method of claim 37, wherein the disease or condition is a pain condition.

39. The method of claim 37, wherein the disease or condition is an autoimmune disease.

40. The method of claim 37, wherein the disease or condition is an inflammatory disease or condition.

41. The method of claim 37, wherein the disease or condition is a neurological or neurodegenerative disease or condition.

42. A method for preventing, treating or ameliorating in a mammal a disease or condition selected from: pain including acute, inflammatory and neuropathic pain, chronic pain, dental pain and headache including migraine, cluster headache and tension headache, Parkinson's disease, multiple sclerosis; diseases and disorders which are mediated by or result in neuroinflammation, traumatic brain injury and encephalitis; centrally-mediated neuropsychiatric diseases and disorders, depression mania, bipolar disease, anxiety, schizophrenia, eating disorders, sleep disorders and cognition disorders; epilepsy and seizure disorders; prostate, bladder and bowel dysfunction, urinary incontinence, urinary hesitancy, rectal hypersensitivity, fecal incontinence, benign prostatic hypertrophy and inflammatory bowel disease; respiratory and airway disease and disorders, allergic rhinitis, asthma and reactive airway disease and chronic obstructive pulmonary disease; diseases and disorders which are mediated by or result in inflammation, arthritis, rheumatoid arthritis and osteoarthritis, myocardial infarction, various autoimmune diseases and disorders, uveitis and atherosclerosis; itch/pruritus, psoriasis; obesity; lipid disorders; cancer; blood pressure; spinal cord injury; and renal disorders which comprises administering to the mammal an effective disease-treating or condition-treating amount of a pharmaceutical composition of claim 32.

43. (canceled)

44. (canceled)

45. (canceled)

46. (canceled)

47. (canceled)

48. (canceled)

49. A method of treating a mammal suffering from at least one symptom selected from the group consisting of symptoms of exposure to capsaicin, symptoms of burns or irritation due to exposure to heat, symptoms of burns or irritation due to exposure to light, symptoms of burns, bronchoconstriction or irritation due to exposure to tear gas, and symptoms of burns or irritation due to exposure to acid, which comprises administering to the mammal an effective disease-treating or condition-treating amount of a pharmaceutical composition of claim 32.

50. The method of claim 49, wherein the pain is associated with a condition selected from the group consisting of postmastectomy pain syndrome, stump pain, phantom limb pain, oral neuropathic pain, Charcot's pain, toothache, venomous snake bite, spider bite, insect sting, postherpetic neuralgia, diabetic neuropathy, reflex sympathetic dystrophy, trigeminal neuralgia, osteoarthritis, rheumatoid arthritis, fibromyalgis, Guillain-Barre syndrome, meralgia paresthetica, burning-mouth syndrome, bilateral peripheral neuropathy, causalgia, sciatic neuritis, peripheral neuritis, polyneuritis, segmental neuritis, Gombault's neuritis, neuronitis, cervicobrachial neuralgia, cranial neuralgia, egniculate neuralgia, glossopharyngial neuralgia, migranous neuralgia, idiopathic neuralgia, intercostals neuralgia, mammary neuralgia, mandibular joint neuralgia, Morton's neuralgia, nasociliary neuralgia, occipital neuralgia, red neuralgia, Sluder's neuralgia splenopalatine neuralgia, supraorbital neuralgia, vidian neuralgia, sinus headache, tension headache, labor, childbirth, intestinal gas, menstruation, cancer, and trauma.

51. A compound according to claim 3 for use as a pharmaceutical.

52. A compound according to claim 3, for use as a pharmaceutical in the treatment or prevention of a disease or condition selected from: pain including acute, inflammatory and neuropathic pain, chronic pain, dental pain and headache including migraine, cluster headache and tension headache, Parkinson's disease, multiple sclerosis; diseases and disorders which are mediated by or result in neuroinflammation, traumatic brain injury, encephalitis; centrally-mediated neuropsychiatric diseases and disorders, depression mania, bipolar disease, anxiety, schizophrenia, eating disorders, sleep disorders and cognition disorders; epilepsy and seizure disorders; prostate, bladder and bowel dysfunction, urinary incontinence, urinary hesitancy, rectal hypersensitivity, fecal incontinence, benign prostatic hypertrophy and inflammatory bowel disease; respiratory and airway disease and disorders, allergic rhinitis, asthma and reactive airway disease and chronic obstructive pulmonary disease; diseases and disorders which are mediated by or result in inflammation, arthritis, rheumatoid arthritis and osteoarthritis, myocardial infarction, various autoimmune diseases and disorders, uveitis and atherosclerosis; itch/pruritus, psoriasis; obesity; lipid disorders; cancer; blood pressure; spinal cord injury; and renal disorders.

53. (canceled)
